(19) United States
(12) Patent Application Publication
(10) Pub. No.: US 2026/0242366 A1
(43) Pub. Date: Aug. 20, 2026

(54) STING AGONISTS CONTAINING HYDRAZIDE, HYDRAZINE, AND HYDROXAMIC ACID FUNCTIONAL GROUPS

(71) Applicant: BioNTech SE, Mainz (DE)

(72) Inventors: Zhengxin Dong, Hopkinton, MA (US); Farrukh Vohidov, Cambridge, MA (US); James Macdonald, Cambridge, MA (US); Griffin Gildersleeve, Cambridge, MA (US); Simon Weising, Mainz (DE)

(21) Appl. No.: 19/160,002

(22) PCT Filed: Feb. 27, 2024
(86) PCT No.: PCT/US2024/017511

§ 371 Date: Aug. 26, 2025

Related U.S. Application Data
(60) Provisional application No. 63/448,566, filed on Feb. 27, 2023.
(60) Provisional application No. 63/618,689, filed on Jan. 8, 2024.

(51) Int. Cl. C07D 413/14; A61K 31/4184; A61K 31/428; A61K 31/429; A61K 31/437; A61K 31/438; A61K 31/496; A61K 31/5377; C07D 403/14; C07D 417/12; C07D 417/14; C07D 471/04; C07D 513/04

(52) U.S. Cl. CPC C07D 413/14; A61K 31/4184; A61K 31/428; A61K 31/429; A61K 31/437; A61K 31/438; A61K 31/496; A61K 31/5377; C07D 403/14; C07D 417/12; C07D 417/14; C07D 471/04; C07D 513/04

(57) ABSTRACT

Compounds disclosed herein have been prepared for use in the treatment of diseases, disorders or conditions treatable by activation of the stimulator of interferon genes (STING) adaptor protein, such as in the treatment or prevention of cancers.

### BACKGROUND

The present invention relates to novel compounds which are STING (Stimulator of Interferon Genes) agonists and are useful for the treatment of disorders that are affected by the modulation of the STING protein. The invention also relates to pharmaceutical compositions comprising one or more of such compounds, processes to prepare such compounds and compositions, and use of such compounds or pharmaceutical compositions for the treatment of various diseases, syndromes and disorders affected by the modulation of the STING protein, such as in the treatment of a number of different cancers.

Stimulator of interferon genes (STING, also known as transmembrane protein 173/TMEM173/MPYS/MITA/ERIS) is a signaling molecule that in humans is encoded by TMEM173 gene. STING is a protein with 379 amino acids, consisting of several transmembrane regions. STING protein is expressed in several endothelial and epithelial cell types, as well as in haematopoietic lineage, which can include or exclude: T cells, dendritic cells (DCs) including plasmacytoid dendritic cells (pDCs) and macrophages. STING is associated with endoplasmic reticulum (ER) in the cell and has a major role in controlling the transcription of numerous host defense genes, including type I interferons (IFNs) and pro-inflammatory cytokines.

Recognition of aberrant DNA species or cyclic dinucleotides (CDNs) in the cytosol of the cell leads to the activation of STING. Cytosolic DNA species can activate STING signaling following binding to cyclic GMP-AMP synthase (cGAS). Binding of cytosolic DNA to cGAS catalyzes the production of a type of CDN known as cGAMP (cyclic GMP-AMP), which contains one 2′,5′-phosphodiester linkage and a canonical 3′,5′ linkage (c[G(2′,5′)pA(3′,5′)p]). The binding of cGAMP and other bacterial CDNs induce changes in the conformation of STING protein and facilitates the binding of TANK-binding kinase 1 (TBK1). STING-TBK1 complex, further transposes to perinuclear regions of the cell to transport TBK1 to endolysosomal compartments where it phosphorylates the transcription factors like, interferon regulatory factor 3 (IRF3). Similarly, STAT6 and nuclear factor-κB (NF-κB) also get activated downstream to STING activation. These transcription factors then translocate into the nucleus to initiate innate immune gene transcription and production of type I IFN and other cytokines. STING is then rapidly degraded, an event that may avoid problems associated with sustained cytokine production. (Nature Reviews Immunol, 2015, 15:760-770; Cell Reports, 2015, 11:1018-1030).

Recent evidence supports findings that once STING is activated by CDN within tumor microenvironment, preferably in tumor-resident dendritic cells, it promotes type I IFN and TNF a release which results in immunity-mediated anti-tumor response. STING-dependent activation of antigen-presenting cells (APC) efficiently drives highly specific T-cell priming against neoantigens (L. Corrales and T F. Gajewski, Clin Cancer Res, 2015, 21 (21), pp. 4774-9). STING activation not only provides generation of tumor-specific killer T cells which directly eradicate tumors, but also results in vaccine-like long-lasting immunity protecting from cancer recurrence.

Studies in mice have shown that type I IFN signaling plays an important role in tumor-initiated T cell priming and tumor control (J. Exp. Med. 2011, 208, 1989-2003). Mice lacking the IFN-α/B receptor in DCs failed to reject immunogenic tumors, and CD8α+ DCs from these mice are defective in antigen cross-presentation to CD8+ T cells. Additionally, transcriptional profiling analyses of melanoma patients has publicized that tumors containing infiltrating activated T cells are characterized by a type I IFN transcriptional signature (Cancer Res. 2009, 69:3077-3085).

Thus, synthetic STING agonist are of special interest as potential anticancer agents. Synthetic STING agonists have been generally disclosed in, for example, PCT Publication Nos. WO2017/175147; WO2017/175156; WO2019/069269; WO2019/069270 and WO2019/069275. The activation or inhibition of type I interferon production is an important strategy for the treatment or prevention of human diseases including viral infections and autoimmune disease. It has been found that compounds activating or inhibiting type I interferon production may be useful not only in infectious disease innate immunity, but also in cancer (L. Zitvogel et al., Nature Reviews Immunology, 2015, vol. 15 (7), pp. 405-414), allergic diseases (J. Moisan et al., Am. J. Physiol. Lung Cell Mol. Physiol., 2006, vol. 290, L987-995), neurodegenerative diseases such as amyotrophic lateral sclerosis and multiple sclerosis (H. Lemos et al., J. Immunol, 2014, vol. 192 (12), pp. 5571-8; E. Cirulli et al., Science, 2015, vol. 347 (6229), pp. 1436-41; A. Freischmidt et al., Nat. Neurosci., vol. 18 (5), 631-6), other inflammatory conditions such as irritable bowel disease (S. Rakoff-Nahoum, Cell, 2004, 23, 118 (2), pp. 229-41), and as vaccine adjuvants (Persing et al., Trends Microbiol. 2002, 10 (10 Suppl), S32-7; Dubensky et al, Therapeutic Advances in Vaccines, published online Sep. 5, 2013).

STING is essential for antimicrobial host defense, including protection against a range of DNA and RNA viruses and bacteria (reviewed in Barber et al., Nat. Rev. Immunol., 2015, vol. 15 (2), pp. 87-103, Ma and Damania, Cell Host & Microbe, 2016, vol. 19 (2), pp. 150-158). Herpesviridae, Flaviviridae, Coronaviridae, Papillomaviridae, Adenoviridae, Hepadnaviridae, ortho- and paramyxoviridae and rhabdoviridae have evolved mechanisms to inhibit STING mediated Type I interferon production and evade host immune control (Holm et al., Nat Comm., 2016, vol. 7, p. 10680; Ma et al., PNAS2015, vol. 112 (31) E4306-E4315; Wu et al., Cell Host Microbe, 2015, vol. 18 (3), pp. 333-44; Liu et al., J Virol, 2016, vol. 90 (20), pp. 9406-19; Chen et al., Protein Cell 2014, vol. 5 (5), pp. 369-81; Lau et al., Science, 2013, vol. 350 (6260), pp. 568-71; Ding et al., J Hepatol, 2013, vol. 59 (1), pp. 52-8; Nitta et al., Hepatology, 2013, vol. 57 (1), pp. 46-58; Sun et al., PloS One, 2012, vol. 7 (2), e30802; Aguirre et al., PloS Pathog, 2012, vol. 8 (10), e1002934; Ishikawa et al., Nature, 2009, vol. 461 (7265), pp. 788-92). Thus, small molecule activation of STING is considered to be beneficial for treatment of these infectious diseases.

In contrast, increased and prolonged type I IFN production is associated with a variety of chronic infections, including Mycobacteria (Collins et al., Cell Host Microbe, 2015, vol. 17 (6), pp. 820-8); Wassermann et al., Cell Host Microbe, 2015, vol. 17 (6), pp. 799-810; Watson et al., Cell Host Microbe, 2015, vol. 17 (6), pp. 811-9), Franciscella (Storek et al., J Immunol., 2015, vol. 194 (7), pp. 3236-45; Jin et al., J Immunol., 2011, vol. 187 (5), pp. 2595-601), Chlamydia (Prantner et al., J Immunol, 2010, vol. 184 (5), pp. 2551-60), Plasmodium (Sharma et al., Immunity, 2011, vol. 35 (2), pp. 194-207), and HIV (Herzner et al., Nat Immunol, 2015, vol. 16 (10), pp. 1025-33; Gao et al., Science, 2013, vol. 341 (6148), pp. 903-6). Similarly, excess type I interferon production is found among patients with complex forms of autoimmune disease. Genetic evidence in humans and support from studies in animal models support the hypothesis that inhibition of STING results in reduced type I interferon that drives autoimmune disease (Y. J. Crow et al., Nat. Genet., 2006, vol. 38 (8), pp. 38917-920, D. B. Stetson et al., Cell, 2008, pp. 134587-598). Therefore, inhibitors of STING provide a treatment to patients with chronic type I interferon and proinflammatory cytokine production associated with infections or complex autoimmune diseases. Allergic diseases are associated with a Th2-biased immune-response to allergens. Th2 responses are associated with raised levels of IgE, which, via its effects on mast cells, promotes a hypersensitivity to allergens, resulting in the symptoms seen, for example, in allergic rhinitis and asthma. In healthy individuals the immune-response to allergens is more balanced with a mixed Th2/Th1 and regulatory T cell response. Induction of Type 1 interferons have been shown to result in reduction of Th2-type cytokines in the local environment and promote Th1/Treg responses. In this context, induction of type 1 interferons by, for example, activation of STING, may offer benefit in treatment of allergic diseases such as asthma and allergic rhinitis (J. P. Huber et al., J Immunol, 2010, vol. 185, pp. 813-817).

In view of the above, compounds modulating STING are useful for treating one or more diseases selected from the group consisting of inflammatory, allergic, and autoimmune diseases, infectious diseases, cancer, pre-cancerous syndromes, and/or as immunogenic composition or vaccine adjuvants. Of particular relevance is the immunotherapy of cancer and viral infections, in particular prostate cancer, renal carcinoma, melanoma, pancreatic cancer, cervical cancer, ovarian cancer, colon cancer, head and neck cancer, lung cancer, fibrosarcoma, breast cancer and hepatitis B.

Accordingly, there is a need for compounds modulating the activity of STING, and accordingly, provide a therapeutic impact in the treatment of diseases, in which the modulation of STING is beneficial.

### SUMMARY

In one aspect, the present disclosure provides a compound of Formula I:

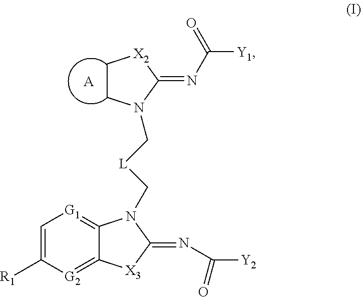

or a solvate, pharmaceutically acceptable salt, or tautomer thereof, wherein:
Ring A is selected from the group consisting of

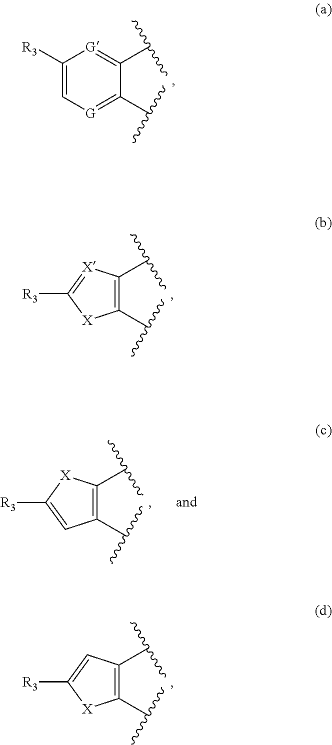

wherein
G and G₁ are independently N, CH, or C—X₁—R₂;
G′ and G₂ are independently N or CH;
X is N—R, O, or S;
X′ is N or CH;
X₁ is CH₂, O or S;
R is hydrogen or a C₁₋₄ alkyl, and
wherein when G and G₁ are each C—X₁—R₂, the R₂ groups are optionally linked to form L₁;
L and L₁ are each independently C₂₋₄ alkylene or C₂₋₄ alkenylene;
R₂ is selected from the group consisting of
hydrogen, a C₂₋₄ cyclic ether, a C₃₋₄ cyclic alkyl, C₁₋₄ alkyl,

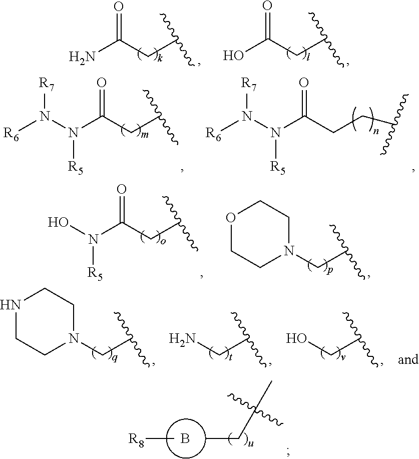

R₁ and R₃ are independently selected from the group consisting of

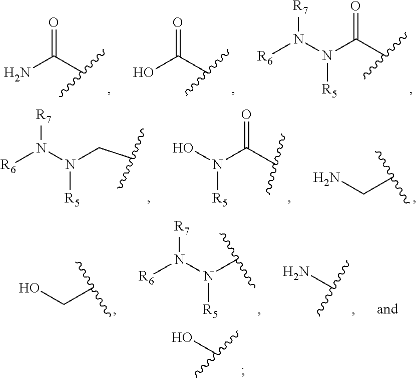

Ring B is a 6-membered aromatic ring or a 5- or 6-membered heteroaromatic ring comprising 1 to 2 heteroatoms selected from N, O, and S;
R₈ is —OH or —NR₉R₁₀;
R₉ and R₁₀ are independently selected from hydrogen and C₁-C₆ alkyl;
X₂ and X₃ are independently NH or S;
Y₁ and Y₂ are independently

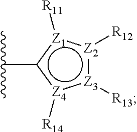

Z₁, Z₂, Z₃, and Z₄ are each independently C, N, O, or S;
R₅, R₆, and R₇ are independently selected from hydrogen, C₁-C₆ alkyl, and C₂-C₆ alkenyl,

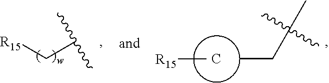

wherein R₅ and Rₑ are optionally connected to form a 5- or 6-membered heterocyclic ring;
R₁₅ is —OH or —NR₉R₁₀;
Ring C is a 6-membered aromatic ring or a 5- or 6-membered heteroaromatic ring comprising 1 to 2 heteroatoms selected from N, O, and S;
R₁₁, R₁₂, R₁₃, and R₁₄ are independently absent, hydrogen, or C₁₋₄ alkyl;
n, p, q, t, and v are independently an integer from 2 to 6; and
k, l, m, o, u, and w are independently an integer from 1 to 6, and
provided that at least one of G and G₁ is C—X₁—R₂, wherein R₂ is

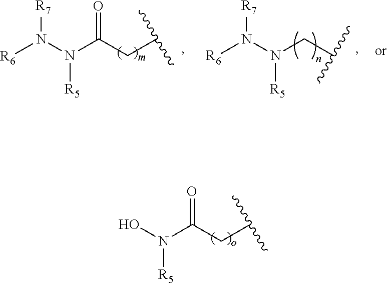

and/or
at least one of R₁ and R₃ is

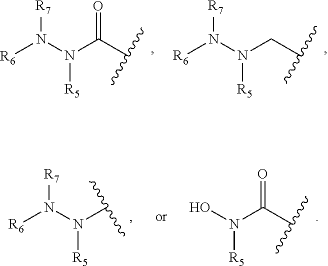

In another aspect, the present disclosure provides a compound of Formula IA:

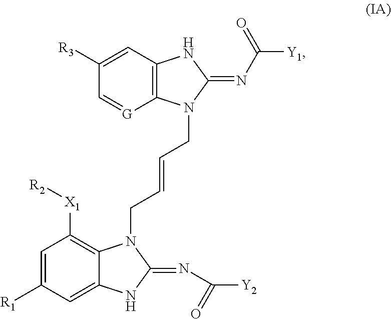

or a solvate, pharmaceutically acceptable salt, or tautomer thereof, wherein:
X₁ is CH₂, O or S;
G is CH, C—SCH₃, C—OCH₃, or N;
R₂ is selected from the group consisting of
hydrogen, a C₂₋₄ cyclic ether, a C₃₋₄ cyclic alkyl, C₁₋₄ alkyl,

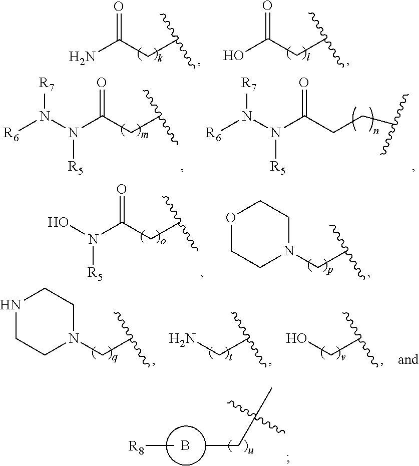

R₁ and R₃ are independently selected from the group consisting of

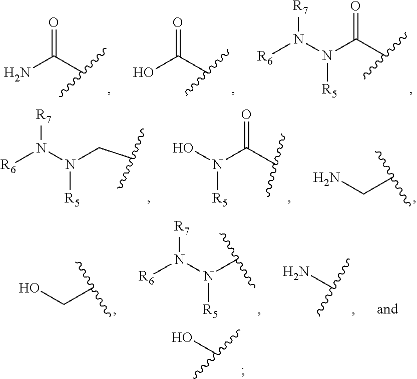

Ring B is a 6-membered aromatic ring or a 5- or 6-membered heteroaromatic ring comprising 1 to 2 heteroatoms selected from N, O, and S;
R₈ is —OH or —NH₂;
Y₁ and Y₂ are independently

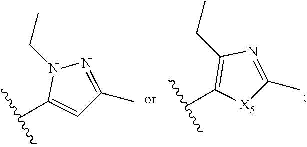

X₅ is S, O, or NR₇;
R₅, R₆, and R₇ are independently selected from hydrogen, C₁-C₆ alkyl, and C₂-C₆ alkenyl,

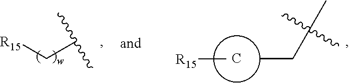

wherein R₅ and Rₑ are optionally connected to form a 5- or 6-membered heterocyclic ring;
R₁₅ is —OH or —NR₉R₁₀;
R₉ and R₁₀ are independently selected from hydrogen and C₁-C₆ alkyl;
Ring C is a 6-membered aromatic ring or a 5- or 6-membered heteroaromatic ring comprising 1 to 2 heteroatoms selected from N, O, and S;
n, p, q, t, and v are independently an integer from 2 to 6; and
k, l, m, o, u, and w are independently an integer from 1 to 6, and provided that at least R₂ is

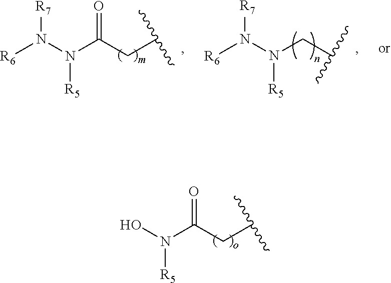

and/or
at least one of R₁ and R₃ is

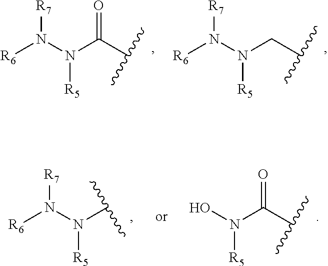

In another aspect, the present disclosure provides a compound of Formula IA′:

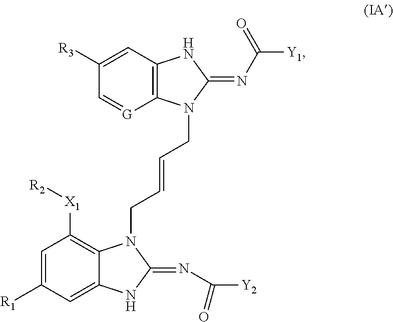

or a solvate, pharmaceutically acceptable salt, or tautomer thereof, wherein:
X₁ is CH₂, O or S;
G is CH, C—SCH₃, C—OCH₃, or N;
R₂ is selected from the group consisting of
hydrogen, a C₂₋₄ cyclic ether, a C₃₋₄ cyclic alkyl, C₁₋₄ alkyl,

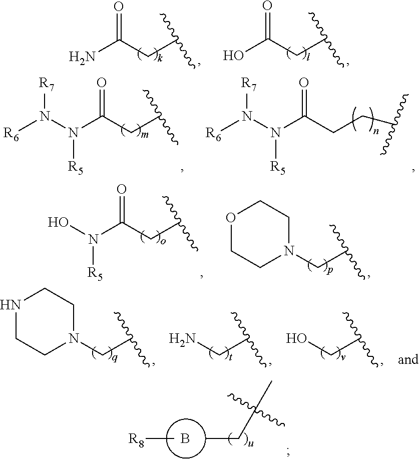

R₁ and R₃ are independently selected from the group consisting of

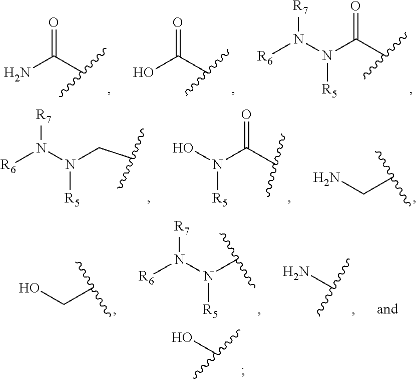

Ring B is a 6-membered aromatic ring or a 5- or 6-membered heteroaromatic ring comprising 1 to 2 heteroatoms selected from N, O, and S;
R₈ is —OH or —NH₂;
Y₁ and Y₂ are independently

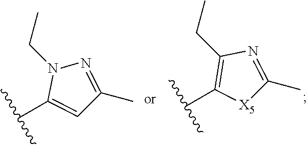

X₅ is S, O, or NR₇;
R₅, R₆, and R₇ are independently selected from hydrogen, —OH, C₁-C₆ alkyl,

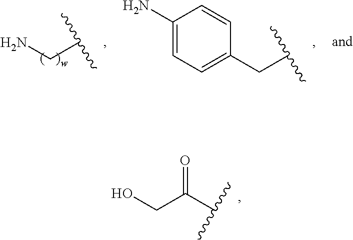

wherein R₅ and R₆ are optionally connected to form a 5- or 6-membered heterocyclic ring;
n, p, q, t, and v are independently an integer from 2 to 6; and
   k, l, m, o, u, and w are independently an integer from 1 to 6, and
   provided that R₂ is

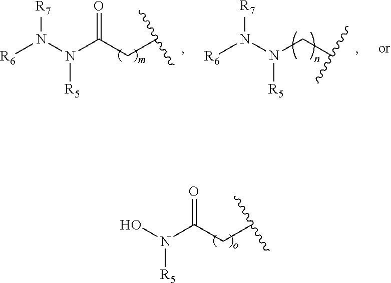

and/or
at least one of R₁ and R₃ is

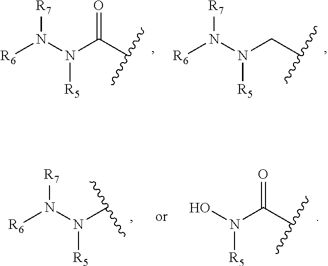

In yet another aspect, the present disclosure provides a compound of Formula IB:

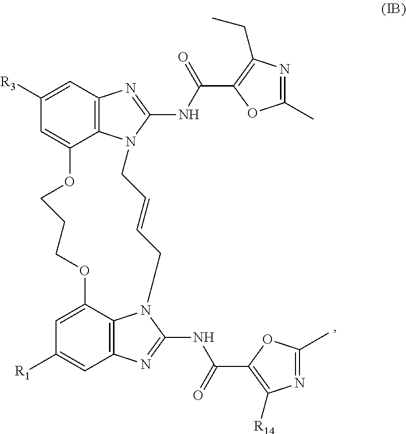

or a solvate, pharmaceutically acceptable salt, or tautomer thereof, wherein:
R₁ and R₃ are independently selected from the group consisting of

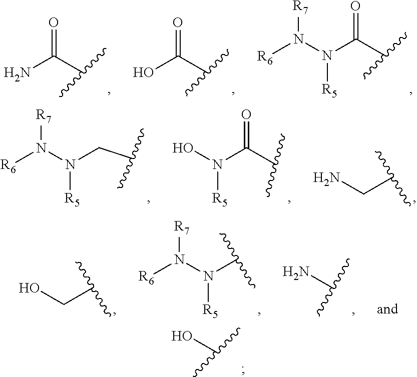

wherein optionally R₅ and R₆ are connected to form a 5- or 6-membered ring;
R₁₄ is hydrogen, or C₁₋₄ alkyl;
R₅, R₆, and R₇ are independently selected from hydrogen, —OH, C₁-C₆ alkyl,

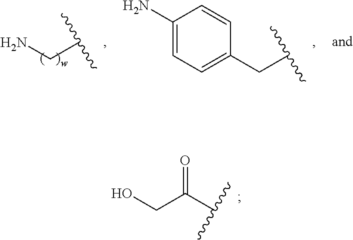

and
   w is an integer from 1 to 6, provided that at least one of R₁ and R₃ is

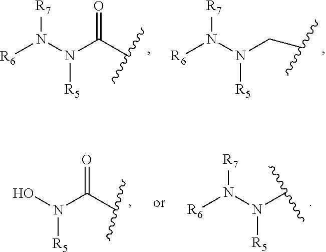

The compounds of the application have been shown to be capable of activating STING protein function. Therefore the compounds of the application are useful for treating diseases, disorders or conditions treatable by activation of STING. Accordingly, the present application also includes a method of treating a disease, disorder or condition treatable by activation of STING, comprising administering a therapeutically effective amount of one or more compounds of the application to a subject in need thereof.

In a further embodiment, the compounds of the application are used as medicaments. Accordingly, the application also includes a compound of the application for use as a medicament.

The present application also includes a use of one or more compounds of the application for treatment of a disease, disorder or condition treatable by activation of STING as well as a use of one or more compounds of the application for the preparation of a medicament for treatment of a disease, disorder or condition treatable by activation of STING. The application further includes one or more compounds of the application for use in treating a disease, disorder or condition treatable by activation of STING.

The compounds of the application are useful for treating diseases, disorders or conditions mediated by STING protein activation. Accordingly, the present application also includes a method of treating a disease, disorder or condition mediated by STING protein activation, comprising administering a therapeutically effective amount of one or more compounds of the application to a subject in need thereof.

The present application also includes a use of one or more compounds of the application for treatment of a disease, disorder or condition mediated by STING protein activation as well as a use of one or more compounds of the application for the preparation of a medicament for treatment of a disease, disorder or condition mediated by STING protein activation. The application further includes one or more compounds of the application for use in treating a disease, disorder or condition mediated by STING protein activation.

In an embodiment, the disease, disorder or condition mediated by STING protein activation, or treatable by activation of STING, is a neoplastic disorder. In an embodiment, the treatment comprises administration or use of an amount of one or compounds of the application that is effective to ameliorate at least one symptom of the neoplastic disorder, for example, reduced cell proliferation or reduced tumor mass in a subject in need of such treatment.

In an embodiment, the disease, disorder or condition mediated by STING protein activation, or treatable by activation of STING, is cancer.

In an embodiment, the disease, disorder or condition mediated by STING protein activation, or treatable by activation of STING, is a disease, disorder or condition associated with an uncontrolled and/or abnormal cellular activity affected directly or indirectly by STING. In another embodiment, the uncontrolled and/or abnormal cellular activity that is affected directly or indirectly by STING is proliferative activity in a cell.

The application also includes a method of inhibiting proliferative activity in a cell, comprising administering an effective amount of one or more compounds of the application to the cell.

In a further embodiment the disease, disorder or condition mediated by STING protein activation, or treatable by activation of STING, is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as other tyrosine-kinase inhibitors, immunotherapy, hormonal therapy and anti-angiogenic therapies.

The application additionally provides a process for the preparation of compounds of Formula I. General and specific processes are discussed in more detail and set forth in the Examples below.

Other features and advantages of the present application will become apparent from the following detailed description. It should be understood, however, that the detailed description and the specific examples while indicating embodiments of the application are given by way of illustration only, since various changes and modifications within the spirit and scope of the application will become apparent to those skilled in the art from this detailed description.

### BRIEF DESCRIPTION OF DRAWINGS

FIGS. 1A and 1B illustrate THP-1 Plots for example compounds I-A and I-D of the disclosure.

FIGS. 2A-2D illustrate STING haplotype curves for example compounds I-A and I-D of the disclosure.

FIGS. 3A and 3B illustrate PBMC representative curves for example compounds I-A and I-D of the disclosure.

FIGS. 4A and 4B illustrate additional PBMC representative curves for example compounds I-A and I-D of the disclosure.

FIGS. 5A and 5B illustrate additional PBMC representative curves for example compounds I-A and I-D of the disclosure.

FIGS. 6A-6E illustrate tumor growth data, as measured using BioVolume, after administration of various doses of example compound I-D or a vehicle control.

FIG. 7A illustrates IFNα levels in tumor after administration of various doses of example compound I-D or a vehicle control.

FIG. 7B illustrates IFNα levels in tumor after administration of various doses of comparative compound diABZI or a vehicle control.

FIG. 8A illustrates IFNγ levels in tumor after administration of various doses of example compound I-D or a vehicle control.

FIG. 8B illustrates IFNγ levels in tumor after administration of various doses of comparative compound diABZI or a vehicle control.

FIGS. 9A-9E illustrate tumor growth data, as measured using calipers, after administration of various doses of comparative compound diABZI or a vehicle control.

FIG. 10 illustrates STING haplotype curves for example compounds XV and XX of the disclosure.

FIG. 11 illustrates THP-1 Plots for example compounds XV and XX of the disclosure.

FIG. 12 illustrates PBMC representative curves for example compounds XV and XX of the disclosure.

FIG. 13 illustrates the activity of example compounds XXVIII-XXXV of the disclosure.

FIG. 14 illustrates THP-1 plots for example compounds XXVIII-XXXV of the disclosure.

### DETAILED DESCRIPTION

### Definitions

Unless defined otherwise, all technical and scientific terms have the same meaning as is commonly understood by one of ordinary skill in the art to which the disclosed embodiments belong.

As used herein, the terms “a” or “an” mean “at least one” or “one or more” unless the context clearly indicates otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by +10% and remain within the scope of the disclosed embodiments.

As used herein, the term “and/or” includes any and all combinations of one or more of the associated listed items.

As used herein, the term “alkenyl” means a straight or branched alkyl group having 2 to 20 carbon atoms and having one or more double carbon-carbon bonds. In some embodiments, the alkenyl group has from 2 to 10 carbon atoms, from 2 to 8 carbon atoms, from 2 to 6 carbon atoms, from 2 to 4 carbon atoms, from 3 to 10 carbon atoms, from 3 to 8 carbon atoms, from 3 to 6 carbon atoms, or 3 or 4 carbon atoms. Examples of alkenyl groups include, but are not limited to, ethenyl, 1-propenyl, 2-methyl-1-propenyl, 2-propenyl, 1-butenyl, 2-butenyl, and the like.

As used herein, the term “alkoxy” means a straight or branched-O-alkyl group having 1 to 20 carbon atoms. In some embodiments, the alkoxy group has from 1 to 10 carbon atoms, from 1 to 8 carbon atoms, from 1 to 6 carbon atoms, from 1 to 4 carbon atoms, from 2 to 10 carbon atoms, from 2 to 8 carbon atoms, from 2 to 6 carbon atoms, or from 2 to 4 carbon atoms. Examples of alkoxy groups include, but are not limited to, methoxy, ethoxy, n-propoxy, isopropoxy, t-butoxy, and the like.

As used herein, the term “alkyl” means a saturated hydrocarbon group which is straight-chained or branched. In some embodiments, the alkyl group has from 1 to 20 carbon atoms, from 2 to 20 carbon atoms, from 1 to 10 carbon atoms, from 2 to 10 carbon atoms, from 1 to 8 carbon atoms, from 2 to 8 carbon atoms, from 1 to 6 carbon atoms, from 2 to 6 carbon atoms, from 1 to 4 carbon atoms, from 2 to 4 carbon atoms, from 1 to 3 carbon atoms, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, octyl, nonyl, 4,4-dimethylpentyl, 2,2,4-trimethylpentyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkylamino” means an amino group substituted by an alkyl group. In some embodiments, the alkyl group is a lower alkyl group having from 1 to 6 carbon atoms. Alkylamino groups include, but are not limited to, —NHCH₂CH₃, —NH(CH₂)₂CH₃, —NH(CH₂)₃CH₃, —NH(CH₂)₄CH₃, and —NH(CH₂)₅CH₃, and the like.

As used herein, the term “alkylene” or “alkylenyl” means a divalent alkyl linking group. Example of alkylenes (or alkylenyls) include, but are not limited to, methylene or methylenyl (—CH₂—), ethylene or ethylenyl (—CH₂—CH₂—), and propylene or propylenyl (—CH₂—CH₂—CH₂—).

As used herein, the term “alkylthio” means an —S-alkyl group having from 1 to 6 carbon atoms. Alkylthio groups include, but are not limited to, —SCH₂CH₃, —S(CH₂)₂CH₃, —S(CH₂)₃CH₃, —S(CH₂)₄CH₃, and —S(CH₂)₅CH₃, and the like.

As used herein, the term “alkynyl” means a straight or branched alkyl group having 2 to 20 carbon atoms and one or more triple carbon-carbon bonds. In some embodiments, the alkynyl group has from 2 to 10 carbon atoms, from 2 to 8 carbon atoms, from 2 to 6 carbon atoms, or from 2 to 4 carbon atoms. Examples of alkynyl groups include, but are not limited to, acetylene, 1-propylene, 2-propylene, and the like.

As used herein, the term “amino” means —NH₂.

As used herein, the term “aminoalkyl” means an alkyl group substituted by an amino group. Examples of aminoalkyl groups include, but are not limited to, —CH₂NH₂,—CH₂CH₂NH₂, —(CH₂)₃NH₂, —(CH₂)₄NH₂, and the like.

As used herein, the term “aminosulfonyl” means —S(═O) 2NH₂.

As used herein, the term “aryl” means a monocyclic, bicyclic, or polycyclic (e.g., having 2, 3 or 4 fused rings) aromatic hydrocarbon. In some embodiments, the aryl group has from 6 to 20 carbon atoms or from 6 to 10 carbon atoms. Examples of aryl groups include, but are not limited to, phenyl, naphthyl, anthracenyl, phenanthrenyl, indanyl, indenyl, and tetrahydronaphthyl, and the like.

As used herein, the term “arylene” means an aryl linking group, i.e., an aryl group that links one group to another group in a molecule.

As used herein, the term “carbamoyl” means —C(═O)—NH₂.

As used herein, the term “carbocycle” means a 5- or 6-membered, saturated or unsaturated, cyclic ring optionally containing O, S, or N atoms as part of the ring. Examples of carbocycles include, but are not limited to, cyclopentyl, cyclohexyl, cyclopenta-1,3-diene, phenyl, and any of the heterocycles recited herein.

As used herein, the term “carrier” means a diluent, adjuvant, or excipient with which a compound is administered in a composition.

As used herein, the term, “compound” means all stereoisomers, tautomers, isotopes, and polymorphs of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive and open-ended and include the options following the terms, and do not exclude additional, unrecited elements or method steps.

As used herein, the term “contacting” means bringing together two compounds, molecules, or entities in an in vitro system or an in vivo system.

As used herein, the term “cycloalkyl” means non-aromatic cyclic hydrocarbons including cyclized alkyl, alkenyl, and alkynyl groups that have up to 20 ring-forming carbon atoms. Cycloalkyl groups have from 3 to 15 ring-forming carbon atoms, from 3 to 10 ring-forming carbon atoms, from 3 to 8 ring-forming carbon atoms, from 3 to 6 ring-forming carbon atoms, from 4 to 6 ring-forming carbon atoms, from 3 to 5 ring-forming carbon atoms, or 5 or 6 ring-forming carbon atoms. Ring-forming carbon atoms of a cycloalkyl group can be optionally substituted by oxo or sulfido. Cycloalkyl groups include, but are not limited to, monocyclic or polycyclic ring systems such as fused ring systems, bridged ring systems, and spiro ring systems. In some embodiments, polycyclic ring systems include 2, 3, or 4 fused rings. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcarnyl, adamantyl, and the like. Cycloalkyl groups can also have one or more aromatic rings fused (having a bond in common with) to the cycloalkyl ring such as, for example, benzo or thienyl derivatives of pentane, pentene, hexane, and the like (e.g., 2,3-dihydro-1H-indene-1-yl, or 1H-inden-2(3H)-one-1-yl).

As used herein, the term “cycloalkylalkyl” means a C₁₋₆alkyl substituted by a cycloalkyl.

As used herein, the term “dialkylamino” means an amino group substituted by two alkyl groups. In some embodiments, one or both of the alkyl groups has from 1 to 6 carbon atoms.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, pyridinyl (including 2-aminopyridine), triazinyl, furyl, quinolyl, isoquinolyl, thienyl, imidazolyl, thiazolyl, indolyl (such as indol-3-yl), pyrryl, oxazolyl, benzofuryl, benzothienyl, pyrazolyl, benzthiazolyl, isoxazolyl, triazolyl (including 1,2,4-triazole, 1,2,3-triazole, and 5-amino-1,2,4-triazole), tetrazolyl, indazolyl, isothiazolyl, 1,2,4-thiadiazolyl, benzothienyl, purinyl, carbazolyl, isoxazolyl, benzimidazolyl, indolinyl, pyranyl, pyrazolyl, triazolyl, oxadiazolyl (including 1,2,3-oxadiazole, 1,2,4-oxadiazole, 1,2,5-oxadiazole, 3-amino-1,2,4-oxadiazole, 1,3,4-oxadiazole), thianthrenyl, indolizinyl, isoindolyl, isobenzofuranyl, pyrrolyl, benzoxazolyl, xanthenyl, 2H-pyrrolyl, 3H-indolyl, 4H-quinolizinyl, phthalazinyl, acridinyl, naphthyridinyl, quinazolinyl, phenanthridinyl, perimidinyl, phenanthrolinyl, phenazinyl, isothiazolyl, phenothiazinyl, isoxazolyl, furazanyl, phenoxazinyl groups, and the like.

As used herein, the term “heteroarylalkyl” means a C₁₋₆alkyl group substituted by a heteroaryl group.

As used herein, the term “heteroarylamino” means an amino group substituted by a heteroaryl group.

As used herein, the term “heteroarylene” means a heteroaryl linking group, i.e., a heteroaryl group that links one group to another group in a molecule.

As used herein, the term “heterocycle” or “heterocyclic ring” means a 5- to 7-membered monocyclic or 7- to 10-membered bicyclic ring system, any ring of which may be saturated or unsaturated, and which ring consists of carbon atoms and from one to three heteroatoms chosen from N, O and S, and wherein the N and S heteroatoms may optionally be oxidized, and the N heteroatom may optionally be quaternized, and including any bicyclic group in which any of the above-defined heterocyclic rings is fused to a benzene ring. Heterocycles include rings containing one oxygen or sulfur, one to three nitrogen atoms, or one oxygen or sulfur combined with one or two nitrogen atoms. The heterocyclic ring may be attached at any heteroatom or carbon atom which results in the creation of a stable structure. Examples of heterocyclic groups include, but are not limited to, piperidinyl, piperazinyl, 2-oxopiperazinyl, 2-oxopiperidinyl, 2-oxopyrrolodinyl, 2-oxoazepinyl, azepinyl, pyrrolyl, 4-piperidonyl, pyrrolidinyl, pyrazolyl, pyrazolidinyl, imidazolyl, imidazolinyl, pyridyl, imidazolidinyl, pyrazinyl, pyrimidinyl, pyridazinyl, oxazolyl, oxazolidinyl, isoxazolyl, isoxazolidinyl, morpholinyl, thiazolyl, thiazolidinyl, isothiazolyl, quinuclidinyl, isothiazolidinyl, indolyl, quinolinyl, isoquinolinyl, benzimidazolyl, thiadiazoyl, benzopyranyl, benzothiazolyl, benzoxazolyl, furyl, tetrahydrofuryl, tetrahydropyranyl, thienyl, benzothienyl, thiamorpholinyl, thiamorpholinyl sulfoxide, thiamorpholinyl sulfone, oxadiazolyl, and the like.

As used herein, the term “heterocycloalkyl” means non-aromatic heterocycles having up to 20 ring-forming atoms including cyclized alkyl, alkenyl, and alkynyl groups, where one or more of the ring-forming carbon atoms is replaced by a heteroatom, such as an O, N, or S atom. Hetercycloalkyl groups can be monocyclic or polycyclic (e.g., fused, bridged, or spiro systems). In some embodiments, the heterocycloalkyl group has from 1 to 20 carbon atoms or from 3 to 20 carbon atoms. In some embodiments, the heterocycloalkyl group contains 3 to 14 ring-forming atoms, 3 to 7 ring-forming atoms, or 5 or 6 ring-forming atoms. In some embodiments, the heterocycloalkyl group has 1 to 4 heteroatoms, 1 to 3 heteroatoms, or 1 or 2 heteroatoms. In some embodiments, the heterocycloalkyl group has 0 to 3 double bonds. In some embodiments, the heterocycloalkyl group has 0 to 2 triple bonds. Examples of heterocycloalkyl groups include, but are not limited to, morpholino, piperazinyl, thiomorpholino, tetrahydrofuranyl, tetrahydrothienyl, 2,3-dihydrobenzofuryl, piperidinyl, 1,3-benzodioxole, benzo-1,4-dioxane, pyrrolidinyl, isoxazolidinyl, oxazolidinyl, isothiazolidinyl, pyrazolidinyl, thiazolidinyl, imidazolidinyl, pyrrolidin-2-one-3-yl, and the like. In addition, ring-forming carbon atoms and heteroatoms of a heterocycloalkyl group can be optionally substituted by oxo or sulfido. For example, a ring-forming S atom can be substituted by 1 or 2 oxo (form a S(O) or S(O)₂). For another example, a ring-forming C atom can be substituted by oxo (form carbonyl). Heterocycloalkyl groups can also have one or more aromatic rings fused (having a bond in common with) to the nonaromatic heterocyclic ring including, but not limited to, pyridinyl, thiophenyl, phthalimidyl, naphthalimidyl, and benzo derivatives of heterocycles such as, for example, indolene, isoindolene, 5,6-dihydrothieno[2,3-c]pyridin-7 (4H)-one-5-yl, isoindolin-1-one-3-yl, 4,5,6,7-tetrahydrothieno[2,3-c]pyridine-5-yl, and 3,4-dihydroisoquinolin-1(2H)-one-3yl groups. Ring-forming carbon atoms and heteroatoms of the heterocycloalkyl group can be optionally substituted by oxo or sulfido.

As used herein, the term “heterocycloalkylalkyl” means an alkyl group substituted by heterocycloalkyl. In some embodiments, the alkyl group is a C₁₋₆alkyl group.

As used herein, the term “hydroxy” or “hydroxyl” means an —OH group.

As used herein, the term “hydroxyalkyl” or “hydroxylalkyl” means an alkyl group substituted by a hydroxyl group. Examples of hydroxylalkyl groups include, but are not limited to, —CH₂OH and —CH₂CH₂OH.

As used herein, the terms “individual,” “subject,” and “patient,” used interchangeably, mean any animal described herein.

As used herein, the phrase “in need thereof” means that the “individual,” “subject,” or “patient” has been identified as having a need for the particular method, prevention, or treatment. In some embodiments, the identification can be by any means of diagnosis. In any of the methods, preventions, and treatments described herein, the “individual,” “subject,” or “patient” can be in need thereof. In some embodiments, the “individual,” “subject,” or “patient” is in an environment or will be traveling to an environment, or has traveled to an environment in which a particular disease, disorder, or condition is prevelant.

As used herein, the term “integer” means a numerical value that is a whole number. For example, an “integer from 1 to 5” means 1, 2, 3, 4, or 5.

All cyclic groups contain one or more than one ring (i.e. are polycyclic). When a cyclic group contains more than one ring, the rings may be fused, bridged or linked by a bond.

A first ring being “fused” with a second ring means the first ring and the second ring share two adjacent atoms there between.

As used herein, the term “isolated” means that the compounds, or pharmaceutically acceptable salts thereof, described herein are separated from other components of either: a) a natural source, such as a plant or cell, such as a bacterial culture, or b) a synthetic organic chemical reaction mixture, such as by conventional techniques.

As used herein, the term “n-membered”, where n is an integer, typically describes the number of ring-forming atoms in a moiety, where the number of ring-forming atoms is n. For example, pyridine is an example of a 6-membered heteroaryl ring and thiophene is an example of a 5-membered heteroaryl ring.

As used herein, the phrase “optionally substituted” means that a substitution is optional and, therefore, includes both unsubstituted and substituted atoms and moieties. A “substituted” atom or moiety indicates that any hydrogen atom on the designated compound or moiety can be replaced with a selection from the indicated substituent groups, provided that the normal valency of the designated compound or moiety is not exceeded, and that the substitution results in a stable compound. For example, if a methyl group is optionally substituted, then 1, 2, or 3 hydrogen atoms on the carbon atom within the methyl group can be replaced with 1, 2, or 3 of the recited substituent groups.

As used herein, the phrase “pharmaceutically acceptable” means that the compounds, materials, compositions, and/or dosage forms are within the scope of sound medical judgment and are suitable for use in contact with tissues of humans and other animals. In some embodiments, “pharmaceutically acceptable” means approved by a regulatory agency of the Federal government or a state government or listed in the U.S. Pharmacopeia or other generally recognized pharmacopeia for use in animals, and more particularly in humans. In some embodiments, the pharmaceutically acceptable compounds, materials, compositions, and/or dosage forms result in no persistent detrimental effect on the subject, or on the general health of the subject being treated. However, it will be recognized that transient effects, such as minor irritation or a “stinging” sensation, are common with administration of medicament and the existence of such transient effects is not inconsistent with the composition, formulation, or ingredient (e.g., excipient) in question.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. Salts also includes quaternary ammonium salts of the compounds described herein, where the compounds have one or more tertiary amine moiety.

As used herein, the term “phenyl” means —C₆H₅. A phenyl group can be unsubstituted or substituted with one, two, or three suitable substituents.

As used herein, the terms “prevention” or “preventing” mean a reduction of the risk of acquiring a particular disease, condition, or disorder.

As used herein, the phrase “solubilizing agent” means agents that result in formation of a micellar solution or a true solution of the drug.

As used herein, the term “solution/suspension” means a liquid composition wherein a first portion of the active agent is present in solution and a second portion of the active agent is present in particulate form, in suspension in a liquid matrix.

As used herein, the phrase “suitable substituent” or “substituent” means a group that does not nullify the synthetic or pharmaceutical utility of the compounds described herein or the intermediates useful for preparing them. Examples of suitable substituents include, but are not limited to: C₁-C₆alkyl, C₁-C₆alkenyl, C₁-C₆alkynyl, C₅-C₆aryl, C₁-C₆alkoxy, C₃-C₅heteroaryl, C₃-C₅cycloalkyl, C₅-C₆aryloxy, —CN, —OH, oxo, halo, haloalkyl, —NO₂, —CO₂H, —NH₂, —NH(C₁-C₈alkyl), —N(C₁-C₈alkyl)₂, —NH(C₆aryl), —N(C₅-C₆aryl)₂, —CHO, —CO(C₁-C₆alkyl), —CO((C₅-C₆)aryl), —CO₂((C₁-C₆)alkyl), and —CO₂((C₅-C₆)aryl). One of skill in art can readily choose a suitable substituent based on the stability and pharmacological and synthetic activity of the compounds described herein.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having alternate stereochemistry. It is intended that any optical isomers, as separated, pure or partially purified optical isomers or racemic or other mixtures thereof are included within the scope of the present application.

The compounds of the present application may also exist in different tautomeric forms and it is intended that any tautomeric forms which the compounds form are included within the scope of the present application.

The compounds of the present application may further exist in varying polymorphic forms and it is contemplated that any polymorphs which form are included within the scope of the present application.

As used herein, the phrase “therapeutically effective amount” means the amount of active compound or pharmaceutical agent that elicits the biological or medicinal response that is being sought in a tissue, system, animal, individual or human by a researcher, veterinarian, medical doctor, or other clinician. The therapeutic effect is dependent upon the disorder being treated or the biological effect desired. As such, the therapeutic effect can be a decrease in the severity of symptoms associated with the disorder and/or inhibition (partial or complete) of progression of the disorder, or improved treatment, healing, prevention or elimination of a disorder, or side-effects. The amount needed to elicit the therapeutic response can be based on, for example, the age, health, size, and sex of the subject. Optimal amounts can also be determined based on monitoring of the subject's response to treatment.

At various places herein, substituents of compounds may be disclosed in groups or in ranges. It is specifically intended that the disclosure include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

For compounds in which a variable appears more than once, each variable can be a different moiety chosen from the Markush group providing options for the variable. For example, where a structure is described having two R groups that are simultaneously present on the same compound, the two R groups can represent different moieties chosen from the Markush group defined for R. In another example, when an optionally multiple substituent “R” is designated in the form, for example,

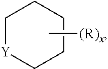

then it should be understood that substituent “R” can occur “x” number of times on the ring at any position(s), and “R” can be a different moiety at each occurrence. Further, in the above example, where the variable “Y” normally would include one or more hydrogens, such as when “Y” is CH₂, NH, etc., any H can be replaced with a substituent.

The term “treating” or “treatment” as used herein and as is well understood in the art, means an approach for obtaining beneficial or desired results, including clinical results. Beneficial or desired clinical results can include, but are not limited to alleviation or amelioration of one or more symptoms or conditions, diminishment of extent of disease, stabilized (i.e. not worsening) state of disease, preventing spread of disease, delay or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. “Treating” and “treatment” as used herein also include prophylactic treatment. For example, a subject with early cancer can be treated to prevent progression, or alternatively a subject in remission can be treated with a compound or composition of the application to prevent recurrence. Treatment methods comprise administering to a subject a therapeutically effective amount of one or more of the compounds of the application and optionally consist of a single administration, or alternatively comprise a series of administrations. For example, in some embodiments, the compounds of the application may be administered at least once a week. In some embodiments, the compounds may be administered to the subject from about one time per three weeks, or about one time per week to about once daily for a given treatment. In another embodiment, the compounds are administered 2, 3, 4, 5 or 6 times daily. The length of the treatment period depends on a variety of factors, such as the severity of the disease, disorder or condition, the age of the subject, the concentration and/or the activity of the compounds of the application, and/or a combination thereof. It will also be appreciated that the effective dosage of the compound used for the treatment may increase or decrease over the course of a particular treatment regime. Changes in dosage may result and become apparent by standard diagnostic assays known in the art. In some instances, chronic administration may be required. For example, the compounds are administered to the subject in an amount and for duration sufficient to treat the patient.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with a disease, disorder or condition mediated by STING protein activation or treatable by activation of STING, or manifesting a symptom associated with a disease, disorder or condition mediated by STING protein activation or treatable by activation of STING.

The “disease, disorder or condition mediated by STING” as used herein refers to a disease, disorder or condition treatable by activation of STING activity and particularly using an STING agonist, such as one or more compounds of the application herein described.

The term “mediated by STING” as used herein means that the disease, disorder or condition to be treated is affected by, modulated by and/or has some biological basis, either direct or indirect, that includes aberrant STING activity, in particular, decreased STING activity such as results from mutation or splice variation and the like. These diseases respond favourably when STING activity associated with the disease is activated by one or more of the compounds of the application.

The term “STING agonist” as used herein refers to compounds that are “activators of STING” (i.e. activate STING activity) and therefore the term “activator” and “agonist” may be used interchangeably in reference to the compounds of the application.

As used herein, the term “effective amount” or “therapeutically effective amount” means an amount of one or more compounds of the application that is effective, at dosages and for periods of time necessary to achieve the desired result. For example in the context of treating a disease, disorder or condition mediated by STING protein activation or treatable by activation of STING, an effective amount is an amount that, for example, increases STING protein activation, or increases STING activity, compared to the activity without administration of the one or more compounds. Effective amounts may vary according to factors such as the disease state, age, sex and/or weight of the subject. The amount of a given compound that will correspond to such an amount will vary depending upon various factors, such as the given drug or compound, the pharmaceutical formulation, the route of administration, the type of condition, disease or disorder, the identity of the subject being treated, and the like, but can nevertheless be routinely determined by one skilled in the art. In an embodiment, the effective amount is one that following treatment therewith manifests as an improvement in or reduction of any disease symptom. When the disease is cancer, amounts that are effective can cause a reduction in, for example, the number, growth rate, size and/or distribution of tumors.

The term “administered” as used herein means administration of a therapeutically effective amount of one or more compounds, or compositions, of the application to a cell either in cell culture or in a subject.

The term “neoplastic disorder” as used herein refers to a disease, disorder or condition characterized by cells that have the capacity for autonomous growth or replication, e.g., an abnormal state or condition characterized by proliferative cell growth. The term “neoplasm” as used herein refers to a mass of tissue resulting from the abnormal growth and/or division of cells in a subject having a neoplastic disorder. Neoplasms can be benign (such as uterine fibroids and melanocytic nevi), potentially malignant (such as carcinoma in situ) or malignant (i.e. cancer). Exemplary neoplastic disorders include but are not limited to carcinoma, sarcoma, metastatic disorders (e.g., tumors arising from the prostate), hematopoietic neoplastic disorders, (e.g., leukemias, lymphomas, myeloma and other malignant plasma cell disorders), metastatic tumors and other cancers. Prevalent cancers include breast, prostate, colon, lung, liver, brain, ovarian and pancreatic cancers.

The term “cancer” as used herein refers to cellular-proliferative disease states, including but not limited to: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor. Metastases of the aforementioned cancers can also be treated in accordance with the methods described herein.

The symbol “ [symbol] ” is used herein to represent the point of attachment of a group to the remainder of a molecule or chemical formula.

Appropriate compounds described herein may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein also include hydrates and solvates, as well as anhydrous and non-solvated forms.

In some embodiments, the compounds, or salts thereof, are substantially isolated. Partial separation can include, for example, a composition enriched in any one or more of the compounds described herein. Substantial separation can include compositions containing at least about 50%, at least about 60%, at least about 70%, at least about 80%, at least about 90%, at least about 95%, at least about 97%, or at least about 99% by weight of any one or more of the compounds described herein, or salt thereof. Methods for isolating compounds and their salts are routine in the art.

### STING Agonist Compounds

Compounds of the present application were prepared and were found to inhibit uncontrolled and/or abnormal cellular activities affected directly or indirectly by activation of the STING protein. In particular, compounds of the present application exhibited activity as STING agonists, and are therefore useful in therapy, for example for the treatment of neoplastic disorders such as cancer.

It will be appreciated by those skilled in the art that the compounds of this invention may exist in tautomeric forms including, but not limited to, Formula (A), Formula (B) and/or Formula (C), or zwitterionic forms including, but not limited to, Formula (D) or Formula (E):

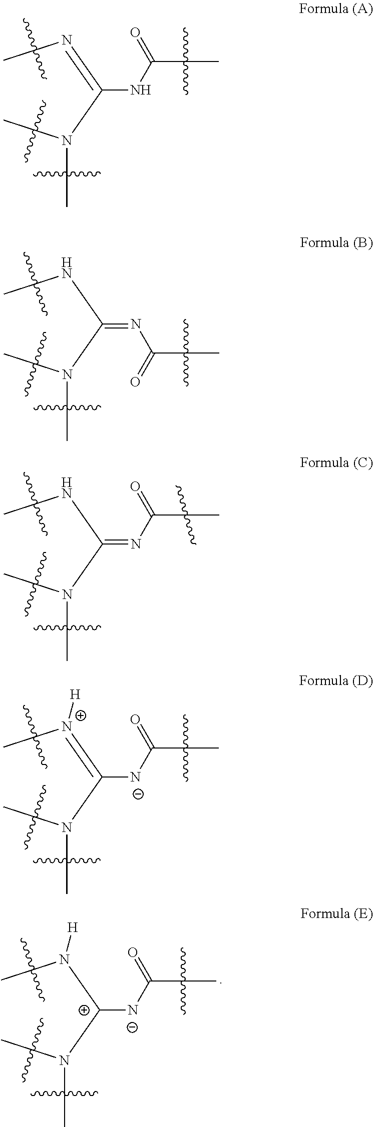 [SMILES: */N=C(/NC(*)=O)N(*)* ; *N/C(=N/C(*)=O)N(*)* ; *N/C(=N\C(*)=O)N(*)* ; */[NH+]=C(/[N-]C(*)=O)N(*)* ; *N[C+]([N-]C(*)=O)N(*)*]

Agonists of stimulator of interferon genes (STING) represent a promising class of immune modulators for potential treatment of cancer. This application is related to a novel class of STING agonists that contain hydrazide and/or hydrazine functional groups.

Accordingly, the present application includes a compound of Formula I or a solvate, pharmaceutically acceptable salt, or tautomer thereof:

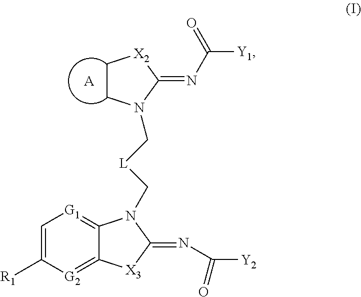

wherein:
Ring A is selected from the group consisting of

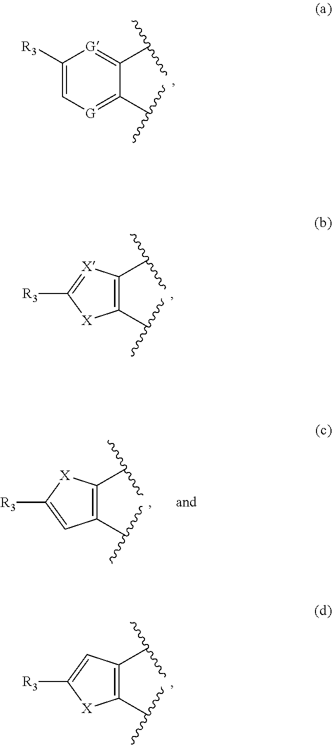

wherein
G and G₁ are independently N, CH, or C—X₁—R₂;
G′ and G₂ are independently N or CH;
X is N—R, O, or S;
X′ is N or CH;
X₁ is CH₂, O or S;
R is hydrogen or a C₁₋₄ alkyl, and
wherein when G and G₁ are each C—X₁—R₂, the R₂ groups are optionally linked to form L₁;
L and L₁ are each independently C₂₋₄ alkylene or C₂₋₄ alkenylene;
R₂ is selected from the group consisting of
hydrogen, a C₂₋₄ cyclic ether, a C₃₋₄ cyclic alkyl, a C₁₋₄ alkyl,

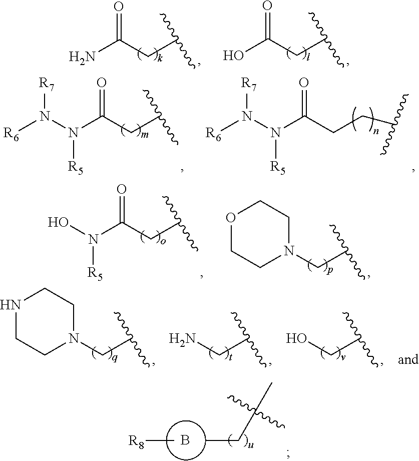

R₁ and R₃ are independently selected from the group consisting of

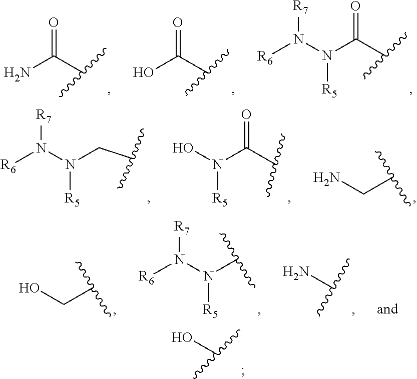

Ring B is a 6-membered aromatic ring or a 5- or 6-membered heteroaromatic ring comprising 1 to 2 heteroatoms selected from N, O, and S;
R₈ is —OH or —NR₉R₁₀;
R₉ and R₁₀ are independently selected from hydrogen, C₁-C₆ alkyl;
X₂ and X₃ are independently NH or S;
Y₁ and Y₂ are independently

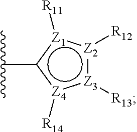

Z₁, Z₂, Z₃, and Z₄ are each independently C, N, O, or S;
R₅, R₆, and R₇ are independently selected from hydrogen, C₁-C₆ alkyl, and C₂-C₆ alkenyl,

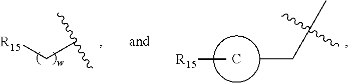

wherein R₅ and R₆ are optionally connected to form a 5- or 6-membered heterocyclic ring;
R₁₅ is —OH or —NR₉R₁₀;
Ring C is a 6-membered aromatic ring or a 5- or 6-membered heteroaromatic ring comprising 1 to 2 heteroatoms selected from N, O, and S;
R₁₁, R₁₂, R₁₃, and R₁₄ are independently absent, hydrogen, or C₁₋₄ alkyl; and
n, p, q, t, and v are independently an integer from 2 to 6; and
k, l, m, o, u, and w are independently an integer from 1 to 6, and
provided that at least one of G and G₁ is C—X₁—R₂, wherein R₂ is

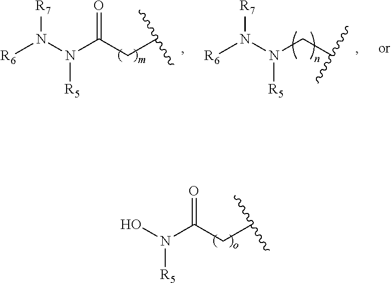

and/or
at least one of R₁ and R₃ is

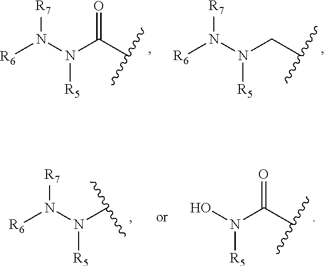

In embodiments, Ring A is selected from the group consisting of

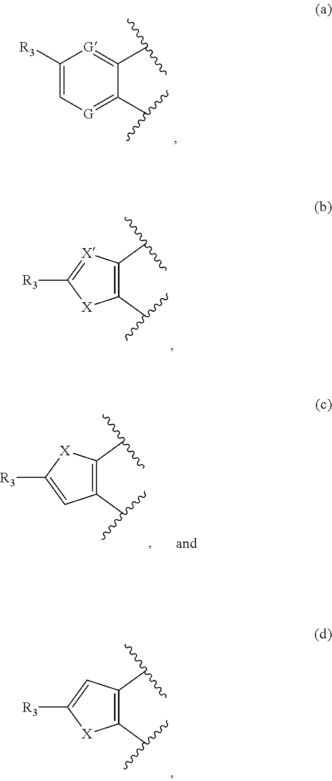

wherein structures (a), (b), (c), and (d) are attached to the X₂ and N of the adjacent 5-membered heterocyclic ring in Formula I to form an 8 or 9-membered heteroaromatic fused ring system of Formula I, wherein G and G₁ are independently N, CH, or C—X₁—R₂; G′ and G₂ are independently N or CH; X is N—R, O, or S; X′ is N or CH; X₁ is CH₂, O or S; R is hydrogen or a C₁₋₄ alkyl; and X₂ and X₃ are each independently NH or S. In some embodiments, both X₂ and X₃ are N. In other embodiments, both X₂ and X₃ are NH. In other embodiments, X₂ is NH and X₃ is N. In yet other embodiments, X₂ is N and X₃ is NH.

In Formula I, when Ring A is structure (c), the X is one carbon removed from the X₂ on the adjacent fused 5-membered heterocyclic ring. When Ring A is structure (d), the X is one carbon removed from the N atom on the adjacent fused 5-membered heterocyclic ring.

In embodiments, Ring A is selected from phenyl, pyridinyl, imidazolyl, thiazolyl, thiophenyl, furanyl, and pyrrolyl, all of which are substituted with R₃, which is selected from the group consisting of

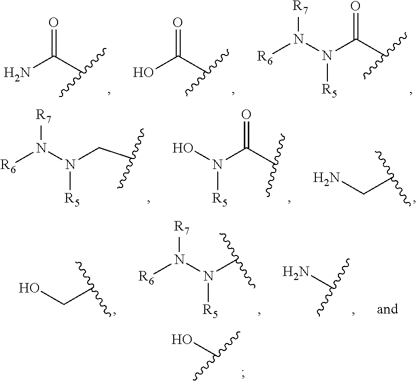

wherein R₅, R₆, and R₇ are independently selected from hydrogen, C₁-C₆ alkyl, C₂-C₆ alkenyl,

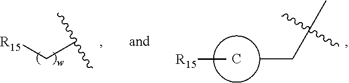

wherein R₅ and Rₑ are optionally connected to form a 5- or 6-membered heterocyclic ring; R₁₅ is —OH or —NR₉R₁₀; Ring C is a 6-membered aromatic ring or a 5- or 6-membered heteroaromatic ring comprising 1 to 2 heteroatoms selected from N, O, and S; and R₁₁, R₁₂, R₁₃, and R₁₄ are independently absent, hydrogen, or C₁₋₄ alkyl. R₉ and R₁₀ in the amino group are independently selected from hydrogen and C₁-C₆ alkyl. In embodiments, Ring C is selected from the group consisting of phenyl, pyrrolyl, furanyl, thiophenyl, imidazolyl, pyrazolyl, oxathiolyl, isoxathiolyl, oxazolyl, isoxazolyl, thiazolyl, and isothiazolyl.

In embodiments, linking group L is C₂₋₄ alkylene or C₂₋₄ alkenylene. Preferably, L is ethylenyl, propylenyl, or butylenyl. In some embodiments, L is ethylenyl. In one embodiment, L is a C₂-alkenylene; and X₂ and X₃ are NH.

In embodiments, R₂ is selected from the group consisting of hydrogen, a C₂₋₄ cyclic ether, a C₃₋₄ cyclic alkyl, C₁₋₄ alkyl,

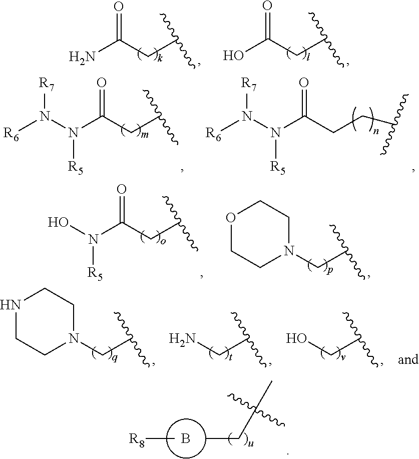

Ring B is a 6-membered aromatic ring or a 5- or 6-membered heteroaromatic ring comprising 1 to 2 heteroatoms selected from N, O, and S. In embodiments, Ring B is selected from the group consisting of phenyl, pyrrolyl, furanyl, thiophenyl, imidazolyl, pyrazolyl, oxathiolyl, isoxathiolyl, oxazolyl, isoxazolyl, thiazolyl, and isothiazolyl. Ring B is substituted with R₈, which is —OH or —NR₉R₁₀.

In some embodiments, at least one of R₅, R₆, and R₇ is a hydrogen atom.

In embodiments, Y₁ and Y₂ are independently

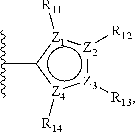

wherein Z₁, Z₂, Z₃, and Z₄ are each independently C, N, O, or S; and R₁₁, R₁₂, R₁₃, and R₁₄ are independently absent, hydrogen, or C₁₋₄ alkyl. In some embodiments, Y₁ and Y₂ are independently selected from the group consisting of pyrrolyl, furanyl, thiophenyl, imidazolyl, pyrazolyl, oxathiolyl, isoxathiolyl, oxazolyl, isoxzolyl, thiazolyl, isothiazolyl, triazolyl, furazonyl, oxadiazolyl, thiadiazolyl, dioxazolyl, dithiazolyl, and tetrazolyl.

In embodiments, R₁ and R₃ are independently selected from the group consisting of

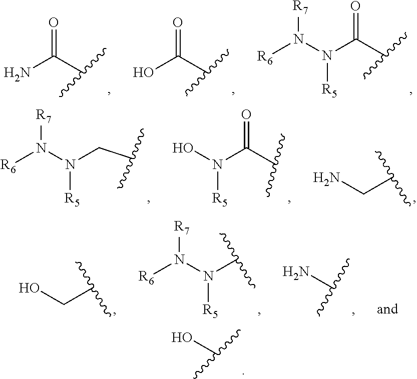

In embodiments disclosed herein, at least one of G and G₁ is C—X₁—R₂, wherein R₂ is

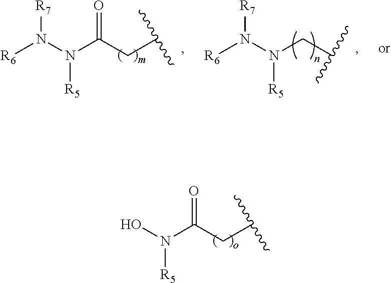

and/or at least one of R₁ and R₃ is

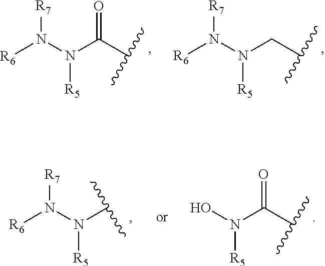

In embodiments, both R₁ and R₃ are independently

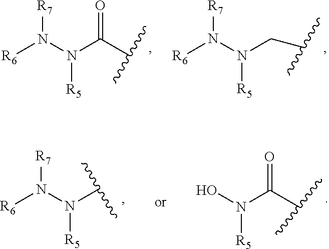

In other embodiments, both R₁ and R₃ are independently

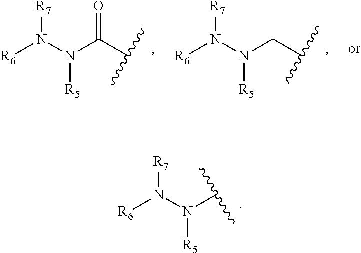

The hydrazide and hydrazine functional groups, including hydroxamic acid, are compatible with different conjugation and linker chemistries that can be used to generate a variety of STING-vector conjugates for developing tumor-targeted immunotherapies. The hydrazide- and hydrazine-derived linkers can be specifically cleaved inside tumor cells and/or in tumor microenvironment (e.g., extracellular matrix) by different cleavage mechanisms, releasing the active STING agonist payload. Such cleavage mechanisms include enzymatic cleavage by cathepsin B or legumain, chemical cleavage at low pH in lysosomes, and reduction cleavage by glutathione.

In the compounds represented by Formula I, the variables n, p, q, t, and v are independently an integer from 2 to 6; and the variables k, l, m, o, u, and w are independently an integer from 1 to 6.

In some embodiments, compounds of Formula I include those selected from the group consisting of

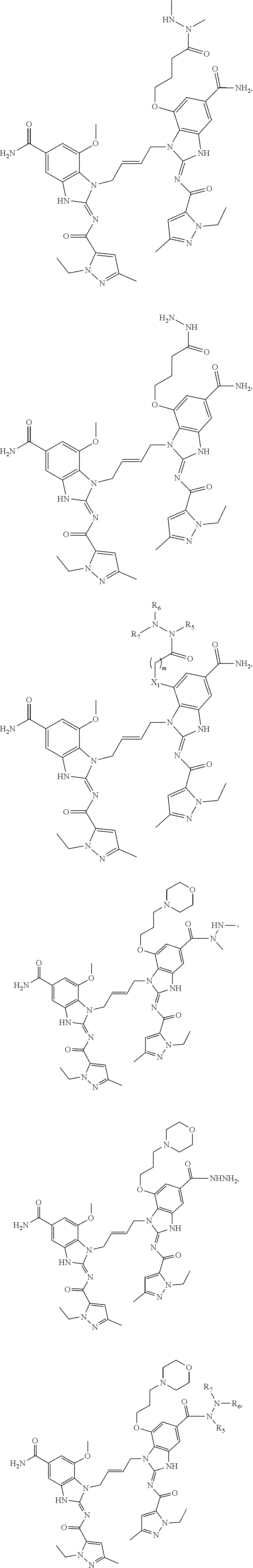

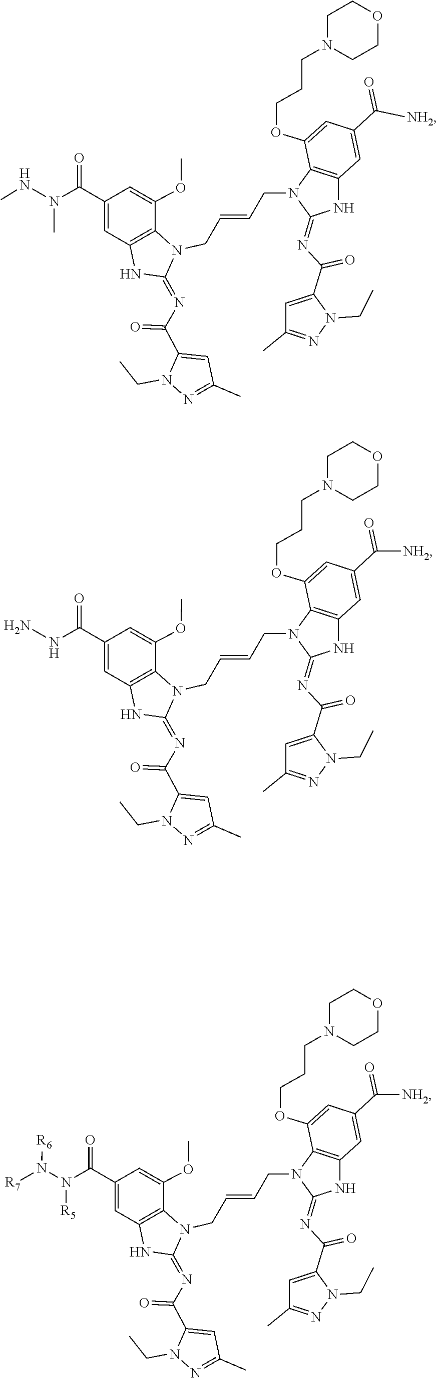

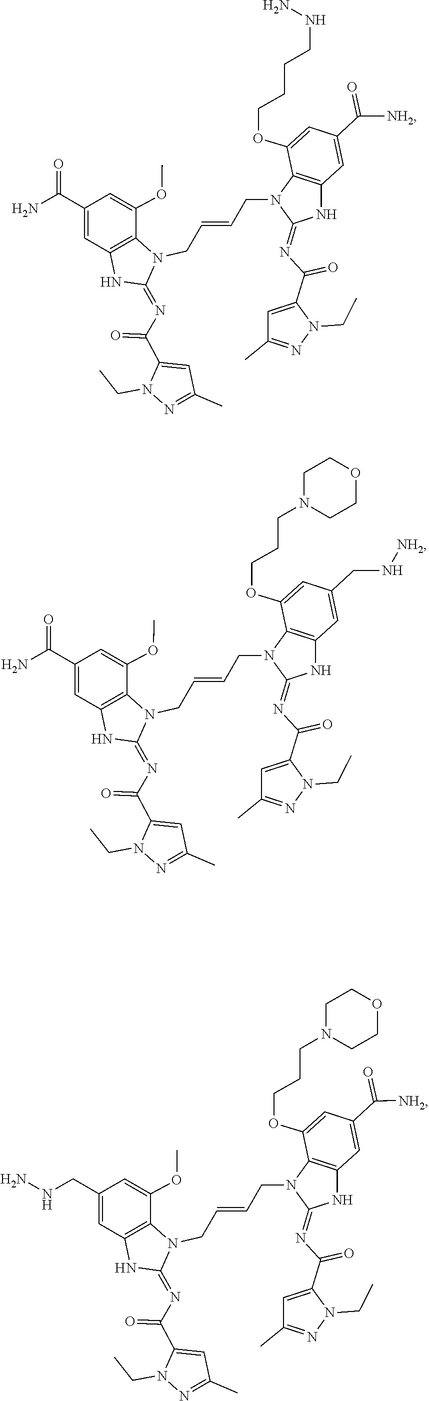 [SMILES: CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(C(N)=O)cc(OCCCCNN)c21 ; CCn1nc(C)cc1C(=O)/N=c1\[nH]c2cc(C(N)=O)cc(OC)c2n1C/C=C/Cn1/c(=N/C(=O)c2cc(C)nn2CC)[nH]c2cc(CNN)cc(OCCCN3CCOCC3)c21 ; CCn1nc(C)cc1C(=O)/N=c1\[nH]c2cc(CNN)cc(OC)c2n1C/C=C/Cn1/c(=N/C(=O)c2cc(C)nn2CC)[nH]c2cc(C(N)=O)cc(OCCCN3CCOCC3)c21]

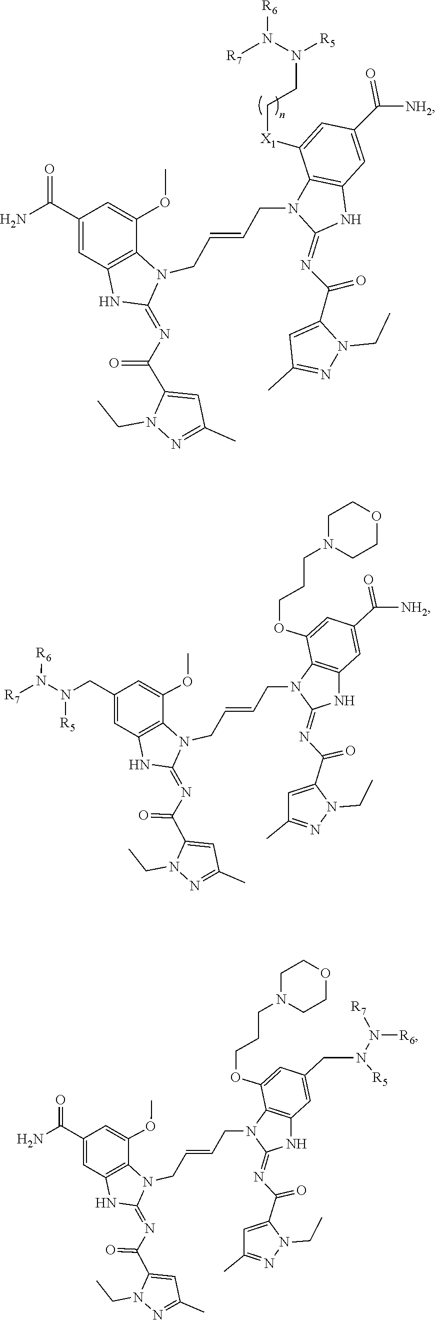

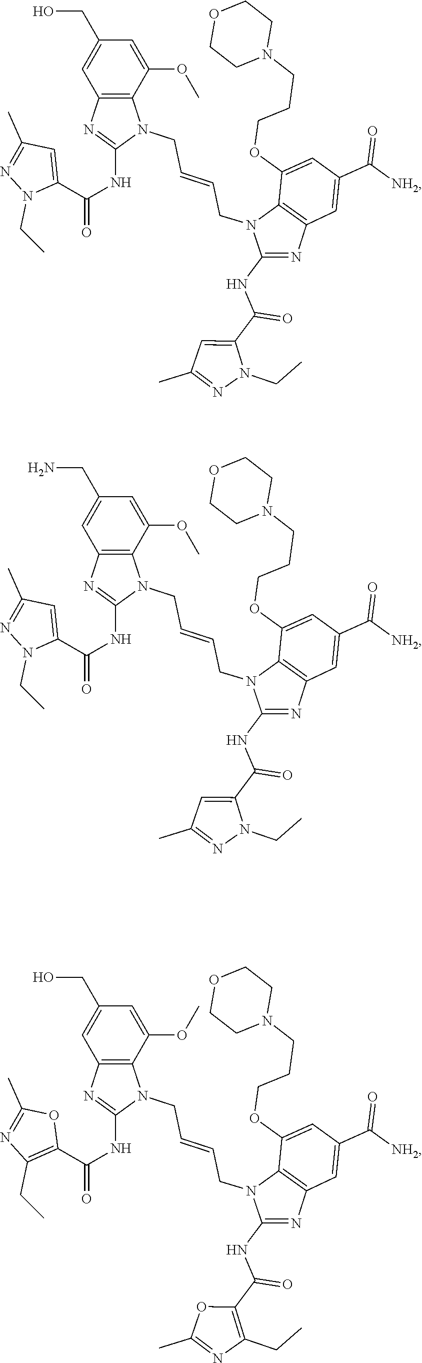 [SMILES: CCn1nc(C)cc1C(=O)Nc1nc2cc(CO)cc(OC)c2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(CN)cc(OC)c2n1C/C=C/Cn1c(NC(=O)c2cc(C)nn2CC)nc2cc(C(N)=O)cc(OCCCN3CCOCC3)c21 ; CCc1nc(C)oc1C(=O)Nc1nc2cc(CO)cc(OC)c2n1C/C=C/Cn1c(NC(=O)c2oc(C)nc2CC)nc2cc(C(N)=O)cc(OCCCN3CCOCC3)c21]

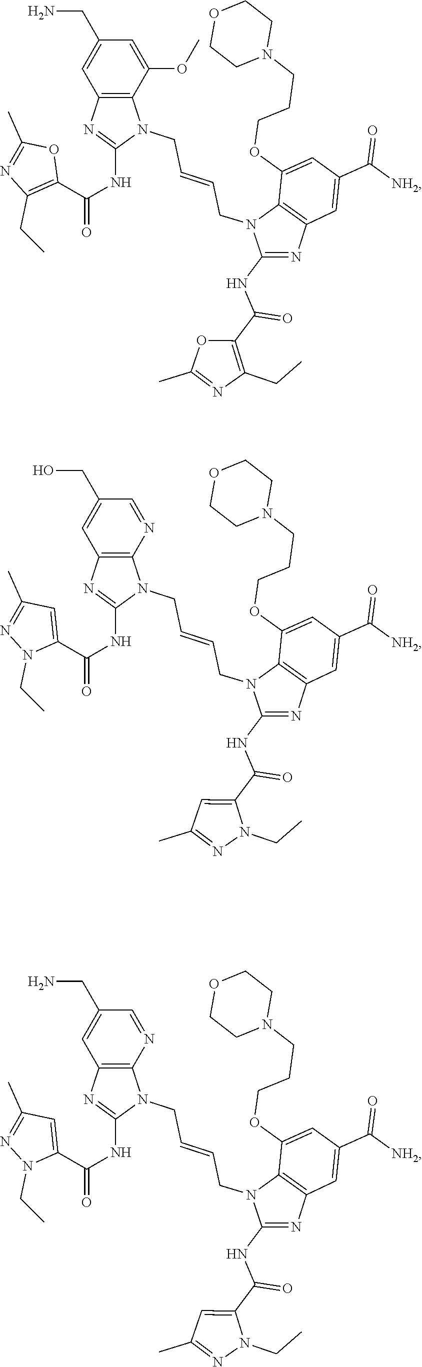 [SMILES: CCc1nc(C)oc1C(=O)Nc1nc2cc(CN)cc(OC)c2n1C/C=C/Cn1c(NC(=O)c2oc(C)nc2CC)nc2cc(C(N)=O)cc(OCCCN3CCOCC3)c21 ; CCn1nc(C)cc1C(=O)Nc1nc2cc(CO)cnc2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(CN)cnc2n1C/C=C/Cn1c(NC(=O)c2cc(C)nn2CC)nc2cc(C(N)=O)cc(OCCCN3CCOCC3)c21]

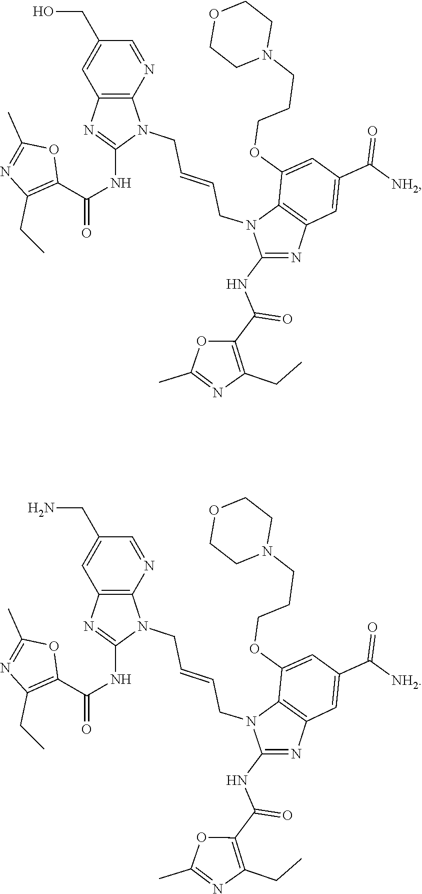 [SMILES: CCc1nc(C)oc1C(=O)Nc1nc2cc(CO)cnc2n1C/C=C/Cn1c(NC(=O)c2oc(C)nc2CC)nc2cc(C(N)=O)cc(OCCCN3CCOCC3)c21 ; CCc1nc(C)oc1C(=O)Nc1nc2cc(CN)cnc2n1C/C=C/Cn1c(NC(=O)c2oc(C)nc2CC)nc2cc(C(N)=O)cc(OCCCN3CCOCC3)c21]

In other embodiments, compounds of Formula I include those selected from the group consisting of

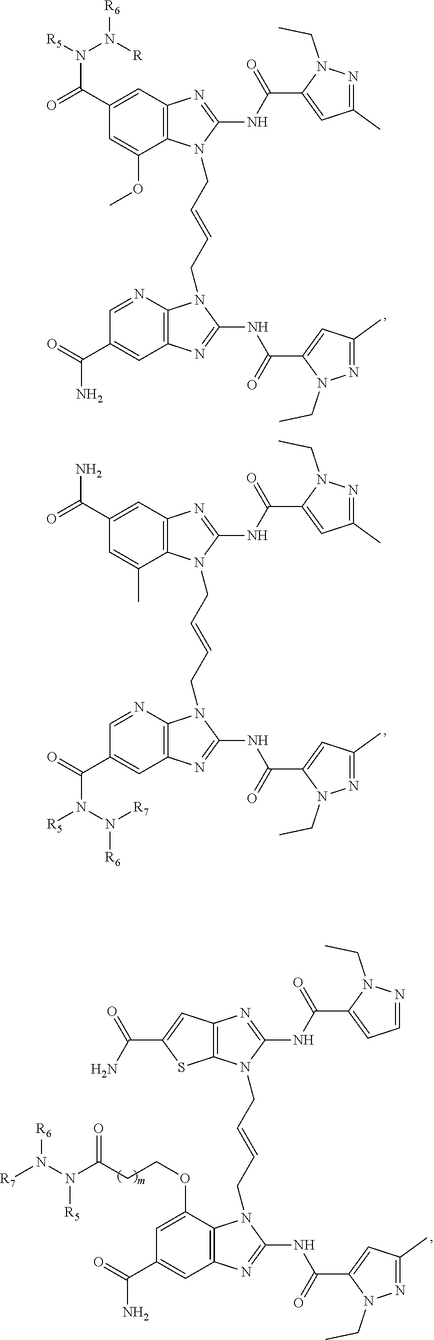

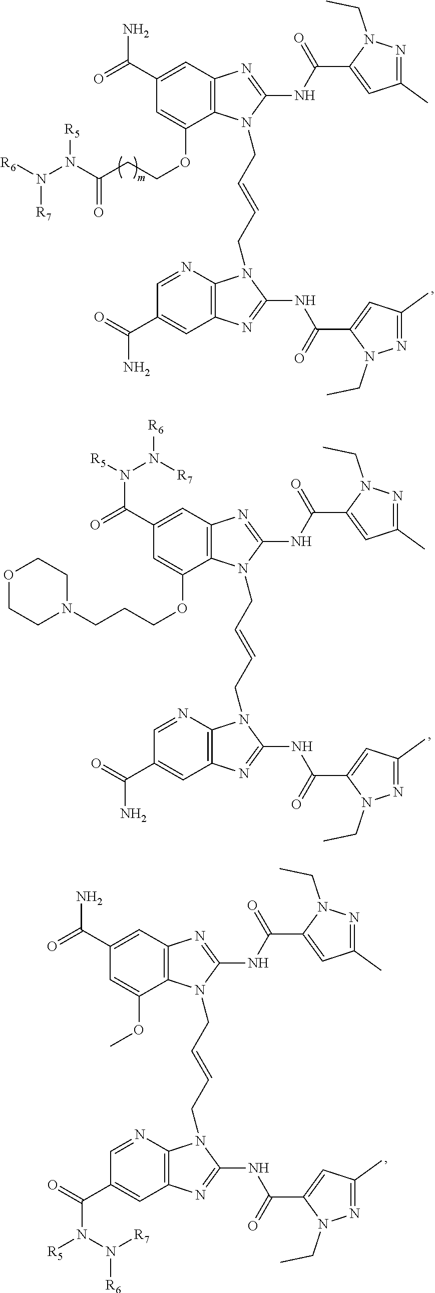

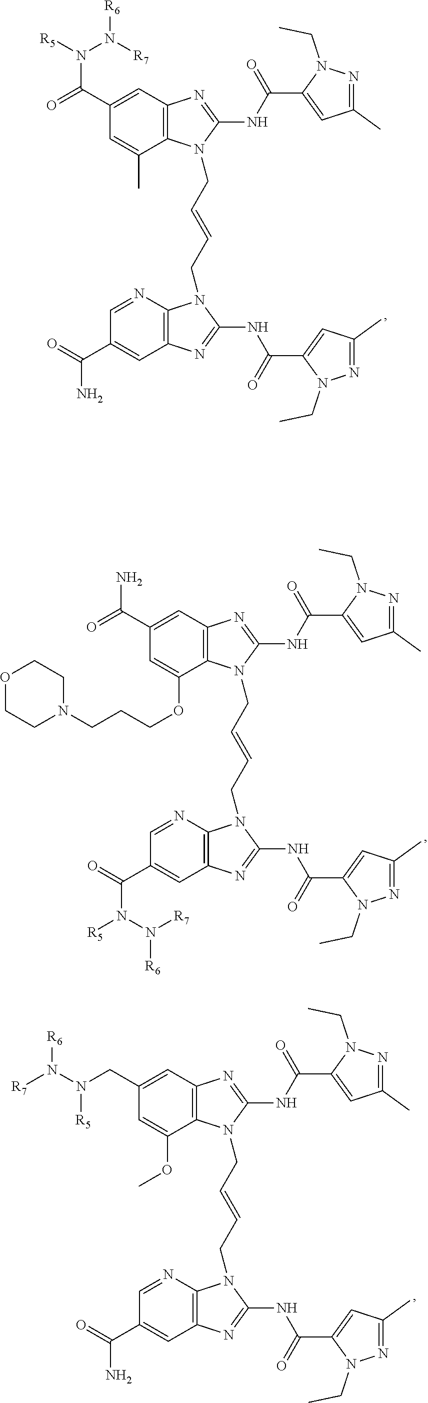

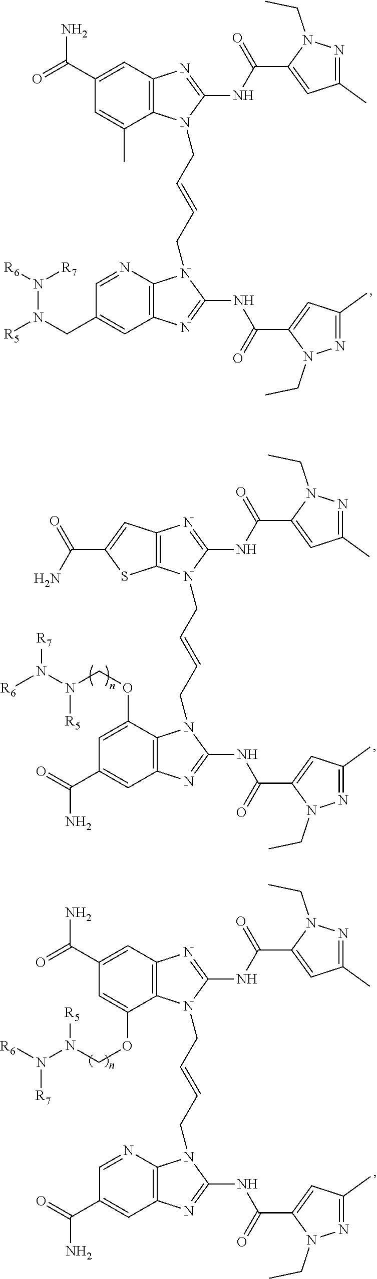

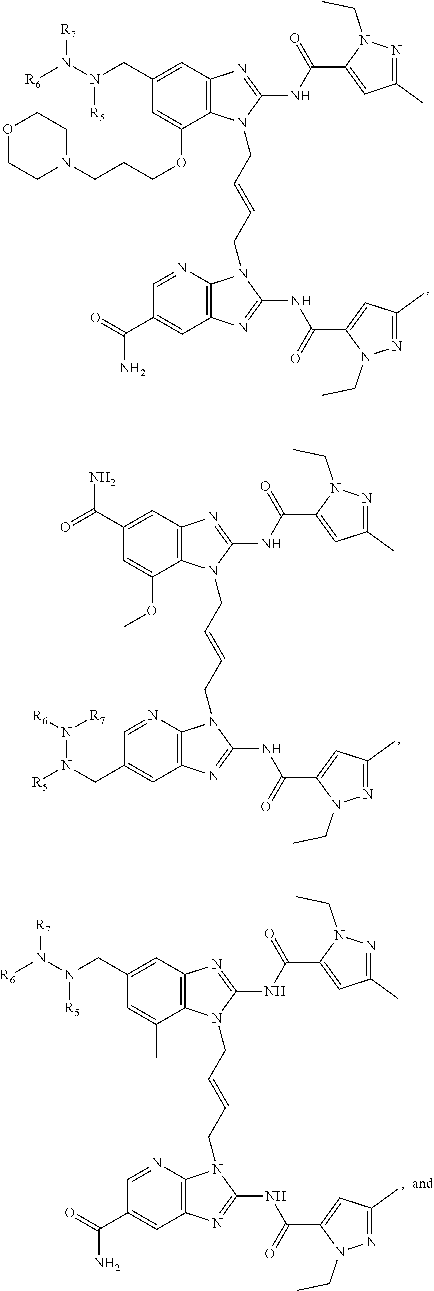

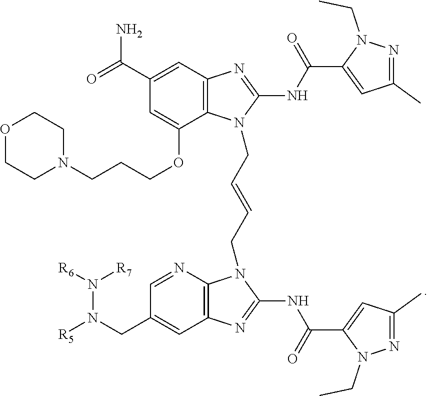

Some embodiments disclosed herein are a subset of Formula I represented by Formula IA:

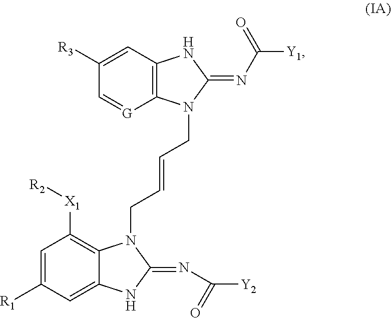

or a solvate, pharmaceutically acceptable salt, or tautomer thereof, wherein:
X₁ is CH₂, O or S;
G is CH, C—SCH₃, C—OCH₃, or N;
R₂ is selected from the group consisting of
hydrogen, a C₂₋₄ cyclic ether, a C₃₋₄ cyclic alkyl, C₁₋₄ alkyl,

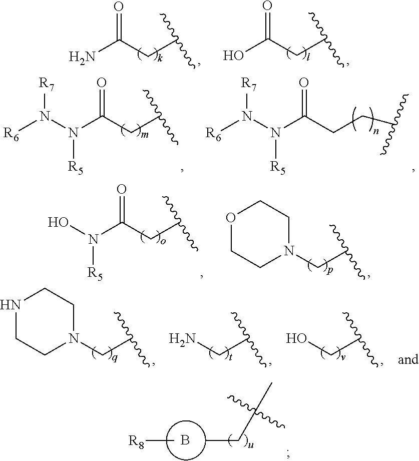

R₁ and R₅ are independently selected from the group consisting of

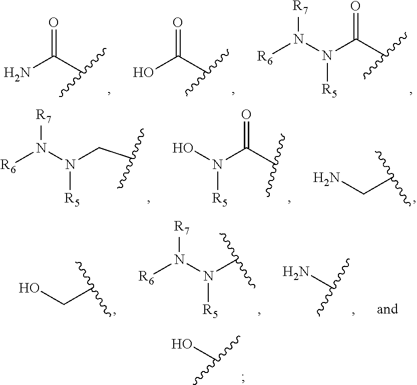

Ring B is a 6-membered aromatic ring or a 5- or 6-membered heteroaromatic ring comprising 1 to 2 heteroatoms selected from N, O, and S;
R₈ is —OH or —NH₂;
Y₁ and Y₂ are independently

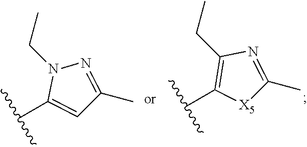

X₅ is S, O, or NR₇;
R₅, R₆, and R₇ are independently selected from hydrogen, C₁-C₆ alkyl, C₂-C₆ alkenyl,

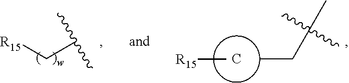

wherein R₅ and R₆ are optionally connected to form a 5- or 6-membered heterocyclic ring;
R₁₅ is —OH or —NR₉R₁₀;
R₉ and R₁₀ are independently selected from hydrogen and C₁-C₆ alkyl;
Ring C is a 6-membered aromatic ring or a 5- or 6-membered heteroaromatic ring comprising 1 to 2 heteroatoms selected from N, O, and S;
n, p, q, t, and v are independently an integer from 2 to 6; and
k, l, m, o, u, and w are independently an integer from 1 to 6, and
provided that at least R₂ is

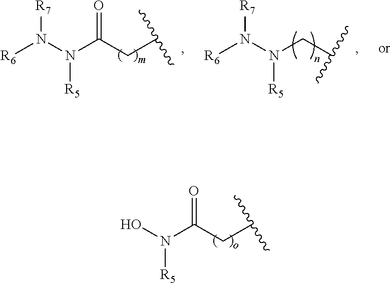

and/or
at least one of R₁ and R₃ is

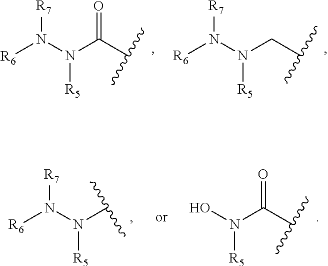

In some embodiments, both R₁ and R₃ are independently

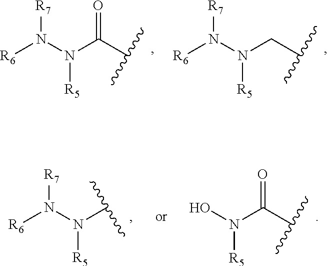

In other embodiments, both R₁ and R₃ are independently

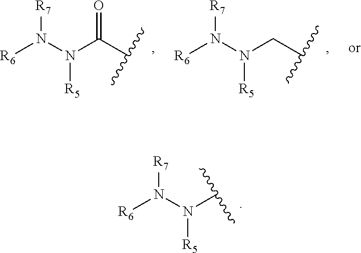

Some embodiments disclosed herein are a subset of Formula I represented by Formula IA′:

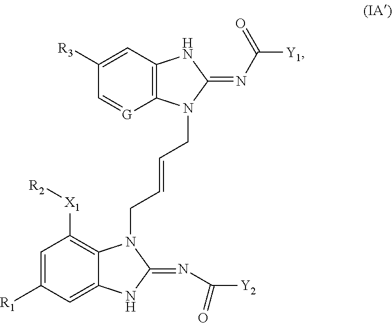

or a solvate, pharmaceutically acceptable salt, or tautomer thereof, wherein:
X₁ is CH₂, O or S;
G is CH, C—SCH₃, C—OCH₃, or N;
R₂ is selected from the group consisting of
hydrogen, a C₂₋₄ cyclic ether, a C₃₋₄ cyclic alkyl, C₁₋₄ alkyl,

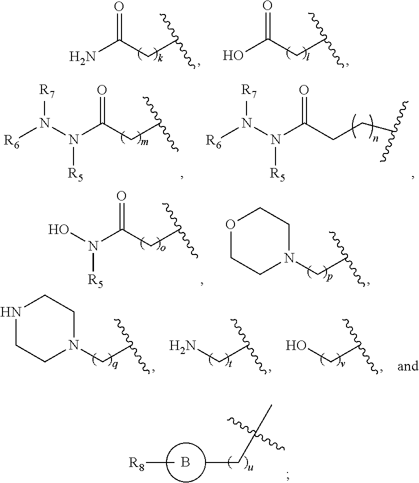

R₁ and R₃ are independently selected from the group consisting of

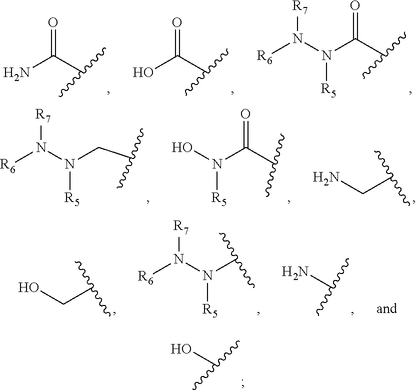

Ring B is a 6-membered aromatic ring or a 5- or 6-membered heteroaromatic ring comprising 1 to 2 heteroatoms selected from N, O, and S;
R₈ is —OH or —NH₂;
Y₁ and Y₂ are independently

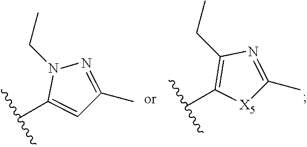

X₅ is S, O, or NR₇;
R₅, R₆, and R₇ are independently selected from hydrogen, —OH, C₁-C₆ alkyl,

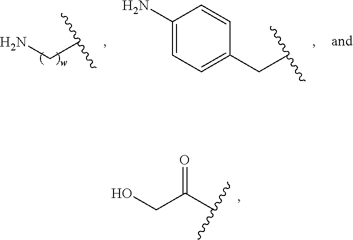

wherein R₅ and R₆ are optionally connected to form a 5- or 6-membered heterocyclic ring;
n, p, q, t, and v are independently an integer from 2 to 6; and
k, l, m, o, u, and w are independently an integer from 1 to 6, and
provided that R₂ is

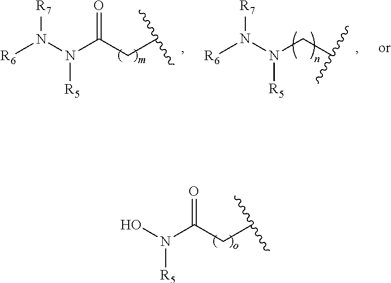

and/or
at least one of R₁ and R₃ is

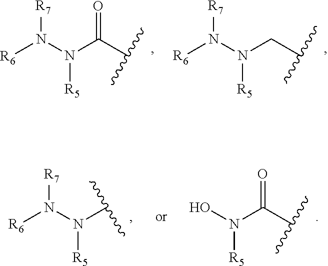

In some embodiments of Formula IA′, G is N; X₁ is O; R₁ and R₃ are each

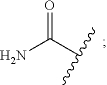 [SMILES: *C(N)=O]

### R₂ is

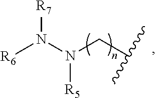

wherein n is 2, 3, or 4; R₅, R₆, and R₇ are each independently selected from H, C₁-C₆ alkyl, and

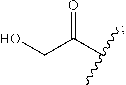 [SMILES: *C(=O)CO]

and Y₁ and Y₂ are each

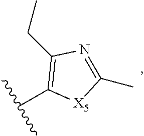

wherein X₅ is O.

In another embodiment of Formula IA′, G is N; X₁ is O; R₁ and R₃ are each

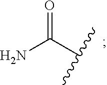 [SMILES: *C(N)=O]

### R₂ is

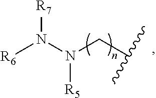

wherein n is 3; R₅ is methyl; R₆ and R₇ are H; and Y₁ and Y₂ are each

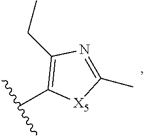

wherein X₅ is O such that the resulting structure is that shown in structure (XX):

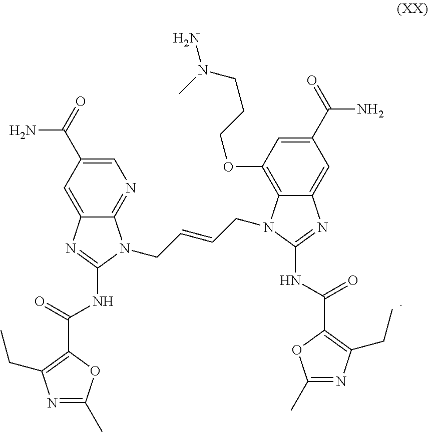 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(C)N)c21]

Derivatives and isomers of structure XX include (relative to Formula IA′) compounds where R₅, R₆, and R₇ are each independently selected from H, methyl, or

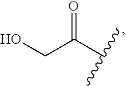 [SMILES: *C(=O)CO]

and include the following structures XXI, XXII, XXIII, and XXIV:

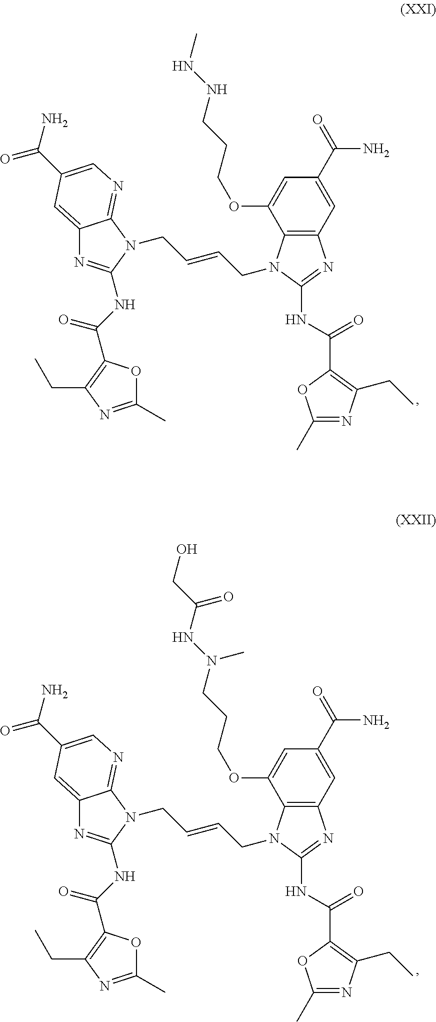 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCNNC)c21 ; CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(C)NC(=O)CO)c21]

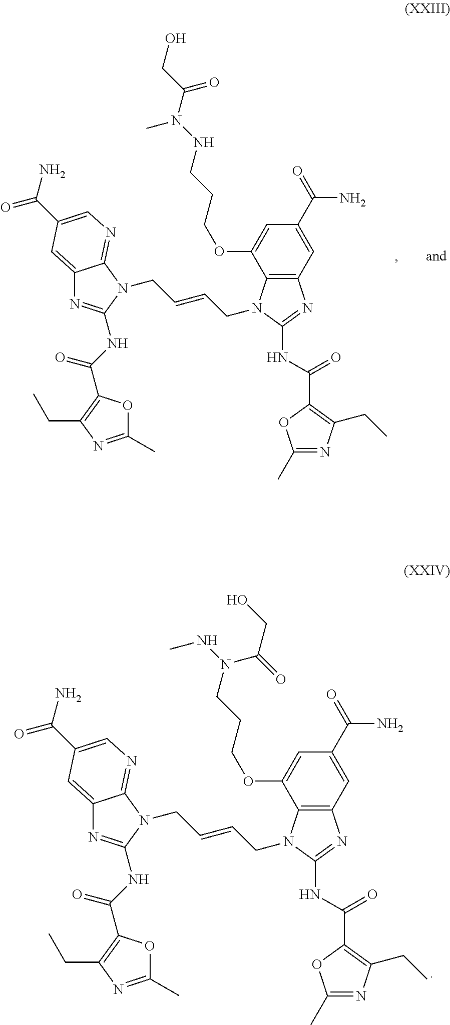 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCNN(C)C(=O)CO)c21 ; CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(NC)C(=O)CO)c21]

In some embodiments, compounds of Formula IA and Formula IA′ include those selected from the group consisting of

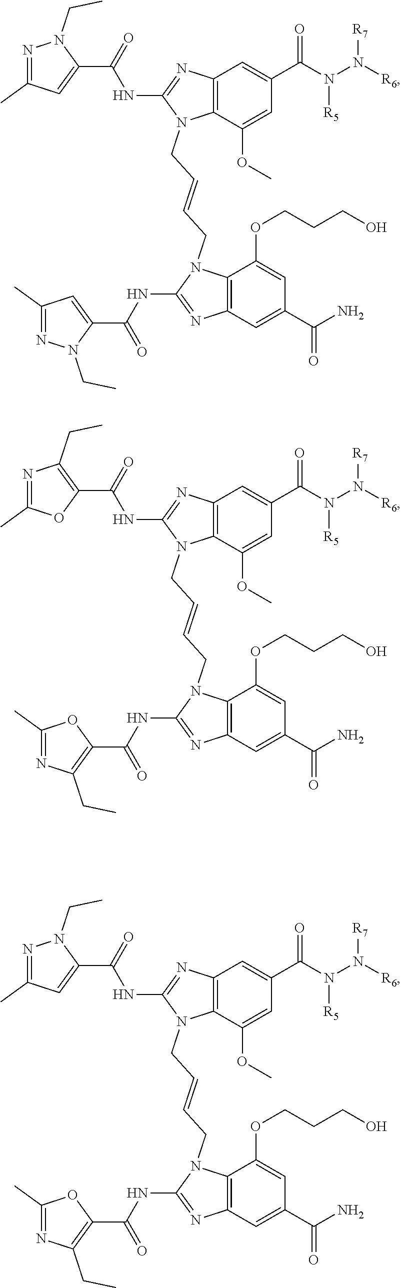

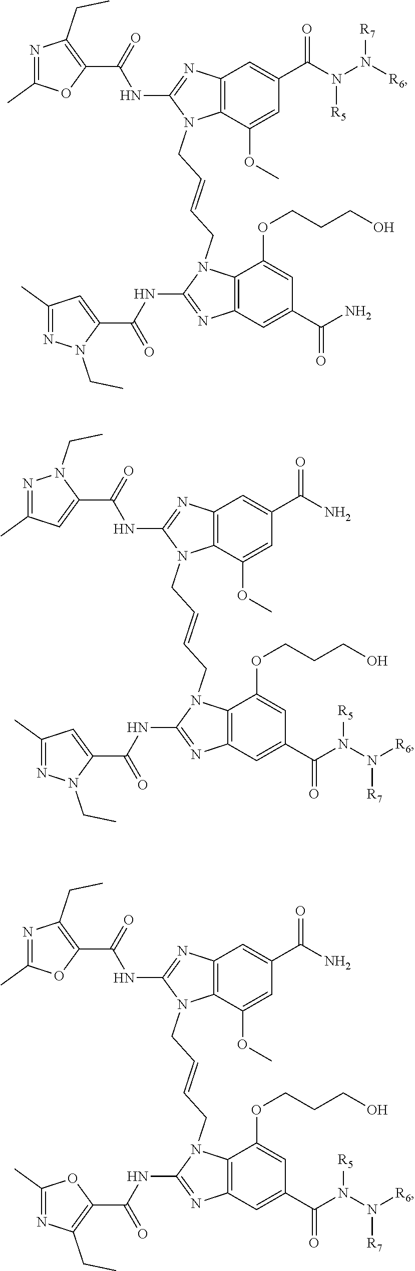

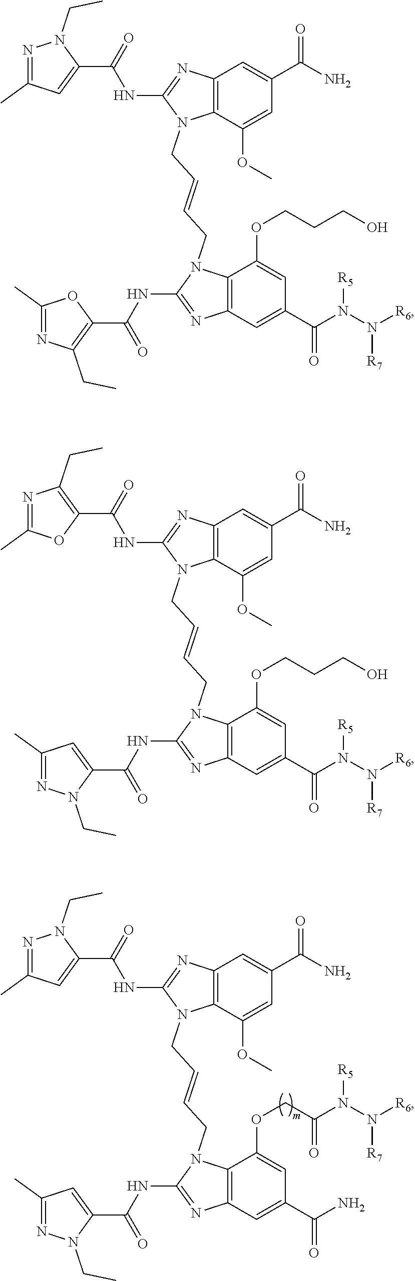

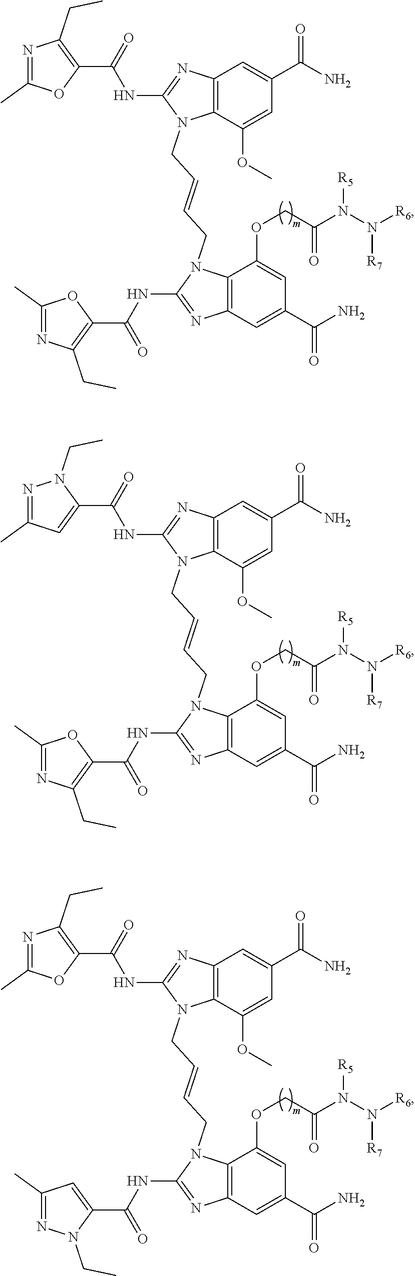

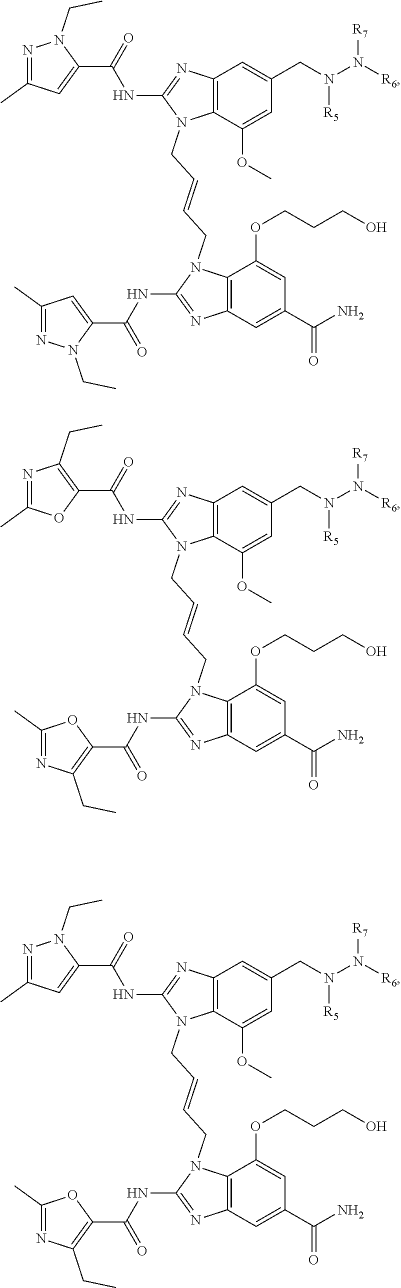

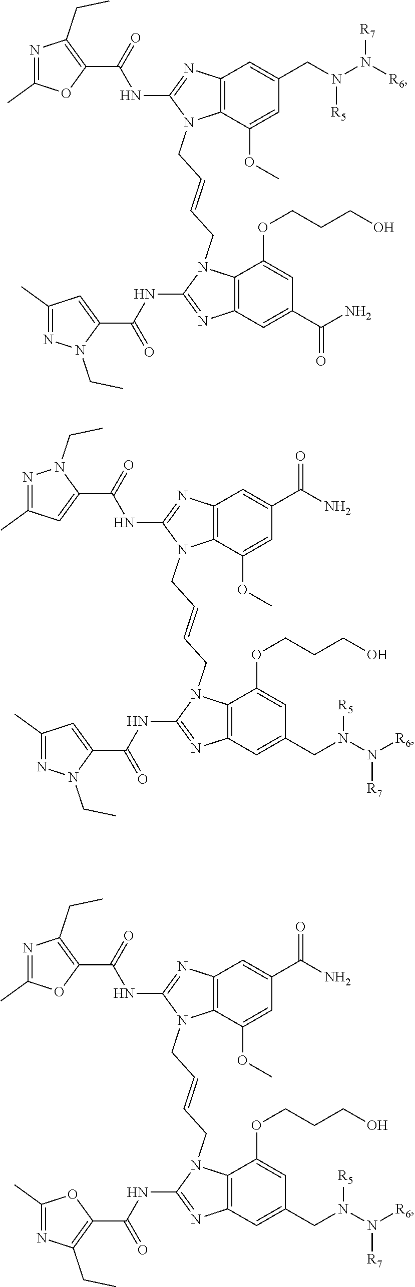

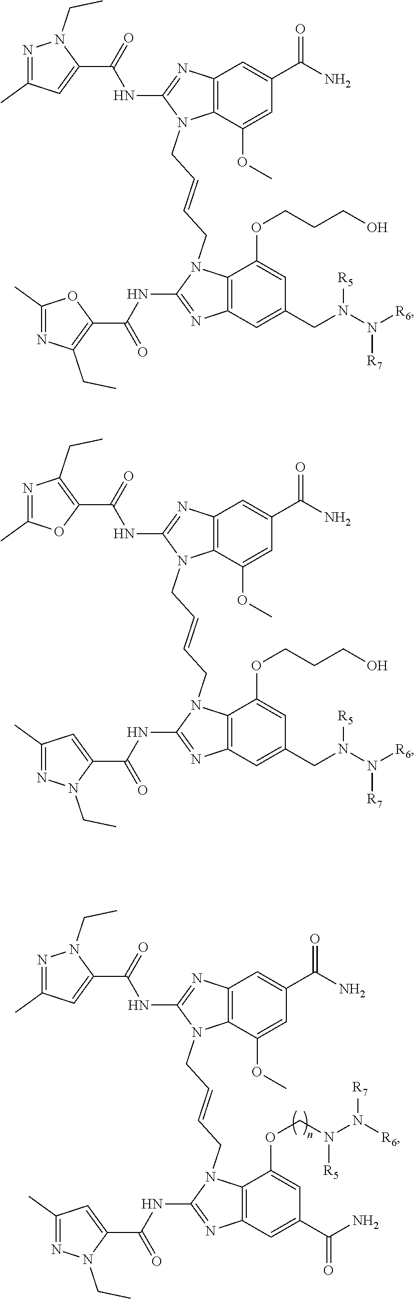

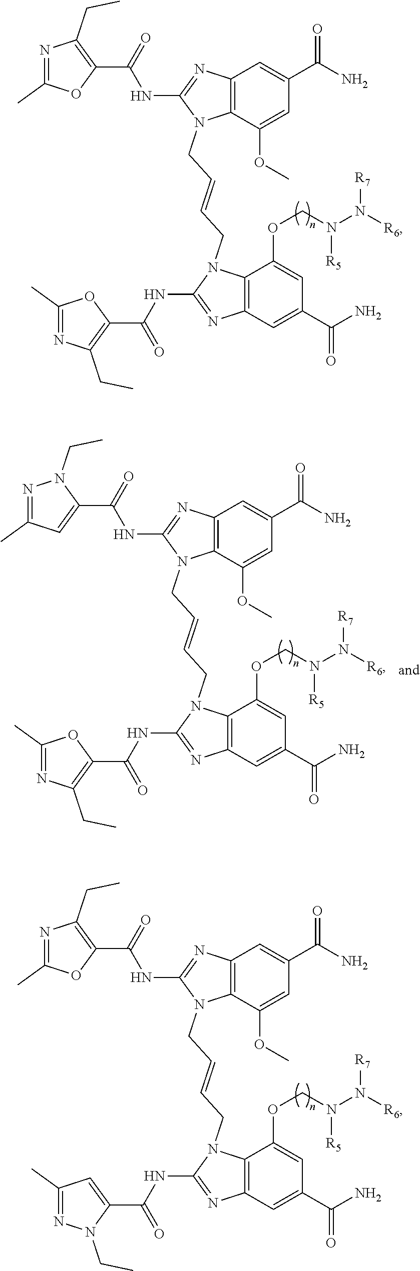

wherein R₅, R₆, R₇, n, and m are as defined above.

In other embodiments, compounds of Formula IA and Formula IA′ include those selected from the group consisting of

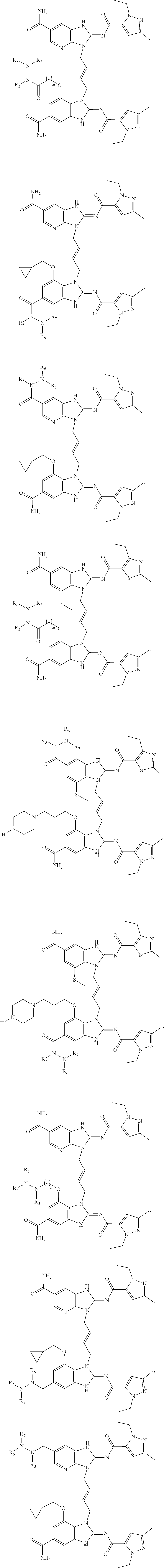

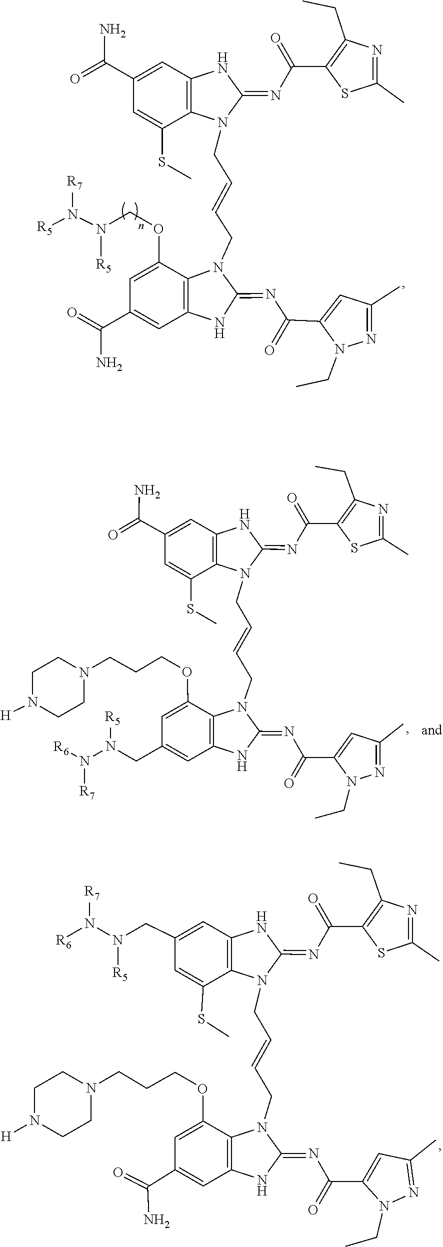

wherein R₅, R₆, R₇, n, and m are as defined above.

Some embodiments disclosed herein are a subset of Formula I represented by Formula IA′:

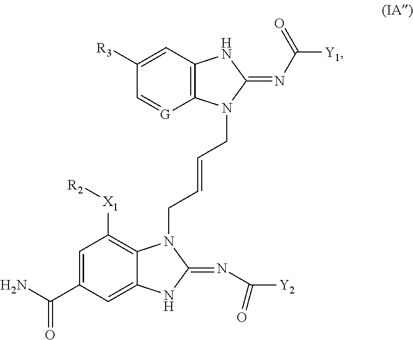

or a solvate, pharmaceutically acceptable salt, or tautomer thereof, wherein:
X₁ is CH₂, O, or S;
G is CH, C—OCH₃, or N;
R₂ is

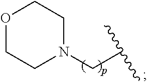

R₃ is

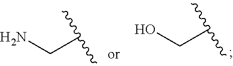

Y₁ and Y₂ are independently

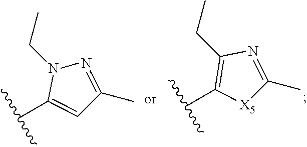

X₅ is O or S; and
p is an integer from 2 to 6.

In some embodiments of Formula IA″, R₃ is

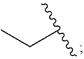 [SMILES: *CC]

G is C—OCH₃; X₁ is O; p is 3; and Y₁ is

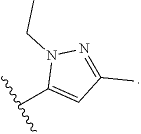 [SMILES: *c1cc(C)nn1CC]

In other embodiments of Formula IA″, R₃ is

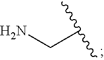 [SMILES: *CN]

G is C—OCH₃; X₁ is O; p is 3; Y₁ is

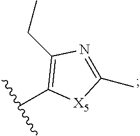

### and X₅ is O.

In other embodiments of Formula IA″, R₃ is

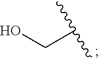 [SMILES: *CO]

G is C—OCH₃; X₁ is O; p is 3; and Y₁ is

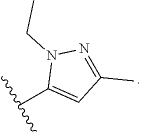 [SMILES: *c1cc(C)nn1CC]

In other embodiments of Formula IA″, R₃ is

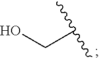 [SMILES: *CO]

G is C—OCH₃; X₁ is O; p is 3; Y₁ is

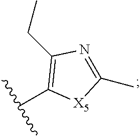

### and X₅ is O.

In yet other embodiments of Formula IA″, R₃ is

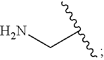 [SMILES: *CN]

G is N; X₁ is O; p is 3; and Y₁ is

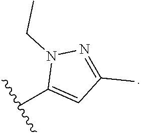 [SMILES: *c1cc(C)nn1CC]

In yet other embodiments of Formula IA″, R₃ is

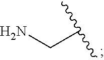 [SMILES: *CN]

G is N; X₁ is O; p is 3; Y₁ is

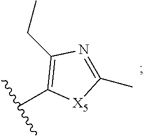

### and X₅ is O.

In yet other embodiments of Formula IA″, R₃ is

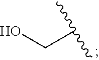 [SMILES: *CO]

G is N; X₁ is O; p is 3; and Y₁ is

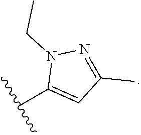 [SMILES: *c1cc(C)nn1CC]

In yet other embodiments of Formula IA″, R₃ is

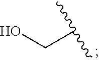 [SMILES: *CO]

G is N; X₁ is O; p is 3; Y₁ is

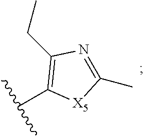

### and X₅ is O.

Embodiments of Formula IA″ include the following compounds:

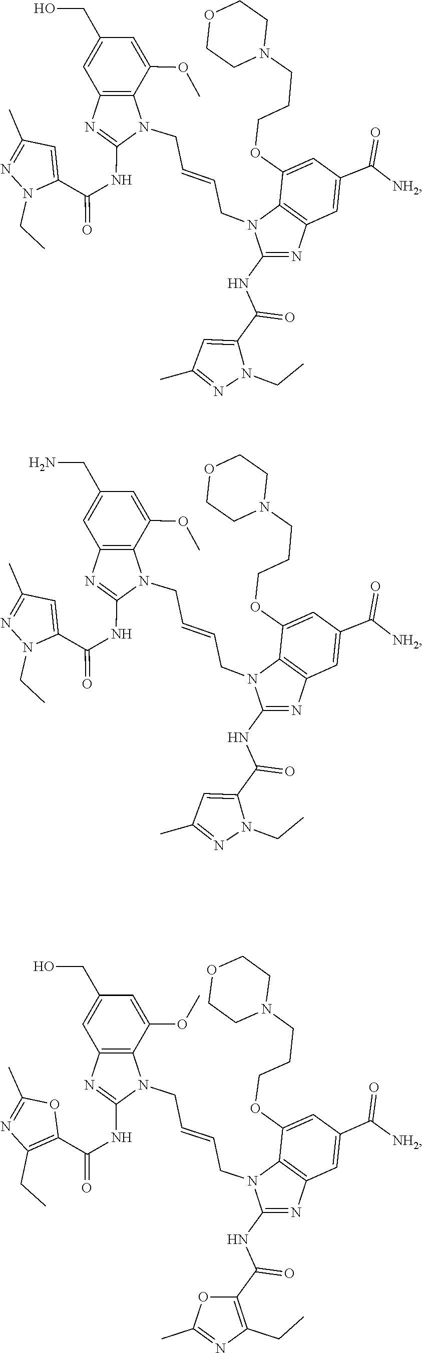 [SMILES: CCn1nc(C)cc1C(=O)Nc1nc2cc(CO)cc(OC)c2n1C/C=C/Cn1c(NC(=O)c2cc(C)nn2CC)nc2cc(C(N)=O)cc(OCCCN3CCOCC3)c21 ; CCn1nc(C)cc1C(=O)Nc1nc2cc(CN)cc(OC)c2n1C/C=C/Cn1c(NC(=O)c2cc(C)nn2CC)nc2cc(C(N)=O)cc(OCCCN3CCOCC3)c21 ; CCc1nc(C)oc1C(=O)Nc1nc2cc(CO)cc(OC)c2n1C/C=C/Cn1c(NC(=O)c2oc(C)nc2CC)nc2cc(C(N)=O)cc(OCCCN3CCOCC3)c21]

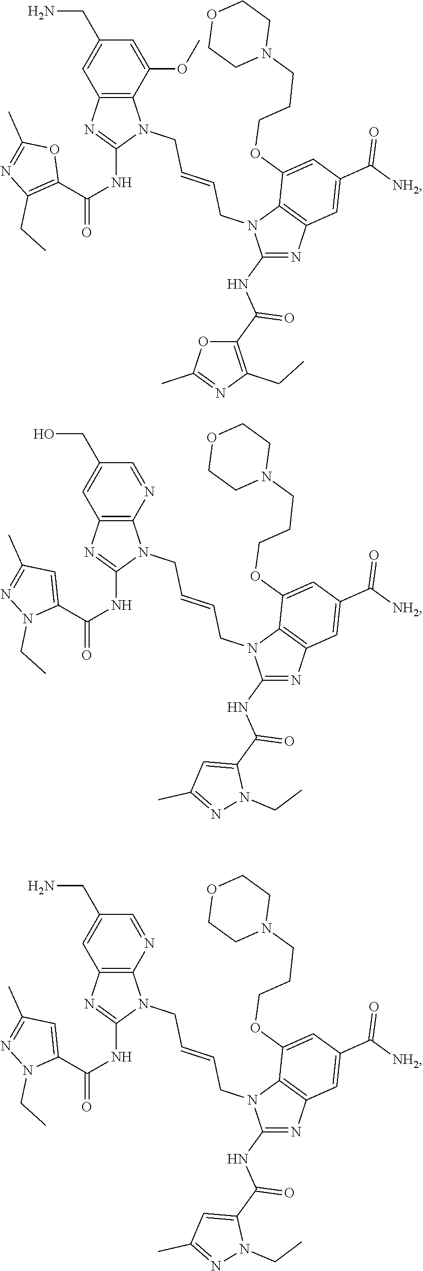

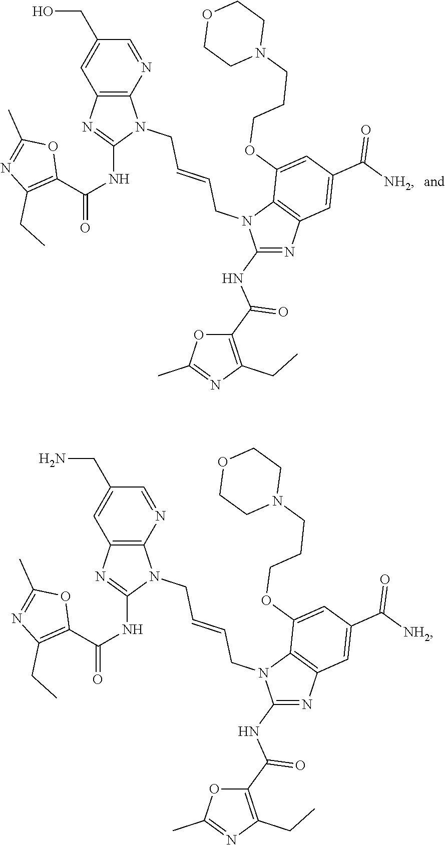 [SMILES: CCc1nc(C)oc1C(=O)Nc1nc2cc(CO)cnc2n1C/C=C/Cn1c(NC(=O)c2oc(C)nc2CC)nc2cc(C(N)=O)cc(OCCCN3CCOCC3)c21 ; "and" ; CCc1nc(C)oc1C(=O)Nc1nc2cc(CN)cnc2n1C/C=C/Cn1c(NC(=O)c2oc(C)nc2CC)nc2cc(C(N)=O)cc(OCCCN3CCOCC3)c21]

or a solvate, pharmaceutically acceptable salt, or tautomer thereof.

In embodiments of Formula I where Ring A is

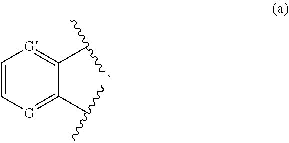

G′ and G₂ are CH, and G and G₁ are each C—X₁—R₂, wherein X₁ is CH₂, O or S, a second linking group, L₁, may be formed through the R₂ groups. Accordingly, in another embodiment, disclosed herein are a subset of Formula I represented by Formula IB:

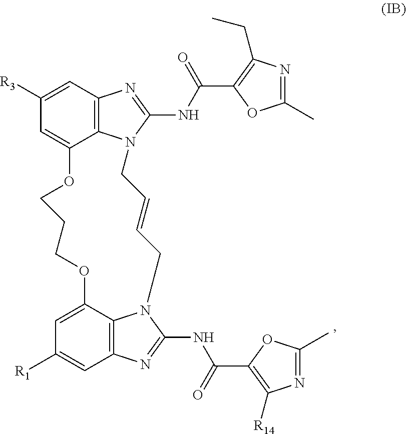

or a solvate, pharmaceutically acceptable salt, or tautomer thereof, wherein:
R₁ and R₃ are independently selected from the group consisting of:

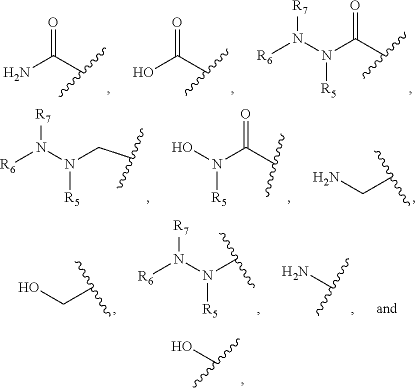

wherein optionally R₅ and R₆ are connected to form a 5- or 6-membered heterocyclic ring;
R₁₄ is hydrogen, or C₁₋₄ alkyl;
R₅, R₆, and R₇ are independently selected from hydrogen, —OH, C₁-C₆ alkyl,

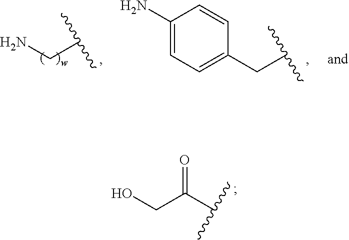

and
w is an integer from 1 to 6, provided that at least one of R₁ and R₃ is

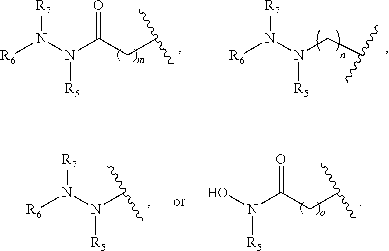

In some embodiments, both R₁ and R₃ are independently

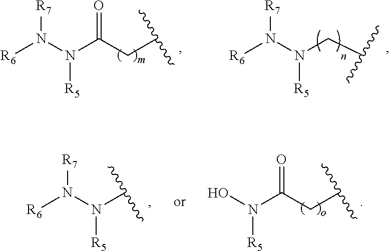

In other embodiments, compounds of Formula IB include those selected from the group consisting of

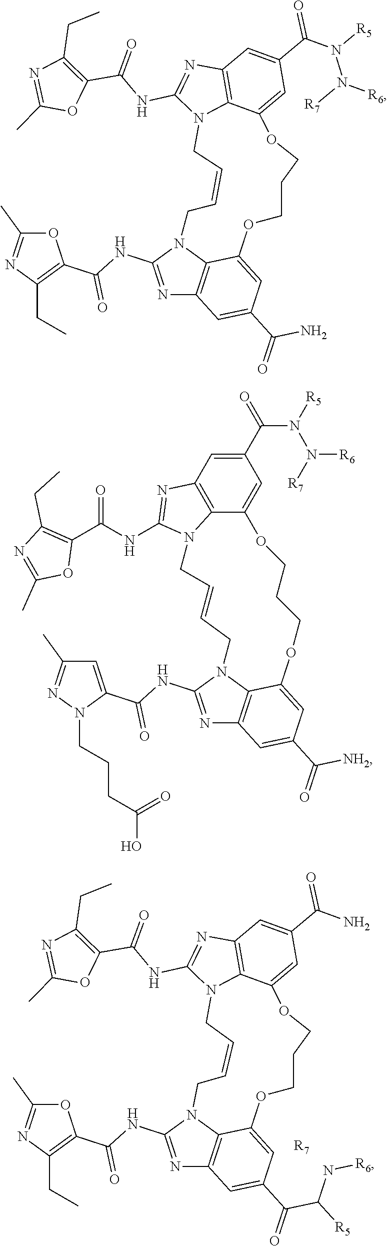

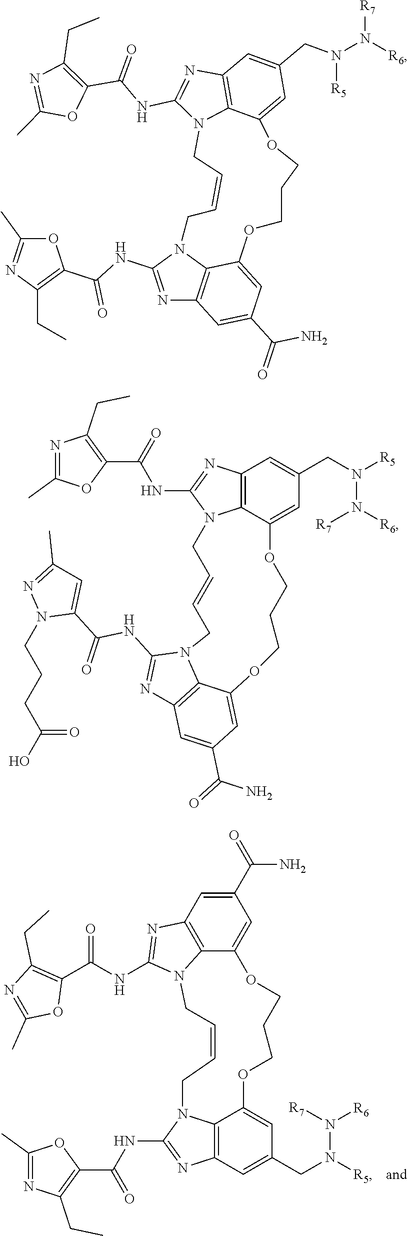

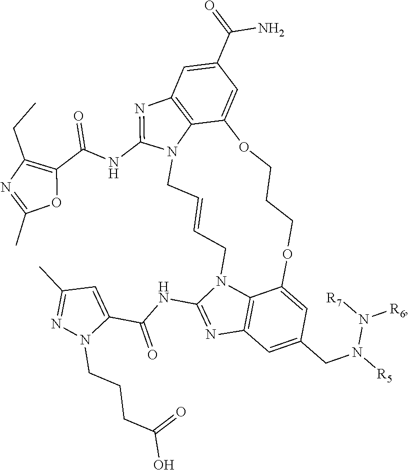

wherein R₅, R₆, and R₇ are as defined above.

In some embodiments, the compound of the present invention is selected from the compounds listed in Table 1. For any compound with a reference number, that reference number corresponds to a reference number in the working examples.

TABLE 1

| | | | | UPLC | |
|---|---|---|---|---|---|
| | | | | method: | |
| | | | MW | RT | LC-MS |
| Ref. | Structure | Formula | (g/mol) | (min) | (M + H)⁺ |
| I-A | 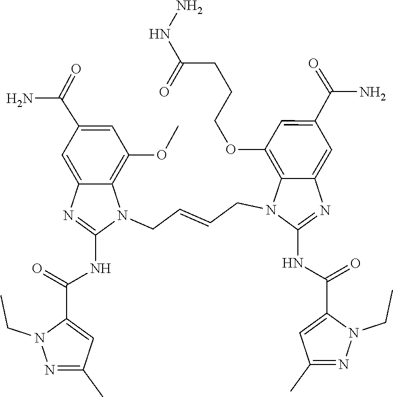 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NN)c21] | C39H46N14O7 | 822.88 | lcms_ long: 3.73 broad peak | 823.52 |
| I-D | 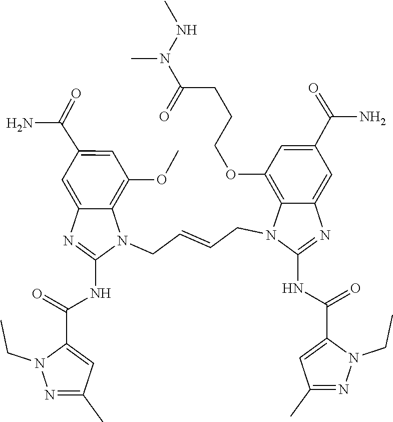 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)NC)c21] | C41H50N14O7 | 850.93 | lcms_ long: 4.01 | 851.56 |
| I-C | 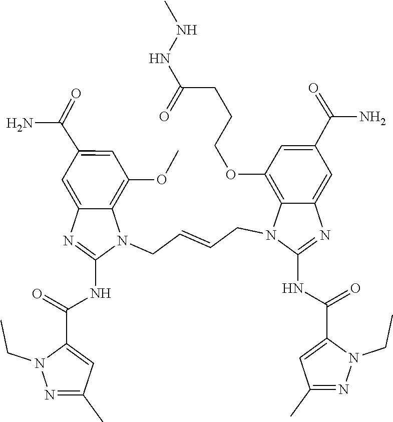 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NNC)c21] | C40H48N14O7 | 836.91 | lcms_ long: 3.87 | 837.59 |
| I-B | 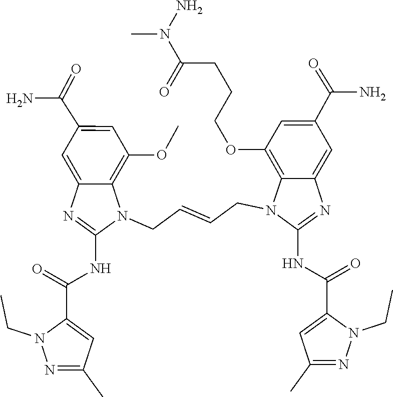 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)N)c21] | C40H48N14O7 | 836.91 | lcms_ long: 3.90 | 837.59 |
| II-A | 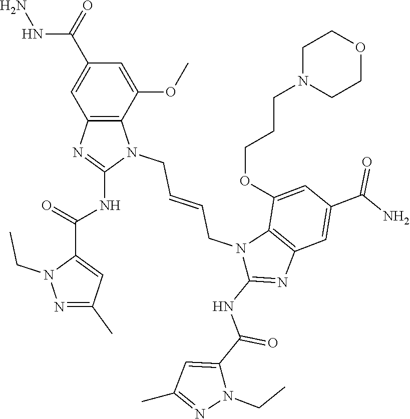 [SMILES: CCn1nc(C)cc1C(=O)Nc1nc2cc(C(=O)NN)cc(OC)c2n1C/C=C/Cn1c(NC(=O)c2cc(C)nn2CC)nc2cc(C(N)=O)cc(OCCCN3CCOCC3)c21] | C42H52N14O7 | 864.96 | lcms_ long: 3.52 broad lcms_ long C4: 3.41 Sharp | 865.65 |
| I-E | 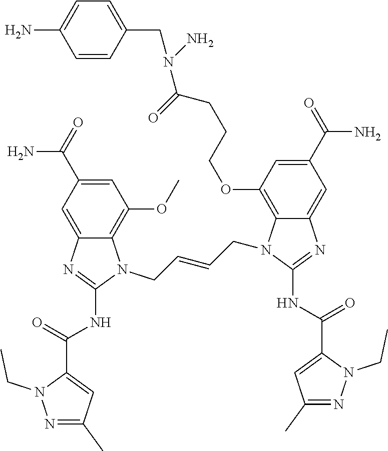 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(N)Cc3ccc(N)cc3)c21] | C46H53N15O7 | 928.02 | lcms_ long: 3.68 | 950.55 (M + Na⁺)⁺ |
| I-F | 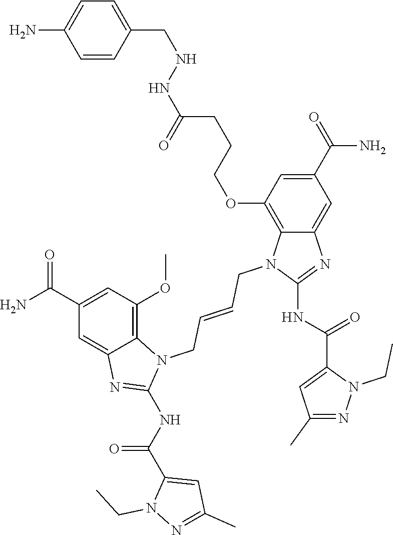 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NNCc3ccc(N)cc3)c21] | C46H53N15O7 | 928.02 | lcms_ long: 3.78 | 950.55 (M + Na⁺)⁺ |
| I-G | 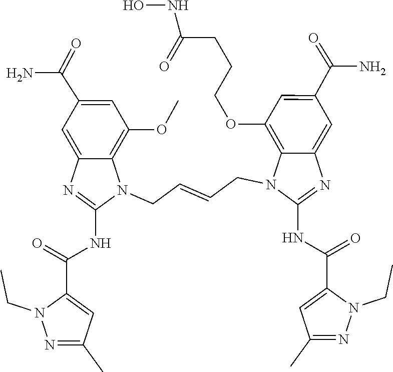 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NO)c21] | C39H45N13O8 | 823.87 | lcms_ long: 3.66 | 824.50 |
| I-H | 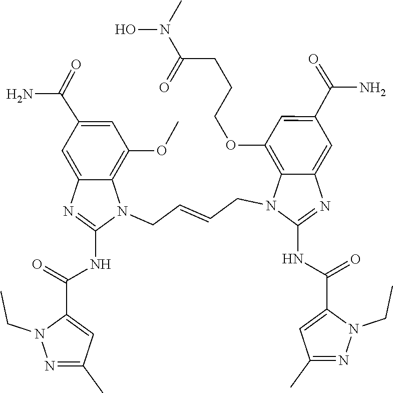 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)O)c21] | C40H47N13O8 | 837.89 | lcms_ long: 4.02 | 838.55 |
| I-I | 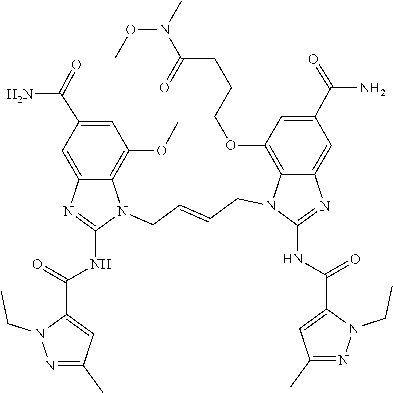 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)OC)c21] | C41H49N13O8 | 851.92 | lcms_ long: 4.36 | 852.55 |
| I-J | 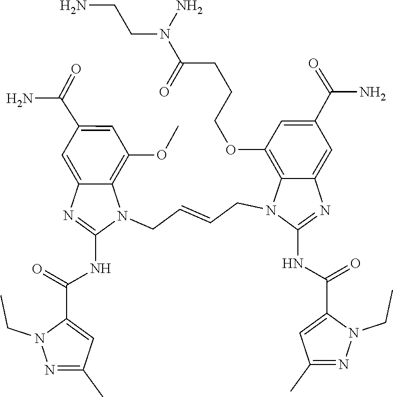 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(N)CCN)c21] | C41H51N15O7 | 865.95 | lcms_ long: 3.71 | 866.54 |
| I-K | 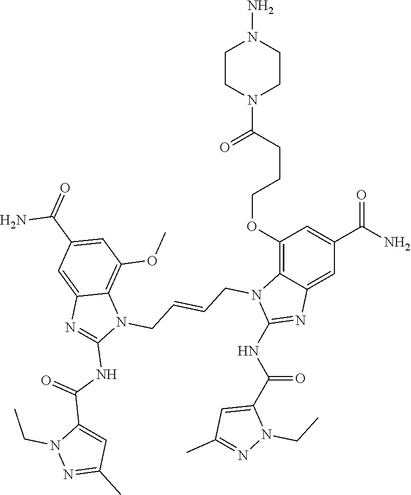 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N3CCN(N)CC3)c21] | C43H53N15O7 | 891.99 | lcms_ long: 3.74 | 892.64 |
| II-B | 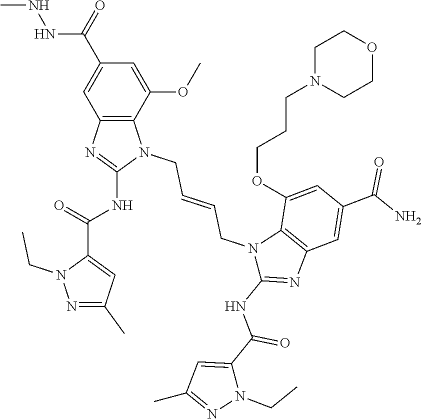 [SMILES: CCn1nc(C)cc1C(=O)Nc1nc2cc(C(=O)NNC)cc(OC)c2n1C/C=C/Cn1c(NC(=O)c2cc(C)nn2CC)nc2cc(C(N)=O)cc(OCCCN3CCOCC3)c21] | C43H54N14O7 | 878.99 | lcms_ long_ C4:3.37 | 879.65 |
| II-C | 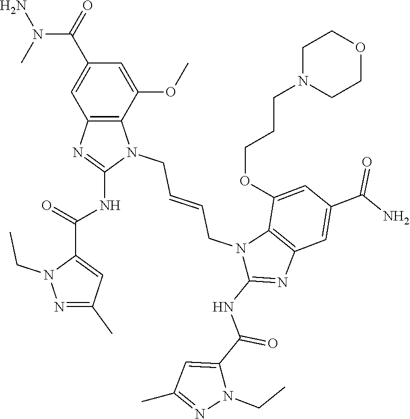 [SMILES: CCn1nc(C)cc1C(=O)Nc1nc2cc(C(=O)N(C)N)cc(OC)c2n1C/C=C/Cn1c(NC(=O)c2cc(C)nn2CC)nc2cc(C(N)=O)cc(OCCCN3CCOCC3)c21] | C43H54N14O7 | 878.99 | lcms_ long: 3.73 broad lcms_ long_ C4:3.53 | 879.63 |
| II-D | 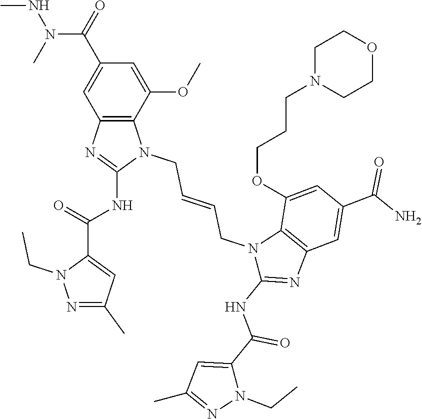 [SMILES: CCn1nc(C)cc1C(=O)Nc1nc2cc(C(=O)N(C)NC)cc(OC)c2n1C/C=C/Cn1c(NC(=O)c2cc(C)nn2CC)nc2cc(C(N)=O)cc(OCCCN3CCOCC3)c21] | C44H56N14O7 | 893.02 | lcms_ short: 3.06 broad | 893.64 |
| III | 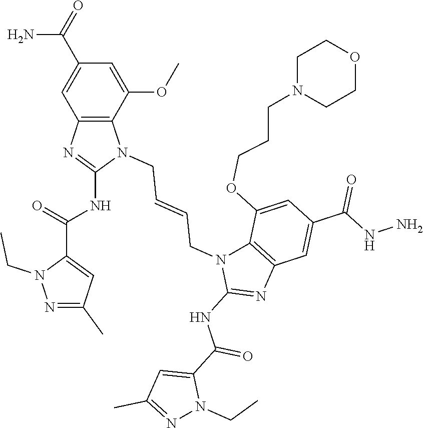 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NN)cc(OCCCN3CCOCC3)c21] | C42H52N14O7 | 864.96 | lcms_ long_ C4:3.46 | 865.59 |
| IV | 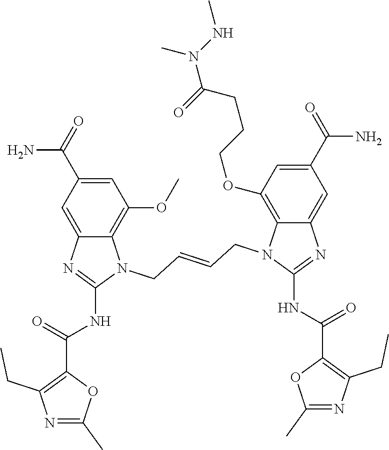 [SMILES: CCc1nc(C)oc1C(=O)Nc1nc2cc(C(N)=O)cc(OC)c2n1C/C=C/Cn1c(NC(=O)c2oc(C)nc2CC)nc2cc(C(N)=O)cc(OCCCC(=O)N(C)NC)c21] | C41H48N12O9 | 852.91 | lcms_ long: 3.69 | 853.51 |
| V | 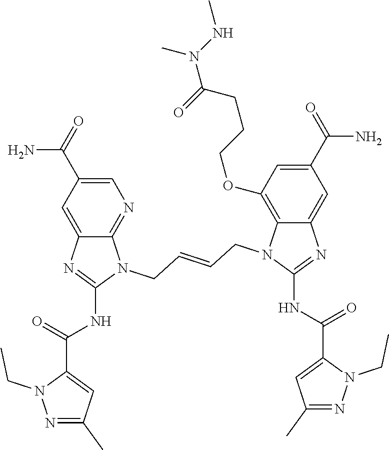 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCC(=O)N(C)NC)c21] | C39H47N15O6 | 821.90 | | |
| VI | 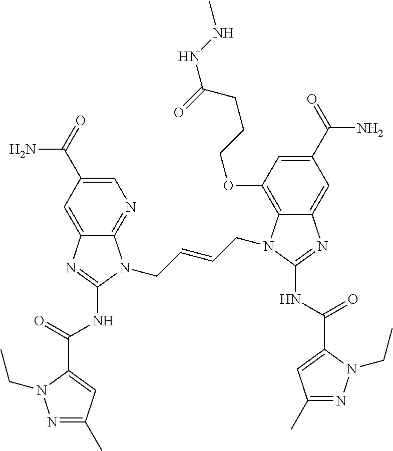 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCC(=O)NNC)c21] | C38H45N15O6 | 807.87 | | |
| VII | 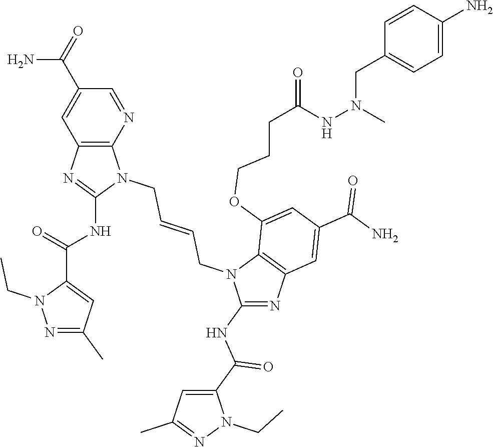 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCC(=O)NN(C)Cc3ccc(N)cc3)c21] | C45H52N16O6 | 913.01 | | |
| VIII | 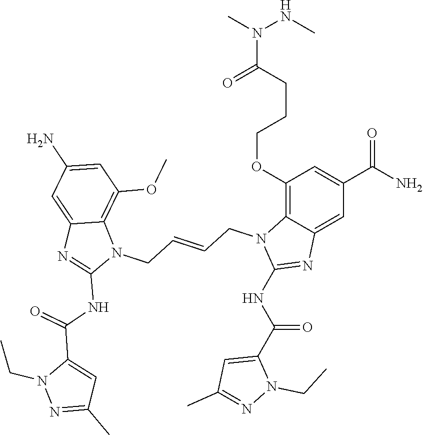 [SMILES: CCn1nc(C)cc1C(=O)Nc1nc2cc(N)cc(OC)c2n1C/C=C/Cn1c(NC(=O)c2cc(C)nn2CC)nc2cc(C(N)=O)cc(OCCCC(=O)N(C)NC)c21] | C40H50N14O6 | 822.93 | | |
| IX | 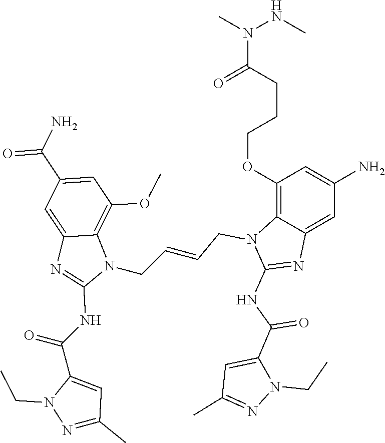 [SMILES: CCn1nc(C)cc1C(=O)Nc1nc2cc(C(N)=O)cc(OC)c2n1C/C=C/Cn1c(NC(=O)c2cc(C)nn2CC)nc2cc(N)cc(OCCCC(=O)N(C)NC)c21] | C40H50N14O6 | 822.93 | | |
| X | 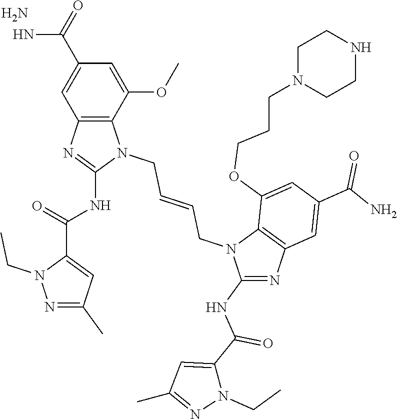 | C42H53N15O6 | 863.99 | | |
| XI | 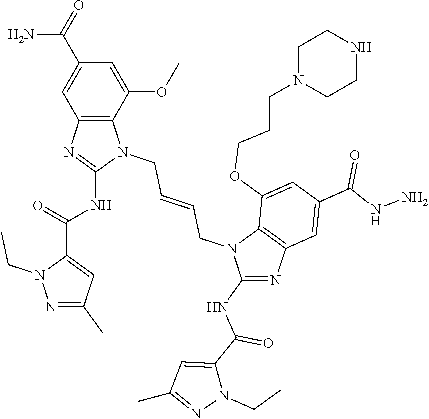 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NN)cc(OCCCN3CCNCC3)c21] | C42H53N15O6 | 863.99 | | |
| XII | 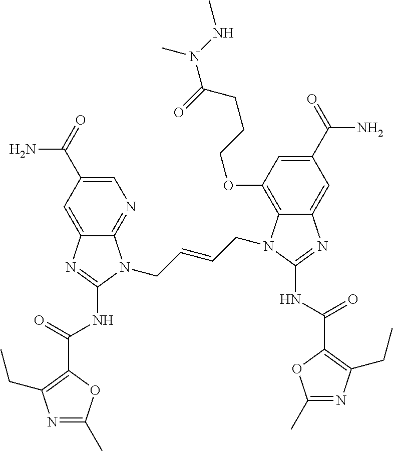 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCC(=O)N(C)NC)c21] | C39H45N13O8 | 823.87 | | |
| XIII | 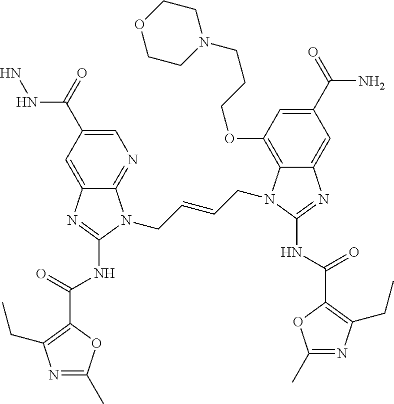 [SMILES: CCc1nc(C)oc1C(=O)Nc1nc2cc(C(=O)NN)cnc2n1C/C=C/Cn1c(NC(=O)c2oc(C)nc2CC)nc2cc(C(N)=O)cc(OCCCN3CCOCC3)c21] | C40H46N13O8 | 836.89 | | |
| XIV | 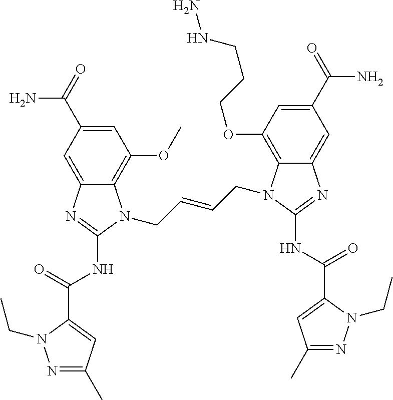 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCNN)c21] | C38H46N14O6 | 794.88 | | |
| XV | 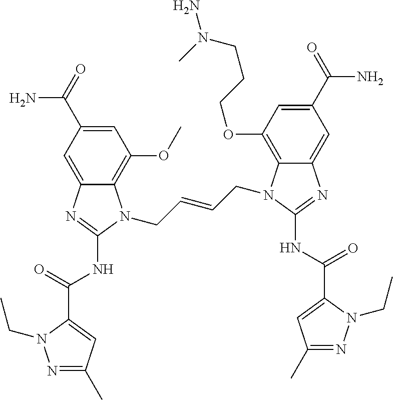 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)N)c21] | C39H48N14O6 | 808.90 | lcms short: 3.03 | 809.57 |
| XVI | 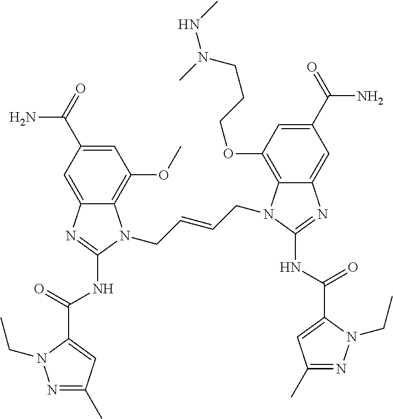 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)NC)c21] | C40H50N14O6 | 822.93 | | |
| XVII | 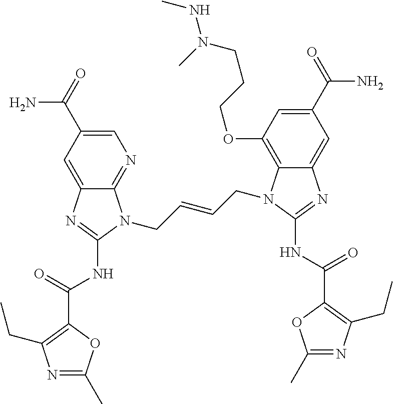 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(C)NC)c21] | C38H45N13O7 | 795.86 | | |
| XVIII | 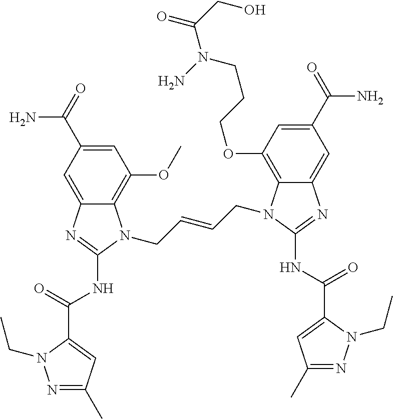 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(N)C(=O)CO)c21] | C40H48N14O8 | 852.91 | | |
| XIX | 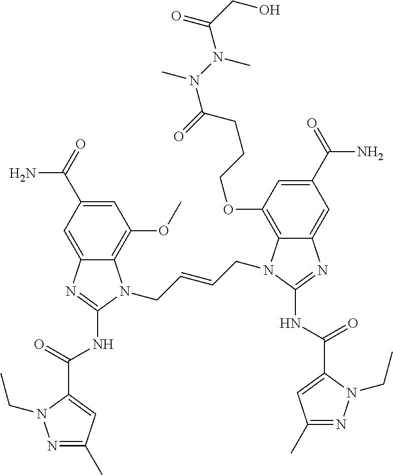 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)N(C)C(=O)CO)c21] | C43H52N14O9 | 908.98 | | |
| XX | 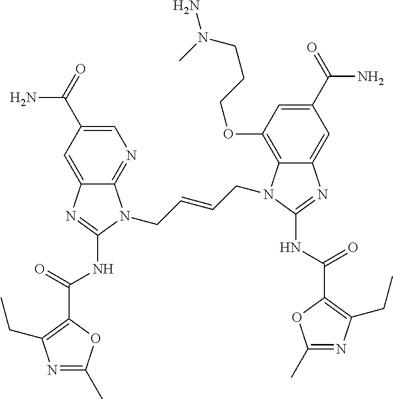 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(C)N)c21] | C37H43N13O7 | 781.84 | lcms_ long C4: 3.02 | 782.55 |
| XXI | 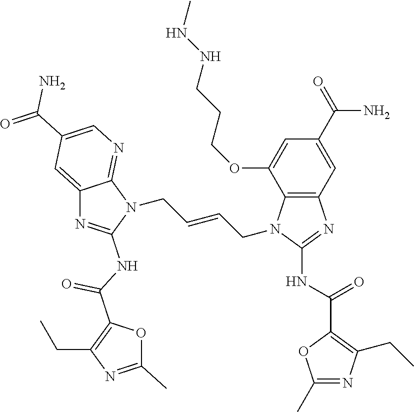 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCNNC)c21] | C37H43N13O7 | 781.84 | | |
| XXII | 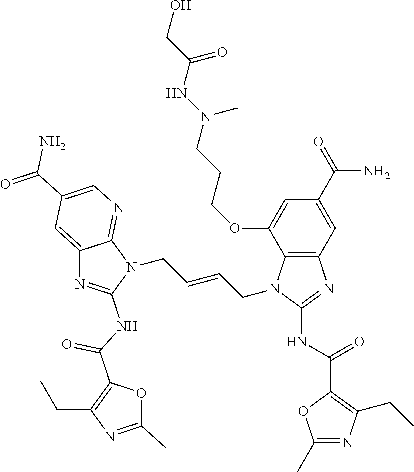 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(C)NC(=O)CO)c21] | C39H45N13O9 | 839.87 | | |
| XXIII | 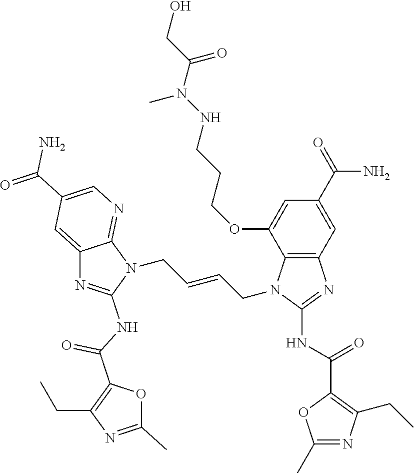 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCNN(C)C(=O)CO)c21] | C39H45N13O9 | 839.87 | | |
| XXIV | 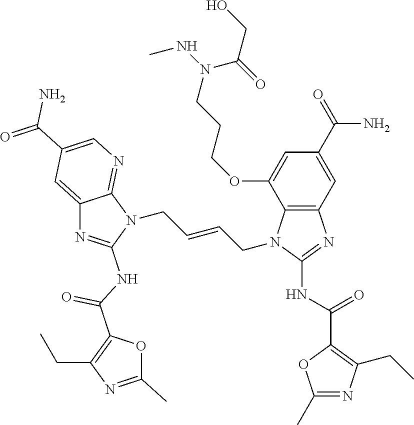 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(NC)C(=O)CO)c21] | C39H45N13O9 | 839.87 | | |
| XXV | 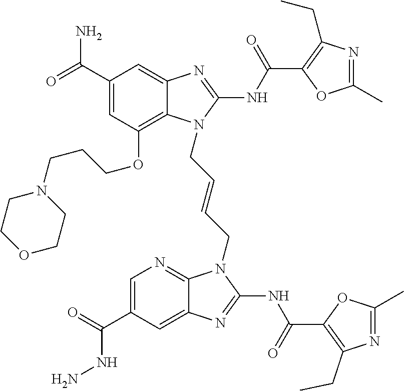 [SMILES: CCc1nc(C)oc1C(=O)Nc1nc2cc(C(=O)NN)cnc2n1C/C=C/Cn1c(NC(=O)c2oc(C)nc2CC)nc2cc(C(N)=O)cc(OCCCN3CCOCC3)c21] | C40H47N13O8 | 837.90 | | |
| XXVI | 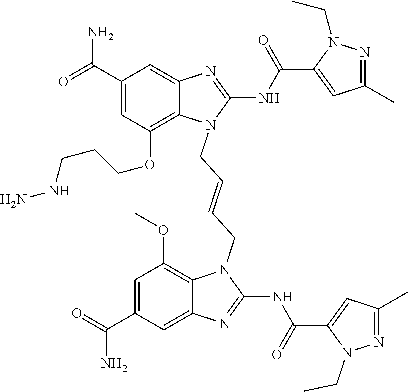 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCNN)c21] | C38H46N14O6 | 794.88 | | |
| XXVII | 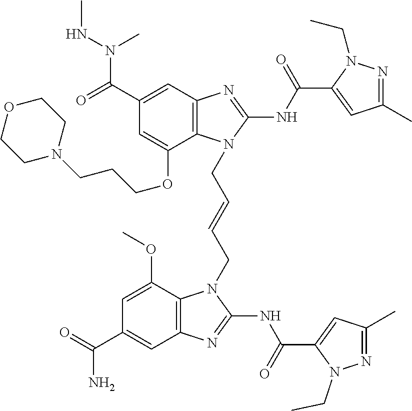 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)N(C)NC)cc(OCCCN3CCOCC3)c21] | C44H56N14O7 | 893.02 | | |
| XXVIII | 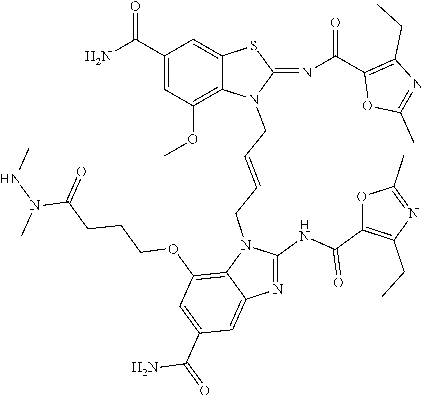 [SMILES: CCc1nc(C)oc1C(=O)/N=c1\sc2cc(C(N)=O)cc(OC)c2n1C/C=C/Cn1c(NC(=O)c2oc(C)nc2CC)nc2cc(C(N)=O)cc(OCCCC(=O)N(C)NC)c21] | C41H47N11O9S | 869.95 | LCMS Method B: 2.74 | 870.2 |
| XXIX | 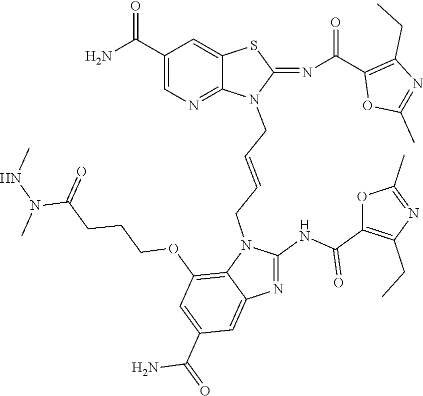 [SMILES: CCc1nc(C)oc1C(=O)/N=c1\sc2cc(C(N)=O)cnc2n1C/C=C/Cn1c(NC(=O)c2oc(C)nc2CC)nc2cc(C(N)=O)cc(OCCCC(=O)N(C)NC)c21] | C39H44N12O8S | 840.92 | LCMS Method D 4.24 | 842.2 |
| XXX | 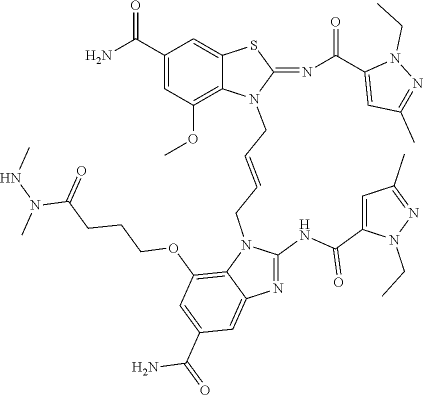 [SMILES: CCn1nc(C)cc1C(=O)/N=c1\sc2cc(C(N)=O)cc(OC)c2n1C/C=C/Cn1c(NC(=O)c2cc(C)nn2CC)nc2cc(C(N)=O)cc(OCCCC(=O)N(C)NC)c21] | C41H49N13O7S | 867.99 | LCMS Method A 2.86 | 868.2 |
| XXXI | 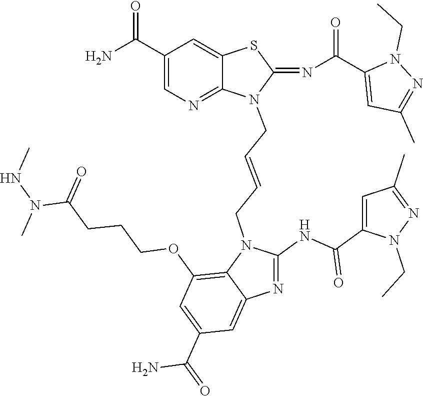 [SMILES: CCn1nc(C)cc1C(=O)/N=c1\sc2cc(C(N)=O)cnc2n1C/C=C/Cn1c(NC(=O)c2cc(C)nn2CC)nc2cc(C(N)=O)cc(OCCCC(=O)N(C)NC)c21] | C39H46N14O6S | 838.95 | LCMS Method D 4.70 | 839.4 |
| XXXII | 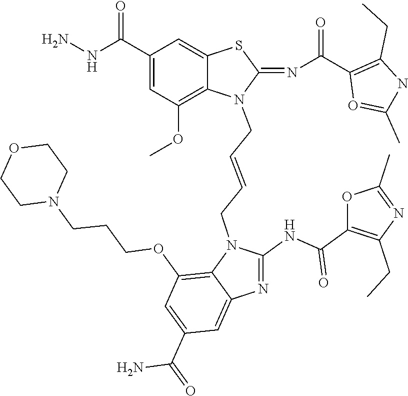 | C42H49N11O9S | 883.98 | LCMS Method C 2.72 | 884.4 |
| XXXIII | 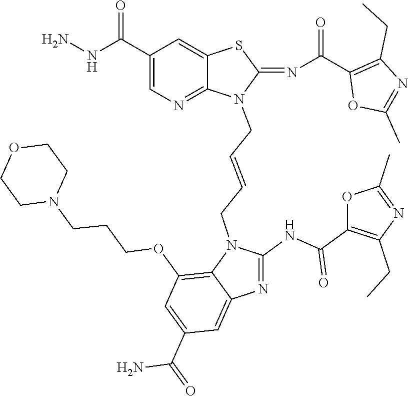 [SMILES: CCc1nc(C)oc1C(=O)/N=c1\sc2cc(C(=O)NN)cnc2n1C/C=C/Cn1c(NC(=O)c2oc(C)nc2CC)nc2cc(C(N)=O)cc(OCCCN3CCOCC3)c21] | C40H46N12O8S | 854.94 | LCMS Method B 1.97 | 855.2 |
| XXXIV | 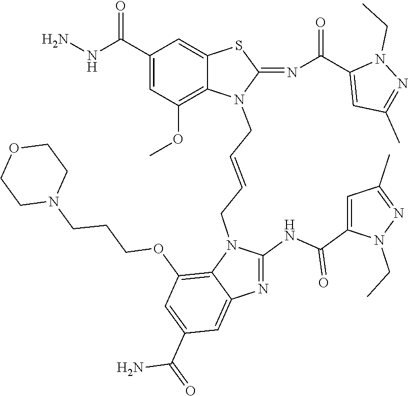 [SMILES: CCn1nc(C)cc1C(=O)/N=c1\sc2cc(C(=O)NN)cc(OC)c2n1C/C=C/Cn1c(NC(=O)c2cc(C)nn2CC)nc2cc(C(N)=O)cc(OCCCN3CCOCC3)c21] | C42H51N13O7S | 882.01 | LCMS Method C 2.94 | 882.5 |
| XXXV | 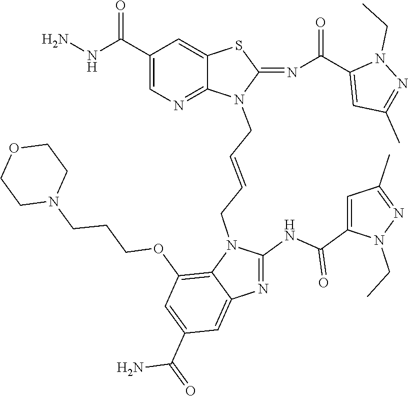 [SMILES: CCn1nc(C)cc1C(=O)/N=c1\sc2cc(C(=O)NN)cnc2n1C/C=C/Cn1c(NC(=O)c2cc(C)nn2CC)nc2cc(C(N)=O)cc(OCCCN3CCOCC3)c21] | C40H48N14O6S | 852.98 | | |
| XXXVI | 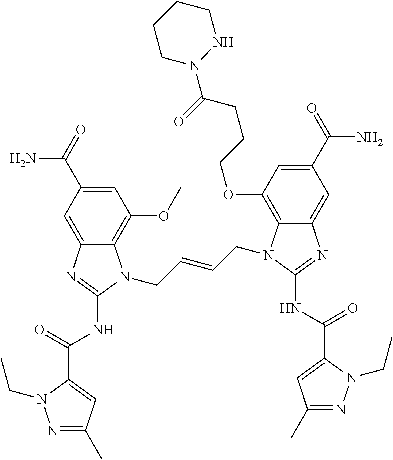 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N3CCCCN3)c21] | C43H52N14O7 | 876.98 | | |
| XXXVII | 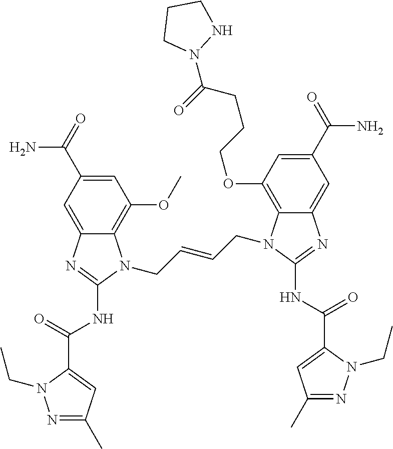 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N3CCCN3)c21] | C42H50N14O7 | 862.95 | | |
| XXXVIII | 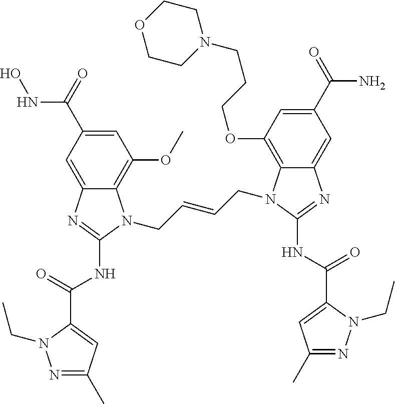 [SMILES: CCn1nc(C)cc1C(=O)Nc1nc2cc(C(=O)NO)cc(OC)c2n1C/C=C/Cn1c(NC(=O)c2cc(C)nn2CC)nc2cc(C(N)=O)cc(OCCCN3CCOCC3)c21] | C42H51N13O8 | 865.95 | | |
| XXXIX | 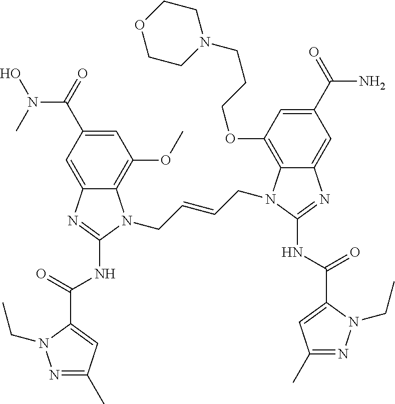 [SMILES: CCn1nc(C)cc1C(=O)Nc1nc2cc(C(=O)N(C)O)cc(OC)c2n1C/C=C/Cn1c(NC(=O)c2cc(C)nn2CC)nc2cc(C(N)=O)cc(OCCCN3CCOCC3)c21] | C43H53N13O8 | 879.98 | | |
| XL | 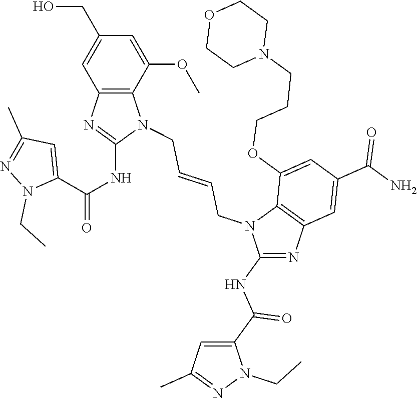 [SMILES: CCn1nc(C)cc1C(=O)Nc1nc2cc(CO)cc(OC)c2n1C/C=C/Cn1c(NC(=O)c2cc(C)nn2CC)nc2cc(C(N)=O)cc(OCCCN3CCOCC3)c21] | | | | |
| XLI | 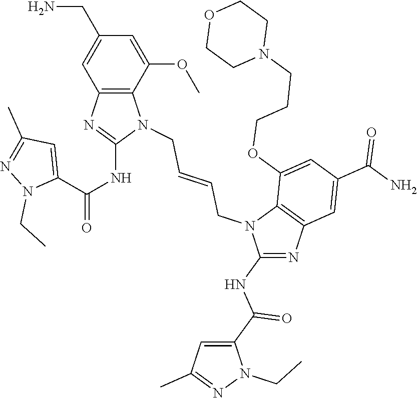 [SMILES: CCn1nc(C)cc1C(=O)Nc1nc2cc(CN)cc(OC)c2n1C/C=C/Cn1c(NC(=O)c2cc(C)nn2CC)nc2cc(C(N)=O)cc(OCCCN3CCOCC3)c21] | | | | |
| XLII | 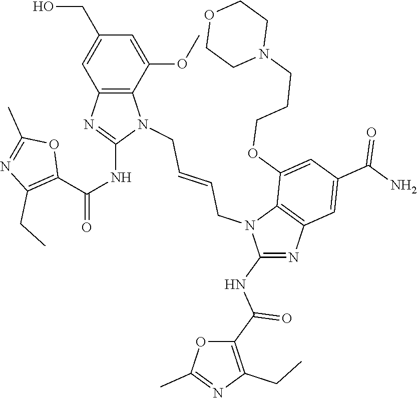 [SMILES: CCc1nc(C)oc1C(=O)Nc1nc2cc(CO)cc(OC)c2n1C/C=C/Cn1c(NC(=O)c2oc(C)nc2CC)nc2cc(C(N)=O)cc(OCCCN3CCOCC3)c21] | | | | |
| XLIII | 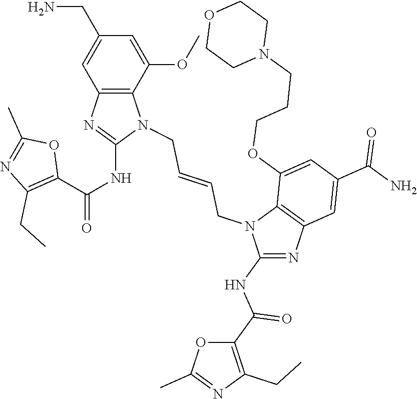 [SMILES: CCc1nc(C)oc1C(=O)Nc1nc2cc(CN)cc(OC)c2n1C/C=C/Cn1c(NC(=O)c2oc(C)nc2CC)nc2cc(C(N)=O)cc(OCCCN3CCOCC3)c21] | | | | |
| XLIV | 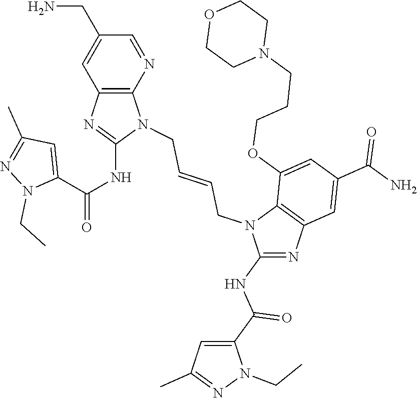 [SMILES: CCn1nc(C)cc1C(=O)Nc1nc2cc(CN)cnc2n1C/C=C/Cn1c(NC(=O)c2cc(C)nn2CC)nc2cc(C(N)=O)cc(OCCCN3CCOCC3)c21] | | | | |
| XLV | 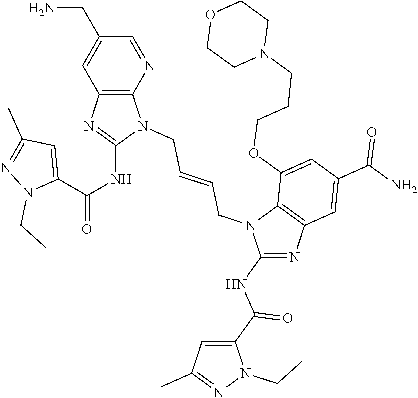 [SMILES: CCn1nc(C)cc1C(=O)Nc1nc2cc(CN)cnc2n1C/C=C/Cn1c(NC(=O)c2cc(C)nn2CC)nc2cc(C(N)=O)cc(OCCCN3CCOCC3)c21] | | | | |
| XLVI | 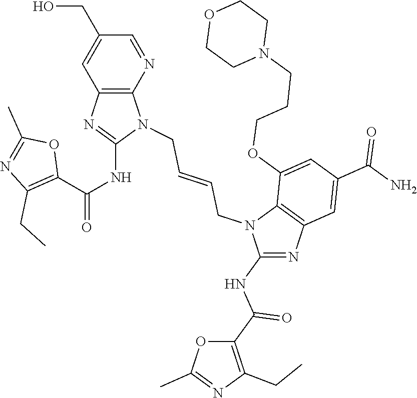 [SMILES: CCc1nc(C)oc1C(=O)Nc1nc2cc(CO)cnc2n1C/C=C/Cn1c(NC(=O)c2oc(C)nc2CC)nc2cc(C(N)=O)cc(OCCCN3CCOCC3)c21] | | | | |
| XLVII | 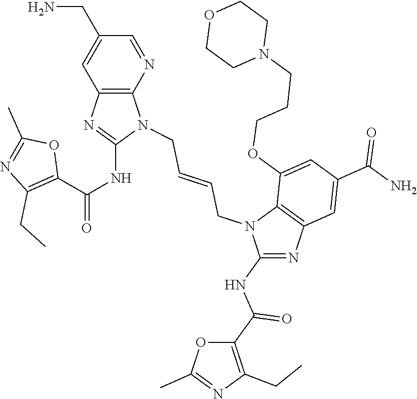 [SMILES: CCc1nc(C)oc1C(=O)Nc1nc2cc(CN)cnc2n1C/C=C/Cn1c(NC(=O)c2oc(C)nc2CC)nc2cc(C(N)=O)cc(OCCCN3CCOCC3)c21] | | | | |
| | 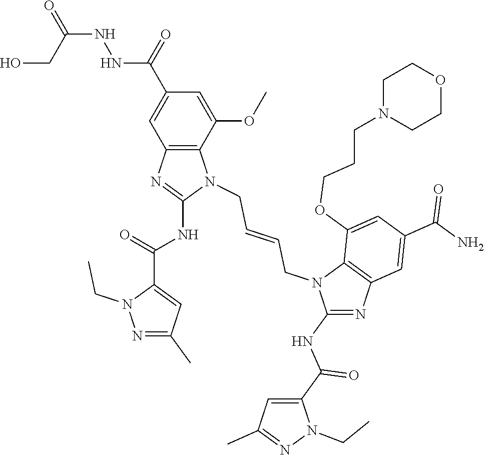 [SMILES: CCn1nc(C)cc1C(=O)Nc1nc2cc(C(=O)NNC(=O)CO)cc(OC)c2n1C/C=C/Cn1c(NC(=O)c2cc(C)nn2CC)nc2cc(C(N)=O)cc(OCCCN3CCOCC3)c21] | C44H54N14O9 | 923.00 | lcms_ long C4: 2.98 | 924.55 |
| | 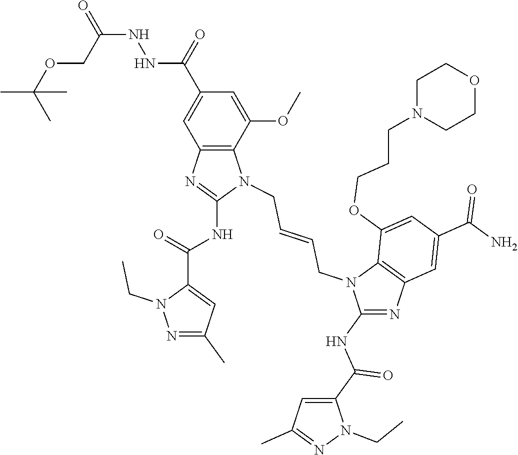 [SMILES: CCn1nc(C)cc1C(=O)Nc1nc2cc(C(=O)NNC(=O)COC(C)(C)C)cc(OC)c2n1C/C=C/Cn1c(NC(=O)c2cc(C)nn2CC)nc2cc(C(N)=O)cc(OCCCN3CCOCC3)c21] | C48H62N14O9 | 979.11 | lcms_ long C4: 3.4 | 981.75 |
| | 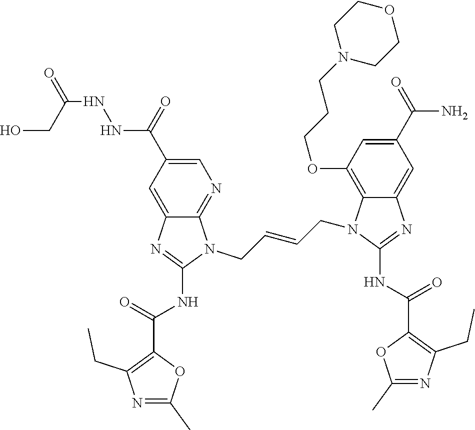 [SMILES: CCc1nc(C)oc1C(=O)Nc1nc2cc(C(=O)NNC(=O)CO)cnc2n1C/C=C/Cn1c(NC(=O)c2oc(C)nc2CC)nc2cc(C(N)=O)cc(OCCCN3CCOCC3)c21] | C42H49N13O10 | 895.94 | | |

The compounds of the present application are suitably formulated in a conventional manner into compositions using one or more carriers. Accordingly, the present application also includes a composition comprising one or more compounds of the application and a carrier. The compounds of the application are suitably formulated into pharmaceutical compositions for administration to subjects in a biologically compatible form suitable for administration in vivo. Accordingly, the present application further includes a pharmaceutical composition comprising one or more compounds of the application and a pharmaceutically acceptable carrier.

The compounds of the application may be administered to a subject in a variety of forms depending on the selected route of administration, as will be understood by those skilled in the art. A compound of the application may be administered, for example, by oral, parenteral, buccal, sublingual, nasal, rectal, patch, pump or transdermal administration and the pharmaceutical compositions formulated accordingly. Administration can be by means of a pump for periodic or continuous delivery.

Parenteral administration includes intravenous, intra-arterial, intraperitoneal, subcutaneous, intramuscular, transepithelial, nasal, intrapulmonary (for example, by use of an aerosol), intrathecal, rectal and topical (including the use of a patch or other transdermal delivery device) modes of administration. Parenteral administration may be by continuous infusion over a selected period of time. Conventional procedures and ingredients for the selection and preparation of suitable compositions are known to those skilled in the art.

A compound of the application may be orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the compound may be incorporated with excipient and used in the form of ingestible tablets, buccal tablets, troches, capsules, caplets, pellets, granules, lozenges, chewing gum, powders, syrups, elixirs, wafers, aqueous solutions and suspensions, and the like. In the case of tablets, carriers that are used include lactose, corn starch, sodium citrate and salts of phosphoric acid. Pharmaceutically acceptable excipients include binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. In the case of tablets, capsules, caplets, pellets or granules for oral administration, pH sensitive enteric coatings designed to control the release of active ingredients are optionally used. Oral dosage forms also include modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. Liposome delivery systems include, for example, small unilamellar vesicles, large unilamellar vesicles and multilamellar vesicles. Liposomes can be formed from a variety of phospholipids, such as cholesterol, stearylamine or phosphatidylcholines. For oral administration in a capsule form, useful carriers or diluents include lactose and dried corn starch.

Liquid preparations for oral administration may take the form of, for example, solutions, syrups or suspensions, or they are suitably presented as a dry product for constitution with water or other suitable vehicle before use. When aqueous suspensions and/or emulsions are administered orally, the compound of the application is suitably suspended or dissolved in an oily phase that is combined with emulsifying and/or suspending agents. If desired, certain sweetening and/or flavoring and/or coloring agents may be added. Such liquid preparations for oral administration may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid). Useful diluents include lactose and high molecular weight polyethylene glycols.

It is also possible to freeze-dry the compounds of the application and use the lyophilizates obtained, for example, for the preparation of products for injection.

A compound of the application may also be administered parenterally. Solutions of a compound of the application can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. For parenteral administration, sterile solutions of the compounds of the application are usually prepared, and the pH of the solutions are suitably adjusted and buffered. For intravenous use, the total concentration of solutes should be controlled to render the preparation isotonic. For ocular administration, ointments or droppable liquids may be delivered by ocular delivery systems known to the art such as applicators or eye droppers. Such compositions can include mucomimetics such as hyaluronic acid, chondroitin sulfate, hydroxypropyl methylcellulose or polyvinyl alcohol, preservatives such as sorbic acid, EDTA or benzyl chromium chloride, and the usual quantities of diluents or carriers. For pulmonary administration, diluents or carriers will be selected to be appropriate to allow the formation of an aerosol.

The compounds of the application may be formulated for parenteral administration by injection, including using conventional catheterization techniques or infusion. Formulations for injection may be presented in unit dosage form, e.g., in ampoules or in multi-dose containers, with an added preservative. The compositions may take such forms as sterile suspensions, solutions or emulsions in oily or aqueous vehicles, and may contain formulating agents such as suspending, stabilizing and/or dispersing agents. In all cases, the form must be sterile and must be fluid to the extent that easy syringability exists. Alternatively, the compounds of the application are suitably in a sterile powder form for reconstitution with a suitable vehicle, e.g., sterile pyrogen-free water, before use.

Compositions for nasal administration may conveniently be formulated as aerosols, drops, gels and powders.

For intranasal administration or administration by inhalation, the compounds of the application are conveniently delivered in the form of a solution, dry powder formulation or suspension from a pump spray container that is squeezed or pumped by the patient or as an aerosol spray presentation from a pressurized container or a nebulizer. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

In some embodiments, compounds of the application may also be coupled with soluble polymers as targetable drug carriers. Such polymers can include polyvinylpyrrolidone, pyran copolymer, polyhydroxypropylmethacrylamide-phenol, polyhydroxy-ethylaspartamide-phenol, or polyethyleneoxide-polylysine substituted with palmitoyl residues. Furthermore, compounds of the application may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and crosslinked or amphipathic block copolymers of hydrogels.

In some embodiments, one or more compounds of the application may also be coupled to suitable antibodies as targetable drug carriers. For example, compounds of application may be coupled to suitable antibodies to form an antibody-drug conjugate (ADC), as described for example in Polakis, P., Pharmacol. Revs., 2016, 68, 3-19. Therefore the present application includes an ADC comprising one or more compounds of the application. An antibody may generally include any polypeptide comprising a framework region from an immunoglobulin or fragments thereof that specifically binds and recognizes an antigen, such as a carbohydrate, polynucleotide, lipid, polypeptide, etc. In some embodiments, the antibody is one that specifically binds to a cancer antigen, for example known cancer antigens CD22, CD33, CD30, HER2, Mesothelin, Melan-A, CD19, CD20, CD79b, Trop2, HER3, MAGE, MART-1. In some embodiments, the antibody is one that specifically binds to an immune cell surface receptor, for example CD80, CD86, GMCSF-R, DC-SIGN, CD36. In some embodiments, the antibody is one that specifically binds to an abnormally expressed protein characteristic of a cancer. In some embodiments, compounds of application may be coupled to suitable antibodies to form an ADC through a linker. The linker may be or comprise a cleavable group that allows the compound of the application to be cleaved from the rest of the conjugate in vivo by the biological environment. In some embodiments, the linker is cleaved in or near the desired target site of action. In some embodiments, the linker is noncleavable.

In some embodiments, compounds of the application may be coupled with suitable viral, non-viral or other vectors. Viral vectors may include retrovirus, lentivirus, adenovirus, herpesvirus, poxvirus, alphavirus, vaccinia virus and/or adeno-associated viruses. Non-viral vectors may include nanoparticles, cationic lipids, cationic polymers, metallic nanoparticles, nanorods, liposomes, micelles, microbubbles, cell-penetrating peptides, and/or lipospheres. Nanoparticles may include silica, lipid, carbohydrate, and/or other pharmaceutically acceptable polymers. The compounds of the application including pharmaceutically acceptable salts and solvates thereof are suitably used on their own but will generally be administered in the form of a pharmaceutical composition in which the one or more compounds of the application (the active ingredient) is in association with a pharmaceutically acceptable carrier. Depending on the mode of administration, the pharmaceutical composition will comprise from about 0.05 wt % to about 99 wt % or about 0.10 wt % to about 70 wt %, of the active ingredient (one or more compounds of the application), and from about 1 wt % to about 99.95 wt % or about 30 wt % to about 99.90 wt % of a pharmaceutically acceptable carrier, all percentages by weight being based on the total composition.

Compounds of the application may be used alone or in combination with other known agents useful for treating diseases, disorders or conditions mediated by STING protein activation, or that are treatable by activation of STING. When used in combination with other agents useful in treating diseases, disorders or conditions mediated by STING protein activation, or that are treatable by activation of STING, it is an embodiment that the compounds of the application are administered contemporaneously with those agents. As used herein, “contemporaneous administration” of two substances to a subject means providing each of the two substances so that they are both biologically active in the individual at the same time. The exact details of the administration will depend on the pharmacokinetics of the two substances in the presence of each other and can include administering the two substances within a few hours of each other, or even administering one substance within 24 hours of administration of the other, if the pharmacokinetics are suitable. Design of suitable dosing regimens is routine for one skilled in the art. In particular embodiments, two substances will be administered substantially simultaneously, i.e., within minutes of each other, or in a single composition that contains both substances. It is a further embodiment of the present application that a combination of agents is administered to a subject in a non-contemporaneous fashion. In an embodiment, a compound of the present application is administered with another therapeutic agent simultaneously or sequentially in separate unit dosage forms or together in a single unit dosage form. Accordingly, the present application provides a single unit dosage form comprising one or more compounds of the application (e.g., a compound of Formula I), an additional therapeutic agent, and a pharmaceutically acceptable carrier.

The dosage of compounds of the application can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the subject to be treated. One of skill in the art can determine the appropriate dosage based on the above factors. Compounds of the application may be administered initially in a suitable dosage that may be adjusted as required, depending on the clinical response. Dosages will generally be selected to maintain a serum level of compounds of the application from about 0.01 μg/cc to about 1000 μg/cc, or about 0.1 μg/cc to about 100 μg/cc. As a representative example, oral dosages of one or more compounds of the application will range between about 1 mg per day to about 1000 mg per day for an adult, suitably about 1 mg per day to about 500 mg per day, more suitably about 1 mg per day to about 200 mg per day. For parenteral administration, a representative amount is from about 0.001 mg/kg to about 10 mg/kg, about 0.01 mg/kg to about 10 mg/kg, about 0.01 mg/kg to about 1 mg/kg or about 0.1 mg/kg to about 1 mg/kg will be administered. For oral administration, a representative amount is from about 0.001 mg/kg to about 10 mg/kg, about 0.1 mg/kg to about 10 mg/kg, about 0.01 mg/kg to about 1 mg/kg or about 0.1 mg/kg to about 1 mg/kg. For administration in suppository form, a representative amount is from about 0.1 mg/kg to about 10 mg/kg or about 0.1 mg/kg to about 1 mg/kg.

In an embodiment of the application, compositions are formulated for oral administration and the compounds are suitably in the form of tablets containing 0.25, 0.5, 0.75, 1.0, 5.0, 10.0, 20.0, 25.0, 30.0, 40.0, 50.0, 60.0, 70.0, 75.0, 80.0, 90.0, 100.0, 150, 200, 250, 300, 350, 400, 450, 500, 550, 600, 650, 700, 750, 800, 850, 900, 950 or 1000 mg of active ingredient per tablet. Compounds of the application may be administered in a single daily, weekly or monthly dose or the total daily dose may be divided into two, three or four daily doses.

To be clear, in the above, the term “a compound” also includes embodiments wherein one or more compounds are referenced.

### Methods and Uses of Disclosed Compounds

The compounds of the application have been shown to be capable of activating STING activity, such as STING protein activity.

Accordingly, the present application includes a method for activating STING in a cell, either in a biological sample or in a patient, comprising administering an effective amount of one or more compounds of the application to the cell. The application also includes a use of one or more compounds of the application for activating STING in a cell as well as a use of one or more compounds of the application for the preparation of a medicament for activating STING in a cell. The application further includes one or more compounds of the application for use in activating STING in a cell.

As the compounds of the application have been shown to be capable of activating STING protein activity, the compounds of the application are useful for treating diseases, disorders or conditions by activating STING. Therefore, the compounds of the present application are useful as medicaments. Accordingly, the present application includes a compound of the application for use as a medicament.

The present application also includes a method of treating a disease, disorder or condition by activation of STING comprising administering a therapeutically effective amount of one or more compounds of the application to a subject in need thereof.

The present application also includes a use of one or more compounds of the application for treatment of a disease, disorder, or condition by activation of STING as well as a use of one or more compounds of the application for the preparation of a medicament for treatment of a disease, disorder or condition by activation of STING. The application further includes one or more compounds of the application for use in treating a disease, disorder, or condition by activation of STING.

In an embodiment, the disease, disorder, or condition is a neoplastic disorder. Accordingly, the present application also includes a method of treating a neoplastic disorder comprising administering a therapeutically effective amount of one or more compounds of the application to a subject in need thereof. The present application also includes a use of one or more compounds of the application for treatment of a neoplastic disorder as well as a use of one or more compounds of the application for the preparation of a medicament for treatment of a neoplastic disorder. The application further includes one or more compounds of the application for use in treating a neoplastic disorder. In an embodiment, the treatment is in an amount effective to ameliorate at least one symptom of the neoplastic disorder, for example, reduced cell proliferation or reduced tumor mass, among others, in a subject in need of such treatment.

Compounds of the application have been demonstrated to be effective against a panel of human tumor cell line. Therefore, in another embodiment of the present application, the disease, disorder, or condition that is treated by activation of STING is cancer. Accordingly, the present application also includes a method of treating cancer comprising administering a therapeutically effective amount of one or more compounds of the application to a subject in need thereof. The present application also includes a use of one or more compounds of the application for treatment of cancer as well as a use of one or more compounds of the application for the preparation of a medicament for treatment of cancer. The application further includes one or more compounds of the application for use in treating cancer. In an embodiment, the compound is administered for the prevention of cancer in a subject such as a mammal having a predisposition for cancer.

In an embodiment, the cancer is selected from a cancer of the skin, blood, prostate, colorectum, pancreas, kidney, ovary, breast, for example mammary, liver, tongue and lung. In another embodiment, the cancer is selected from leukaemia, lymphoma, non-Hodgkin's lymphoma and multiple myeloma. In a further embodiment of the present application, the cancer is selected from leukemia, melanoma, lung cancer, colon cancer, brain cancer, ovarian cancer, breast cancer, prostate cancer and kidney cancer.

In an embodiment, the disease, disorder or condition that is treated by activation of STING is a disease, disorder or condition associated with an uncontrolled and/or abnormal cellular activity affected directly or indirectly by activation of STING. In another embodiment, the uncontrolled and/or abnormal cellular activity that is affected directly or indirectly by activation of STING is proliferative activity in a cell. Accordingly, the application also includes a method of inhibiting proliferative activity in a cell, comprising administering an effective amount of one or more compounds of the application to the cell. The present application also includes a use of one or more compounds of the application for inhibition of proliferative activity in a cell as well as a use of one or more compounds of the application for the preparation of a medicament for inhibition of proliferative activity in a cell. The application further includes one or more compounds of the application for use in inhibiting proliferative activity in a cell.

The present application also includes a method of inhibiting uncontrolled and/or abnormal cellular activities affected directly or indirectly by STING protein in a cell, either in a biological sample or in a subject, comprising administering an effective amount of one or more compounds of the application to the cell. The application also includes a use of one or more compounds of the application for inhibition of uncontrolled and/or abnormal cellular activities affected directly or indirectly by STING protein in a cell as well as a use of one or more compounds of the application for the preparation of a medicament for inhibition of uncontrolled and/or abnormal cellular activities affected directly or indirectly by STING protein in a cell. The application further includes one or more compounds of the application for use in inhibiting uncontrolled and/or abnormal cellular activities affected directly or indirectly by STING protein in a cell.

Accordingly, the present application also includes a method of treating a disease, disorder or condition that is treatable by activation of STING comprising administering a therapeutically effective amount of one or more compounds of the application in combination with another known agent useful for treatment of a disease, disorder or condition treatable by activation of STING to a subject in need thereof. The present application also includes a use of one or more compounds of the application in combination with another known agent useful for treatment of a disease, disorder or condition treatable by activation of STING for treatment of a disease, disorder or condition treatable by activation of STING, as well as a use of one or more compounds of the application in combination with another known agent useful for treatment of a disease, disorder or condition treatable by activation of STING for the preparation of a medicament for treatment of a disease, disorder or condition treatable by activation of STING. The application further includes one or more compounds of the application in combination with another known agent useful for treatment of a disease, disorder or condition treatable by activation of STING for use in treating a disease, disorder or condition treatable by activation of STING. In an embodiment, the disease, disorder or condition treatable by activation of STING is cancer such as acute myeloid leukemia (AML), acute lymphocytic leukemia (ALL), melanoma, prostate cancer, endometrial cancer, schwannoma, mantel cell lymphoma, rhabdomyosarcoma, glioma, glioblastoma, B-ALL, T-ALL, lung cancer, gastric cancer, pancreatic cancer and breast cancer.

In a further embodiment, the disease, disorder or condition treatable by activation of STING is cancer and the one or more compounds of the application are administered in combination with one or more additional cancer treatments. In another embodiment, the additional cancer treatment is selected from radiotherapy, chemotherapy, targeted therapies such as antibody therapies and small molecule therapies such as tyrosine-kinase inhibitors, immunotherapy, hormonal therapy and anti-angiogenic therapies.

In some embodiments, the subject is a mammal. In some embodiments, the subject is human.

### General Synthesis

Compounds disclosed herein can be prepared using known organic synthesis techniques and can be synthesized according to any of numerous possible synthetic routes, examples of which are described in the following working examples.

### EXAMPLES

As depicted in the Examples below, in certain exemplary aspects, compounds are prepared according to the following general procedures. It will be appreciated that, although the general methods depict the synthesis of certain compounds of the present disclosure, the following general methods, and other methods known to one of ordinary skill in the art, can be applied to all compounds and subclasses and species of each of these compounds, as described herein.

### Materials and Methods (Examples 1-19 and 29)

UPLC-MS Instrument and Methods

Instrument: Waters H-Class UPLC with QSM, sample organizer, column heater, PDa UV detector and Qda mass spectrometer.

Column: Waters BEH C₁₈ Column, 100×2.1 mm, 1.7 μm, 130 Å pore size

Lcms_long method: 40° C. column temperature. UV absorption wavelength: 214 nm. MS range: 200-1250 Da. Mobile phase A: 0.1% TFA in water. Mobile phase B: 0.085% TFA in acetonitrile. Flowrate: 0.5 mL/min. Gradient:

| Time (min) | % B |
|---|---|
| 0.0 | 10 |
| 8.0 | 80 |
| 8.1 | 90 |
| 9.0 | 90 |
| 9.1 | 10 |
| 11.0 | 10 |

Column: Waters BEH C₁₈ Column, 50×2.1 mm, 1.7 μm, 130 Å pore size

Lcms_short method: 40° C. column temperature. UV absorption wavelength: 214 nm. MS range: 200-1250 Da. Mobile phase A: 0.1% TFA in water. Mobile phase B: 0.085% TFA in acetonitrile. Flowrate: 0.5 mL/min. Gradient:

| Time (min) | % B |
|---|---|
| 0.0 | 0 |
| 5 | 80 |
| 5.1 | 90 |
| 6.0 | 90 |
| 6.1 | 0 |
| 7.0 | 0 |

Column: ACQUITY UPLC Protein BEH C₄ Column, 300 Å, 1.7 μm, 2.1 mm×100 mm

Lcms_long_C4 method: 40° C. column temperature. UV absorption wavelength: 214 nm. MS range: 200-1250 Da. Mobile phase A: 0.1% TFA in water. Mobile phase B: 0.085% TFA in acetonitrile. Flowrate: 0.5 mL/min. Gradient:

| Time (min) | % B |
|---|---|
| 0.0 | 10 |
| 8.0 | 80 |
| 8.1 | 90 |
| 9.0 | 90 |
| 9.1 | 10 |
| 11.0 | 10 |

HPLC instrument and methods

Instrument: Waters 2767 Autopure with mass spectrometer. Mass spectrometer range: 200-3000 Da. UV Detector wavelength: 214 nm.

Column: Phenomenex Luna C₅ Column, 250×21.2 mm, 10 μm particle size, 100 Å pore size.

Method: 20 mL/min flowrate. Mobile phase A: 0.05% TFA in water. Mobile phase B: 0.05% TFA in acetonitrile. Gradient:

| Time (min) | % B |
|---|---|
| 0.0 | 5 |
| 6.0 | 5 |
| 8.0 | 15 |
| 43.0 | 30 |
| 43.1 | 90 |
| 46.0 | 90 |
| 46.1 | 5 |
| 50.0 | 5 |

Column: Phenomenex Luna 18 Column, 250×30 mm, 10 μm particle size, 100 Å pore size.

Method: 30 mL/min flowrate. Mobile phase A: 0.05% TFA in water. Mobile phase B: 0.05% TFA in acetonitrile. Gradient:

| Time (min) | % B |
|---|---|
| 0.0 | 5 |
| 6.0 | 5 |
| 8.0 | 30 |
| 43.0 | 45 |
| 43.1 | 90 |
| 46.0 | 90 |
| 46.1 | 5 |
| 50.0 | 5 |

### Example 1: Synthetic Scheme I

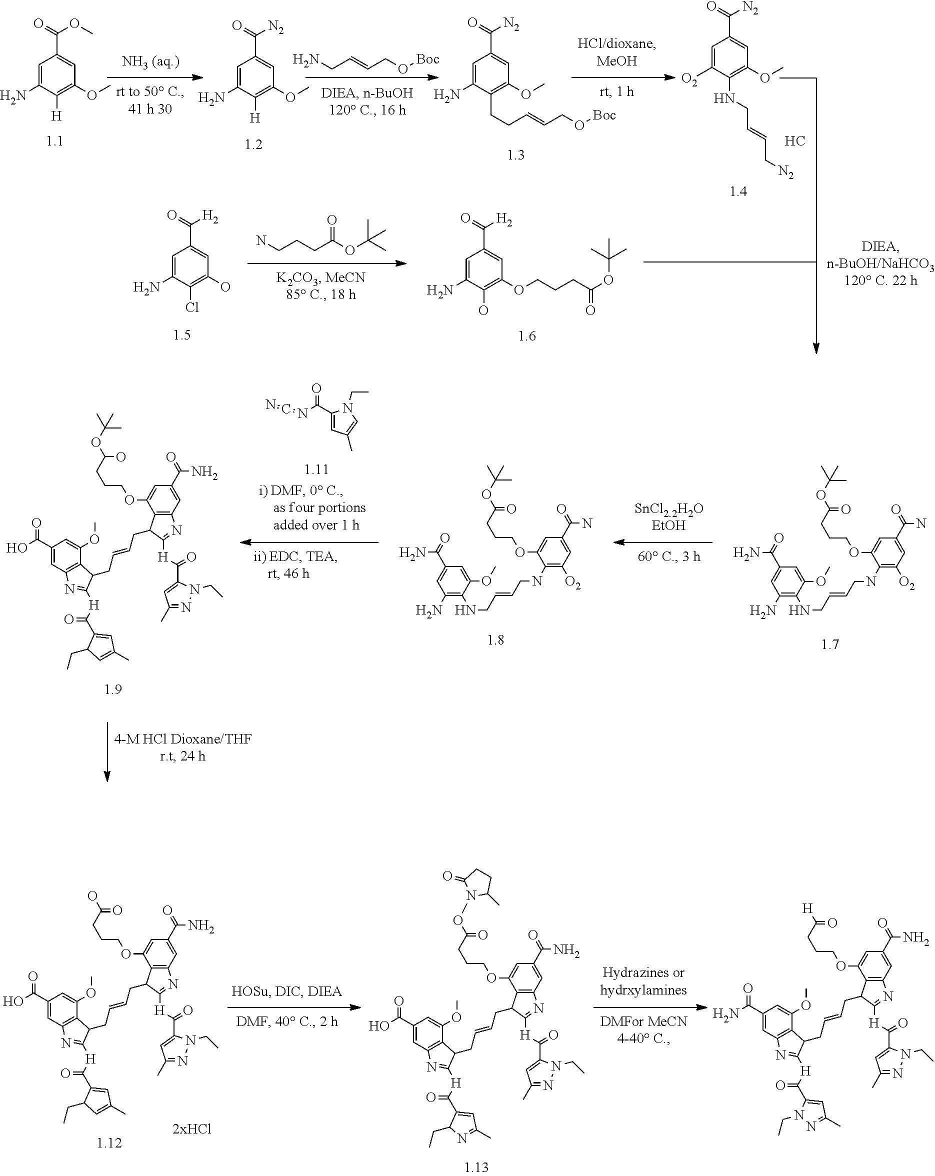

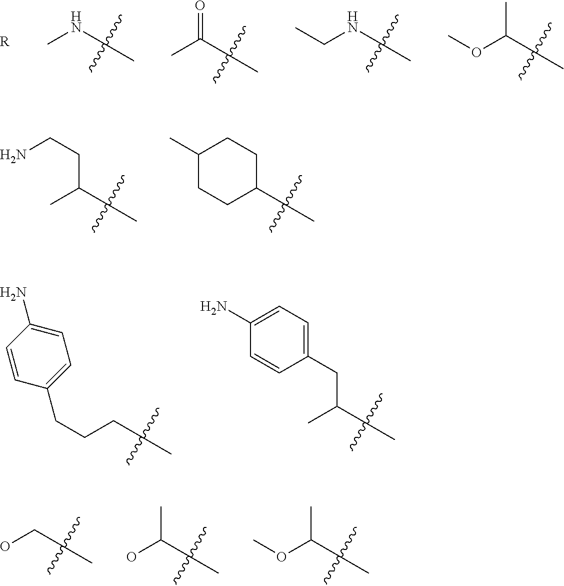

### Preparation of 4-Chloro-3-methoxy-5-nitrobenzamide (1.2)

A suspension of methyl 4-chloro-3-methoxy-5-nitrobenzoate (1.1) (50.0 g; 204 mmol; 1.0 eq.) in 30% aqueous ammonium hydroxide solution (733 mL) was stirred at 50° C. for 1.5 hours (over pressure) then at room temperature for 16 hours. The mixture was then stirred at 50° C. for 24 hours (open system). The reaction mixture was allowed to cool to room temperature and filtered. The solid was washed with water (500 mL), diethyl ether (400 mL) and dried (50° C., 61 h) to afford pure compound 1.2 (40.55 g; 176 mmol; 86%) as a yellow solid. ¹H NMR (DMSO-d₆): δ 4.02 (s, 3H), 7.78 (br s, 1H), 7.88 (d, 1H, J=1.7 Hz), 8.05 (d, 1H, J=1.7 Hz), 8.29 (br s, 1H). LCMS (2-100 ACN/H₂O+0.1% FA): Tr=2.66 min; purity=100%; [M+H⁺]⁺=231.2.

### Preparation of tert-Butyl N-[(2E)-4-[(4-carbamoyl-2-methoxy-6-nitrophenyl)amino]but-2-en-1-yl]carbamate (1.3)

To a suspension of tert-butyl N-[(2E)-4-aminobut-2-en-1-yl]carbamate (10.9 g; 58.5 mmol; 1.5 eq.) and compound 1.2 (9.0 g; 39.0 mmol; 1.0 eq.) in n-butanol (105 mL) was added DIPEA (21.5 mL; 123 mmol; 3.2 eq.). The reaction mixture was stirred at 120° C. for 16 hours. The mixture was allowed to cool to room temperature, then to 0° C. and filtered. The solid was washed with cold ethanol (3×80 mL), and dried under vacuum to afford pure compound 1.3 (13.6 g; 35.9 mmol; 92%) as a brick red crystalline solid. ¹H NMR (DMSO-d₆): δ 1.35 (s, 9H), 3.40-3.52 (m, 2H), 3.87 (s, 3H), 4.05-4.09 (m, 2H), 5.49-5.58 (m, 2H), 6.56 (br s, 0.2H), 6.93 (t, 0.8H, J=5.3 Hz), 7.32 (br s, 1H), 7.55 (d, 1H, J=1.6 Hz), 7.74 (d, 1H, J=6.0 Hz), 8.01 (br s, 1H), 8.18 (d, 1H, J=1.9 Hz). LCMS (2-100 ACN+0.1% FA/H₂O+0.1% FA): Tr=2.85 min; purity=100%; [M+Na⁺]⁺=403.4.

### Preparation of 4-{[(2E)-4-Aminobut-2-en-1-yl]amino}-3-methoxy-5-nitrobenzamide hydrochloride (1.4)

To a suspension of compound 1.3 (12.4 g; 32.5 mmol; 1.0 eq.) in methanol (30.9 mL) was added dropwise 4 M HCl in dioxane (61.8 mL; 247 mmol; 7.6 eq.). The resulting solution was stirred at room temperature for 1 hour. The formed solid was filtered and washed with diethyl ether (3×100 mL), dried under reduced pressure (40° C., 16 h) to afford crude compound 1.4 (10.3 g; 32.5 mmol; quantitative) as an orange solid. ¹H NMR (DMSO-d₆): δ 3.36-3.42 (m, 2H), 3.89 (s, 3H), 4.16-4.18 (m, 2H), 4.71 (br s, 1H), 5.59-5.66 (m, 1H), 5.84-5.90 (m, 1H), 7.37 (br s, 1H), 7.59 (d, 1H, J=1.9 Hz), 8.00-8.07 (m, 4H), 8.21 (d, 1H, J=1.9 Hz). LCMS (2-100 ACN/H₂O+0.1% FA): Tr=2.12 min; purity=10%; [M−HCl+H⁺]⁺=281.3.

### Preparation of 4-Chloro-3-hydroxy-5-nitrobenzamide (1.5)

To a solution of compound 1.2 (31.5 g; 136 mmol; 1.0 eq.) in dichloromethane (400 mL) cooled to 0° C. was added over 20 minutes boron tribromide (1 M in DCM; 545 mL; 545 mmol; 4.0 eq.) and this mixture was stirred at 40° C. for 42 hours. The reaction mixture was allowed to reach room temperature and was poured into 500 ml of ice/water and stirred for 10 minutes (until ice melts). The precipitate was filtered, washed two times with water, two times with n-pentane, dried under reduced pressure (50° C., 16 h) to afford a white solid (41.4 g). The residue was crushed and triturated in water (400 mL) for 1 hour and filtered. The filter cake was washed with water (500 mL) and n-pentane (2×500 mL), dried under vacuum to afford a white solid (25.3 g). The residue was crushed and triturated in water (400 mL) for 2 hours, then filtered. The filter cake was washed with water (500 mL) and n-pentane (2×500 mL), and dried under vacuum to afford crude compound 1.5 (23.5 g; 108.5 mmol; 80%) as a white solid. ¹H NMR (DMSO-d₆): δ 7.67 (br s, 1H), 7.72 (d, 1H, J=1.8 Hz), 7.93 (d, 1H, J=1.9 Hz), 8.18 (br s, 1H), 11.53 (br s, 1H). LCMS (2-100 ACN+0.1% AF/H₂O+0.1% AF): Tr=2.49 min; purity=90.58%; [M+H⁺]⁺=217.2.

### Preparation of tert-Butyl 4-(5-carbamoyl-2-chloro-3-nitrophenoxy)butanoate (1.6)

To a solution of compound 1.5 (23.5 g; 108.5 mmol; 1.0 eq.) in acetonitrile (285 mL) was added tert-butyl 4-bromobutanoate (48.4 g; 38.5 mL; 217 mmol; 2.0 eq.) followed by potassium carbonate (30.0 g; 217 mmol; 2.0 eq.). The resulting solution was stirred at reflux. After 30 minutes, apparition of solids (no more stirring). The solid was broken and acetonitrile (200 mL) was added, the reaction mixture was refluxed for 18 hours. The reaction mixture was concentrated under reduced pressure, diluted with water (700 mL) and ethyl acetate (500 mL), stirred for 10 minutes to dissolve the solid and extracted with ethyl acetate (2×500 mL). The organic phase was washed with water (300 mL), brine (300 mL), dried over sodium sulfate, filtered and concentrated under reduced pressure to afford a pale brownish solid (60.2 g). The residue was suspended in n-pentane (30 mL), sonicated for 10 minutes (break big chunks of solid with spatula beforehand to obtain a homogeneous powder). The suspension was filtered over a sintered glass funnel and washed with n-pentane (4×20 mL). The resulting solid was dried under reduced pressure to afford pure compound 1.6 (36 g; 100 mmol; 92%) as a white solid. ¹H NMR (DMSO-d₆): δ 1.10 (s, 9H), 1.98-2.05 (m, 2H), 2.43 (t, 2H, J=7.4 Hz), 4.25 (t, 2H, J=6.2 Hz), 7.78 (br s, 1H), 7.87 (d, 1H, J=1.8 Hz), 8.05 (d, 1H, J=1.8 Hz), 8.29 (br s, 1H). LCMS (2-100 ACN/H₂O+0.1% AF): Tr=3.11 min; purity=96.02%; [M−tBu+H⁺]⁺=303.2.

### Preparation of tert-Butyl 4-(5-carbamoyl-2-{[(2E)-4-[(4-carbamoyl-2-methoxy-6-nitrophenyl)amino]but-2-en-1-yl]amino}-3-nitrophenoxy)butanoate (1.7)

To a stirred solution of compound 1.4 (1.96 g; 5.57 mmol; 1.0 eq.) in n-butanol (37.8 mL) was added DIPEA (4.61 mL; 27.9 mmol; 5.0 eq.) and sodium bicarbonate (937 mg; 11.1 mmol; 2.0 eq.) and the mixture was stirred at room temperature for 10 minutes. Compound 1.6 (2.00 g; 5.57 mmol; 1.0 eq.) was then added and the reaction mixture was stirred at 120° C. for 22 hours. The reaction mixture was quenched with water (150 mL) at room temperature and extracted with a mixture of dichloromethane/methanol (9/1, 3×150 mL). The organic layers were dried over anhydrous sodium sulfate, filtered and concentrated to afford crude compound 1.7 (3.36 g; 5.58 mmol; considered quantitative) as an orange oil. The crude compound 1.7 was used as such into the next step. LCMS (2-100 ACN/H₂O+0.1% AF): Tr=3.01 min; purity=61.46%; [M+H⁺]⁺=603.5.

### Preparation of tert-Butyl 4-(3-amino-2-{[(2E)-4-[(2-amino-4-carbamoyl-6-methoxyphenyl)amino]but-2-en-1-yl]amino}-5-carbamoylphenoxy)butanoate (1.8)

To a solution of crude impure compound 1.7 (3.34 g; 5.54 mmol; 1.0 eq.) in ethanol (45 mL) was added dichlorostannane dihydrate (10.0 g; 44.3 mmol; 8.0 eq.). The mixture was then heated to 60° C. and stirred for 3 hours. The reaction was allowed to cool down, diluted with an aqueous saturated solution of potassium carbonate (100 mL) and water (100 mL), and extracted with a mixture of dichloromethane/methanol (85:15, 3×250 mL). The combined organic phases were dried over anhydrous sodium sulfate, filtered and concentrated under reduced pressure to afford a white solid (2.67 g). The residue was purified by flash chromatography on silica gel (50 μm irregular, 80 g) using dichloromethane/methanol (98:2 to 80:20 in 50 minutes) to afford pure compound 1.8 (1.28 g; 2.36 mmol; 43% over 2 steps) as a yellow solid. ¹H NMR (DMSO-d₆): δ 1.38 (s, 9H), 1.89-1.93 (m, 2H), 2.39 (t, 2H, J=7.4 Hz), 3.48-3.52 (m, 4H), 3.73 (s, 3H), 3.79 (dt, 2H, J=26.6, 6.8 Hz), 3.93 (t, 2H, J=6.2 Hz), 4.64 (d, 4H, J=9.7 Hz), 5.62-5.71 (m, 2H), 6.76 (dd, 2H, J=8.4, 1.8 Hz), 6.85 (t, 2H, J=1.7 Hz), 6.96 (br s, 2H), 7.60 (br s, 2H). LCMS (2-100 ACN/H₂O+0.1% AF): Tr=2.45 min; purity=99.23%; [M+H⁺]+=543.6.

### Preparation of tert-Butyl 4-({5-carbamoyl-1-[(2E)-4-[5-carbamoyl-2-(1-ethyl-3-methyl-1H-pyrazole-5-amido)-7-methoxy-1H-1,3-benzodiazol-1-yl]but-2-en-1-yl]-2-(1-ethyl-3-methyl-1H-pyrazole-5-amido)-1H-1,3-benzodiazol-7-yl}oxy)butanoate (1.9)

To a solution of compound 1.8 (5.60 g; 10.3 mmol; 1.0 eq.) in N,N-dimethylformamide (110 mL) at 0° C. was added compound 1.11 (0.2 M in dioxane; 20.6 mL; 10.3 mmol; 1.0 eq.) and the reaction mixture was stirred for 15 minutes. Compound 1.11 (0.2 M in dioxane; 8.26 mL; 4.13 mmol; 0.4 eq.) was added and the reaction mixture was stirred for 15 minutes. Compound 1.11 (0.2 M in dioxane; 4.13 mL; 2.06 mmol; 0.2 eq.) was added and the reaction mixture stirred for 15 minutes. Compound 1.11 (0.2 M in dioxane; 8.26 mL; 4.13 mmol; 0.4 eq.) was added and the reaction mixture was stirred for 15 minutes. EDC·HCl (4.95 g; 25.8 mmol; 2.5 eq.) followed by triethylamine (7.17 mL; 51.6 mmol; 5.0 eq.) were added to the reaction at 0° C., allowed to warmed up and stirred for 18 hours at room temperature. Triethylamine (7.17 mL; 51.6 mmol; 5.0 eq.) and EDC·HCl (4.95 g; 25.8 mmol; 2.5 eq.) were added and the reaction mixture was stirred at room temperature for 24 hours. The reaction was quenched with a mixture of water/saturated aqueous ammonium chloride (3:1, 400 mL) and extracted with a mixture of dichloromethane/methanol (3:1, 3×400 mL). The combined organic layers were washed with water (100 mL), dried over anhydrous sodium sulfate, filtered and concentrated under reduced pressure to afford an orange oil (32 g). The residue was purified by flash chromatography on silica gel (50 μm irregular, 330 g) using dichloromethane/methanol (90/10 for 15 minutes then to 80/20 in 25 minutes) to afford impure compound 1.9 (8.03 g) as a yellow solid. The residue was triturated in acetonitrile (2×125 mL) at 45° C. for 2 hours, then filtered. The solid was dissolved in a mixture of dichloromethane/methanol (3:1, 700 mL) and washed with water (2×400 mL). The aqueous layer was extracted with a mixture of dichloromethane/methanol (3:1, 300 mL). The combined organic layers were dried over anhydrous sodium sulfate, filtered, concentrated and dried under vacuum (40° C., 16 hours) to afford pure compound 1.9 (6.00 g; 6.94 mmol; 67%) as a beige solid. ¹H NMR (DMSO-d₆): δ 1.26 (t, 6H, J=7.0 Hz), 1.39 (s, 9H), 1.76-1.83 (m, 2H), 2.09 (s, 3H), 2.10 (s, 3H), 2.25 (t, 2H, J=7.2 Hz), 3.72 (s, 3H), 3.98 (t, 2H, J=6.1 Hz), 4.52 (q, 4H, J=7.0 Hz), 4.90-4.93 (m, 4H), 5.76-5.89 (m, 2H), 6.50 (d, 2H, J=5.3 Hz), 7.30 (d, 2H, J=5.3 Hz), 7.34 (br s, 2H), 7.64 (s, 2H), 7.95 (br s, 2H), 12.81 (br s, 2H). LCMS (2-100 ACN/H₂O+0.1% AF): Tr=9.55 min; purity=94.58%; [M+H⁺]⁺=865.46.

### Preparation of Compound 1.12

To a suspension of compound 1.9 (400 mg; 0.46 mmol) in THF (4 mL) at room temperature was added HCl (4 M in dioxane; 3.0 mL; 12.0 mmol) and the forming mixture was stirred at room temperature for 24 hours. Complete conversion to compound 1.12 was confirmed by UPLC-MS monitoring. The reaction mixture was titrated into cold diethyl ether (100 mL), and forming precipitate was isolated via centrifugation. Then supernatant was removed, and the forming pellet was resuspended in cold diethyl ether (100 mL) and centrifuged. This step was repeated twice. Finally, the pellet was dried in vacuo to give compound 1.12 as HCl salt, a beige solid (386.5 mg; 0.44 mmol; 95%). Product was used for the next step without further purification. UPLC-MS retention time: 3.26 min (Icms_long); [M+H⁺]⁺=809.51.

### Preparation of Compound 1.13

To a suspension of compound 1.12 (250.1 mg; 0.28 mmol; 1.0 eq., HCl salt) in DMF (1 mL) at 40° C. was added DIEA (148.7 μL; 0.85 mmol; 3.0 eq.) followed by N-Hydroxysuccinimide (161 mg; 1.4 mmol; 5.0 eq.) and N,N′-Diisopropylcarbodiimide (212.0 mg; 1.68 mmol; 6.0 eq.). The forming mixture was stirred for 2 hours. Complete conversion to compound 1.13 was confirmed by UPLC-MS monitoring. The reaction mixture was titrated into cold diethyl ether (100 mL), and forming precipitate was isolated via centrifugation. The supernatant was then removed, and the forming pellet was resuspended in cold diethyl ether (100 mL) and centrifuged. This step was repeated twice. Finally, the pellet was dried in vacuo to give of compound 1.13 as a beige solid (220.7 mg; 0.24 mmol; 87%). The product was used for the next step without further purification. UPLC-MS retention time: 4.40 min (Icms_long), [M+H⁺]⁺=906.54.

### Preparation of Compound I-A

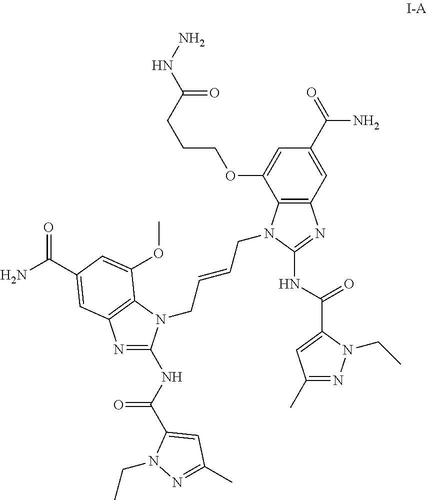 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NN)c21]

General method A: To a solution of compound 1.13 (45.0 mg; 0.05 mmol; 1.0 eq.) in DMF (3 mL) at room temperature was added hydrazine hydrate (20 μL; 0.49 mmol; 10.0 eq.). The forming mixture was stirred for 1 hour. Complete consumption of the starting material and formation of compound I-A was confirmed by UPLC-MS monitoring. The reaction mixture was diluted with water, filtered with a 0.22 μm syringe filter, and directly injected into RP-HPLC for purification (Phenomenex Luna C18 250×30 mm column; 30 mL/min flowrate; 0.05% TFA; 30-45% MeCN/H₂O gradient over 35 min; 50 min total run time). Fractions with pure product were pooled, frozen, and lyophilized to provide compound I-A as white solid (25 mg; 0.03 mmol; 60%). UPLC-MS retention time: 3.73 min broad peak (Icms_long); [M+H⁺]⁺=823.52.

### Preparation of Compound I-B

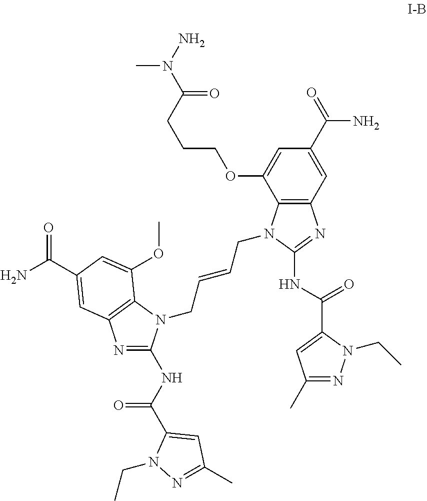 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)N)c21]

Compound I-B was prepared using monomethyl hydrazine following modified general method A where the reaction solvent was MeCN and the reaction was run at 4° C. to provide compound I-B as a beige solid. UPLC-MS retention time: 3.90 min (Icms_long); [M+H⁺]⁺=837.59.

### Preparation of Compound I-C

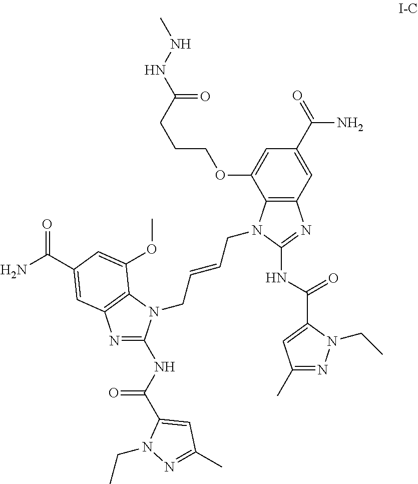 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NNC)c21]

General method B: To a solution of compound 1.13 (30 mg; 0.033 mmol; 1.0 eq.) in MeCN (1 mL) at room temperature was added 1-Boc-1-methylhydrazine (48.4 mg; 0.33 mmol; 10.0 eq.). The forming mixture was stirred at 40° C. for 16 hours. Complete consumption of the starting material and formation of intermediate compound I-C-Boc was confirmed by UPLC-MS monitoring. The reaction mixture was allowed to cool down to room temperature and then trifluoroacetic acid was added (400 μL) followed by HCl (4 M in dioxane; 200 μL). The forming mixture was stirred at room temperature for 1 h during which time the mixture turned turbid. Complete Boc removal and formation of desired compound I-C was confirmed by UPLC-MS monitoring and then the reaction mixture was further diluted with water to 7 mL, filtered with a 0.22 μm syringe filter, and directly injected into RP-HPLC for purification (Phenomenex Luna C18 250×30 mm column; 30 mL/min flowrate; 0.05% TFA; 30-45% MeCN/H₂O gradient over 35 min; 50 min total run time). Fractions with pure product were pooled, frozen, and lyophilized to provide compound I-C as a white solid (3.5 mg; 0.0042 mmol; 12%). UPLC-MS retention time: 3.87 min (lcms_long); [M+H⁺]⁺=837.59.

### Preparation of Compound I-D

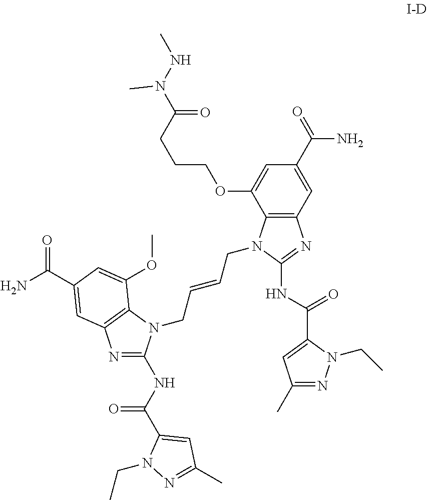 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)NC)c21]

General method C: To a solution of compound 1.13 (170 mg; 0.187 mmol; 1.0 eq.) in DMF (3 mL) at room temperature was added N,N′-Dimethylhydrazine dihydrochloride (42.4 mg; 0.32 mmol; 1.7 eq.) followed by DIEA (130.4 μL; 0.75 mmol; 4.0 eq). The forming mixture was stirred at room temperature for 5 minutes. Complete consumption of the starting material and formation of desired compound I-D was confirmed by UPLC-MS monitoring and the reaction mixture was then diluted with water to 6 mL, filtered with a 0.22 μm syringe filter, and directly injected into RP-HPLC for purification (Phenomenex Luna C18 250×30 mm column; 30 mL/min flowrate; 0.05% TFA; 30-45% MeCN/H₂O gradient over 35 min; 50 min total run time). Fractions with pure product were pooled, frozen, and lyophilized to provide compound I-D as a white solid (107.4 mg; 0.12 mmol; 67%). UPLC-MS retention time: 4.01 min (Icms_long); [M+H⁺]⁺=851.56.

### Preparation of Compounds I-E and I-F

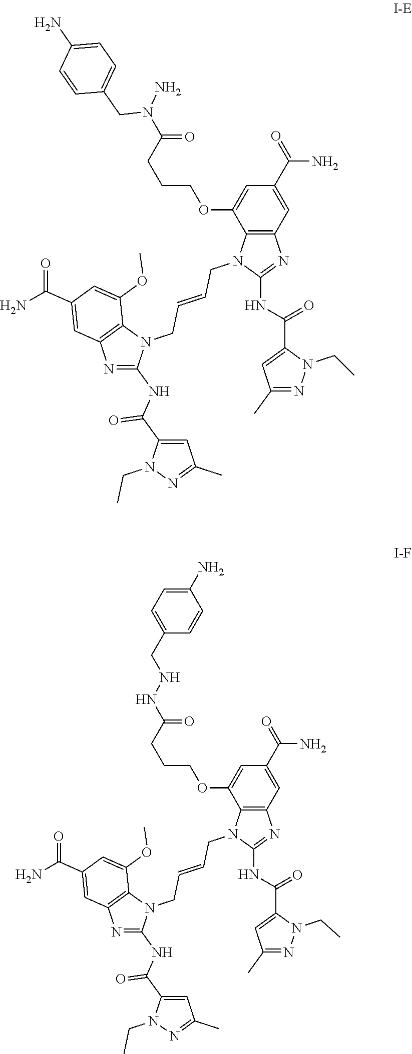 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(N)Cc3ccc(N)cc3)c21 ; CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NNCc3ccc(N)cc3)c21]

Compounds I-E and I-F were both isolated as beige solids from one reaction mixture using tert-butyl (4-(hydrazineylmethyl)phenyl)carbamate and following modified general method B where the reaction was run at room temperature.

Compound I-E (major product): UPLC-MS retention time: 3.68 min (Icms_long); [M+Na⁺]⁺=950.55.

Compound I-F (minor product): UPLC-MS retention time: 3.78 min (Icms_long); [M+Na⁺]⁺=950.55.

### Preparation of Compound I-G

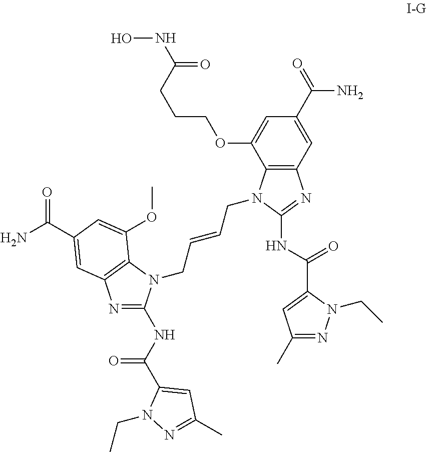 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NO)c21]

Compound I-G was prepared using hydroxylamine hydrochloride (2.0 eq.) and N-Methylmorpholine (4.0 eq.) instead of DIEA following general method C. UPLC-MS retention time: 3.66 min (Icms_long); [M+H⁺]⁺=824.50 m/z.

### Preparation of Compound I-H

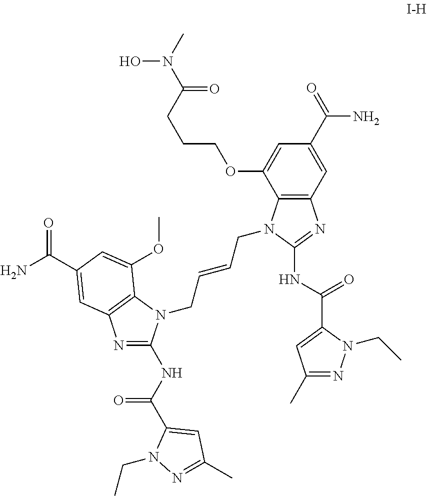 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)O)c21]

Compound I-H was prepared using N-Methylhydroxylamine hydrochloride (2.0 eq.) and N-Methylmorpholine (4.0 eq.) instead of DIEA following general method C. UPLC-MS retention time: 4.02 min (Icms_long); [M+H⁺]⁺=838.55 m/z.

### Preparation of Compound I-I

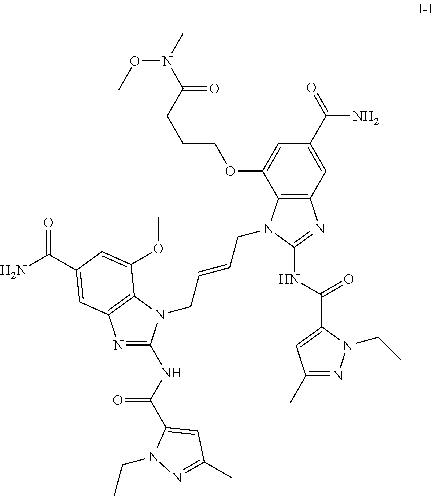 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)OC)c21]

Compound I-I was prepared using N, O-Dimethylhydroxylamine hydrochloride (2.0 eq.) and N-methylmorpholine (4.0 eq.) instead of DIEA following general method C. UPLC-MS retention time: 4.36 min (Icms_long); [M+H⁺]⁺=852.55.

### Preparation of Compound I-J

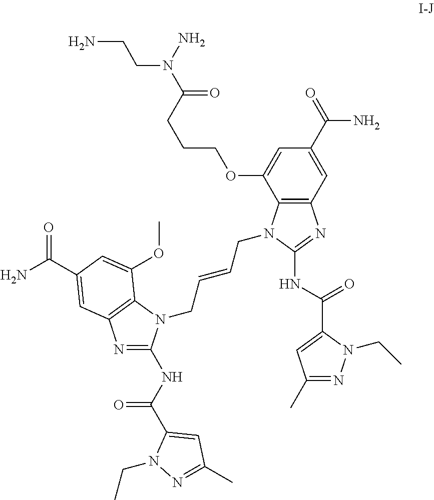 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(N)CCN)c21]

Compound I-J was prepared as a white solid using tert-butyl(2-hydrazinoethyl)carbamate and following modified general method B where the reaction was run at room temperature. The compound was isolated as the only product of the reaction. UPLC-MS retention time: 3.71 min (lcms_long); [M+H⁺]⁺=866.54.

### Preparation of Compound I-K

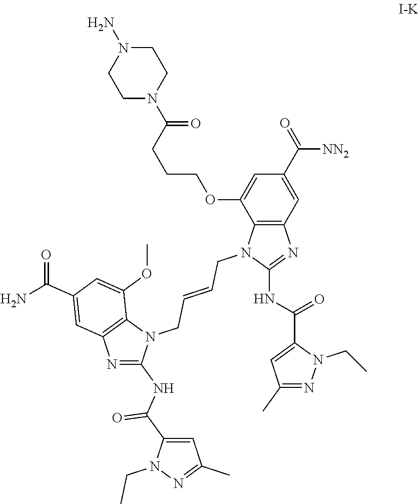

Compound I-K was prepared as a white solid using tert-Butyl piperazin-1-ylcarbamate and following modified general method B where the reaction was run at room temperature. UPLC-MS retention time: 3.74 min (Icms_long); [M+H⁺]⁺=892.64.

### Example 2: Synthetic Scheme II

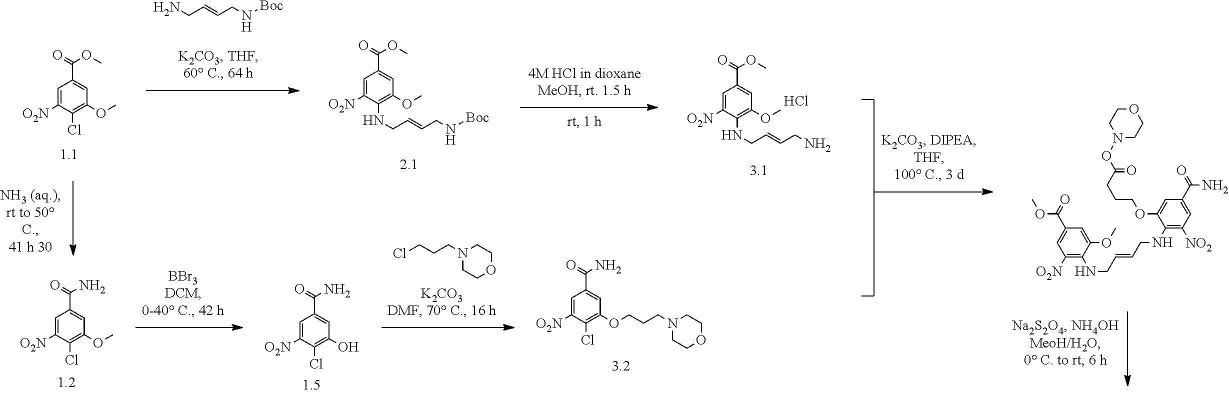

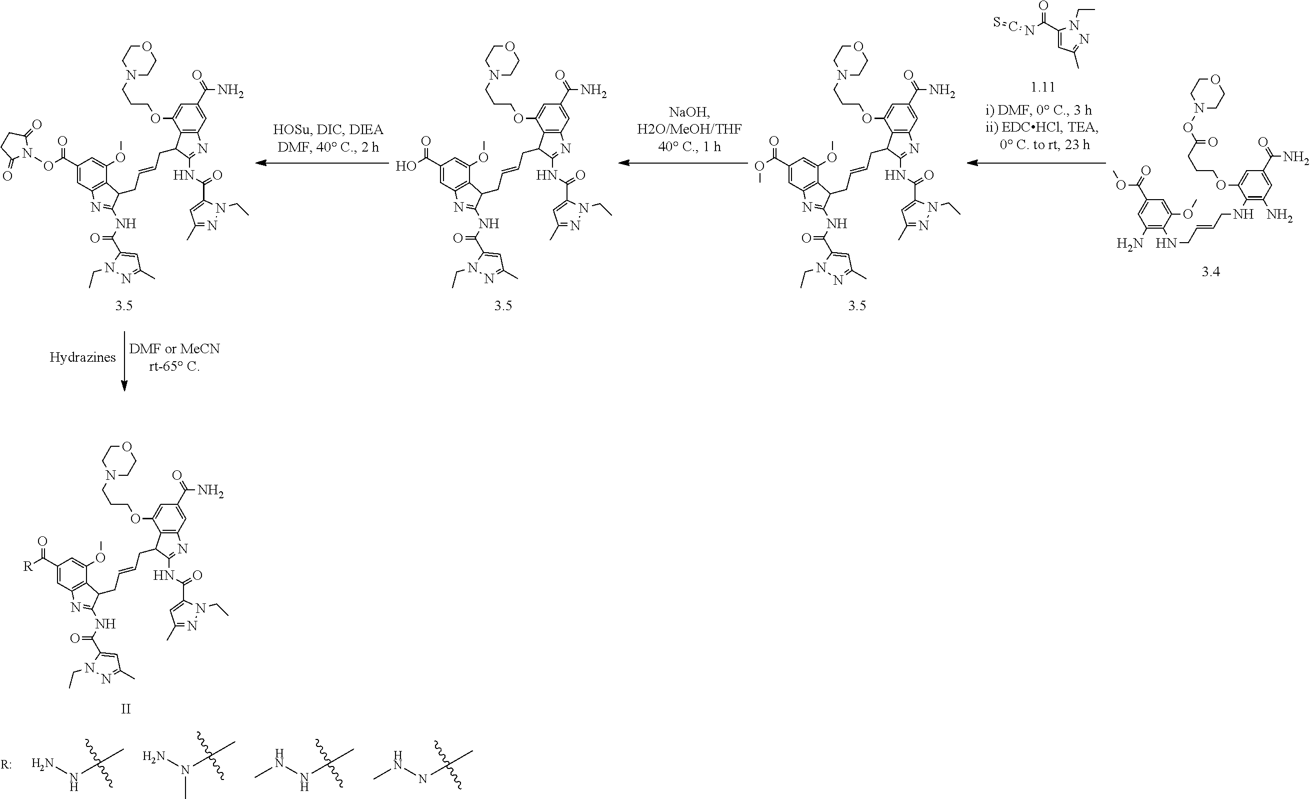

Preparation of Methyl 4-{[(2E)-4-{[(tert-butoxy)carbonyl]amino}but-2-en-1-yl]amino}-3-methoxy-5-nitrobenzoate (2.1)

To a solution of methyl 4-chloro-3-methoxy-5-nitrobenzoate (1.1) (1.00 g; 4.07 mmol; 1.00 eq.) and tert-butyl N-[(2E)-4-aminobut-2-en-1-yl]carbamate (834 mg; 4.48 mmol; 1.10 eq.) in tetrahydrofuran (15 mL) was added potassium carbonate (1.13 g; 8.14 mmol; 2.00 eq.) at room temperature. The reaction mixture was stirred at 60° C. for 64 hours. The resulting mixture was concentrated in vacuo. The residue was re-dissolved in ethyl acetate (100 mL), washed with water (2×50 mL) and saturated aqueous sodium chloride (100 mL). The organic extracts were dried over anhydrous sodium sulfate and concentrated in vacuo to afford pure compound 2.1 (1.61 g; 4.07 mmol; quantitative) as an orange solid. ¹H NMR (DMSO-d₆): 1.34 (s, 9H), 3.42-3.50 (m, 2H), 3.83 (s, 3H), 3.89 (s, 3H), 4.11-4.13 (m, 2H), 5.52-5.53 (m, 2H), 6.56 (br s, 0.1H), 6.92 (t, 0.9H, J=5.4 Hz), 7.43 (d, 1H, J=1.8 Hz), 8.01 (t, 1H, J=6.1 Hz), 8.16 (t, 1H, J=1.8 Hz). LCMS (2-100 ACN/H₂O+0.1% FA): Tr=3.23 min; purity=98.1%; [M+Na⁺]=418.4.

### Preparation of Methyl 4-{[(2E)-4-aminobut-2-en-1-yl]amino}-3-methoxy-5-nitrobenzoate hydrochloride (3.1)

To a suspension of compound 2.1 (3.25 g; 7.23 mmol; 1.00 eq.) in MeOH (7.15 mL) was added HCl (4N in dioxane) (14.3 mL; 57.2 mmol; 7.90 eq.) dropwise at room temperature. The reaction mixture was stirred at room temperature for 1.5 hours. The precipitate was filtered and washed with diethyl ether (3×20 mL). The filtrate was re-filtered and washed with diethyl ether (10 mL). The solids were combined and dried under reduced pressure to afford pure crude compound 3.1 (2.38 g; 7.11 mmol; 98%) as an orange solid. ¹H NMR (DMSO-d₆): 3.38-3.42 (m, 2H), 3.84 (s, 3H), 3.90 (s, 3H), 4.21 (t, 2H, J=5.8 Hz), 5.59-5.66 (m, 1H), 5.83-5.90 (m, 1H), 7.46 (d, 1H, J=1.8 Hz), 7.99 (br s, 3H), 8.13 (t, 1H, J=6.4 Hz), 8.19 (d, 1H, J=1.8 Hz). LCMS (2-100 ACN/H₂O+0.1% FA): Tr=2.43 min; purity=100%; [M−HCl+H⁺]=296.4.

### Preparation of 4-Chloro-3-[3-(morpholin-4-yl) propoxy]-5-nitrobenzamide (3.2)

A mixture of compound 1.5 (1.00 g; 4.62 mmol; 1.00 eq.), 4-(3-chloropropyl) morpholine (907 mg; 5.54 mmol; 1.20 eq.), potassium carbonate (830 mg; 6.00 mmol; 1.30 eq.) in N,N-dimethylformamide (6.00 mL) was stirred at 70° C. for 16 hours. The solvent was removed in vacuo to give a yellow solid (2.71 g). The residue was purified by flash chromatography on silica gel (50 μm irregular, 80 g) using dichloromethane/methanol (100:0 to 85:15 in 30 minutes and then 85:15 for 10 minutes) to afford pure compound 3.2 (1.17 g; 3.40 mmol; 74%) as a pale yellow solid. ¹H NMR (DMSO-d₆): δ 1.92-1.98 (m, 2H), 2.35-2.37 (m, 4H), 2.45 (t, 2H, J=7.1 Hz), 3.56 (t, 4H, J=4.6 Hz), 4.28 (t, 2H, J=6.3 Hz), 7.77 (br s, 1H), 7.87 (d, 1H, J=1.7 Hz), 8.04 (d, 1H, J=1.7 Hz), 8.27 (br s, 1H). LCMS (2-100 ACN/H₂O+0.1% AF): Tr=2.37 min; purity=100%; [M+H⁺]=344.3.

### Preparation of 4-{[(2E)-4-({4-Carbamoyl-2-[3-(morpholin-4-yl) propoxy]-6-nitrophenyl}amino)but-2-en-1-yl]amino}-3-methoxy-5-nitrobenzamide (3.3)

To a stirred solution of compound 3.1 (1.06 g; 3.20 mmol; 1.10 eq.) and compound 3.2 (1.00 g; 2.91 mmol; 1.00 eq.) in tetrahydrofuran (10.7 mL) was added potassium carbonate (804 mg; 5.82 mmol; 2.00 eq.) and DIPEA (1.68 mL; 10.2 mmol; 3.49 eq.). The reaction mixture was stirred at 100° C. for 3 days. The reaction mixture was cooled down to room temperature, diluted with water (40 mL) and extracted with a mixture of dichloromethane/methanol (8/2, 3×40 mL). The combined organic layers were dried over anhydrous sodium sulfate, filtered and concentrated to afford an orange solid (2.17 g). The residue was purified by flash chromatography on silica gel (50 μm irregular, 80 g) using dichloromethane/methanol (98:2 for 10 minutes, to 92:8 in 20 minutes and then 92:8 for 10 minutes) to afford pure compound 3.3 (1.17 g; 1.95 mmol; 67%) as an orange solid. ¹H NMR (DMSO-d₆): δ 1.85-1.91 (m, 2H), 2.32-2.34 (m, 4H), 2.38 (t, 2H, J=7.1 Hz), 3.55 (t, 4H, J=4.5 Hz), 3.80 (s, 3H), 3.84 (s, 3H), 4.00 (t, 2H, J=6.3 Hz), 4.07-4.11 (m, 4H), 5.52-5.62 (m, 2H), 7.30 (br s, 1H), 7.35 (d, 1H, J=1.8 Hz), 7.47 (d, 1H, J=1.8 Hz), 7.73 (t, 1H, J=6.2 Hz), 7.95-7.98 (m, 2H), 8.10 (d, 1H, J=1.8 Hz), 8.14 (d, 1H, J=1.8 Hz). LCMS (2-100 ACN/H₂O+0.1% AF): Tr=2.59 min; purity=100%; [M+H⁺]=603.5.

### Preparation of Methyl 3-amino-4-{[(2E)-4-({2-amino-4-carbamoyl-6-[3-(morpholin-4-yl) propoxy]phenyl}amino)but-2-en-1-yl]amino}-5-methoxybenzoate (3.4)

To a stirred solution of compound 3.3 (11.7 g; 19.5 mmol; 1.00 eq.) in MeOH (305 mL) at 0° C. was added sodium dithionite (33.9 g; 195 mmol; 10.0 eq.) dissolved in water (105 mL). To this stirred mixture was added a 30% aqueous ammonia solution (19.4 mL; 146 mmol; 7.50 eq.) at 0° C. The mixture was warmed to room temperature and stirred for 6 hours. The reaction mixture was quenched with water (500 mL). Dichloromethane was added to the mixture (700 mL). The layers were separated and the aqueous layer was extracted with a mixture of dichloromethane/methanol (9:1, 2×300 mL). The combined organic layers were washed with brine (500 mL), dried over anhydrous sodium sulfate, filtered and concentrated in vacuo to afford pure compound 3.4 (7.34 g; 13.5 mmol; 69%) as a brown foam. The crude product was used in the next step. ¹H NMR (DMSO-d₆): δ 1.82-1.88 (m, 2H), 2.32-2.34 (m, 4H), 2.40 (t, 2H, J=7.0 Hz), 3.51-3.56 (m, 6H), 3.60-3.62 (m, 2H), 3.73 (s, 3H), 3.76 (s, 3H), 3.80 (t, 1H, J=7.1 Hz), 3.95 (t, 2H, J=6.2 Hz), 4.07 (t, 1H, J=7.0 Hz), 4.65 (s, 2H), 4.79 (s, 2H), 5.59-5.70 (m, 2H), 6.76 (d, 1H, J=1.8 Hz), 6.81 (d, 1H, J=1.8 Hz), 6.84 (d, 1H, J=1.8 Hz), 6.95 (br s, 1H), 7.01 (d, 1H, J=1.8 Hz), 7.60 (br s, 1H). LCMS (2-100 ACN/H₂O+0.1% AF): Tr=2.22 min; purity=100%; [M+H⁺]=543.5.

### Preparation of Methyl 1-[(2E)-4-[5-carbamoyl-2-(1-ethyl-3-methyl-1H-pyrazole-5-amido)-7-[3-(morpholin-4-yl) propoxy]-1H-1,3-benzodiazol-1-yl]but-2-en-1-yl]-2-(1-ethyl-3-methyl-1H-pyrazole-5-amido)-7-methoxy-1H-1,3-benzodiazole-5-carboxylate (3.5)

To a solution of compound 3.4 (2.00 g; 3.69 mmol; 1.00 eq.) in N,N-dimethylformamide (40 mL) at 0° C. was added dropwise compound 1.11 (0.2 M in dioxane; 18.4 mL; 3.69 mmol; 1.00 eq.) and the reaction mixture was stirred for 30 minutes. Compound 1.11 (0.2 M in dioxane; 7.37 mL; 1.47 mmol; 0.40 eq.) was added dropwise at 0° C. and the reaction mixture was stirred for 15 minutes. Compound 1.11 (0.2 M in dioxane; 3.69 mL; 0.74 mmol; 0.20 eq.) was added dropwise at 0° C. and the reaction mixture was stirred for 15 minutes. Compound 1.11 (0.2 M in dioxane; 7.37 mL; 1.47 mmol; 0.40 eq.) was added dropwise at 0° C. and the reaction mixture was stirred for 2 hours. EDC·HCl (1.77 g; 9.21 mmol; 2.50 eq.) followed by triethylamine (2.56 mL; 18.4 mmol; 5.00 eq.) were added to the reaction at 0° C. The reaction mixture was stirred at room temperature for 17 hours. EDC·HCl (0.18 g; 0.92 mmol; 0.25 eq.) and triethylamine (0.064 mL; 0.46 mmol; 0.13 eq.) were added at room temperature. The reaction mixture was stirred at room temperature for 6 hours. The reaction was quenched with a mixture of water/saturated aqueous ammonium chloride (3:1, 80 mL) and extracted with a mixture of dichloromethane/methanol (3:1, 3×80 mL). The combined organic phases were washed with water (100 mL), dried over anhydrous magnesium sulfate, filtered and concentrated under reduced pressure to afford an orange oil. The residue was purified by flash chromatography on silica gel (50 μm irregular, 220 g) using dichloromethane/methanol (96:4 during 5 minutes, to 80:20 in 30 minutes and then to 80:20 in 25 minutes) as eluent to afford impure compound 3.5 (1.83 g) as a beige solid. The residue was triturated in dichloromethane (40 mL) to afford pure compound 3.5 (1.74 g; 2.01 mmol; 55%) as a white solid. ¹H NMR (DMSO-d₆): 0 1.27-1.32 (m, 6H), 1.60-1.66 (m, 2H), 2.11 (s, 3H), 2.13 (s, 3H), 2.17-2.19 (m, 4H), 2.23 (t, 2H, J=7.2 Hz), 3.45 (t, 4H, J=4.5 Hz), 3.65 (s, 3H), 3.67-3.90 (m, 5H), 4.51-4.58 (m, 4H), 4.90-4.92 (m, 4H), 5.75-5.87 (m, 2H), 6.54 (m, 2H), 7.23 (d, 2H, J=5.2 Hz), 7.31 (br s, 1H), 7.63 (s, 1H), 7.77 (d, 1H, J=1.2 Hz), 7.92 (br s, 1H), 12.82 (br s, 1H), 12.86 (br s, 1H). LCMS (2-100 ACN/H₂O+0.1% AF): Tr=7.06 min; purity=94.95%; [M+H⁺]=865.75.

### Preparation of Compound 3.6

To solution of compound 3.5 (400 mg; 0.46 mmol; 1.00 eq.) in a mixture of MeOH (3.2 mL), THF (6.5 mL), and water (1.6 mL) was added NaOH (5 M aq. solution; 920 μ, 4.46 mmol; 10 eq.). The forming mixture was stirred at 40° C. for 1 hour. Complete consumption of the starting material and formation of desired compound 3.6 was confirmed by UPLC-MS monitoring and then to the reaction mixture was added HCl (1 M HCl aq. solution) until pH 6. The turbid mixture was diluted with water, frozen, and lyophilized. The resulting solids were triturated in 90% DCM, 9% MeOH, 1% DIEA. Solids were removed via filtration, and flowthrough was concentrated in vacuo to give a compound 3.6 as a beige solid (356 mg; 0.41; 91%). UPLC-MS retention time: 4.12 min (Icms_long), [M+H+]+=851.56. UPLC-MS retention time: 4.12 min (Icms_long), [M+H+]+=851.56.

### Preparation of Compound 3.7

To a suspension of compound 3.6 (100 mg; 0.117 mmol; 1.0 eq.) in DMF (1 mL) at 40° C. was added DIEA (61.5 μL; 0.35 mmol; 3.0 eq.) followed by N-Hydroxysuccinimide (67 mg; 0.58 mmol; 5.0 eq.) and N,N′-Diisopropylcarbodiimide (88.3 mg; 0.7 mmol; 6.0 eq.). The forming mixture was stirred for 2 hours. Complete conversion to compound 3.7 was confirmed by UPLC-MS monitoring. The reaction mixture was titrated into cold dietheyl ether (100 mL), and forming precipitate was isolated via centrifugation. The supernatant was then removed, and the forming pellet was resuspended in cold diethyl ether (100 mL) and centrifuged. This step was repeated twice. Finally, the pellet was dried in vacuo to give of compound 3.7 as a beige solid (90.0 mg; 0.094 mmol; 80%). The product was used for the next step without further purification. UPLC-MS retention time: 4.41 min (lcms_long), [M+H⁺]⁺=948.62.

### Preparation of Compound II-A

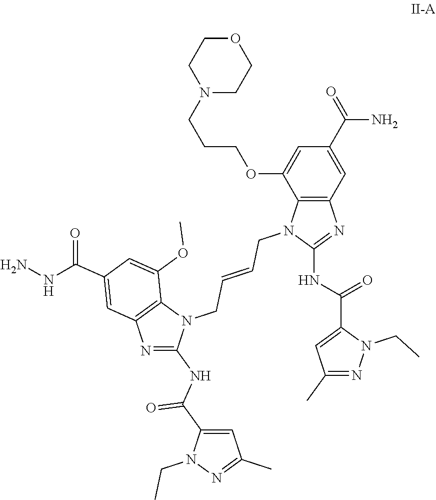 [SMILES: CCn1nc(C)cc1C(=O)Nc1nc2cc(C(=O)NN)cc(OC)c2n1C/C=C/Cn1c(NC(=O)c2cc(C)nn2CC)nc2cc(C(N)=O)cc(OCCCN3CCOCC3)c21]

Compound II-A was prepared using anhydrous hydrazine following general method A to provide compound II-A as a beige solid. UPLC-MS retention time: 3.52 min broad (Icms_long); 3.41 min sharp (Icms_long_C4); [M+H⁺]⁺=865.65.

### Preparation of Compound II-B

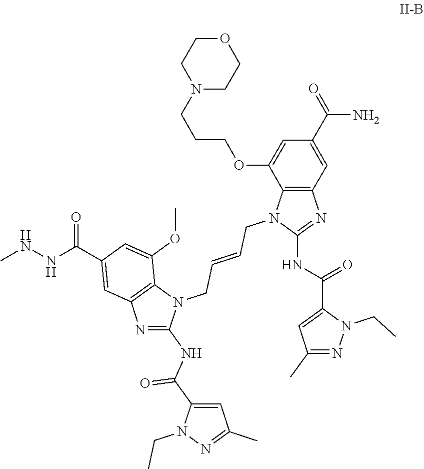 [SMILES: CCn1nc(C)cc1C(=O)Nc1nc2cc(C(=O)NNC)cc(OC)c2n1C/C=C/Cn1c(NC(=O)c2cc(C)nn2CC)nc2cc(C(N)=O)cc(OCCCN3CCOCC3)c21]

Compound II-B was prepared using 1-Boc-1-methylhydrazine following modified general method B where 4-Dimethylaminopyridine (1 eq.) was added for catalysis, and the reaction temperature was increased to 60° C. to provide compound II-B as a beige solid. UPLC-MS retention time: 3.37 min (lcms_long_C₄); [M+H⁺]⁺=879.65.

### Preparation of Compound II-C

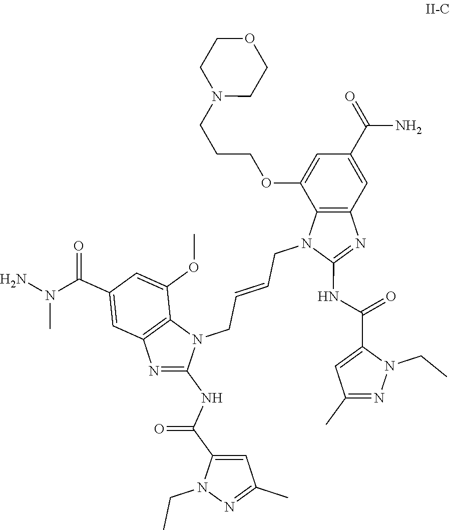 [SMILES: CCn1nc(C)cc1C(=O)Nc1nc2cc(C(=O)N(C)N)cc(OC)c2n1C/C=C/Cn1c(NC(=O)c2cc(C)nn2CC)nc2cc(C(N)=O)cc(OCCCN3CCOCC3)c21]

Compound II-C was prepared as a beige solid using monomethyl hydrazine following a modified general method A where the reaction solvent was MeCN and the reaction was run at 4° C. UPLC-MS retention time: 3.73 min broad (Icms_long); 3.53 min (Icms_long_C4); [M+H⁺]⁺=879.63.

### Preparation of Compound II-D

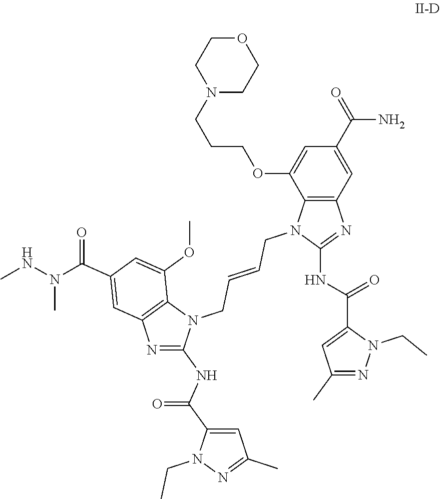 [SMILES: CCn1nc(C)cc1C(=O)Nc1nc2cc(C(=O)N(C)NC)cc(OC)c2n1C/C=C/Cn1c(NC(=O)c2cc(C)nn2CC)nc2cc(C(N)=O)cc(OCCCN3CCOCC3)c21]

Compound II-D was prepared using N,N′-Dimethylhydrazine dihydrochloride following general method C to provide a beige solid. UPLC-MS retention time: 3.06 min broad (Icms_short); [M+H⁺]⁺=893.64.

### Example 3: Synthetic Scheme III

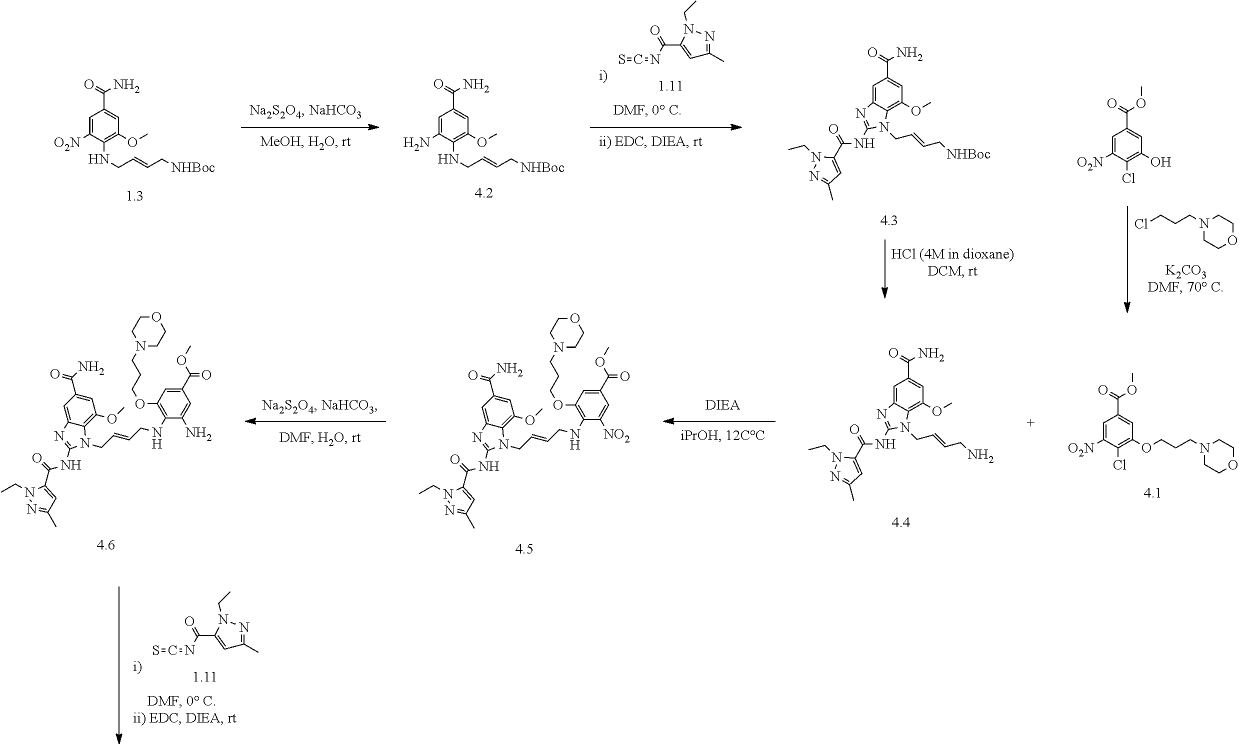

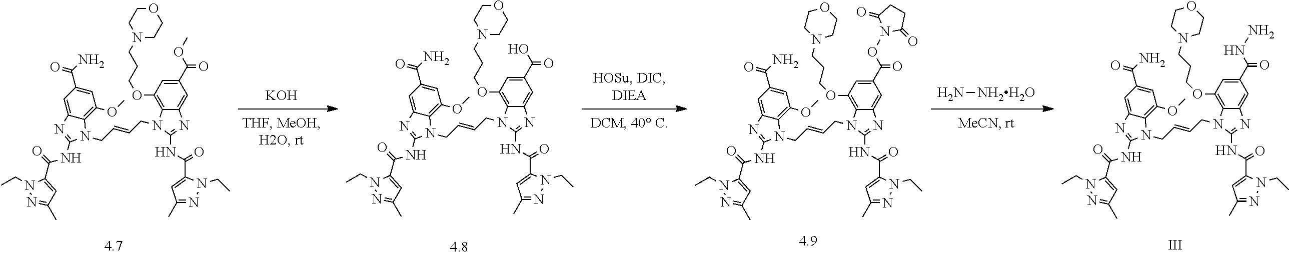

### Preparation of Compound 4.2

To a stirring solution of compound 1.3 (300 mg, 0.79 mmol) in 10 mL MeOH in a 50 mL round bottom flask was added Na₂S₂O₄ (14 equiv, 1.926 g, 11.04 mmol) as a solution in 10 mL water. The reaction mixture was allowed to stir for 15 minutes, then NaHCO₃ (43 equiv, 2.85 g, 33.93 mmol) was added. The reaction was judged complete after 10 minutes by UPLC-MS monitoring. The reaction mixture was diluted with water (100 mL), extracted twice with ethyl acetate (100 mL), washed with water (100 mL), dried over anhydrous sodium sulfate, and concentrated in vacuo to give crude compound 4.2 (225.83 mg; 0.644 mmol; 82%). The product was used for the next step without further purification. UPLC-MS retention time: 2.41 min (Icms_short), [M+H⁺]⁺=351.3.

### Preparation of Compound 4.3

To a stirring solution of compound 4.2 (180.6 mg, 0.5156 mmol) in 5 mL DMF at 0° C. in a 20 mL glass scintillation vial was added dropwise 0.058 mL of a 0.5-g/mL solution of compound 1.11 (28.8 mg, 0.148 mmol) in dioxane. After 15 minutes a second equal portion of compound 1.11 was added, followed 15 minutes later by a third equal portion. Reaction monitoring by UPLC-MS showed 87% conversion to the thiourea intermediate, so another 0.030 mL of compound 1.11 solution was added. Upon complete conversion to the thiourea intermediate, the reaction was allowed to warm to room temperature and EDC. HCl (1.3 equiv, 128.5 mg, 0.67 mmol) was added as a solution in 3 mL DMF followed by DIEA (3 equiv, 0.27 mL, 1.55 mmol). Reaction was allowed to stir at room temperature overnight. After 16 h UPLC-MS monitoring showed 88% conversion. Another 30 mg EDC·HCl (0.3 equiv, 0.156 mmol) was added. 5 minutes after the second addition of EDC, the reaction was complete. The reaction mixture was diluted with water (100 mL), extracted twice with ethyl acetate (100 mL), then washed with water (100 mL), dried over anhydrous sodium sulfate, and concentrated in vacuo to give crude compound 4.3. A yield was not obtained for this step. UPLC-MS retention time: 3.58 min (Icms_short), [M+H⁺]⁺=512.39.

### Preparation of Compound 4.4

To a stirring solution of compound 4.3 (200.1 mg, 0.391 mmol) in DCM (3 mL) in a 20 mL glass scintillation vial was added 0.98 mL HCl (4 M in dioxane, 10 equiv, 3.91 mmol). As the reaction proceeded, the product precipitated out of solution. Upon reaction completion by UPLC-MS, the reaction mixture was filtered and the solid product was rinsed with diethyl ether (20 mL) and left to air dry overnight to give crude compound 4.4 as an HCl salt. The crude compound was taken to the next step without further purification. A yield was not obtained for this step. UPLC-MS retention time: 2.51 min (Icms_short), [M+H⁺]⁺=412.32.

### Preparation of Compound 4.1

To a stirring solution of Methyl4-chloro-3-hydroxy-5-nitrobenzoate (1.15 g, 5 mmol, CAS #180031-12-3) in DMF (6 mL) in a 20 mL glass scintillation vial was added 4-(3-Chloropropyl) morpholine (1.1 equiv, 896 mg, 5.5 mmol, CAS #57616-74-7) and K₂CO₃ (2 equiv, 1.4 g, 10 mmol). The reaction mixture was brought to 70° C. and allowed to stir overnight. After 16 h, complete conversion to compound 4.1 was observed by UPLC-MS. The reaction mixture was diluted with water (100 mL, extracted 3 times with methyl tert-butyl ether, then washed 3 times with saturated sodium bicarbonate in water. The organic phase was dried over anhydrous sodium sulfate and concentrated in vacuo to give compound 4.1 (1.55 g; 4.32 mmol; 86%) as an orange solid. UPLC-MS retention time: 3.08 min (Icms_short), [M+H⁺]⁺=359.22.

### Preparation of Compound 4.5

To a solution of compound 4.4 (202.8 mg, 0.453 mmol) in iPrOH (2 mL) in a Discover 2.0 glass 10 mL vial was added compound 4.1 (1.05 eq, 170.6 mg, 0.475 mmol) then DIEA (0.248 mL, 1.42 mmol). The reaction vessel was sealed then stirred at 120° C. under microwave heating (CEM Discover 2.0) for 5 h until the reaction was no longer progressing (maximum conversion of 95%). The reaction mixture was diluted with water (100 mL), and extracted 3 times with ethyl acetate (100 mL). The combined organic phase was washed with water (100 mL), dried over anhydrous sodium sulfate and concentrated in vacuo to afford crude compound 4.5 (243.5 mg) as an orange solid. Half of the product was used for the next step without purification, the other half was purified by silica column chromatography (25G Sfar silica column) using 5→20% MeOH in DCM. Column fractions were concentrated to give compound 4.5 (61 mg; 0.0831 mmol; 18%) as an orange solid. UPLC-MS retention time: 3.32 min (Icms_short), [M+H⁺]+=734.50.

### Preparation of Compound 4.6

To a stirring solution of compound 4.5 (61 mg, 0.0831 mmol) in DMF (1.1 mL) in a 20 mL glass scintillation vial was added Na₂S₂O₄ (14 equiv, 202.6 mg, 1.163 mmol) as a solution in water (0.89 mL). After 15 minutes, NaHCO₃ was added (43 equiv, 300 mg, 3.57 mmol). After 1 h, very little progress was observed, and some solids were not in solution. 1 mL of DMF was added. Another 200 mg of Na₂S₂O₄ and 100 mg of NaHCO₃were added. The reaction mixture was left to stir overnight at room temperature. After 16 h, complete conversion was observed by UPLC-MS monitoring. The reaction mixture was diluted with water (75 mL) and extracted twice with ethyl acetate (75 mL). The combined organic layers were washed with saturated sodium bicarbonate in water (50 mL), dried over anhydrous sodium sulfate, and concentrated in vacuo to give crude compound 4.6 (27 mg; 0.0384 mmol; 46%) as a yellow solid. Crude product from the organic phase was used for the next step without further purification. UPLC-MS retention time: 2.76 min (Icms_short), [M+H⁺]⁺=704.55.

### Preparation of Compound 4.7

To a stirring solution of compound 4.6 (27 mg, 0.0384 mmol) at 0° C. in DMF (0.5 mL) in a 4 mL glass vial was added 23 μL of a 0.33 mg/μL solution of compound 1.11 (1 equiv, 7.49 mg, 0.0384 mmol) in dioxane. The compound 1.11 solution was added a total of 3 times, each 15 minutes apart (7.6 μL each). After UPLC-MS monitoring showed complete conversion to the thiourea intermediate, DIEA (5 equiv, 33.5 μL 0.192 mmol) and EDC (2 equiv, 14.7 mg, 0.0768 mmol) were added and the reaction mixture was allowed to warm to room temperature and react overnight. After 18 h the reaction solution went from a transparent orange solution to a cloudy yellow solution and the reaction was judged complete by UPLC-MS monitoring. The reaction solution was precipitated in cold diethyl ether, centrifuged, and decanted to give compound 4.7 as a yellow pellet. A yield was not obtained for this step. UPLC-MS retention time: 4.87 min (lcms_long), [M+H⁺]⁺=865.62.

### Preparation of Compound 4.8

To a stirring solution of compound 4.7 (47.3 mg, 0.0547 mmol) in MeOH (0.3 mL) and THF (0.6 mL) in a 4 mL glass vial was added KOH (10 equiv, 36.1 mg, 0.547 mmol) as a solution in 0.16 mL water. After 2 h, another 19 mg KOH was added in 75 μL water to accelerate the reaction. 3 h after the second KOH addition, the reaction was judged complete by UPLC-MS monitoring. 1 M HCl in dioxane was added to the reaction mixture until a pH of 3 was obtained (1 mL). The reaction mixture was diluted with water, frozen, and lyophilized. The resulting solids were resuspended in 90% DCM, 9% MeOH, 1% DIEA and solids removed by filtration. The filtrate was concentrated in vacuo to give compound 4.8 (40.5 mg; 0.0476 mmol; 87%) as a solid. UPLC-MS retention time: 4.06 min (lcms_long), [M+H⁺]⁺=851.58.

### Preparation of Compound 4.9

To a stirring solution of compound 4.8 (40.5 mg, 0.0476 mmol) in DCM (0.6 mL) in a 4 mL glass vial was added DIEA (6 equiv, 49.7 μL, 0.2855 mmol), HOSu (1.4 equiv, 7.66 mg, 0.0666 mmol) and DIC (1.4 equiv, 10.3 L, 0.0666 mmol). The reaction mixture was brought to 40° C. to stir for 8 h until UPLC-MS monitoring showed maximum conversion (reaction stopped progressing after 95% conversion). The reaction mixture was concentrated in vacuo to give compound 4.9 (42.8 mg; 0.0451 mmol; 95%) as a solid. The product was used for the next step without further purification. UPLC-MS retention time: 4.33 min (Icms_long), [M+H⁺]⁺=948.53.

### Preparation of Compound III

To a stirring solution of compound 4.9 (30 mg, 0.03168 mmol) in MeCN (2 mL) in a 20 mL glass scintillation vial was added hydrazine monohydrate (3 equiv, 7.32 mg, 0.095 mmol). After 5 minutes, the starting material was all consumed as observed by UPLC-MS monitoring. Reaction mixture was diluted with 1 mL of 1:1 MeCN: H₂O and purified by RP-HPLC (Phenomenex Luna C5 250×21.2 mm column; 20 mL/min flowrate; 0.05% TFA; 15-30% MeCN/H2O gradient over 35 min; 50 min total run time). Pure fractions were combined and lyophilized to give compound III (6.2 mg; 0.00717 mmol; 23%) as a white powder. UPLC-MS retention time: 3.46 min (Icms_long_C4), [M+H⁺]⁺=865.59.

### Example 4: Synthetic Scheme IV

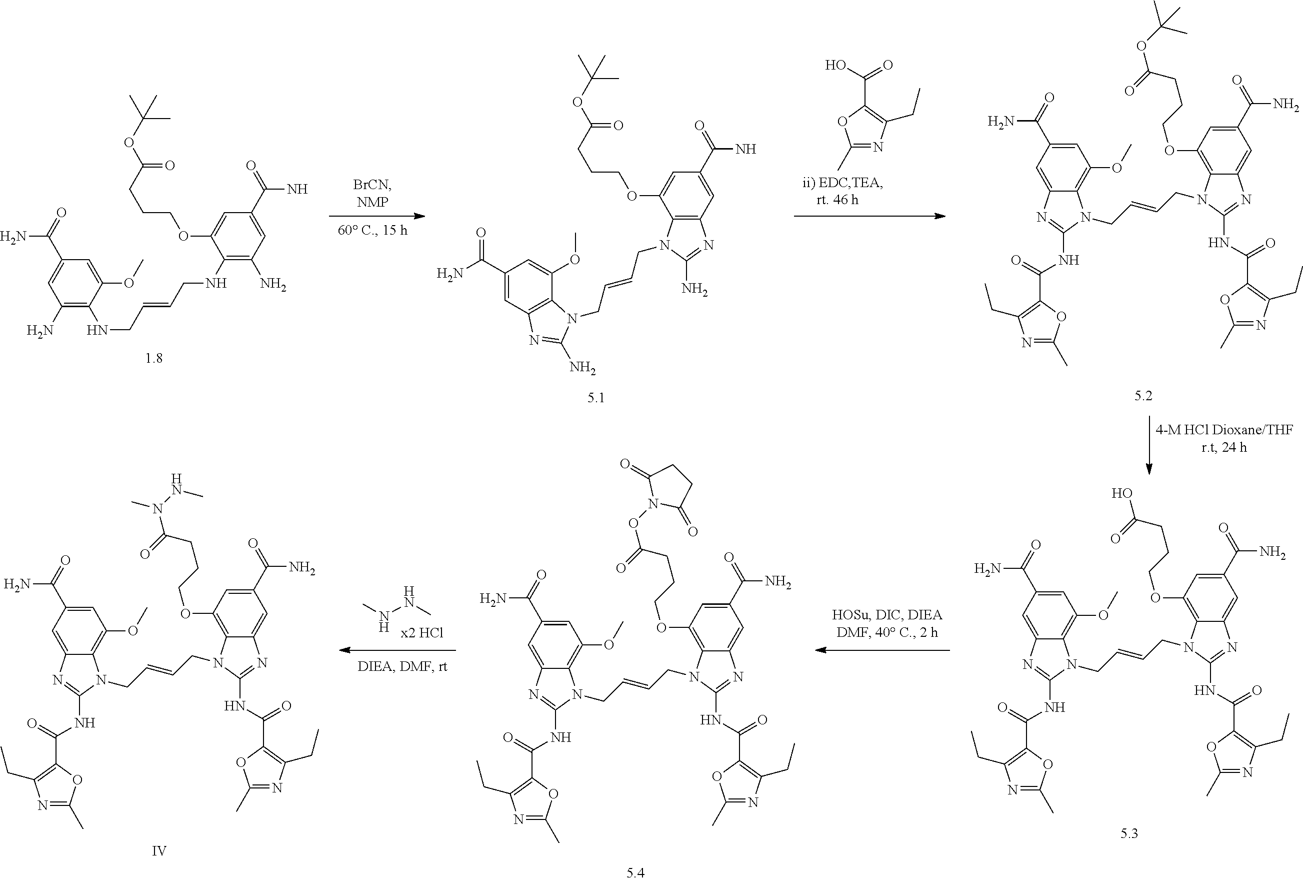

### Preparation of tert-Butyl 4-({2-amino-1-[(2E)-4-(2-amino-5-carbamoyl-7-methoxy-1H-1,3-benzodiazol-1-yl)but-2-en-1-yl]-5-carbamoyl-1H-1,3-benzodiazol-7-yl}oxy)butanoate (5.1)

To a solution of compound 1.8 (1.58 g; 2.90 mmol; 1.0 eq.) in N-methyl-2-pyrrolidone (25.2 mL) was added at room temperature cyanogen bromide (770 mg; 7.26 mmol; 2.5 eq.). The reaction mixture was stirred at 60° C. for 15 hours, cooled to room temperature and diluted with diethyl ether (75 mL). The mixture was filtered and the cake was washed with diethyl ether (2×25 mL). The solids collected by filtration afforded a beige solid (3.41 g). The residue was purified by flash chromatography over silica gel (50 μm irregular, 120 g) using dichloromethane/(methanol/ammonium hydroxyde 9/1) as eluent (95/5 for 5 min then to 70/30 over 30 min and 70/30 for 15 min) to afford impure compound 5.1 (1.31 g) as a beige solid after co-evaporation with acetonitrile (3×50 mL). The residue was purified by flash chromatography over silica gel (50 μm irregular, 40 g) using dichloromethane/(methanol/ammonium hydroxyde 9/1) as eluent (90/10 to 70/30 over 30 min and 70/30 for 20 min) to afford impure compound 5.1 (1.30 g) as a beige solid after co-evaporation with acetonitrile (3×50 mL). The residue was suspended in methanol (100 mL) and activated Amberlyst A26-OH⁻ (3.0 g) was added. The suspension was turned on the rotary evaporator at room temperature for 15 hours. Water (100 mL) was added to the mixture and it was turned on the rotary evaporator for 30 minutes. The suspension was filtered on cotton and the filtrate was concentrated to dryness to afford pure compound 5.1 (851 mg; 1.44 mmol; 49%) as a beige solid after co-evaporation with acetonitrile (3×50 mL). ¹H NMR (DMSO-d₆): δ 1.38 (s, 9H), 1.82-1.88 (m, 2H), 2.31 (t, J=7.2 Hz, 2H), 3.69 (s, 3H), 3.96 (t, J=6.3 Hz, 2H), 4.73 (dd, J=4.8, 11.7 Hz, 4H), 5.58-5.71 (m, 2H), 6.36 (d, J=10.5 Hz, 4H), 7.04 (d, J=3.8 Hz, 4H), 7.37 (s, 2H), 7.77 (br s, 2H). LCMS (2-100 ACN/H₂O+0.1% AF): Tr=6.28 min; purity=99.57%; [M+H⁺]⁺=593.18.

Compound 5.1 was obtained pure after only flash chromatography with a yield of 63%.

### Preparation of Compound 5.2

To a solution of compound 5.1 (150 mg; 0.25 mmol; 1.0 eq.) in DMSO (3.0 mL) was added 4-ethyl-2-methyl-1,3-oxazole-5-carboxylic acid (CAS #1564709-36-9; 98.1 mg; 0.63 mmol; 2.5 eq.), HATU (240 mg; 0.63 mmol; 2.5 eq.) and DIEA (348.6 μL; 2.0 mmol; 8 eq.). The reaction mixture was stirred at 105° C. for 6 hours, cooled to room temperature and crushed into cold diethyl ether (75 mL). Forming precipitate was isolated via centrifugation. The residue was purified by flash chromatography over silica gel (60 μm irregular, 25 g) (5→20% MeOH/DCM) to afford compound 5.2 as a beige solid (95 mg; 0.11 mmol; 44%). UPLC-MS retention time: 5.08 min (Icms_long); [M+H⁺]⁺=867.54.

### Preparation of Compound 5.3

To a suspension of compound 5.2 (95 mg; 0.11 mmol) in THF (4 mL) at room temperature was added HCl (4 M in dioxane; 4.0 mL; 16.0 mmol) and the forming mixture was stirred at room temperature for 24 hours. Complete conversion to compound 5.3 was confirmed by UPLC-MS monitoring. The reaction mixture was titrated into cold diethyl ether (100 mL), and forming precipitate was isolated via centrifugation. The supernatant was then removed, and the forming pellet was resuspended in cold diethyl ether (100 mL) and centrifuged. This step was repeated twice. Finally, the pellet was dried in vacuo to give compound 5.3 as HCl salt, a beige solid (80 mg; 0.098 mmol; 90%). The product was used for the next step without further purification. UPLC-MS retention time: 3.82 min (Icms_long); [M+H⁺]⁺=811.53.

### Preparation of Compound 5.4

To a suspension of compound 5.3 (20 mg; 0.024 mmol; 1.0 eq., HCl salt) in DMF (1 mL) at 40° C. was added DIEA (12.9 μL; 0.074 mmol; 3.0 eq.) followed by N-Hydroxysuccinimide (13.8 mg; 0.12 mmol; 5.0 eq.) and N,N′-Diisopropylcarbodiimide (18.4 mg; 0.144 mmol; 6.0 eq.). The forming mixture was monitoring by UPLC-MS until the reaction was no longer progressing (maximum conversion of 25%). The reaction mixture was titrated into cold diethyl ether (40 mL), and forming precipitate was isolated via centrifugation. The pellet was dried in vacuo to give of compound 5.4 as a beige solid. The crude product (21 mg; mixture of 25% compound 5.4 and 75% compound 5.3) was used for the next step without further purification. UPLC-MS retention time: 4.1 min (lcms_long), [M+H⁺]⁺=908.55.

### Preparation of Compound IV

Compound IV was prepared using N,N′-Dimethylhydrazine dihydrochloride following general method C to provide a beige solid. UPLC-MS retention time: 3.69 min (Icms_long); [M+H⁺]⁺=853.51.

### Example 5

Compound V below was prepared using the general methods disclosed herein.

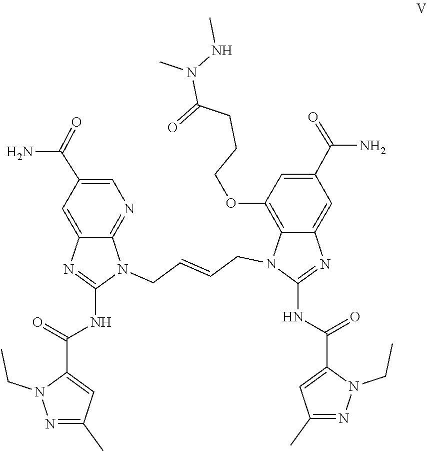 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCC(=O)N(C)NC)c21]

### Example 6

Compound VI below was prepared using the general methods disclosed herein.

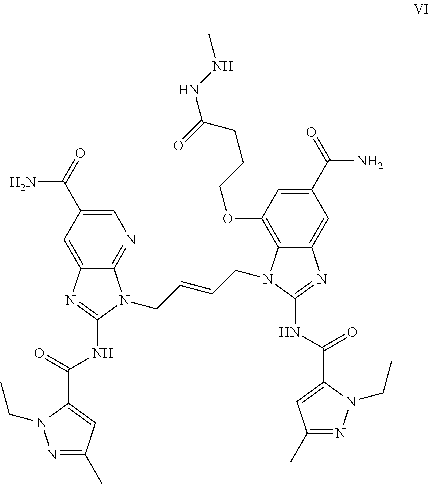 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCC(=O)NNC)c21]

### Example 7

Compound VII below was prepared using the general methods disclosed herein.

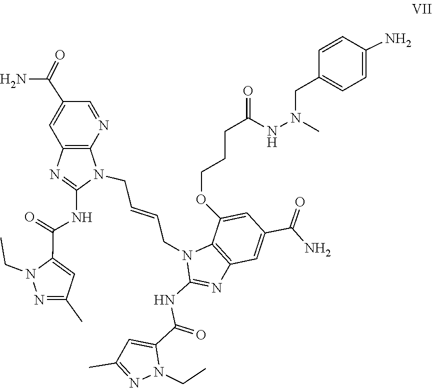 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCC(=O)NN(C)Cc3ccc(N)cc3)c21]

### Example 8

Compound VIII below was prepared using the general methods disclosed herein.

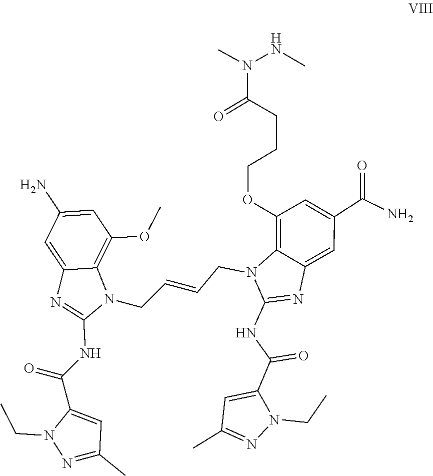 [SMILES: CCn1nc(C)cc1C(=O)Nc1nc2cc(N)cc(OC)c2n1C/C=C/Cn1c(NC(=O)c2cc(C)nn2CC)nc2cc(C(N)=O)cc(OCCCC(=O)N(C)NC)c21]

### Example 9

Compound IX below was prepared using the general methods disclosed herein.

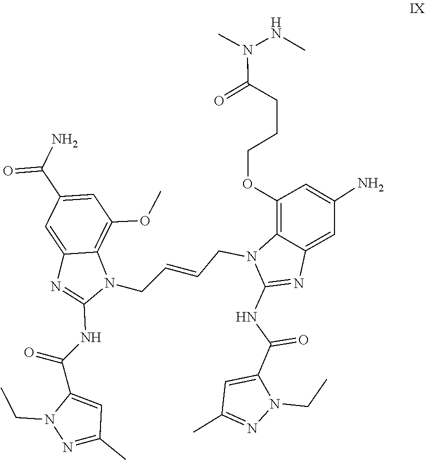 [SMILES: CCn1nc(C)cc1C(=O)Nc1nc2cc(C(N)=O)cc(OC)c2n1C/C=C/Cn1c(NC(=O)c2cc(C)nn2CC)nc2cc(N)cc(OCCCC(=O)N(C)NC)c21]

### Example 10

Compound X below was prepared using the general methods disclosed herein.

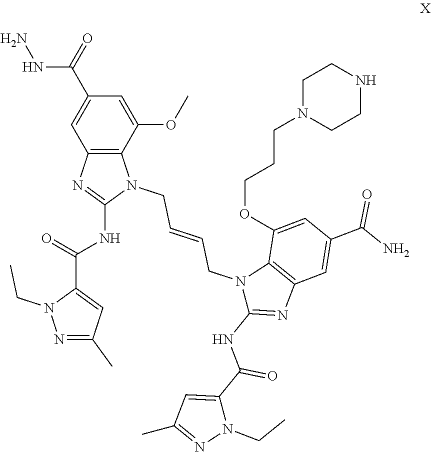 [SMILES: CCn1nc(C)cc1C(=O)Nc1nc2cc(C(=O)NN)cc(OC)c2n1C/C=C/Cn1c(NC(=O)c2cc(C)nn2CC)nc2cc(C(N)=O)cc(OCCCN3CCNCC3)c21]

### Example 11

Compound XI below was prepared using the general methods disclosed herein.

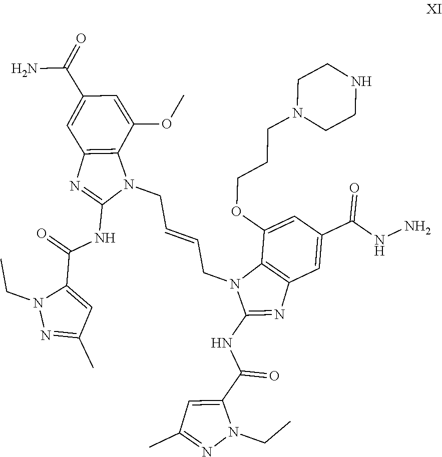 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NN)cc(OCCCN3CCNCC3)c21]

### Example 12

Compound XII below was prepared using the general methods disclosed herein.

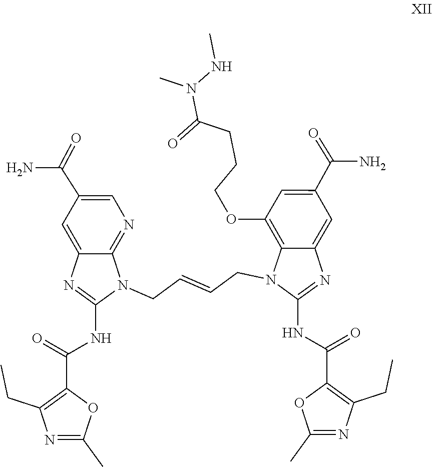 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCC(=O)N(C)NC)c21]

### Example 13

Compound XIII below was prepared using the general methods disclosed herein.

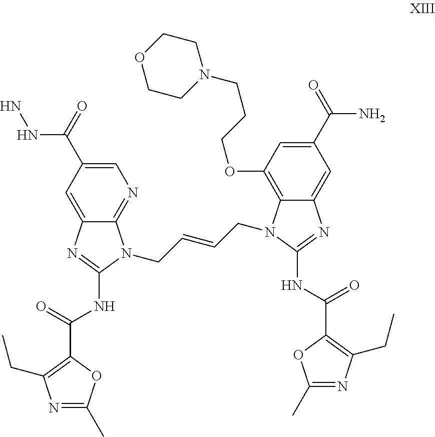 [SMILES: CCc1nc(C)oc1C(=O)Nc1nc2cc(C(=O)NN)cnc2n1C/C=C/Cn1c(NC(=O)c2oc(C)nc2CC)nc2cc(C(N)=O)cc(OCCCN3CCOCC3)c21]

### Example 14

Compound XIV below was prepared using the general methods disclosed herein.

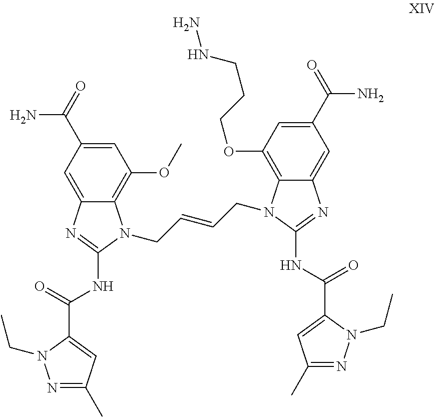 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCNN)c21]

### Example 15

Compound XV below was prepared using the general methods disclosed herein.

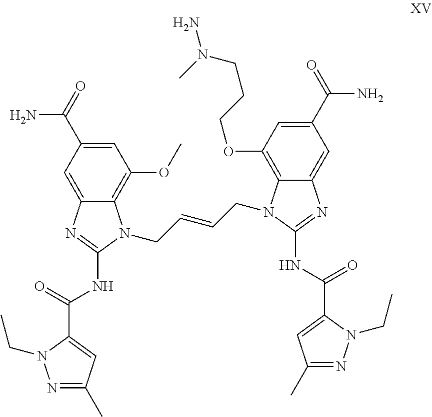 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)N)c21]

### Example 16

Compound XVI below was prepared using the general methods disclosed herein.

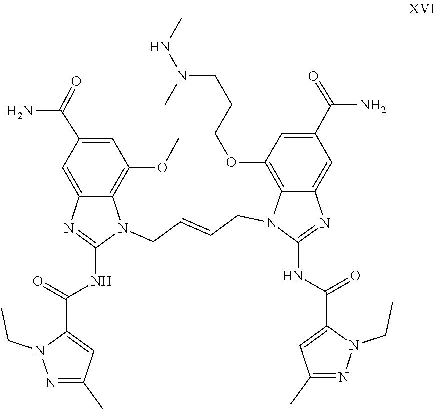 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)NC)c21]

### Example 17

Compound XVII below was prepared using the general methods disclosed herein.

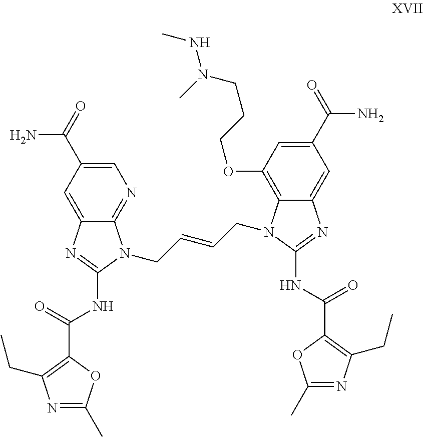 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(C)NC)c21]

### Example 18

Compound XVIII below was prepared using the general methods disclosed herein.

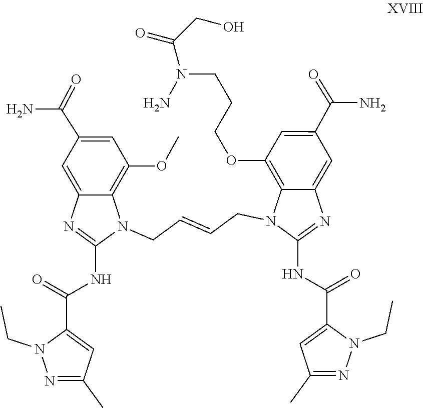 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(N)C(=O)CO)c21]

### Example 19

Compound XIX below was prepared using the general methods disclosed herein.

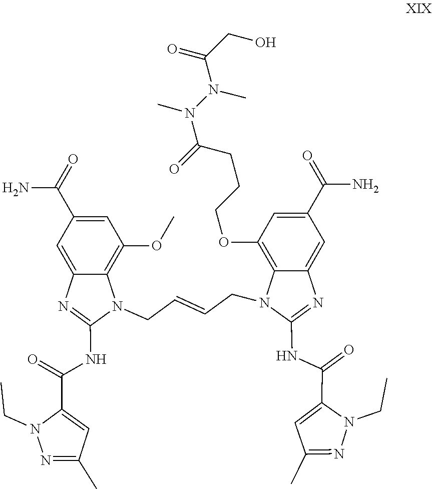 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)N(C)C(=O)CO)c21]

### Example 20: THP-1 Assay

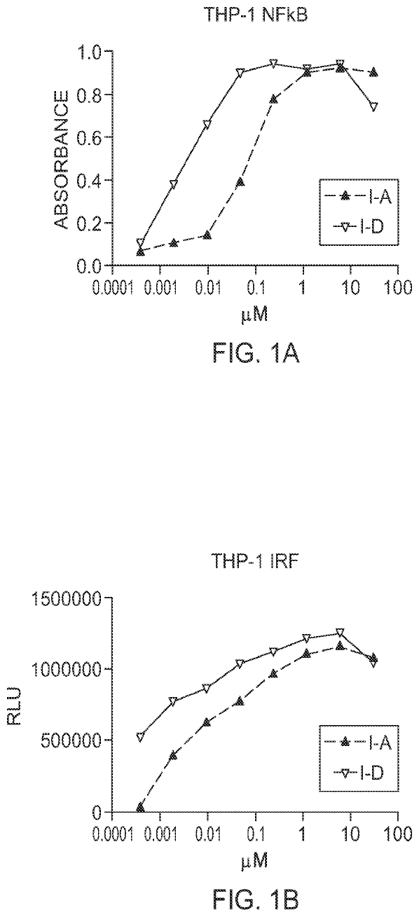

THP1 Dual Cells™, were purchased from Invivogen (Cat. thpd-nfis), and used to determine the biological activity of the compounds on a molecular level and to determine downstream activation of STING signaling (IRF and NFKB-pathway activation). Briefly, cultured cells were seeded on the day of the experiment with a density of 1×10⁵ cells per well (96-well format in RPMI supplemented with 25 mM HEPES, 10% heat inactivated FCS,). A serial dilution of the compounds was prepared and added to the cells for a total incubation time of 24 hours. The test items were tested in 8 concentrations to determine a dose-dependent activation of the NFKB and the IRF pathway. After 24 h cells were centrifuged and supernatants harvested for the detection of secreted alkaline phosphatase (measured by QUANTI-Blue™ detection solution; Cat. rep-qbs Invivogen) or lucia luciferase (measured by QUANTI-Luc™ reagent; Cat. rep-qlc1, Invivogen), respectively. The read outs were be performed according to the manufacturer's instructions. EC50 values were calculated using GraphPad Prism 9. THP-1 Plots for compounds I-A and I-D are shown in FIGS. 1A and 1B.

### Example 21: HEK STING Assay with PFO

HEK293 cells with a knocked-in STING receptor were purchased from Invivogen (Cat. 293dh232), and used to determine the biological activity of compounds with and without the cell membrane-permeabilizing agent PFO. Activity was determined by analysis of downstream activation fo STING signaling (IRF and IFNβ-pathway activation). Briefly, cultured cells were seeded at 2×10⁴ cells per well (96-well format in DMEM) and rested over night. The next day, a serial dilution of components was prepared and added to the cells for a total incubation time of 24 hours. The test items were tested in 8 concentrations to determine a dose-dependent activation of the IRF pathway and IFNβ secretion. In addition to the test items, 50 ng/ml of PFO were added to the wells. After 24 hour incubation of test items with and without PFO (US Biological Life Sciences, Cat. 370743) the cells were centrifuged and supernatants harvested for the detection of secreted alkaline phosphatase (measured by QUANTI-Blue™ detection solution; Cat. rep-qbs Invivogen) or lucia luciferase (measured by QUANTI-Luc™ reagent; Cat. rep-qlc1, Invivogen), respectively. The read outs were performed according to the manufacturer's instructions.

### Example 22: STING Haplotype Assays

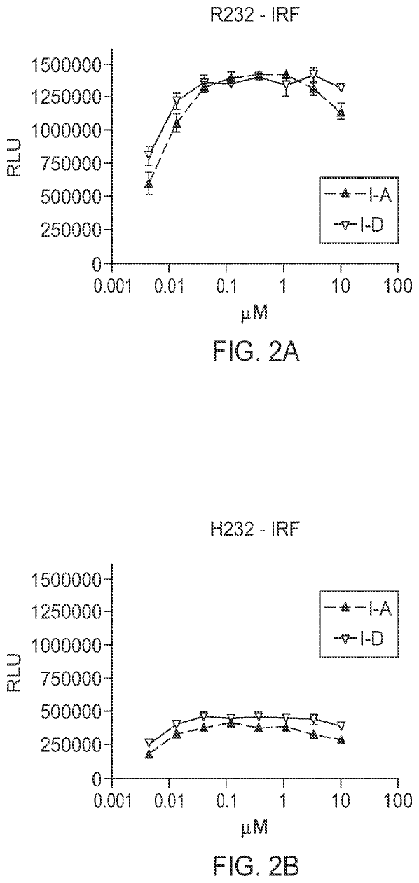

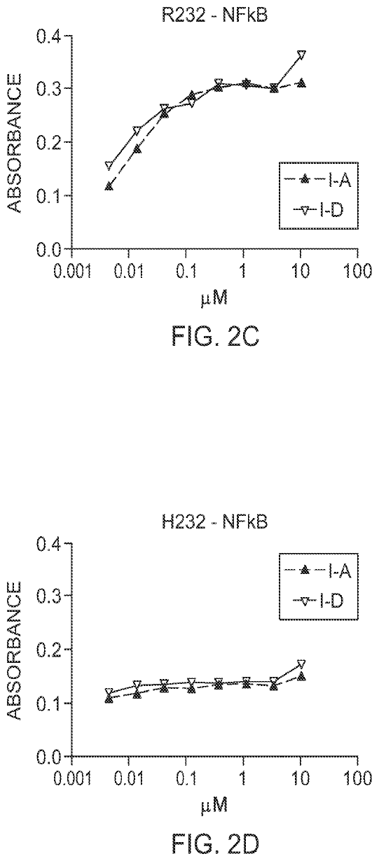

THP1 Dual Cells™ with knock-in of different human STING variants, namely THP-1Dual™ KI-hSTING-H232 Cells (Cat. Thpd-h232) and THP-1Dual™ KI-hSTING-R232 Cells (Cat. thpd-r232), were purchased from Invivogen. Within these cell lines naturally expressed STING was first knocked out and followingly the coding sequence of the human STING variant was knocked in. R232 is described by Invivogen to occur in approximately 45-58% of human population, while occurrence of h232 is at approximately 14%. Biological activity of compounds was evaluated as described in Example 20. STING haplotype curves for compounds I-A and I-D are shown in FIGS. 2A-2D.

### Example 23: PBMC Assay

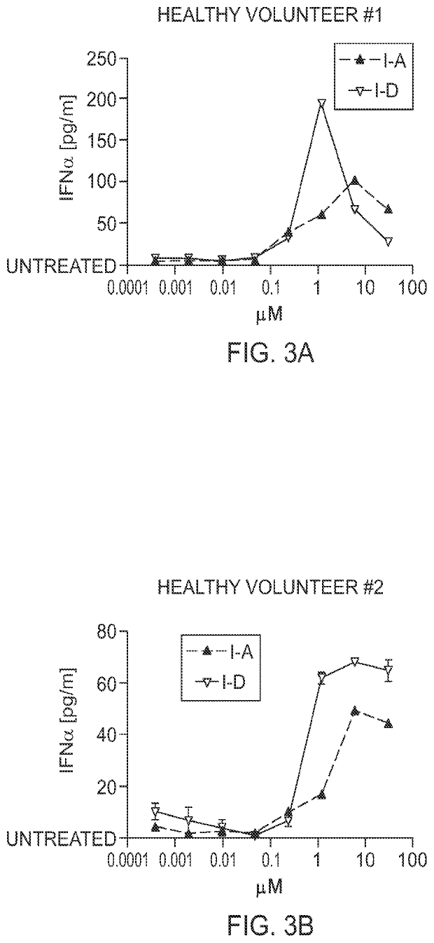

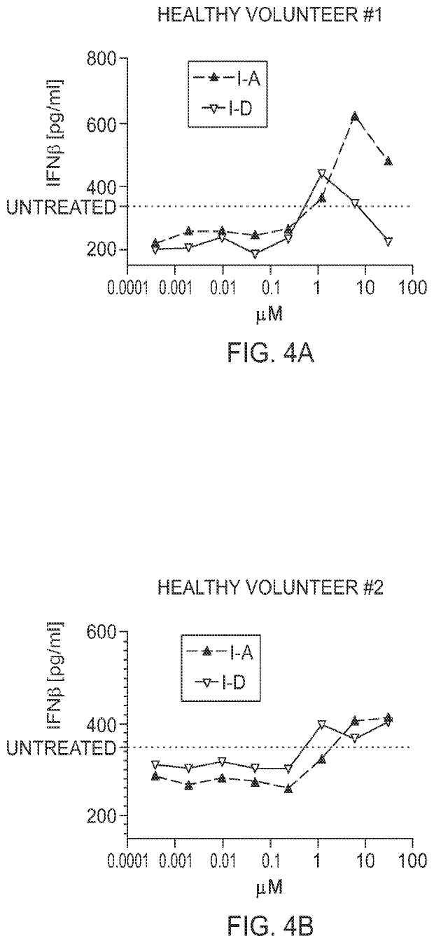

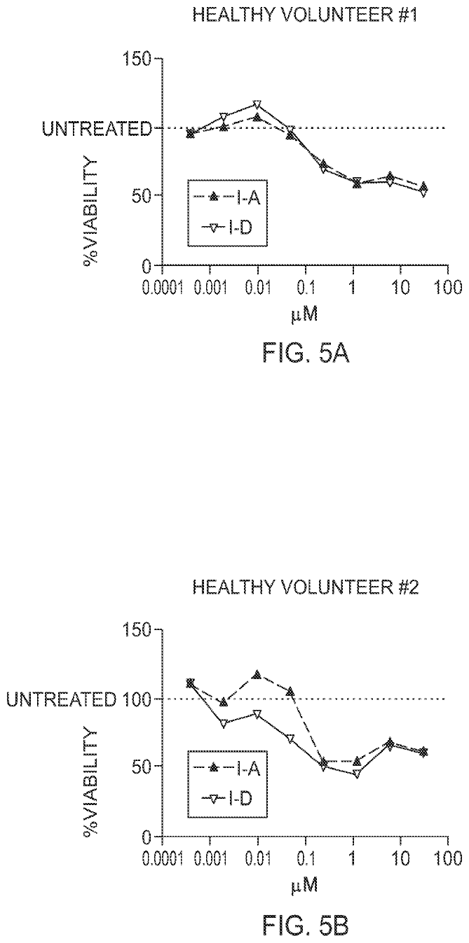

Compounds I-A to IV were additionally tested on PBMCs from healthy donors to determine the dose-dependent cytokine induction. PBMCs from buffy coats were thawed on the day of the experiment and seeded with a density of 5×10⁵ cells per well (96-well format, RPMI with 5% Human Serum+1% MEM Non-Essential Amino Acids+1% Sodium Pyruvate). In line with the experiment above, a serial dilution of the compounds was prepared and added to the cells for 24 hours. On the next days, cells were centrifuged and supernatants harvested for a cytokine readout based on a multiplex assay kit from mesoscale discovery (customized multiplex panel for the cytokines: IFNα2a, IFNβ, TNFα and IL6). Impact on cell viability was determined by CellTiter Glo2.0 from promega (Cat. G9241). Both experimental read outs were performed according to the manufacturer's instructions. PBMC representative curves for compounds I-A and I-D are shown in FIGS. 3A-5B.

### Example 24: Human and Murine STING Production

T7 Shuffle E. coli cells, transformed with an expression vector encoding either a truncated version of human or murine His-tagged STING, were cultivated overnight in 50 mL LB media. 200 mL of LB media were inoculated to an OD600 of 0.05 and cells were grown at 37° C. at 120 rpm until an OD600 of 0.5 was reached. Protein expression was induced using 1 mM IPTG and cells were further cultivated at 30° C. over night. Cells were harvested by centrifugation at 4000 rpm for 30 min at 4° C. and frozen at −80° C. Subsequently, cells were thawed and resuspended in 30 mL IMAC-Buffer A (10 mM HEPES, 50 mM NaCl, 10 mM Imidazole, 250 mM L-Arginine, 10% Glycerol, pH 7.1) supplemented with 15 U/mL benzonase, 0.2 mg/mL MgCl₂ and 0.1 mg/mL lysozyme. Lysis was performed by five sonification cycles with an amplitude of 25% and a pulse for 2 s, with 5 min incubation on ice between cycles. The lysate was centrifuged for 15 min at 16.000 g and the supernatant was filtrated (0.22 μm). Purification of STING was done using an ÄKTA prime chromatography system. A 1 mL His Trap excel column was equilibrated with IMAC-Buffer A before lysate was applied. After washing with multiple column volumes of buffer, STING was eluted using a linear gradient of IMAC-Buffer B (10 mM HEPES, 50 mM NaCl, 500 mM Imidazole, 250 mM L-Arginine, 10% Glycerol, pH 7.1). Elution fractions were analyzed by SDS-PAGE and STING containing fractions were mixed. Using the Äkta pure system in combination with a HiLoad Superdex 16/600 75 pg column, STING was further purified by preparative size-exclusion chromatography. Fractions were analyzed by SDS-PAGE and STING containing fractions were combined.

Purified proteins were biotinylated using EZ-Link Sulfo-NHS-LC-Biotin (Life Technologies GmbH) in ten-fold excess for 2 h on ice. The reaction was quenched by addition of 100 mM Tris pH8 and 1 h incubation on ice. Remaining biotin was removed by performing buffer exchange to PBS using Zeba Spin Desalting Columns, 7K MWCO, 0.5 mL (Thermo Fischer). Protein concentrations were determined using the Nanodrop system.

### Example 25: Affinity Measurement Assay

Affinity measurement of STING-binding compounds were performed using the Biacore T200 SPR system (GE Healthcare, Biacore T200 control Software 3.2). Therefore, the Biotin CAPture kit, series S (Cytiva Europe, Cat.no. 28920234) was utilized. A freshly docked Biotin CAPture chip was rehydrated overnight in the instrument (standby mode). The following day, the chip surface was conditioned three times with regeneration solution (6 M guanidine-HCl, 0.25 M NaOH) for 60 s at 10 μL/min on flow cell (FC) 1 and FC2. Subsequently, the Biotin CAPture reagent (Cytiva Europe, Cat.no. 29423383) was applied for 300 s at 2 μL/min on FC1 and FC2, followed by capturing of 100 nM biotinylated murine or 150 nM biotinylated human STING, respectively. The immobilization of STING was performed for 600 s at 10 μL/min on FC2 followed by quenching of unbound streptavidin by 0.1 mM biocytin for 60 s and 30 μL/min on both flow cells. Five injections of 10 nM, 5 nM, 2.5 nM, 1.25 nM, 0.625 nM and 0 nM (blank control) of STING-binding compounds were performed over both flow cells for 120 s with a flow rate of 30 μL/min, followed 900 s dissociation time. After each cycle, the surface was regenerated for 120 s with 10 μL/min using regeneration solution. All measurements were performed at 25° C. using HBS-EP+ buffer (Cytiva Europe, Cat.no BR100669). The control responses from FC1 were subtracted by the sample measurement in FC2 (FC2-FC1) followed by subtraction of the 0 nM blank injection. Resulting binding curves of this multicycle kinetic experiment were fitted using the Biacore T200 Evaluation Software 3.2 and a 1:1 binding model.

### Example 26: Tables of Activity and Binding Affinities of Compounds I-A to IV

The activity and binding affinities of compounds I-A to IV are shown in Tables 2-4 below.

TABLE 2
THP-1 EC50 (IRF)

| Entry | Compound | THP-1 EC50 (IRF) |
|---|---|---|
| 1 | I-J | 0.02336 |
| 2 | I-K | 0.00974 |
| 3 | I-L | 0.0134 |
| 4 | II-A | 0.08487 |
| 5 | II-B | 2.743 |
| 6 | II-C | 0.2673 |

TABLE 3
PBMC EC50 (μM) for IFNα and IFNβ

| | | PBMC EC50 (μM) |
|---|---|---|
| Entry | Compound | (for IFNα, IFNb) |
| 1 | I-A | IFNα: 0.3335 |
| | | IFNβ: 1.249 |
| 2 | I-B | IFNβ: 0.4031 |
| 3 | I-C | IFNβ: 1.0955 |
| 4 | I-D | IFNα: 0.2601 |
| | | IFNβ: 0.2646 |
| 5 | I-E | IFNα: 0.762 |
| | | IFNβ: 0.13945 |
| 6 | I-F | IFNβ: 0.20945 |
| 7 | I-G | IFNα: 2.024 |
| | | IFNβ: 0.3111 |
| 8 | I-H | IFNα: 0.6648 |
| | | IFNβ: 0.24925 |
| 9 | I-I | IFNα: 0.4096 |
| | | IFNβ: 0.21165 |
| 10 | I-J | IFNα: 0.321 |
| | | IFNβ: 0.2928 |
| 11 | I-K | IFNα: 0.3017 |
| | | IFNβ: 0.234 |
| 12 | I-L | IFNβ: 0.3344 |
| 13 | II-A | IFNα: 0.87465 |
| | | IFNβ: 0.4031 |
| 14 | II-B | IFNα: 1.9505 |
| | | IFNβ: 1.1645 |
| 15 | II-C | IFNα: 1.148 |
| | | IFNβ: 0.9102 |
| 16 | II-D | IFNα: 1.1405 |
| | | IFNβ: 0.8208 |
| 17 | III | IFNβ: 0.38725 |

TABLE 4
Affinity (K_{d}) rHSTING (M)

| | | Affinity (K_{d}) |
|---|---|---|
| Entry | Compound | rhSTING (M) |
| 1 | I-D | 4.133E−12 |
| 2 | II-B | 9.587E−10 |
| 3 | II-D | 2.913E−9 |
| 4 | III | 3.190E−10 |

### Example 27: Anti-Tumoral Efficacy for Comparative Compound diABZI in 4T1

Anti-tumoral efficacy of a comparative compound diABZI (CAS #: 2138299-33-7), having the following structure:

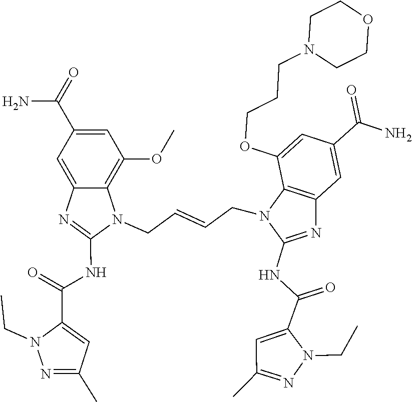 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN3CCOCC3)c21]

was tested in a murine 4T1 breast cancer model. Balb/c RJ mice were injected subcutaneously with 1×10⁵ 4T1 cells. Three doses of agonist diluted in PBS (31 nmol, 3.1 nmol and 0.31 nmol) were administered at day 11, 16 and 21 after inoculation and compared to an untreated control group. Tumor size was monitored using caliper measurement (volume was calculated by

$$\frac{tumor width * tumor width * tumor length}{2} )$$

until day 21, when animals were sacrificed and tumor cytokines were analyzed compared to serum 3 h after final agonist injection. To evaluate statistical significance of tumor volume reduction compared to untreated, a 2way ANOVA test was used: *** p<0.0001.

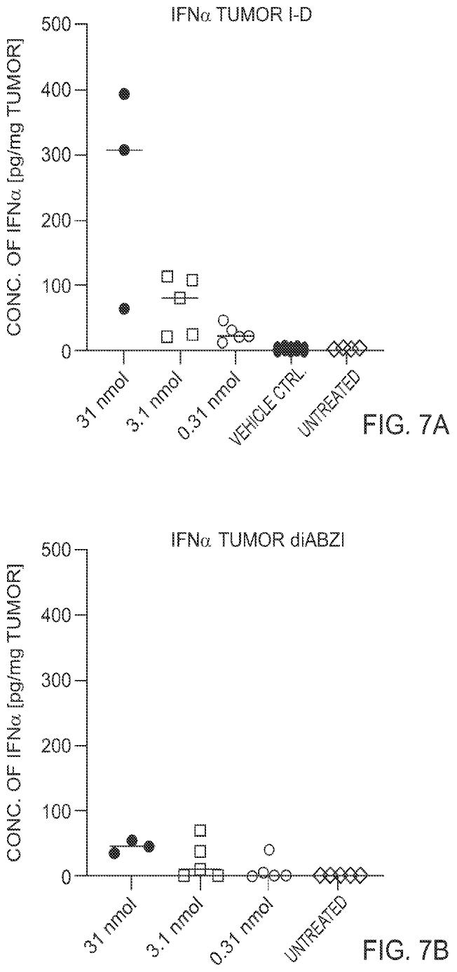

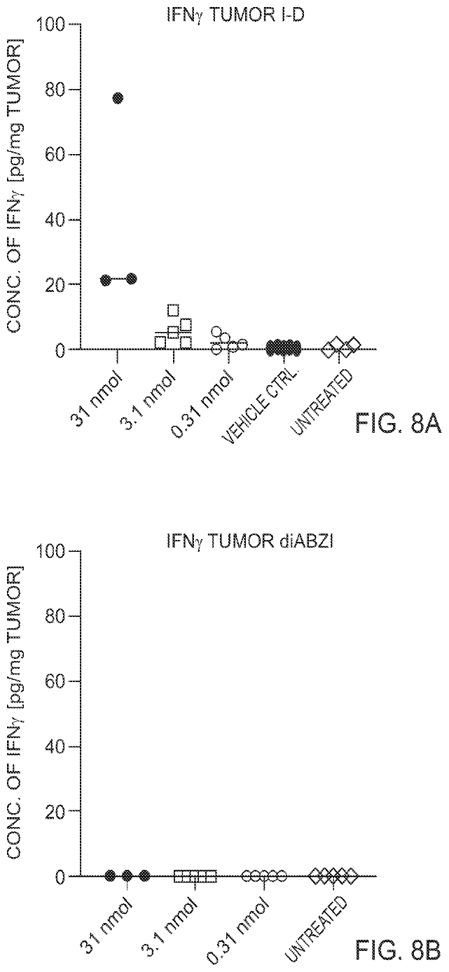

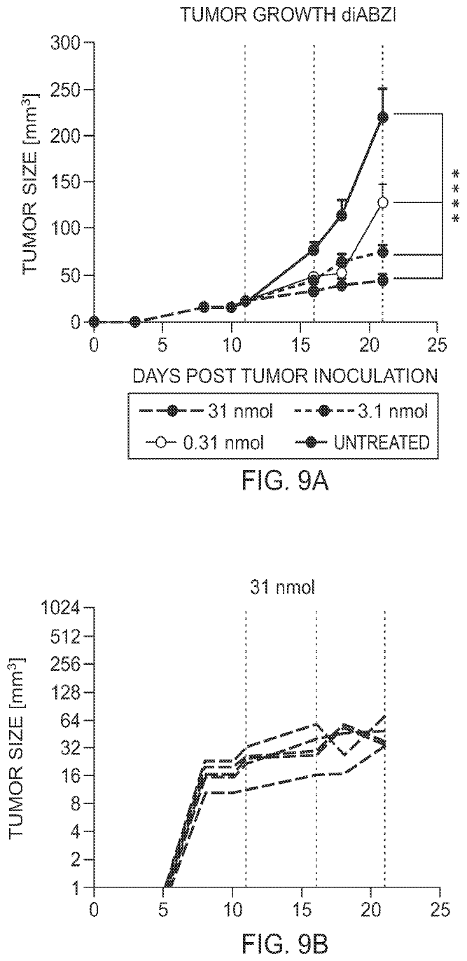

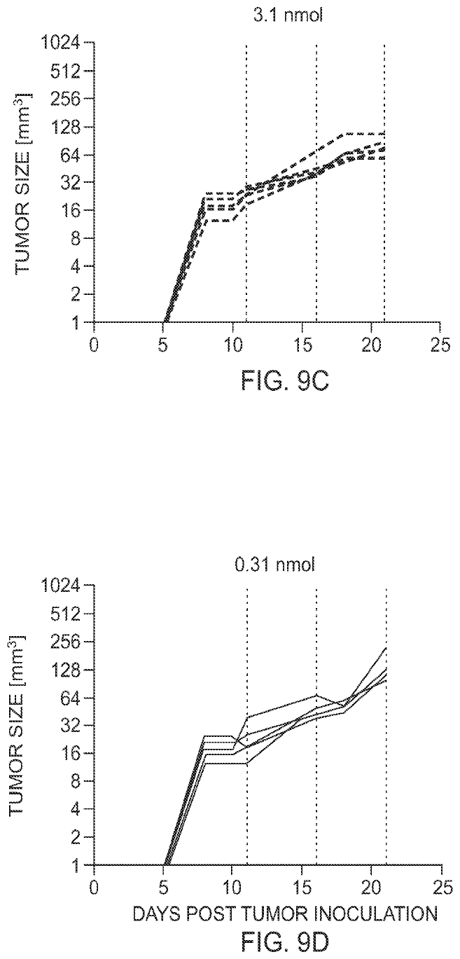

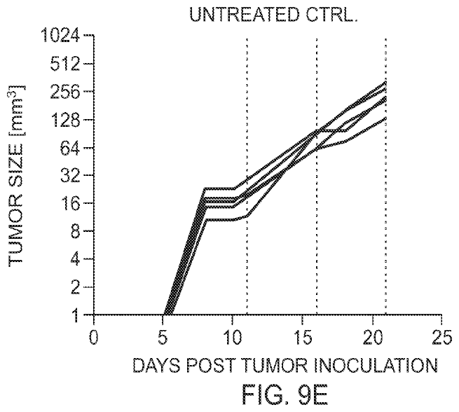

Tumor growth data for diABZI is shown in FIGS. 9A-9E. Cytokine data for diABZI is shown in FIGS. 7B and 8B.

### Example 28: Anti-Tumoral Efficacy for Compound I-D in 4T1

The anti-tumoral efficacy of compound I-D was tested in a murine 4T1 breast cancer model. Balb/c Rj mice were injected subcutaneously with 1×10⁵ 4T1 cells. Three doses of compound I-D formulated in 5% Solutol in 2.5% glucose (31 nmol, 3.1 nmol and 0.31 nmol) or the vehicle control (5% Solutol in 2.5% glucose) were administered intratumorally at days 11, 16 and 21 after inoculation. Tumor size was monitored using BioVolume® (calculating the tumor size based on RGB, thermal and 3D model data) until day 21, when animals were sacrificed and tumor cytokines were analyzed compared to serum 3 h after final agonist injection. To evaluate the statistical significance of tumor volume reduction compared to the vehicle control, a 2way ANOVA test was used: *** p<0.0001.

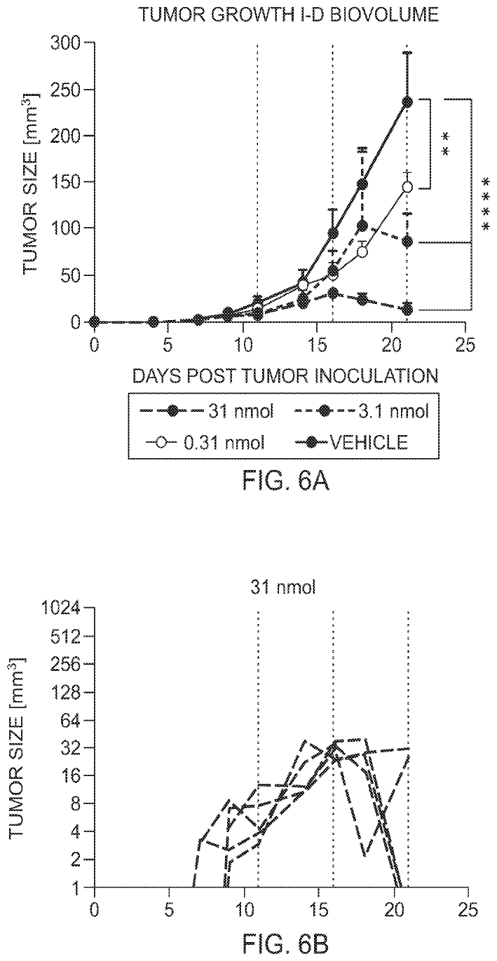

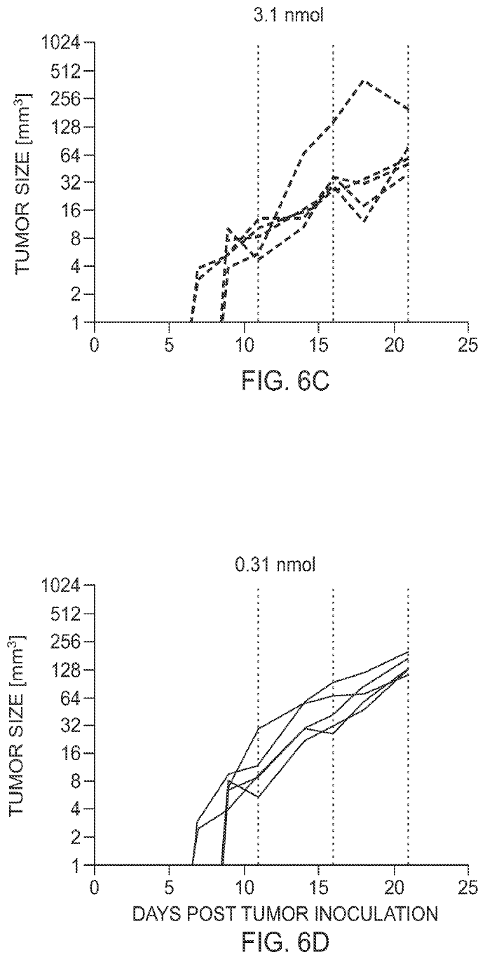

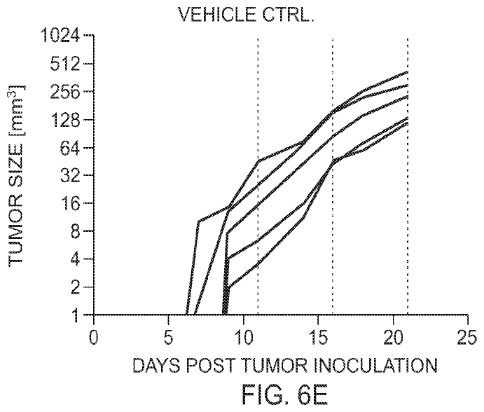

Tumor growth data for each treatment group, as measured using BioVolume, is shown in FIGS. 6A-6E. As can be seen from these Figures, administration of the various dosages of compound I-D led to a marked decrease in tumor size when compared to the vehicle control.

Concentrations of IFNα and IFNγ in the tumor for each treatment group and an untreated control are shown in FIGS. 7A and 8A. As shown in FIGS. 7A-8B, compound I-D induced higher levels of IFNα and IFNγ cytokines in the tumor, indicating a better activation of the immune system in the tumor when compared to comparative compound diABZI.

### Example 29: Synthetic Scheme for Compound XX

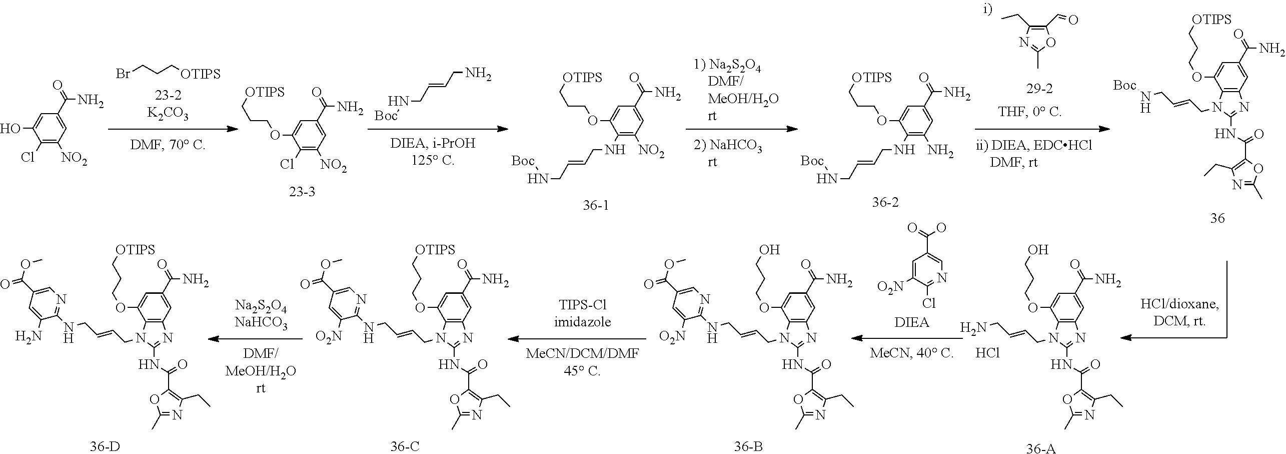

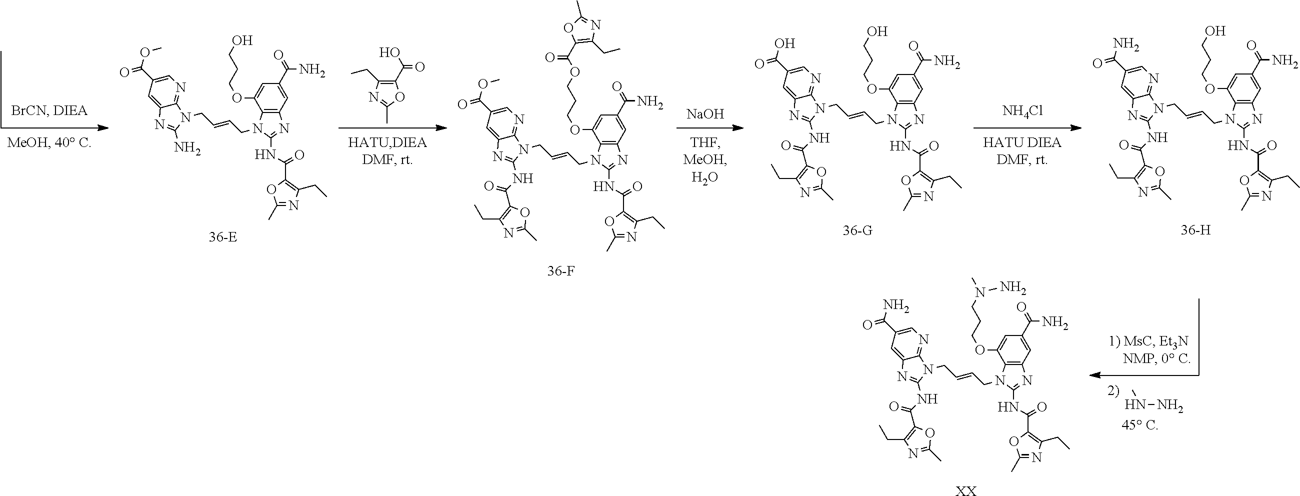

### Preparation of (3-bromopropoxy)tris(propan-2-yl)silane (23-2)

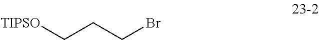

In a flask under argon, triisopropylsilyl chloride (76.9 mL; 360 mmol; 1.00 eq.) followed by imidazole (61.2 g; 899 mmol; 2.50 eq.) were added to a solution of 3-bromopropan-1-ol (50.0 g; 360 mmol; 1.00 eq.) in dichloromethane (360 mL). The reaction mixture was stirred for 2.5 days at room temperature. Dichloromethane (75 mL) and water (75 mL) were added to the reaction mixture. The reaction mixture was diluted with water (500 mL). The layers were separated and the aqueous layer was extracted with dichloromethane (3×500 mL). The combined organics layers were dried over anhydrous sodium sulfate, filtered and concentrated under reduced pressure to afford an opaque oil (109 g). The residue was purified by silica gel chromatography (Silica: 970 g; liquid loading in cyclohexane) using cyclohexane as eluent to afford the pure compound 23-2 (80.3 g; 272 mmol; 76%) as a colorless oil.

Note: The product is only detected by TLC (Cyclohexane/Ethyl acetate 9:1; Stain: potassium permanganate).

¹H NMR (CDCl₃): δ 1.02-1.32 (s, 21H), 2.04 (tt, 2H, J=6.0, 6.0 Hz), 3.53 (t, 2H, J=6.0 Hz), 3.80 (t, 2H, J=6.0 Hz).

### Preparation of 4-chloro-3-nitro-5-(3-{[tris(propan-2-yl)silyl]oxy}propoxy)benzamide (23-3)

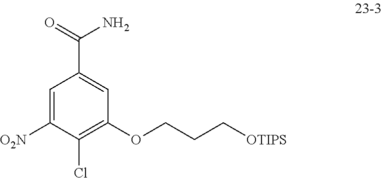

In a flask under argon, a suspension of compound 4-Chloro-3-hydroxy-5-nitrobenzamide 10-5 (10.6 g; 46.2 mmol; 1.00 eq.), compound 23-2 (16.4 g; 55.4 mmol; 1.20 eq.) and anhydrous potassium carbonate (8.30 g; 60.0 mmol; 1.30 eq.) in anhydrous N,N-dimethylformamide (59 mL) was stirred at 70° C. for 18 hours. The reaction mixture was concentrated under vacuum. The residue was suspended in dichloromethane (200 mL) and sonicated for 20 minutes at room temperature. The solid was collected by filtration to afford a beige solid (25.4 g). The residue was purified by flash chromatography over silica gel in two portions of 12.7 g each (330 g, 50 μm irregular; solid loading over silica) using dichloromethane/methanol as eluent (100:0 for 5 minutes, to 97.5:2.5 over 30 minutes and 97.5:2.5 for 10 minutes) to afford compound 23-3 (20.4 g; 45.2 mmol; 98%) as a pale yellow solid contaminated with 4.64 wt % of N,N-dimethylformamide.

¹H NMR (DMSO-d₆): δ 0.95-1.36 (m, 21H), 2.00 (dd, 2H, J=5.9, 5.9 Hz), 3.88 (d, 2H, J=5.9 Hz), 4.33 (d, 2H, J=5.9 Hz), 7.76 (s, 1H), 7.88 (d, 1H, J=1.8 Hz), 8.04 (d, 1H, J=1.8 Hz), 8.28 (s, 1H).

LCMS (2-100 ACN/H₂O+0.1% FA, 5 min): Tr=3.85 min; purity=100%; [M+H⁺]=443.2.

### Preparation of tert-Butyl N-[(2E)-4-{[4-carbamoyl-2-nitro-6-(3-{[tris(propan-2-yl)silyl]oxy}propoxy)phenyl]amino}but-2-en-1-yl]carbamate (36-1)

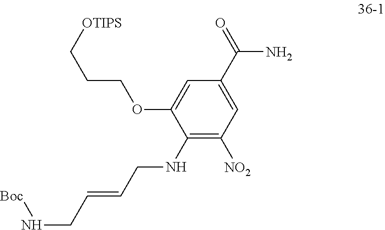

In a sealed flask purged with argon, N,N-diisopropylethylamine (4.00 eq., 16.9 mL, 96.9 mmol) was added to a stirred solution of compound 23-3 (1.00 eq., 12.0 g, 24.2 mmol) and tert-butyl N-[(2E)-4-aminobut-2-en-1-yl]carbamate (1.50 eq., 6.77 g, 36.3 mmol) in isopropanol (80 mL) at room temperature. The flask was sealed and the reaction mixture was stirred at 125° C. for 18 hours. The reaction mixture was diluted with water (400 mL) and ethyl acetate (400 mL). The organic layer was dried over anhydrous sodium sulfate, filtered and concentrated under vacuum to afford the crude product (16.5 g) as an orange oil. The residue was purified by flash chromatography over silica gel (Column: Interchim 15 μm 330 g; Loading: Solid (Silica, 45 g)) using cyclohexane/ethyl acetate as eluents (gradient from 100:00 to 70:30 over 30 minutes then 70:30 for 45 minutes then gradient to 20:80 over 25 minutes) to afford the desired compound 36-1 (11.2 g, 19.1 mmol, 79%, contaminated with 1.0 wt % of ethyl acetate) as a red solid.

LC/MS (CSH, water+0.1% HCO₂H/MeCN 2-100 gradient, 3.5 min): Rt=2.04 min, 100%, [M-tBu+H]⁺=525.85

¹H NMR (DMSO-d₆, 400 MHz): δ ppm 8.19 (d, J=1.8 Hz, 1H), 8.01 (s, 1H), 7.73 (t, J=6.1 Hz, 1H), 7.56 (d, J=2.0 Hz, 1H), 7.29 (s, 1H), 6.91 (t, J=6.0 Hz, 1H), 5.63-5.48 (m, 2H), 4.24-4.08 (m, 4H), 3.86 (t, J=6.2 Hz, 2H), 3.55-3.41 (m, 2H), 2.07-1.93 (m, 2H), 1.35 (s, 9H), 1.16-0.96 (m, 21H).

### Preparation of tert-butyl N-[(2E)-4-{[2-amino-4-carbamoyl-6-(3-{[tris(propan-2-yl)silyl]oxy}propoxy)phenyl]amino}but-2-en-1-yl]carbamate (36-2)

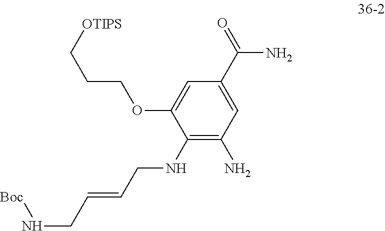

In a flask, compound 36-1 (1.00 eq., 8.82 g, 15.2 mmol) was suspended in methanol (23 mL), N,N-dimethylformamide (15 mL) and water (35 mL) at room temperature. A solution of sodium dithionite (6.00 eq., 15.9 g, 91.1 mmol) in water (55 mL) was added at room temperature and the suspension was stirred for 5 minutes. Then, sodium bicarbonate (12.0 eq., 15.3 g, 182 mmol) was added. The reaction mixture was stirred at room temperature for 3 hours. The reaction mixture was diluted with water (200 mL) and ethyl acetate (200 mL). The aqueous layer was extracted with ethyl acetate (2×150 mL). The organic layer was dried over anhydrous sodium sulfate, filtered and concentrated under vacuum to afford the crude product (12.4 g) as a yellow oil. The crude product was purified by flash chromatography over silica gel (Column: Interchim 50 μm 330 g; Loading: Solid (Silica, 36 g)) using dichloromethane/methanol as eluents (10:0 for 5 minutes then gradient to 95:5 over 25 minutes then 95:5 for 25 minutes) to afford the desired compound 36-2 (6.17 g, 9.89 mmol, 65%, contaminated with 11.7 wt % of dichloromethane) as an off white solid.

Note: If the reaction is not complete, more sodium dithionite in water can be added to reach completion (indicated by the loss of the orange color for a thick white or very pale yellow solution).

LC/MS (CSH, water+0.1% HCO₂H/MeCN 2-100 gradient, 3.5 min): Rt=1.90 min, 97%, [M+H]⁺=552.36)

¹H NMR (DMSO-d₆, 400 MHz): δ ppm 7.58 (s, 1H), 7.00-6.44 (m, 4H), 5.69-5.44 (m, 2H), 4.65 (s, 2H), 4.04 (t, J=6.0 Hz, 2H), 3.85 (t, J=6.3 Hz, 2H), 3.74 (d, J=8.2 Hz, 1H), 3.52 (dd, J=13.2, 6.3 Hz, 4H), 1.94 (p, J=6.2 Hz, 2H), 1.37 (s, 9H), 1.18-0.91 (m, 21H).

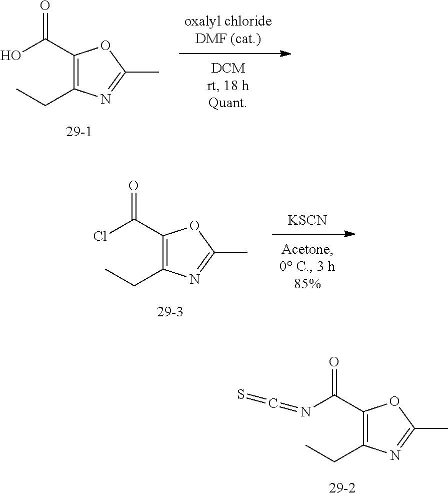

### Preparation of 4-Ethyl-2-methyl-1,3-oxazole-5-carbonyl chloride (29-3)

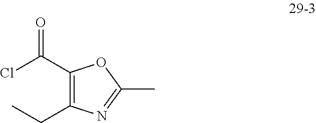

In a flask under argon, oxalyl chloride (1.20 eq., 4.98 mL, 58.0 mmol) was added to a suspension of compound 29-1 (1.00 eq., 7.50 g, 48.3 mmol) in anhydrous dichloromethane (250 mL) at room temperature (gentle bubbling occurring). Then, anhydrous N,N-dimethylformamide (0.10 eq., 0.37 mL, 4.83 mmol) was added dropwise (vigorous bubbling) and the solution was stirred at room temperature for 18 hours. (White suspension turned into a light yellow solution and bubbling stopped). All volatiles (except N,N-dimethylformamide) were removed under vacuum and the crude product (8.39 g, 48.3 mmol, 100%) was used without further purification in the next step

LC/MS samples were prepared by diluting an aliquot of the reaction in a vial of anhydrous methanol which was then heated for a few seconds.

LC/MS (CSH, water+0.1% HCO₂H/MeCN 2-100 gradient, 3.5 min): Rt=1.24 min, 100%, [M+H]⁺=170.13 (as the methyl ester of 29-3)

### Preparation of 4-Ethyl-2-methyl-1,3-oxazole-5-carbonyl isothiocyanate (29-2)

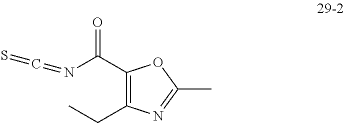

In a flask under argon, potassium thiocyanate (1.30 eq., 6.11 g, 62.8 mmol) was suspended in acetone (70 mL) and cooled down to 0° C. Then, a solution of compound 29-3 (1.00 eq., 8.39 g, 48.3 mmol) in acetone (145 mL) was added dropwise at 0° C. The reaction mixture was stirred at 0° C. for 3 hours. The reaction mixture was concentrated under vacuum and co-evaporated with n-hexane (3×50 mL). The residue was suspended in n-hexane (100 mL), filtered off, washed with n-hexane (3×150 mL) and the filtrate was concentrated under vacuum to afford the crude product (8.75 g) as a yellow oil. The crude product was purified by flash chromatography over silica gel (Column: Interchim 50 μm 330 g; Loading: Solid (Celite, 24 g)) using cyclohexane/ethyl acetate as eluents (gradient from 10:0 to 7:3 over 30 minutes) to afford the desired compound 29-2 (8.26 g, 40.9 mmol, 85%, contaminated with 2.9 wt % of dichloromethane) as a pale yellow oil.

¹H NMR (CDCl₃, 400 MHz): δ ppm 2.89 (q, J=7.6 Hz, 2H), 2.53 (s, 3H), 1.26 (t, J=7.6 Hz, 3H).

### Preparation of tert-butyl N-[(2Z)-4-[5-carbamoyl-2-(4-ethyl-2-methyl-1,3-oxazole-5-amido)-7-(3-{[tris(propan-2-yl)silyl]oxy}propoxy)-1H-1,3-benzodiazol-1-yl]but-2-en-1-yl]carbamate (36)

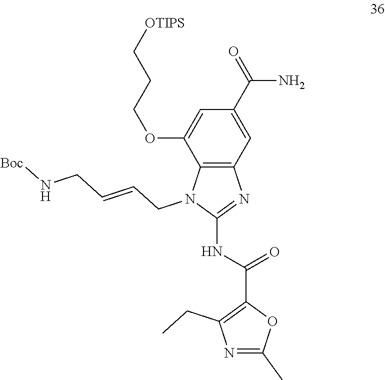

In a flask under argon, a solution of compound 36-2 (1.00 eq., 5.40 g, 9.80 mmol) in N,N-dimethylformamide (95 mL) was cooled down to 0° C. Then, a solution of compound 29-2 ((0.2M in dioxane, 1.00 eq., 49.0 mL, 9.80 mmol) was added dropwise over 45 minutes via an addition funnel. The reaction mixture was then stirred at 0° C. for 1 hour. Then, N-(3-Dimethylaminopropyl)-N-ethylcarbodiimide hydrochloride (2.00 eq., 3.76 g, 19.60 mmol) and triethylamione (4.00 eq., 3.97 g, 5.45 mL, 39.2 mmol) were added at 0° C. The reaction mixture was allowed to warm up to room temperature and was stirred for 18 hours. The reaction mixture was diluted with dichloromethane (300 mL) and a water/saturated aqueous ammonium chloride solution (3:1, 200 mL). The organic layer was dried over anhydrous sodium sulfate, filtered and concentrated under vacuum to afford the crude product (10.1 g) as a waxy beige solid. The crude product was purified by flash chromatography over silica gel (Column: Interchim 15 μm 330 g; Loading: Solid (Silica, 30 g)) using dichloromethane/methanol as eluents (100:0 for 5 minutes then gradient to 90:10 over 45 minutes) to afford the desired compound 36 (6.40 g, 8.72 mmol, 89%, contaminated with 2.8 wt % of dichloromethane) as a pale yellow foamy solid.

Note: If the cyclization step is not complete, it is possible to add more N-(3-Dimethylaminopropyl)-N′-ethylcarbodiimide hydrochloride and triethylamine to achieve complete conversion of the intermediate into the desired product. The reaction is robust and tolerates to be stirred at room temperature for a couple days if needed.

LC/MS (CSH, water+0.1% HCO₂H/MeCN 50-100 gradient, 12.0 min): Rt=6.59 min, 98.00%, [M+H]⁺=714.03

¹H NMR (DMSO-d₆, 400 MHz): δ ppm 12.73 (s, 1H), 7.96 (s, 1H), 7.65 (d, J=1.3 Hz, 1H), 7.46-7.16 (m, 2H), 6.87 (s, 1H), 5.78-5.66 (m, 1H), 5.64-5.50 (m, 1H), 4.91 (d, J=5.8 Hz, 2H), 4.27 (t, J=6.1 Hz, 2H), 3.89 (t, J=6.2 Hz, 2H), 3.48 (s, 2H), 2.99 (q, J=7.5 Hz, 2H), 2.44 (s, 3H), 2.05 (tt, J=6.2, 6.1 Hz, 2H), 1.31 (s, 8H), 1.19 (t, J=7.5 Hz, 4H), 1.10-0.99 (m, 21H).

### Preparation of 36-A

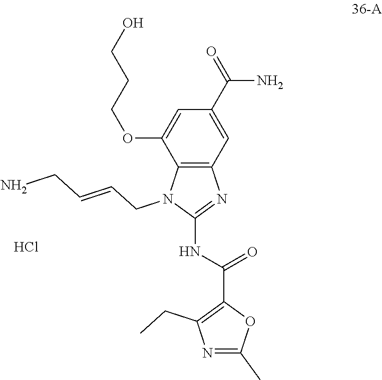 [SMILES: Cl ; CCc1nc(C)oc1C(=O)Nc1nc2cc(C(N)=O)cc(OCCCO)c2n1C/C=C/CN]

To a solution of compound 36 (1.08 g, 1.51 mmol, 1.0 eq.) in dichloromethane (20.0 mL) was added dropwise 4 M HCl in dioxane (3 mL, 12.1 mmol, 8 eq.). The resulting solution was stirred at room temperature for 30 min. Complete removal of Boc and TIPS protecting groups was confirmed by UPLC-MS analysis. The reaction mixture was titrated into cold diethyl ether (100 mL) and forming precipitate was isolated by centrifugation. The solids were rinsed with diethyl ether (50 mL) and dried under reduced pressure to afford 36-A (quantitative) as an off-white solid.

[M−HCl+H⁺]⁺=457.3.

### Preparation of 36-B

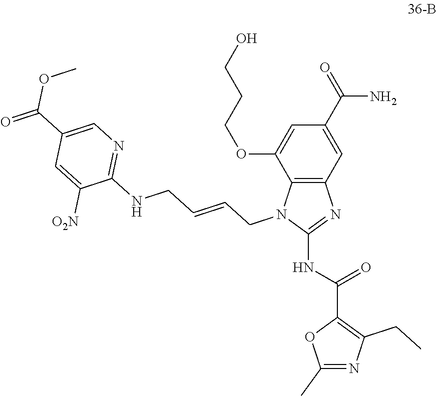 [SMILES: CCc1nc(C)oc1C(=O)Nc1nc2cc(C(N)=O)cc(OCCCO)c2n1C/C=C/CNc1ncc(C(=O)OC)cc1[N+](=O)[O-]]

To a suspension of compound 36-A (2.1 g, 3.98 mmol, 1.0 eq.) in acetonitrile (10.0 mL) was added methyl 6-chloro-5-nitronicotinate as solid (1.0 g; 4.78 mmol, 1.2 eq.) followed by N,N-diisopropylethylamine (3.0 eq., 2.1 mL, 12 mmol). The resulting suspension was stirred at 45° C. temperature for 1 hour. Clean conversion to the desired product was confirmed by UPLC-MS analysis. The reaction mixture was titrated into cold diethyl ether (100 mL) and forming precipitated was isolated by centrifugation. The solids were rinsed with diethyl ether (50 mL) and dried under reduced pressure to crude afford 36-B as yellow which was used directly in the next step without further purification.

[M+H⁺]⁺=637.3.

### Preparation of 36-C

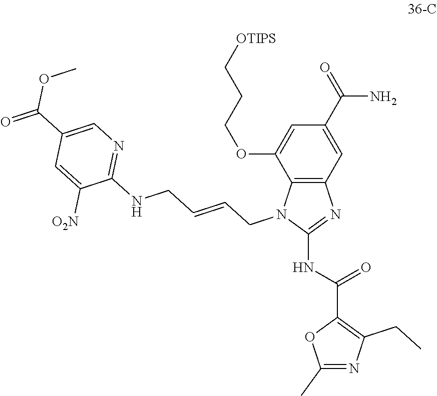

To a solution of compound 36-B (ca. 3.90 mmol, 1.0 eq.) in acetonitrile, dichloromethane, and N,N-dimethylformamide (30.0, 10, and 10 mL, respectively) was added triisopropylsilyl chloride (5.3 g, 27.3 mmol, 7.0 eq.) followed by imidazole (2.5 eq., 0.68 g, 10.0 mmol) and N,N-diisopropylethylamine (5.9 eq., 4 mL, 22.9 mmol). The resulting solution was stirred at 45° C. temperature for 1 hour. Conversion to the desired product was confirmed by UPLC-MS analysis. Volatiles were removed under reduced pressure and the residue diluted with water (200 mL) and extracted with ethyl acetate (2×150 mL). The organic layer was dried over anhydrous sodium sulfate, filtered and concentrated under vacuum to afford the crude product (1.56 g, 1.9 mmol) as a yellow solid which was used directly in the next step without further purification.

[M+H⁺]⁺=793.5.

### Preparation of 36-D

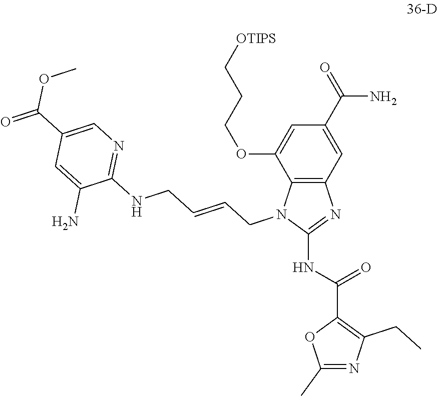

In a 100-mL round-bottom flask, compound 36-C(1.00 eq., 1.56 g, 1.9 mmol) was suspended in methanol (10 mL), N,N-dimethylformamide (20 mL) and water (10 mL) at room temperature. A solution of sodium dithionite (30.0 eq., 10.0 g, 57.4 mmol) in water (20 mL) was added at room temperature and the forming heterogenous mixture was stirred for 10 minutes. Then, sodium bicarbonate (62.0 eq., 10.0 g, 118.9 mmol) was added. The reaction mixture was stirred at room temperature for 1 hour. The reaction mixture was diluted with water (200 mL) and ethyl acetate (200 mL). The aqueous layer was extracted with ethyl acetate (2×150 mL). The organic layer was dried over anhydrous sodium sulfate, filtered and concentrated under vacuum and the residue was titrated into cold diethyl ether (100 mL) to afford the crude product. The product was isolated as beige solid after purification by silica column chromatography (25G Sfar silica column) using 5→15% MeOH in DCM (1.1 g, 1.38 mmol, 73%).

[M+H⁺]⁺=763.6.

### Preparation of 36-E

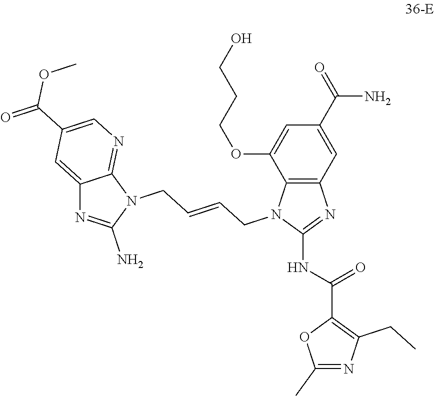 [SMILES: CCc1nc(C)oc1C(=O)Nc1nc2cc(C(N)=O)cc(OCCCO)c2n1C/C=C/Cn1c(N)nc2cc(C(=O)OC)cnc21]

To a solution of compound 36-D (0.44 g, 0.57 mmol; 1.0 eq.) in methanol (12 mL) was added cyanogen bromide (10.0 eq., 0.611 g, 5.77 mmol) followed by N,N-diisopropylethylamine (1.0 eq., 0.1 mL, 0.57 mmol). The resulting solution was stirred at 40° C. temperature for 16 hours. Conversion to the product with loss of TIPS protecting group was confirmed by UPLC-MS analysis. Volatiles were removed under reduced pressure and the residue was titrated into cold diethyl ether (100 mL) and the forming precipitate was dried under reduced pressure to afford the crude product (0.4 g, ca. 75% purity by UPLC-MS) as a brownish solid. Crude product was used in the next step without further purification.

[M+H⁺]⁺=632.4.

### Preparation of 36-F

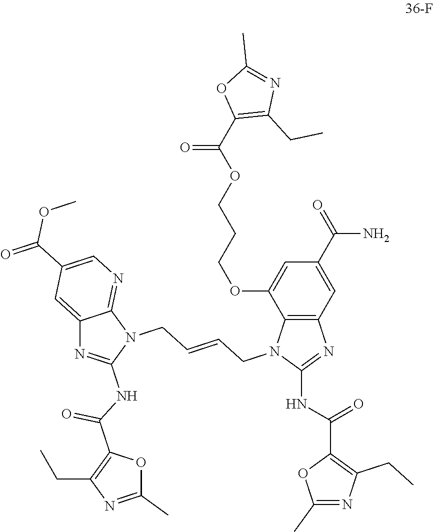 [SMILES: CCc1nc(C)oc1C(=O)Nc1nc2cc(C(=O)OC)cnc2n1C/C=C/Cn1c(NC(=O)c2oc(C)nc2CC)nc2cc(C(N)=O)cc(OCCCOC(=O)c3oc(C)nc3CC)c21]

To a solution of compound 36-E (0.3 g, 0.47 mmol; 1.0 eq.) in N,N-dimethylformamide (4 mL) was added 4-ethyl-2-methyloxazole-5-carboxylic acid (2.7 eq., 196.5 mg, 1.26 mmol), HATU (2.7 eq., 481.2 mg, 1.26 mmol) and N,N-diisopropylethylamine (5.3 eq., 0.44 mL, 2.51 mmol). The resulting solution was stirred at room temperature for 16 hours. Complete conversion to the product was confirmed by UPLC-MS analysis. The reaction mixture was titrated into cold diethyl ether (25 mL) and the forming precipitate was purified by silica column chromatography (10G Sfar silica column) using 5→10% MeOH in DCM to afford beige solid (205 mg, ca. 75% purity by UPLC-MS).

[M+H⁺]⁺=906.4.

### Preparation of 36-G

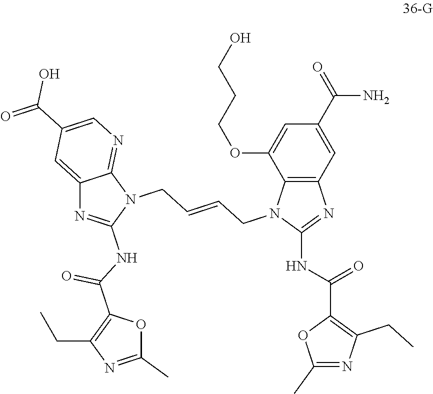 [SMILES: CCc1nc(C)oc1C(=O)Nc1nc2cc(C(=O)O)cnc2n1C/C=C/Cn1c(NC(=O)c2oc(C)nc2CC)nc2cc(C(N)=O)cc(OCCCO)c21]

To a solution of compound 36-F (153.7 mg, 0.17 mmol; 1.0 eq.) in tetrahydrofuran, methanol, and water (1 mL each) was added 5-M solution of NaOH in water (5.9 eq., 0.2 mL, 1 mmol). The resulting solution was stirred at 40° C. temperature for 1 hour. Complete conversion to the product was confirmed by UPLC-MS analysis and volatiles were removed under reduced pressure. The residue was diluted with water (5 mL) and the pH was adjusted to 6 by addition of 1-M HCl solution in water and then the mixture was lyophilized to yield beige solid that was used in the next step without further purification.

[M+H⁺]⁺=755.4.

### Preparation of 36-H

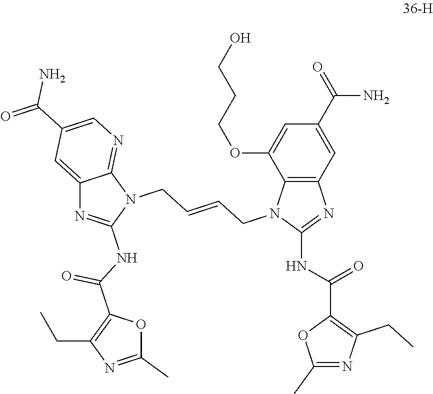 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCO)c21]

To a solution of compound 36-G (ca. 0.17 mmol; 1.0 eq.) in N,N-dimethylformamide (8 mL) was added ammonium chloride (10 eq., 90.9 mg, 1.70 mmol), HATU (2.5 eq., 161.6 mg, 0.42 mmol) and N,N-diisopropylethylamine (12.0 eq., 0.35 mL, 2.04 mmol). The resulting solution was stirred at room temperature for 5 min. Complete conversion to the product was confirmed by UPLC-MS analysis. Volatiles were removed under reduced pressure and then residue was purified by silica column chromatography (10G Sfar silica column) using 5→25% MeOH in DCM to afford beige solid (86 mg, 0.11 mmol, 67%).

[M+H⁺]⁺=754.5.

### Preparation of Compound XX

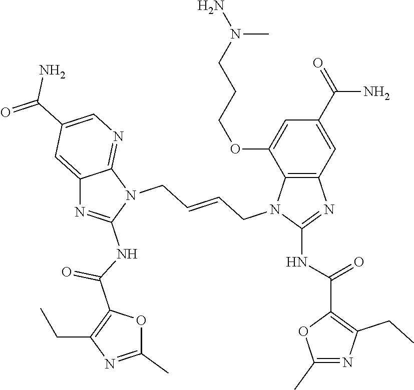 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(C)N)c21]

To a solution of 36-H (1.00 eq., 76 mg, 0.1 mmol) in N-methyl-2-pyrrolidone (0.7 mL) at 0° C. was added triethylamine (9 equiv., 123.1 μL, 0.9 mmol) followed by methanesulfonyl chloride (7 equiv., 54.1 μL, 0.7 mmol). After 1 hour, UPLC-MS reaction monitoring showed 90% conversion to mesylate. The reaction mixture was titrated into cold diethyl ether (20 mL) and the forming precipitate collected via centrifugation. To the solids was added N-methyl-2-pyrrolidone (0.7 mL) and methylhydrazine (38.2 equiv., 200 μL, 3.8 mmol). The forming solution was stirred at 45° C. for 1 hour and was complete as indicated by UPLC-MS. Purification was carried out using RP-HPLC to afford title compound as white solid as trifluoroacetic acid salt (15 mg, 0.017 mmol, 17% over two steps).

[M+H⁺]⁺=782.5.

### Example 30: Activity Upon Membrane Permeabilization

Assay tests were performed as detailed in Example 20 and IRF pathway activation was measured between compound XV of Table 1 versus compound XX of Table 1 as detailed in Example 21 to show activity upon membrane permeabilization.

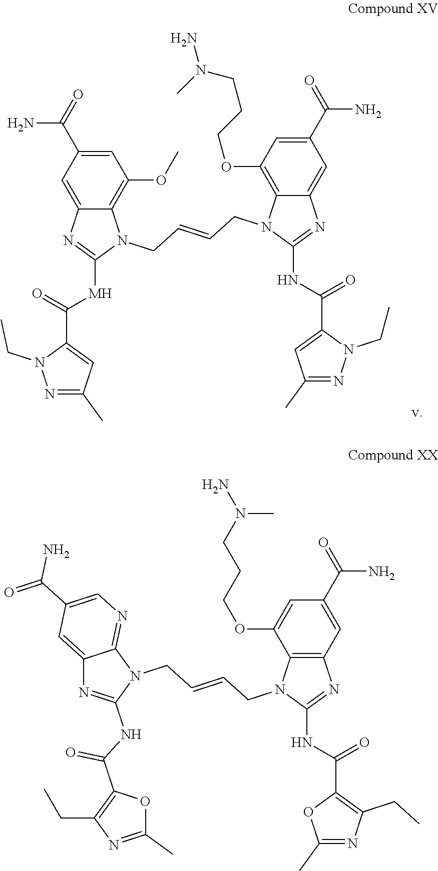

The primary differences are that Compound XX is less hydrophobic as a result of the introduction of oxazolyl heterocyclic ring in place of the pyrazolyl heterocyclic ring of Compound XV and as a result of the introduction of an imidazopyridine ring in place of the benzimidazole ring of Compound XV. This substitution results in a 1-log reduction in cLogP and slightly better solubility as shown in Table 5:

TABLE 5

| | | | PBS Buffer | | HEPES Buffer | |
|---|---|---|---|---|---|---|
| | | | pH 7.4 | | pH 7.0 | |
| ID | MW | cLogP* | μM | mg/ml | μM | mg/ml |
| Compound | 808.91 | 1.3 | <LOQ^{#} | <LOQ | 5.9 | 0.005 |
| XV | | | | | | |
| Compound | 781.84 | −0.4 | 3.6 | 0.003 | 11.5 | 0.01 |
| XX | | | | | | |

*Calculated LogP using ChemDraw software.
^{#}Lower limit of quantitation, 1 μM.

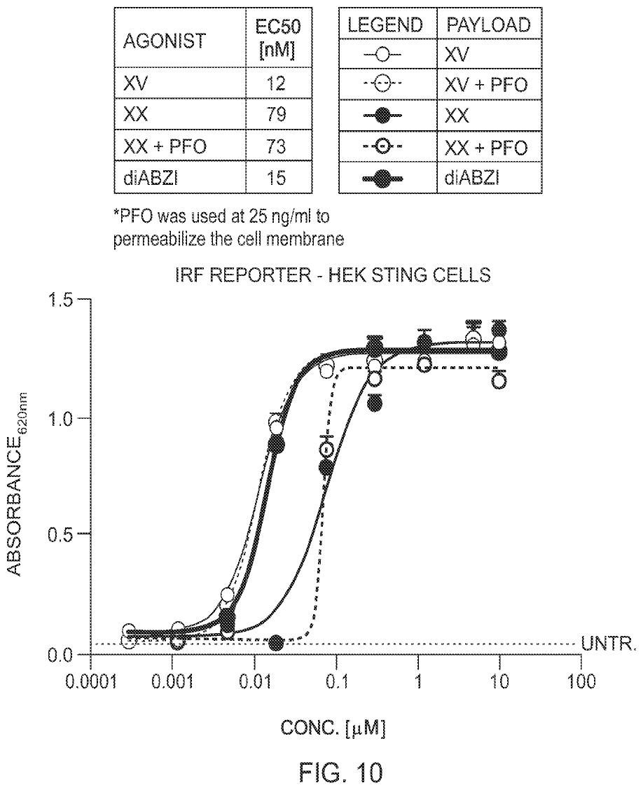

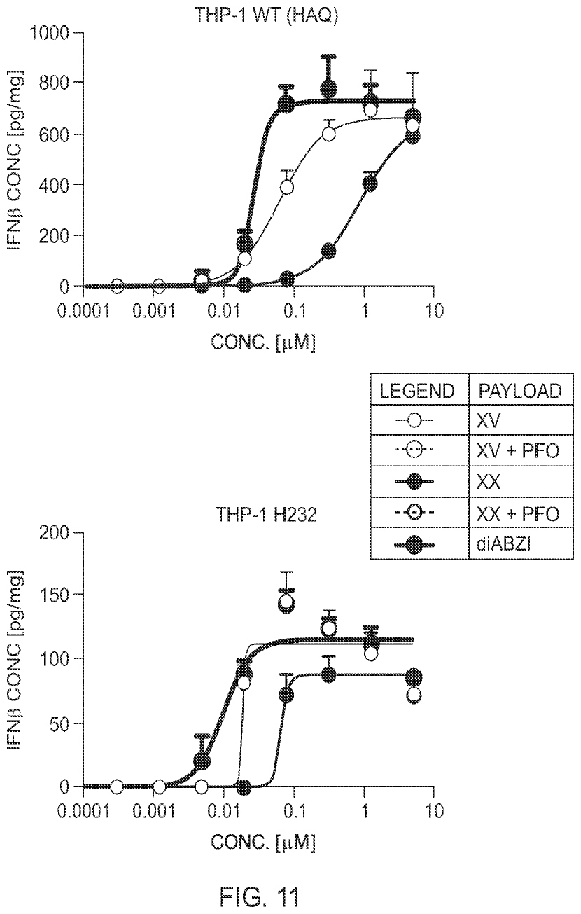

EC50 values were calculated using GraphPad Prism 9. Plots of IRF pathway activation for compounds XV and XX are shown in FIG. 10 in comparison with diABZI.

### Example 31: STING Allele Activity

Assays were performed as described in Example 21. Biological activity of Compounds XV and XX was evaluated as described in Example 20. STING haplotype curves for compounds XV and XX are shown in FIG. 11.

### Example 32: PBMC Assay

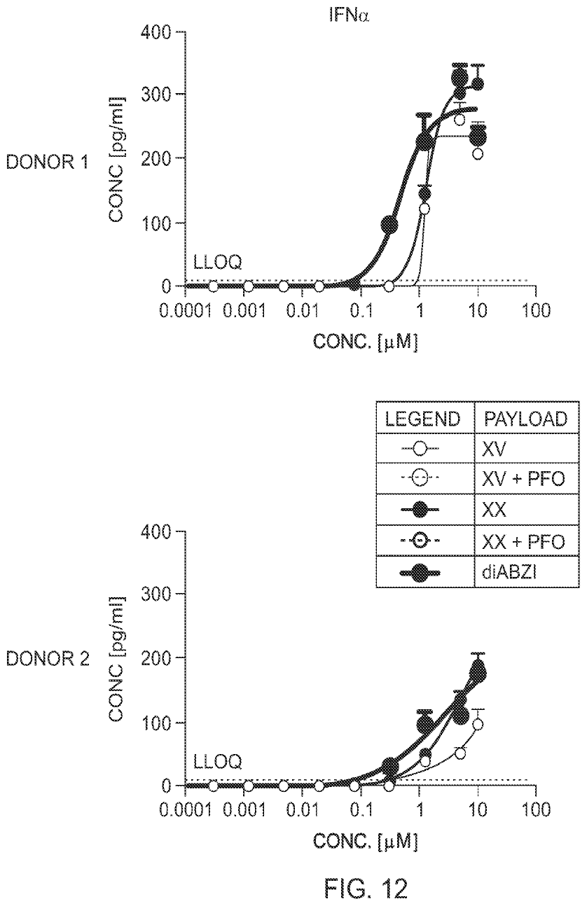

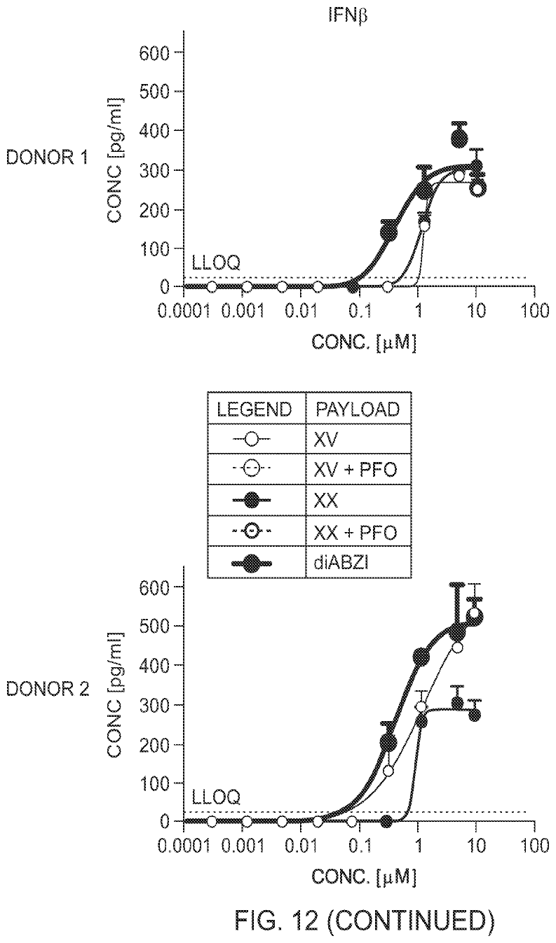

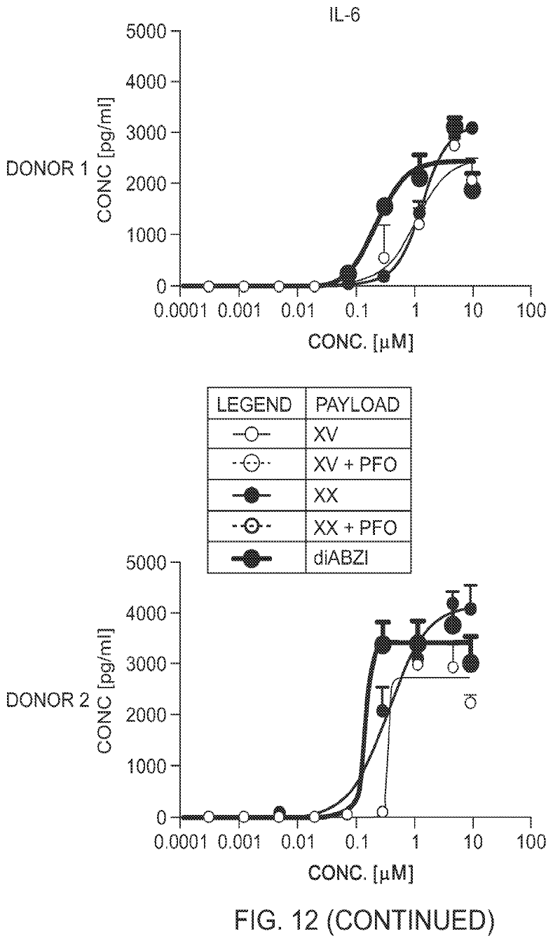

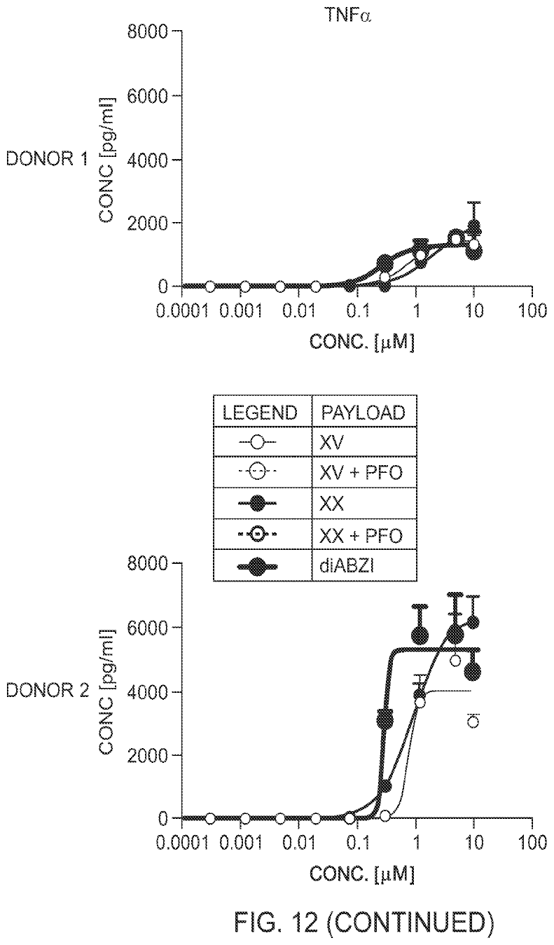

Compounds XV and XX were additionally tested on PBMCs from healthy donors to determine the dose-dependent cytokine induction as described in Example 23. PBMC representative curves for compounds XV and XX are shown in FIG. 12.

### Materials and Methods (Examples 33-44)

### LCMS: System I

Waters HPLC-System CTC Pal Autosampler. 1× Waters 1525 Multisolvent Delivery System 10 μl sample loop. Waters Micromass ZQ single quadrupol mass spectrometer with electrospray source. MS method: positive/negative ion mode scanning, m/z 80-800 or 80-2000 in 1 s; capillary voltage, 3.0 kV; cone voltage, 20-50 V; multiplier voltage, 700 V; probe and desolvation gas temperature, 120° C. and 300° C., respectively. Waters 996 PDA detector, set to 254 nm. Software, Waters Masslynx V 4.1.

### LCMS System II

Shimadzu LCMS-2050: SCL-40 system controller; DGU-405 degassing unit, 2× LC-40D XR solvent delivery pump; SUL-40C XR autosampler; CTO-40C column oven. MS LCMS-2050: single quadrupol mass spectrometer with ESI/APCI DUIS source. MS method: positive/negative ion mode scanning, m/z 100-2000 in 0.45 s per event, desolvation temperature 450° C., desolvation line temperature 200° C., interface voltage +3.0 KV and −2.0 kV. PDA detector: Shimadzu SPD-M40, start wavelength 190 nm, end wavelength 600 nm, analyzed at 254 nm, 200 nm, 220 nm, 260 nm, cell temperature 40° C. Software Shimadzu LabSolutions Version 5.118.

### Method A: 4.3 min

Column: Waters Phenomenex Onyx Monolythic C18 50×2 mm, with stainless steel 2 μm prefilter
UV-Detector: 254 nm
Column temperature: room temperature
Pump modus: Gradient

TABLE 6
Standard-Gradient (Eluent A = H₂O +
0.1% HCOOH, Eluent B = MeCN)

| Step | Time | Flow | A | B |
|---|---|---|---|---|
| Nr. | [min] | [mL/min] | [%] | [%] |
| 1 | 0.00 | 0.60 | 95.0 | 5.00 |
| 2 | 3.80 | 0.60 | 0.00 | 100 |
| 3 | 3.81 | 1.20 | 0.00 | 100 |
| 4 | 4.00 | 1.20 | 0.00 | 100 |
| 5 | 4.07 | 1.20 | 95.0 | 5.00 |
| 6 | 4.10 | 1.20 | 95.0 | 5.00 |
| 7 | 4.30 | 0.60 | 95.0 | 5.00 |

### Method B: 7.5 min

Column: Waters Phenomenex Onyx Monolythic C18 50×2 mm, with stainless steel 2 μm prefilter
UV-Detector: 254 nm
Column temperature: room temperature
Pump modus: Gradient

TABLE 7
Standard-Gradient (Eluent A = H₂O +
0.1% HCOOH, Eluent B = MeCN)

| Step | Time | Flow | A | B |
|---|---|---|---|---|
| Nr. | [min] | [mL/min] | [%] | [%] |
| 1 | 0.00 | 0.60 | 95.0 | 5.00 |
| 2 | 6.50 | 0.60 | 0.00 | 100 |
| 3 | 6.51 | 1.20 | 0.00 | 100 |
| 4 | 6.91 | 1.20 | 0.00 | 100 |
| 5 | 7.00 | 1.20 | 95.0 | 5.00 |
| 6 | 7.50 | 0.60 | 95.0 | 5.00 |

### Method C: 10 min

Column: Waters Phenomenex Onyx Monolythic C18 50×2 mm, with stainless steel 2 μm prefilter
UV-Detector: 254 nm
Column temperature: room temperature
Pump modus: Gradient

TABLE 8
Standard-Gradient (Eluent A = H₂O +
0.1% HCOOH, Eluent B = MeCN)

| Step | Time | Flow | A | B |
|---|---|---|---|---|
| Nr. | [min] | [mL/min] | [%] | [%] |
| 1 | 0.00 | 0.60 | 95.0 | 5.00 |
| 2 | 9.00 | 0.60 | 0.00 | 100 |
| 3 | 9.01 | 1.20 | 0.00 | 100 |
| 4 | 9.40 | 1.20 | 0.00 | 100 |
| 5 | 9.47 | 1.20 | 95.0 | 5.00 |
| 6 | 10.0 | 0.60 | 95.0 | 5.00 |

### Method D: 15 min

Column: Waters Phenomenex Onyx Monolythic C18 50×2 mm, with stainless steel 2 μm prefilter
UV-Detector: 254 nm
Column temperature: room temperature
Pump modus: Gradient

TABLE 9
Standard-Gradient (Eluent A = H₂O +
0.1% HCOOH, Eluent B = MeCN)

| Step | Time | Flow | A | B |
|---|---|---|---|---|
| Nr. | [min] | [mL/min] | [%] | [%] |
| 1 | 0.00 | 0.60 | 95.0 | 5.00 |
| 2 | 13.00 | 0.60 | 0.00 | 100 |
| 3 | 13.01 | 1.20 | 0.00 | 100 |
| 4 | 14.40 | 1.20 | 0.00 | 100 |
| 5 | 14.47 | 1.20 | 95.0 | 5.00 |
| 6 | 15.00 | 1.20 | 95.0 | 5.00 |

### HPLC

Dionex Ultimate3000 with solvent Rack and vacuum degasser SRD-3600, binary pump DPG-3600SD, autosampler WPS-3000TFC: 15.0 μL needle volume, 50 μL sample loop, 6.2 μL bridge tubing, 250 μL syringe. Column compartment: TCC-3200; Detector: DAD-3000; CAD-Detector Corona Veo. HPLC-Software: Chromeleon Datasystem 7.3.

### Method A

Column: Waters Phenomenex Onyx Monolythic C18 2 mm×50 mm
UV-Detector: 190-400 nm
Injection volume: 10 UL
Column temperature: 30° C.
Pump modus: Gradient

TABLE 10
Standard-Gradient (Eluent A = H₂O +
0.1% TFA, Eluent B = MeCN)

| Time [min] | A [%] | B [%] | Flow [mL/min] |
|---|---|---|---|
| 0.00 | 100.0 | 0.0 | 1.20 |
| 2.10 | 100.0 | 0.0 | 1.20 |
| 9.00 | 60.0 | 40.0 | 1.20 |
| 9.10 | 60.0 | 40.0 | 1.20 |
| 10.00 | 5.0 | 95.0 | 1.20 |
| 12.00 | 5.0 | 95.0 | 1.20 |
| 12.10 | 100.0 | 0.0 | 1.20 |
| 14.00 | 100.0 | 0.0 | 1.20 |

### Method B

Column: Waters XSelect CSH C18, 130 Å, 3.5 μm, 4.6 mm×100 mm
UV-Detector: 190-400 nm
Injection volume: 10 ML
Column temperature: 30° C.
Pump modus: Gradient

TABLE 11
Standard-Gradient (Eluent A = H₂O +
0.1% TFA, Eluent B = MeCN)

| Time [min] | A [%] | B [%] | Flow [mL/min] |
|---|---|---|---|
| 0.00 | 100.0 | 0.0 | 1.00 |
| 2.10 | 100.0 | 0.0 | 1.00 |
| 3.00 | 60.0 | 40.0 | 1.00 |
| 10.00 | 5.0 | 95.0 | 1.00 |
| 12.00 | 5.0 | 95.0 | 1.00 |
| 12.10 | 0.0 | 100.0 | 1.00 |
| 14.00 | 0.0 | 100.0 | 1.00 |

### Method C

Column: Waters XBridge BEH C18, 130 Å, 3.5 μm, 2.1 mm×100 mm
UV-Detector: 190-400 nm
Injection volume: 10 μL
Column temperature: 30° C.
Pump modus: Gradient

TABLE 12
Standard-Gradient (Eluent A = H₂O +
0.1% TFA, Eluent B = MeCN)

| Time [min] | A [%] | B [%] | Flow [mL/min] |
|---|---|---|---|
| 0.00 | 100.0 | 0.0 | 0.40 |
| 2.10 | 100.0 | 0.0 | 0.40 |
| 3.00 | 80.0 | 20.0 | 0.40 |
| 9.00 | 50.0 | 50.0 | 0.40 |
| 9.10 | 50.0 | 50.0 | 0.40 |
| 10.00 | 5.0 | 95.0 | 0.40 |
| 12.00 | 5.0 | 95.0 | 0.40 |
| 12.10 | 100.0 | 0.0 | 0.40 |
| 14.00 | 100.0 | 0.0 | 0.40 |

### Preparative HPLC

Waters Autopurification System: Waters 3767 Autosampler (equipped with 5 mL syringe and 10 ml sample loop), Waters System Fluid Organizer, Waters 2525 Binary Gradient Modul, Waters 515 Make-Up Pump (50% acetonitrile in water+0.1% formic acid, 1 mL/min), Waters 515 At-Column-Dilution Pump (different solvent mixtures, individually adapted to sample, 5 mL/min for first 2 min of gradient), Waters 2998 Photo Diode Array Detector, Waters QDA Mass Spectrometer (for mass-triggered fractionation, Scan mode, positive and negative polarization). Software: Waters MassLynx V 4.2.

### Method I

Column: Waters Atlantis T3 OBD Prep Column, 100 Å, 5 μm, 19 mm×150 mm, Part. No.: 186003698, Flow 20 mL/min

TABLE 13
Standard-Gradient (Eluent A = H₂O +
0.1% TFA, Eluent B = MeCN)

| Time (min) | Flow (mL/min) | % A | % B |
|---|---|---|---|
| 0.00 | 15 | 95 | 5 |
| 2.00 | 15 | 95 | 5 |
| 2.10 | 20 | 95 | 5 |
| 25.0 | 20 | 60 | 40 |
| 27.0 | 20 | 5 | 95 |
| 29.0 | 20 | 5 | 95 |
| 29.1 | 20 | 95 | 5 |
| 30.0 | 20 | 95 | 5 |

### Method II

Column: Waters Atlantis T3 OBD Prep Column, 100 Å, 5 μm, 19 mm×150 mm, Part. No.: 186003698, Flow 20 mL/min

TABLE 14
Standard-Gradient (Eluent A = H₂O +
0.1% TFA, Eluent B = MeCN)

| Time (min) | Flow (mL/min) | % A | % B |
|---|---|---|---|
| 0.00 | 15 | 95 | 5 |
| 2.00 | 15 | 95 | 5 |
| 2.10 | 20 | 85 | 15 |
| 25.0 | 20 | 55 | 45 |
| 27.0 | 20 | 5 | 95 |
| 29.0 | 20 | 5 | 95 |
| 29.1 | 20 | 95 | 5 |
| 30.0 | 20 | 95 | 5 |

### Method III

Column: Waters XBridge BEH C18 OBD Prep Column, 130 Å, 5 μm, 19 mm×150 mm, Part. No.: 186002979, Flow 20 mL/min

TABLE 15
Standard-Gradient (Eluent A = 5 mM
NH₄HCO₃ in H₂O, Eluent B = MeCN)

| Time (min) | Flow (mL/min) | % A | % B |
|---|---|---|---|
| 0.00 | 15 | 100 | 0 |
| 2.00 | 15 | 100 | 0 |
| 2.10 | 20 | 100 | 0 |
| 25.0 | 20 | 50 | 50 |
| 27.0 | 20 | 5 | 95 |
| 29.0 | 20 | 5 | 95 |
| 29.1 | 20 | 100 | 0 |
| 30.0 | 20 | 100 | 0 |

### NMR

NMR spectra were collected at 302 K (~ 29° C.) by a Bruker UltraShield 300 MHz spectrometer, equipped with a Nanobay AV III console, a B-ACS 60 autosampler, and a PH BBI 300 S1 H-BB-D-05 Z probe. Deuterated solvents as mentioned in the experimental procedure. All spectra were processed and analyzed using TopSpin 3.6.0 and/or MestreNova 14.2.

### Synthesis of 1-Ethyl-3-methyl-1H-pyrazole-5-carbonyl isothiocyanate

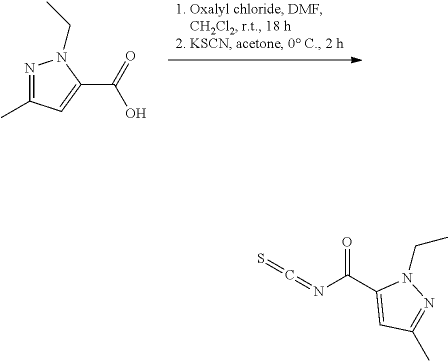

1-Ethyl-3-methyl-1H-pyrazole-5-carboxylic acid (3.50 g, 22.7 mmol) was suspended under argon in anhydrous CH₂Cl₂ (60 mL). Then, oxalyl chloride (2.4 mL, 27.2 mmol) was added dropwise at room temperature followed by DMF (170 μL, 2.27 mmol) which was added dropwise over one minute at room temperature. The mixture was stirred under argon at room temperature for 18 h. The reaction mixture was then evaporated to afford 1-ethyl-3-methyl-1H-pyrazole-5-carbonyl chloride as yellow oil. Potassium thiocyanate (2.87 g, 29.5 mmol) was suspended under argon in acetone (25 mL). The suspension was then cooled to 0° C. via an ice bath. The previously obtained 1-ethyl-3-methyl-1H-pyrazole-5-carbonyl chloride was taken up under argon in dry acetone (45 mL) and then slowly added to the reaction mixture via a dropping funnel over a period of 45 min. The resulting suspension was stirred at 0° C. for 1.5 h. Control by TLC (n-hexane/EtOAc=9:1) showed product. The solvent was removed under reduced pressure. The residue was suspended in n-hexane (50 mL) and sonicated. The solid was then filtered off and thoroughly washed with n-hexane. The filtrate was evaporated to give the crude product as yellow liquid. Purification by automated flash column chromatography on Silica: Biotage Selekt (Biotage Sfär Silica HC, 20 UM, 25 g, 80 mL/min; 3CV n-hexane->10 CV gradient from n-hexane to n-hexane/EtOAc=9:1->10 CV n-hexane/EtOAc=9:1) to afford 1-Ethyl-3-methyl-1H-pyrazole-5-carbonyl isothiocyanate (3.67 g, 18.8 mmol, 83%) as pale yellow liquid which was stored in the freezer until usage. ¹H NMR (CDCl₃, 300 MHz): 1.32 (t, 3H, J=7.2 Hz), 2.21 (d, 3H, J=0.6 Hz), 4.42 (q, 2H, J=7.2 Hz), 6.65 (q, 1H, J=0.7 Hz).

### Synthesis of 4-ethyl-2-methyloxazole-5-carbonyl isothiocyanate

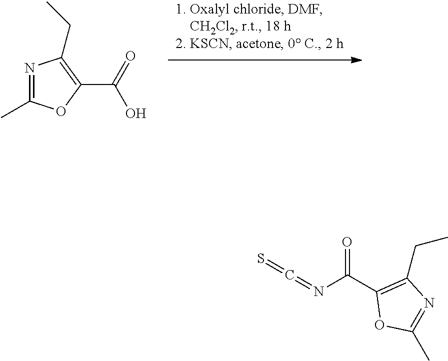

4-Ethyl-2-methyl-1,3-oxazole-5-carboxylic acid (5.00 g, 32.2 mmol) was suspended under argon in anhydrous CH₂Cl₂ (80 mL). Then, oxalyl chloride (3.44 mL, 38.7 mmol) was added dropwise at room temperature followed by DMF (248 μL, 3.22 mmol) which was added dropwise over one minute at room temperature. The mixture was stirred under argon at room temperature for 3 h. The solvent was removed under reduced pressure to afford crude 4-Ethyl-2-methyloxazole-5-carbonyl chloride. Potassium thiocyanate (4.07 g, 41.9 mmol) was suspended under argon in acetone (25 mL). The suspension was cooled to 0° C. and a suspension of previously obtained crude 4-ethyl-2-methyloxazole-5-carbonyl chloride (5.59 g, 32.2 mmol) in acetone (50 mL) was added dropwise over 10 min via an additional funnel. The resulting mixture was stirred at 0° C. for 3 h. The product formation was controlled by TLC (EtOAc/n-hexane: 1/9) The solvent was removed under vacuo (cooling trap). The residue was purified on silica by automated flash column chromatography on a Biotage Selekt (Biotage Sfär HC 50 g, loading: solid (Extrelut), 120 mL/min, 0-20% EtOAc in n-hexane in 30 min) to afford 4-ethyl-2-methyloxazole-5-carbonyl isothiocyanate (3.61 g, 18.3 mmol, 57%) as yellow liquid which was stored in the freezer until usage. ¹H NMR (CDCl₃, 300 MHz): 1.27 (t, 3H, J=7.6 Hz), 2.54 (s, 3H), 2.90 (q, 2H, J=7.5 Hz).

### Example 33: Synthetic Scheme for Compound XXVIII

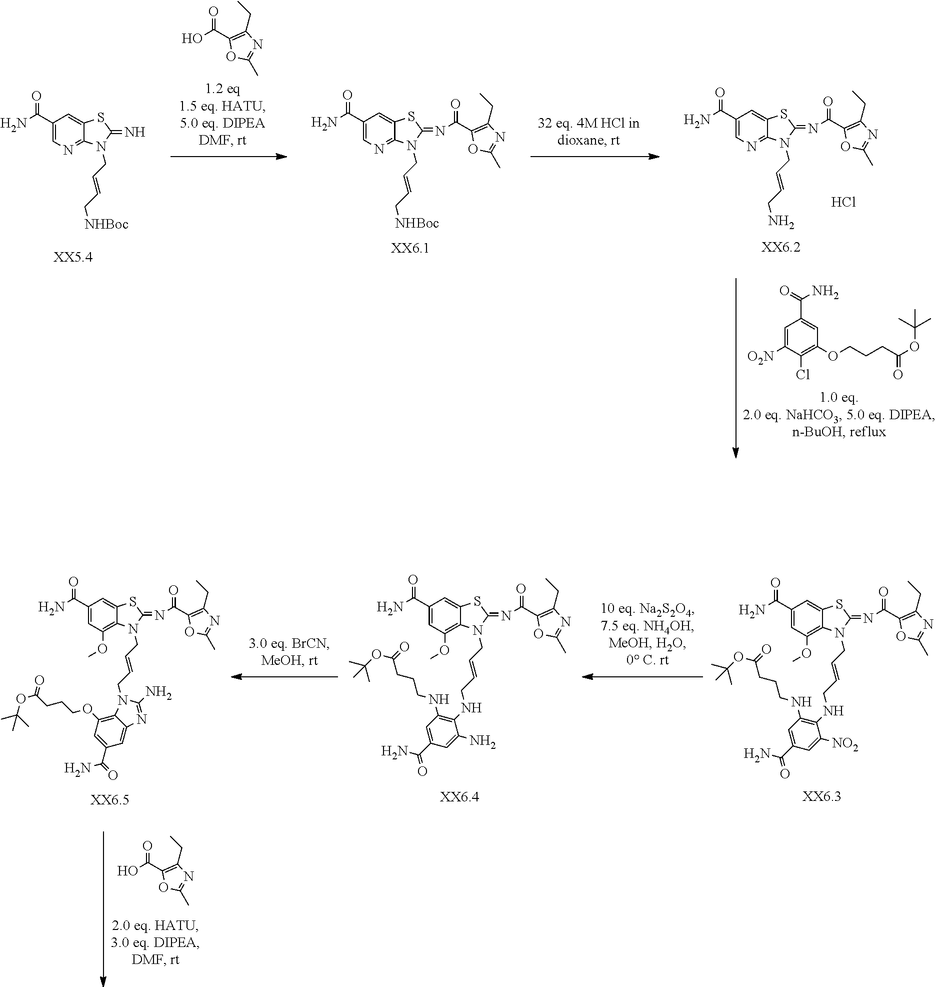

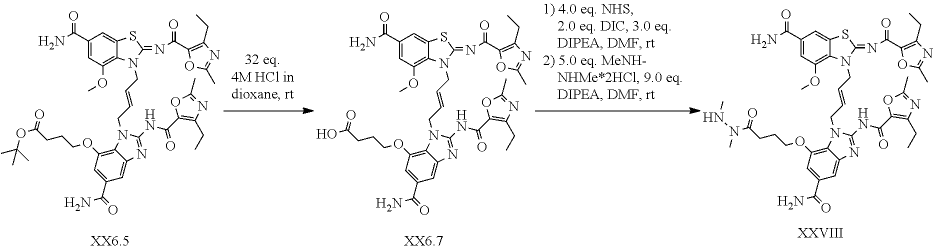

### Preparation of tert-Butyl ((E)-4-((Z)-6-carbamoyl-2-((4-ethyl-2-methyloxazole-5-carbonyl)imino)-4-methoxybenzo[d]thiazol-3(2H)-yl)but-2-en-1-yl)carbamate (XX6.1)

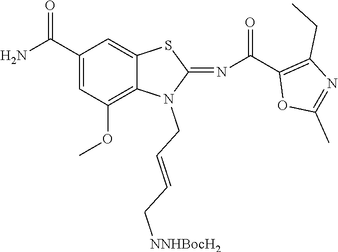

Compound XX5.4 (2.0 g, 5.1 mmol) and 4-ethyl-2-methyl-1,3-oxazole-5-carboxylic acid (949 mg, 6.11 mmol) were dissolved in DMF (15 mL). Afterwards, HATU (2.9 g, 7.6 mmol) and DIPEA (4.48 mL, 25.4 mmol) were added and the reaction mixture was stirred at room temperature for 30 min. The product formation was controlled by LC/MS. The mixture was quenched with water (30 mL) and the resulting solid was filtered off as well as washed with water. The product was dried in vacuo to yield 1.75 g (3.30 mmol, 64%) of an off-white solid. LCMS (Method A) retention time 2.58 min, [M+H]+=530.1. ¹H-NMR (300 MHz, DMSO-d6) δ [ppm]=8.05 (brs, 1H), 8.01-7.96 (m, 1H), 7.65-7.54 (m, 1H), 7.47 (brs, 1H), 6.89 (t, J=5.9 Hz, 1H), 5.82-5.55 (m, 2H), 5.29 (d, J=5.5 Hz, 2H), 3.98 (s, 3H), 3.50 (t, J=5.5 Hz, 2H), 3.05-2.86 (m, 2H), 2.46 (s, 3H), 1.30 (s, 9H), 1.19 (t, J=6.5 Hz, 3H).

### Preparation of N-((Z)-3-((E)-4-aminobut-2-en-1-yl)-6-carbamoyl-4-methoxybenzo[d]thiazol-2(3H)-ylidene)-4-ethyl-2-methyloxazole-5-carboxamide hydrochloride (XX6.2)

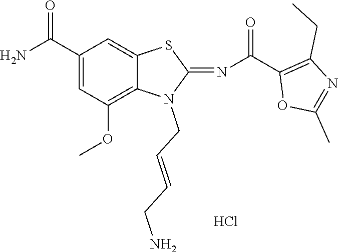 [SMILES: CCc1nc(C)oc1C(=O)/N=c1\sc2cc(C(N)=O)cc(OC)c2n1C/C=C/CN ; Cl]

Compound XX6.1 (1.75 g, 3.30 mmol) was suspended in 4 M HCl in dioxane (26.4 mL, 105 mmol). The resulting suspension was stirred at room temperature for 30 min. The product formation was controlled by LC/MS. The formed solid was filtered off, washed with diethyl ether and dried in vacuo to yield 1.4 g (3.3 mmol, 100%) of an off-white solid, which was used without further purification. LCMS (Method A) retention time 1.28 min, [M+H]+=430.1. ¹H-NMR (300 MHz, DMSO-d6) δ [ppm]=8.13 (brs, 1H), 8.06-7.93 (m, 3H), 7.68-7.58 (m, 1H), 7.58-7.43 (m, 1H), 6.15-5.99 (m, 1H), 5.82-5.57 (m, 1H), 5.37 (d, J=5.6 Hz, 2H), 4.02 (s, 3H), 3.52-3.35 (m, 2H), 2.99 (q, J=7.5 Hz, 2H), 2.48 (s, 3H), 1.22 (t, J=7.5 Hz, 3H).

### Preparation of tert-Butyl 4-(5-carbamoyl-2-(((E)-4-((Z)-6-carbamoyl-2-((4-ethyl-2-methyloxazole-5-carbonyl)imino)-4-methoxybenzo[d]thiazol-3(2H)-yl)but-2-en-1-yl)amino)-3-nitrophenoxy)butanoate (XX6.3)

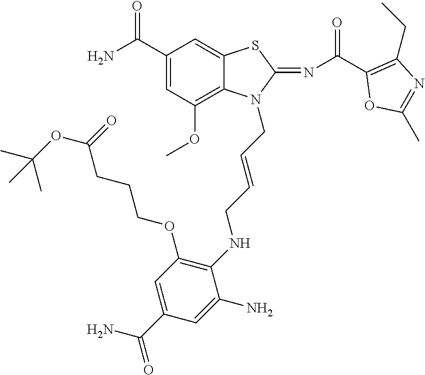 [SMILES: CCc1nc(C)oc1C(=O)/N=c1\sc2cc(C(N)=O)cc(OC)c2n1C/C=C/CNc1c(N)cc(C(N)=O)cc1OCCCC(=O)OC(C)(C)C]

Compound XX6.2 (1.40 g, 3.00 mmol) was suspended in n-butanol (36 mL), DIPEA (2.58 mL, 15.1 mmol) and sodium bicarbonate (505 mg, 6.01 mmol) and the mixture was stirred at room temperature for 10 min. Then, tert-butyl 4-(5-carbamoyl-2-chloro-3-nitrophenoxy)butanoate (1.08 g, 3.00 mmol) was added to the mixture and the reaction was stirred at 130° C. for 26 h and at room temperature for 18 h. The product formation was controlled by LC/MS. The reaction was cooled to room temperature and diluted with water (50 mL). The aqueous layer was extracted with CH₂Cl/CH₃OH (9:1, 70 mL). The organic layer was dried over sodium sulfate and filtered. The solvent was removed under reduced pressure and the residue was purified on silica by automated flash column chromatography on a Biotage Selekt (Biotage Sfär HC 50 g, 120 mL/min, 0-20% CH₃OH in CH₂Cl₂ in 30 min). Product containing fractions were combined to obtain 603 mg (802 μmol, 27%) of a yellowish solid after removal of the solvent. LCMS (Method B) retention time 3.46 min, [M+H]+=752.2. ¹H-NMR (300 MHz, DMSO-d6) δ [ppm]=8.08-8.01 (m, 2H), 7.98-7.94 (m, 1H), 7.91 (brs, 1H), 7.63 (t, J=6.3 Hz, 1H), 7.57-7.53 (m, 1H), 7.46 (brs, 1H), 7.42-7.39 (m, 1H), 7.26 (brs, 1H), 5.85-5.61 (m, 2H), 5.26 (d, J=5.2 Hz, 2H), 4.10 (t, J=5.8 Hz, 2H), 3.94-3.82 (m, 5H), 2.89 (q, J=7.5 Hz, 2H), 2.44 (s, 3H), 2.26 (t, J=7.4 Hz, 2H), 1.84 (p, J=6.8 Hz, 2H), 1.35 (s, 9H), 1.13 (t, J=7.5 Hz, 3H).

### Preparation of tert-Butyl 4-(3-amino-5-carbamoyl-2-(((E)-4-((Z)-6-carbamoyl-2-((4-ethyl-2-methyloxazole-5-carbonyl)imino)-4-methoxybenzo[d]thiazol-3(2H)-yl)but-2-en-1-yl)amino)phenoxy)butanoate (XX6.4)

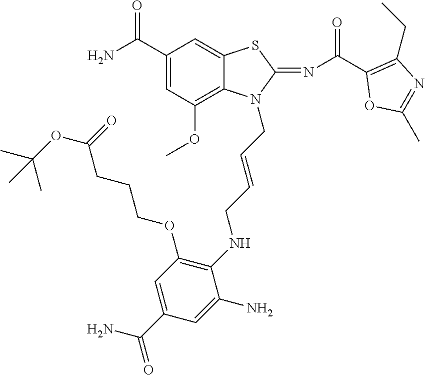 [SMILES: CCc1nc(C)oc1C(=O)/N=c1\sc2cc(C(N)=O)cc(OC)c2n1C/C=C/CNc1c(N)cc(C(N)=O)cc1OCCCC(=O)OC(C)(C)C]

Compound XX6.3 (600 mg, 798 μmol) was dissolved in CH₃OH (15 mL) and cooled to 0° C. Afterwards, sodium dithionite (1.39 g, 7.98 mmol) dissolved in water (2 mL) was added. Then, NH₃ (30% aqueous solution, 790 μL, 5.99 mmol) was added to this mixture at 0° C. The reaction was allowed to warm to room temperature and stirred at room temperature for 3 h. Again, NH₃ (30% aqueous solution, 211 μL, 1.60 mmol) was added and the reaction was stirred at room temperature for 16 h. Then, NH₃ (30% aqueous solution, 211 μL, 1.60 mmol) and sodium dithionite (280 mg, 1.60 mmol) were added and the reaction was stirred at room temperature for 3 h. The reaction progress was controlled by LC/MS. The reaction mixture was diluted with H₂O (75 mL) and afterwards CH₂Cl₂ (100 mL) was added. The aqueous layer was extracted with CH₂Cl₂/CH₃OH (8:2, 50 mL). The combined organic layers were dried over sodium sulfate and filtered. The solvent was removed under reduced pressure to obtain 450 mg (623 μmol, 78%) of a white solid, which was used without further purification. LCMS (Method A) retention time 1.94 min, [M+H]⁺=722.4. ¹H-NMR (300 MHz, DMSO-d₆) δ [ppm]=8.05 (brs, 1H), 8.00-7.93 (m, 1H), 7.60-7.51 (m, 2H), 7.44 (brs, 1H), 6.92 (brs, 1H), 6.86-6.77 (m, 1H), 6.73-6.65 (m, 1H), 5.89-5.60 (m, 2H), 5.28 (d, J=5.4 Hz, 2H), 4.59 (s, 2H), 3.92-3.85 (m, 4H), 3.79 (t, J=6.3 Hz, 2H), 3.56 (s, 2H), 2.95 (q, J=7.6 Hz, 2H), 2.45 (s, 3H), 2.25 (t, J=7.3 Hz, 2H), 1.87-1.71 (m, 2H), 1.34 (s, 9H), 1.15 (t, J=7.5 Hz, 3H).

### Preparation of tert-Butyl 4-((2-amino-5-carbamoyl-1-((E)-4-((Z)-6-carbamoyl-2-((4-ethyl-2-methyloxazole-5-carbonyl)imino)-4-methoxybenzo[d]thiazol-3(2H)-yl)but-2-en-1-yl)-1H-benzo[d]imidazol-7-yl)oxy)butanoate (XX6.5)

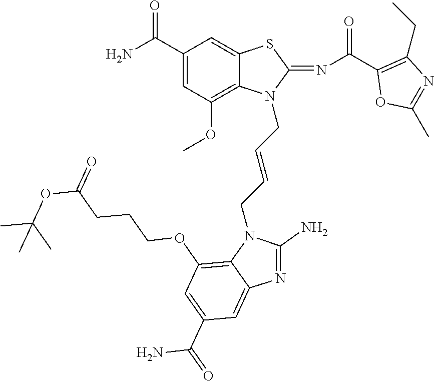 [SMILES: CCc1nc(C)oc1C(=O)/N=c1\sc2cc(C(N)=O)cc(OC)c2n1C/C=C/Cn1c(N)nc2cc(C(N)=O)cc(OCCCC(=O)OC(C)(C)C)c21]

Compound XX6.4 (450 mg, 623 μmol) was dissolved in CH₃OH (10 mL) and cooled to 0° C. Afterwards, BrCN (132 mg, 1.24 mmol) was added. The reaction mixture was stirred at room temperature for 5 h. Again, BrCN (33 mg, 0.31 mmol) was added and the mixture was stirred at room temperature for 19 h. Then, BrCN (33 mg, 0.31 mmol) was added and the reaction was stirred at room temperature for 2.5 h. The product formation was controlled by LC/MS. The solvent was removed under reduced pressure. The residue was suspended in petroleum ether and the resulting solid filtered off. The product was dried in vacuo to yield 436 mg (583 μmol, 93%) of a yellowish solid, which was used without further purification. LCMS (Method A) retention time 2.05 min, [M+H]⁺=747.3. ¹H-NMR (300 MHz, DMSO-d₆) δ [ppm]=8.57 (brs, 2H), 8.11-8.01 (m, 2H), 8.01-7.97 (m, 1H), 7.61-7.53 (m, 1H), 7.52-7.38 (m, 3H), 7.37-7.31 (m, 1H), 5.94-5.66 (m, 2H), 5.30 (d, J=5.3 Hz, 2H), 4.86 (d, J=5.1 Hz, 2H), 3.99 (t, J=6.5 Hz, 2H), 3.81 (s, 3H), 2.79 (q, J=7.5 Hz, 2H), 2.43 (s, 3H), 2.29-2.11 (m, 2H), 1.92-1.70 (m, 2H), 1.35 (s, 9H), 1.04 (t, J=7.5 Hz, 3H).

### Preparation of tert-Butyl 4-((5-carbamoyl-1-((E)-4-((Z)-6-carbamoyl-2-((4-ethyl-2-methyloxazole-5-carbonyl)imino)-4-methoxybenzo[d]thiazol-3(2H)-yl)but-2-en-1-yl)-2-(4-ethyl-2-methyloxazole-5-carboxamido)-1H-benzo[d]imidazol-7-yl)oxy)butanoate (XX6.6)

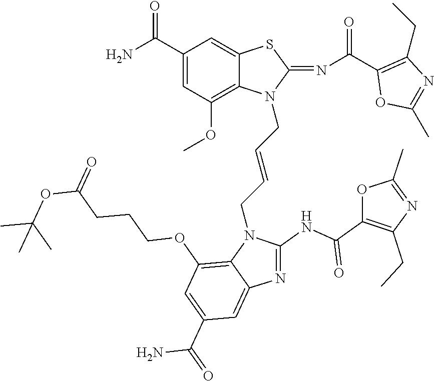 [SMILES: CCc1nc(C)oc1C(=O)/N=c1\sc2cc(C(N)=O)cc(OC)c2n1C/C=C/Cn1c(NC(=O)c2oc(C)nc2CC)nc2cc(C(N)=O)cc(OCCCC(=O)OC(C)(C)C)c21]

Compound XX6.5 (425 mg, 569 μmol) and 4-ethyl-2-methyl-1,3-oxazole-5-carboxylic acid (132 mg, 853 μmol) were dissolved in DMF (5 mL). Afterwards, HATU (327 mg, 853 μmol) and DIPEA (150 μL, 853 μmol) were added and the reaction mixture was stirred at room temperature for 4 h. 4-ethyl-2-methyl-1,3-oxazole-5-carboxylic acid (44.1 mg, 284 μmol) was again added and the reaction was stirred at room temperature for 2 h. Then, DIPEA (50.0 μL, 284 μmol) was added and the reaction was stirred at room temperature for 21 h. DIPEA (100 μL, 568 μmol) was again added and the reaction was stirred at room temperature for 2 h. Afterwards, HATU (109 mg, 284 μmol) was added and the mixture was stirred at room temperature for 19 h. The product formation was controlled by LC/MS. The solvent was removed under reduced pressure. The mixture was quenched with water (15 mL), and the solid was filtered off and washed with water. The product was dried in vacuo. The residue was again suspended in water, filtered off and dried in vacuo to yield 377 mg (426 μmol, 75%) of an orange solid, which was used without further purification. LCMS (Method A) retention time 2.31 min, [M+H]⁺=884.3.

### Preparation of 4-((5-carbamoyl-1-((E)-4-((Z)-6-carbamoyl-2-((4-ethyl-2-methyloxazole-5-carbonyl)imino)-4-methoxybenzo[d]thiazol-3(2H)-yl)but-2-en-1-yl)-2-(4-ethyl-2-methyloxazole-5-carboxamido)-1H-benzo[d]imidazol-7-yl)oxy) butanoic acid (XX6.7)

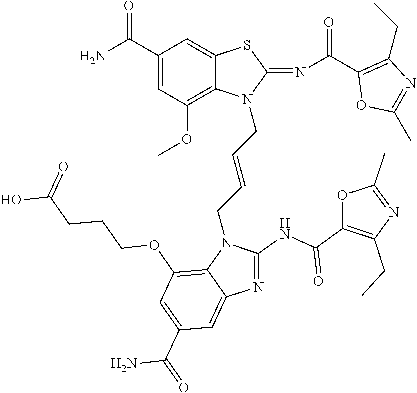 [SMILES: CCc1nc(C)oc1C(=O)/N=c1\sc2cc(C(N)=O)cc(OC)c2n1C/C=C/Cn1c(NC(=O)c2oc(C)nc2CC)nc2cc(C(N)=O)cc(OCCCC(=O)O)c21]

Compound XX6.6 (365 mg, 413 μmol) was suspended in dioxane (2 mL) and 4 M HCl in dioxane (3.3 mL, 13.2 mmol) was added. The resulting suspension was stirred at room temperature for 1 h. The product formation was controlled by LC/MS. The formed solid was filtered off, washed with diethyl ether and dried in vacuo to yield 310 mg (358 μmol, 87%) of an off-white solid, which was used without further purification. LCMS (Method A) retention time 1.94 min, [M+H]⁺=828.3.

### Preparation of N-(5-carbamoyl-1-((E)-4-((Z)-6-carbamoyl-2-((4-ethyl-2-methyloxazole-5-carbonyl)imino)-4-methoxybenzo[d]thiazol-3(2H)-yl)but-2-en-1-yl)-7-(4-(1,2-dimethylhydrazineyl)-4-oxobutoxy)-1H-benzo[d]imidazol-2-yl)-4-ethyl-2-methyloxazole-5-carboxamide (Compound XXVIII)

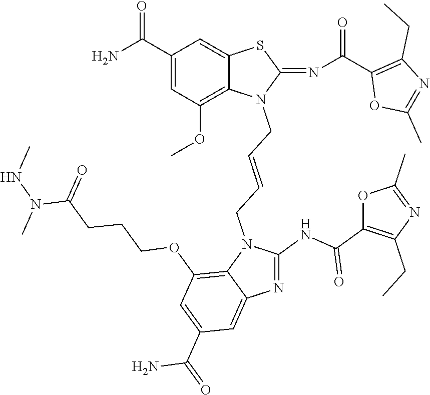 [SMILES: CCc1nc(C)oc1C(=O)/N=c1\sc2cc(C(N)=O)cc(OC)c2n1C/C=C/Cn1c(NC(=O)c2oc(C)nc2CC)nc2cc(C(N)=O)cc(OCCCC(=O)N(C)NC)c21]

Compound XX6.7 (300 mg, 347 μmol) was suspended in DMF (5 mL). Afterwards, DIPEA (181 μL, 1.04 mmol), NHS (81.5 mg, 694 μmol) and DIC (107 L, 694 μmol) were added and the resulting reaction mixture was stirred at 40° C. for 4 h. NHS (81.5 mg, 694 μmol) was again added and the mixture was stirred at 40° C. for 17.5 h. The reaction progress was controlled by LC/MS. Afterwards, 1,2-dimethylhydrazine dihydrochloride (231 mg, 1.73 mmol) and DIPEA (181 μL, 1.04 mmol) were added and the resulting reaction mixture was stirred at 40° C. for 4.5 h. Again, DIPEA (60.4 μL, 347 μmol) was added and the reaction was stirred at 40° C. for 19 h. Then, DIPEA (302 μL, 1.74 mmol) was added and the reaction was stirred at 40° C. for 6 h. The reaction progress was controlled by LC/MS. The solvent was removed under reduced pressure and the residue was purified on RP18 silica by prepHPLC (Method II). Product containing fractions were freeze-dried to obtain 4 mg (4.1 μmol, purity 90.9% by HPLC Method C) and 21 mg (21 μmol, purity 84.5% by HPLC Method C) each as a white solid. LCMS (Method B) retention time 2.74 min, [M+H]⁺=870.2.

### Example 34: Synthetic Scheme for Compound XXIX

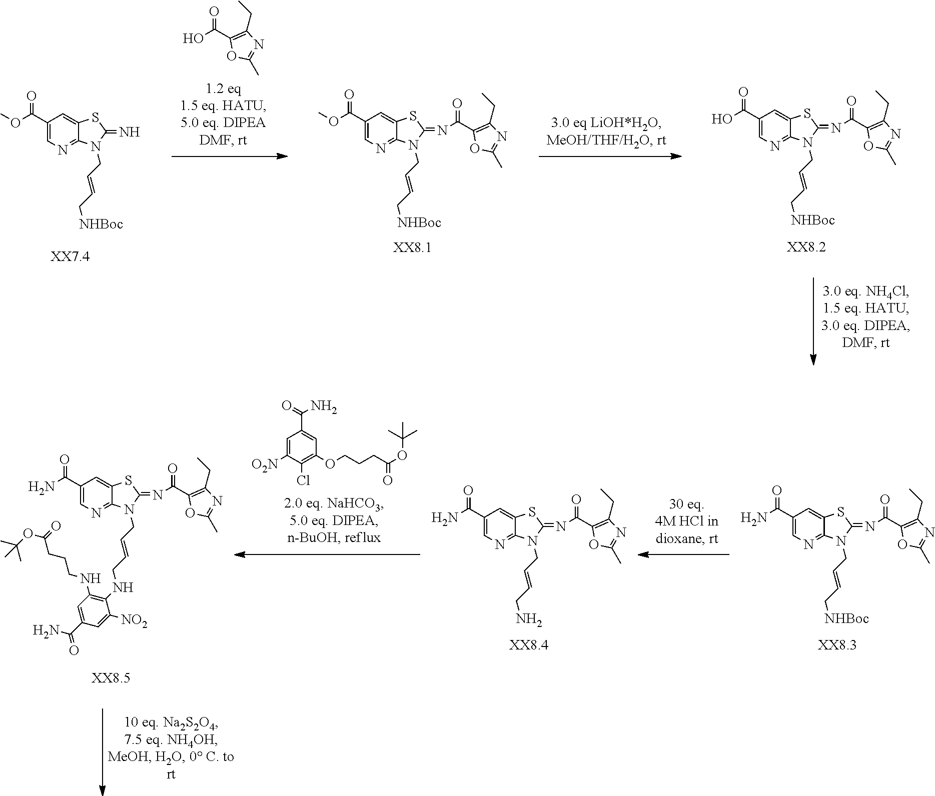

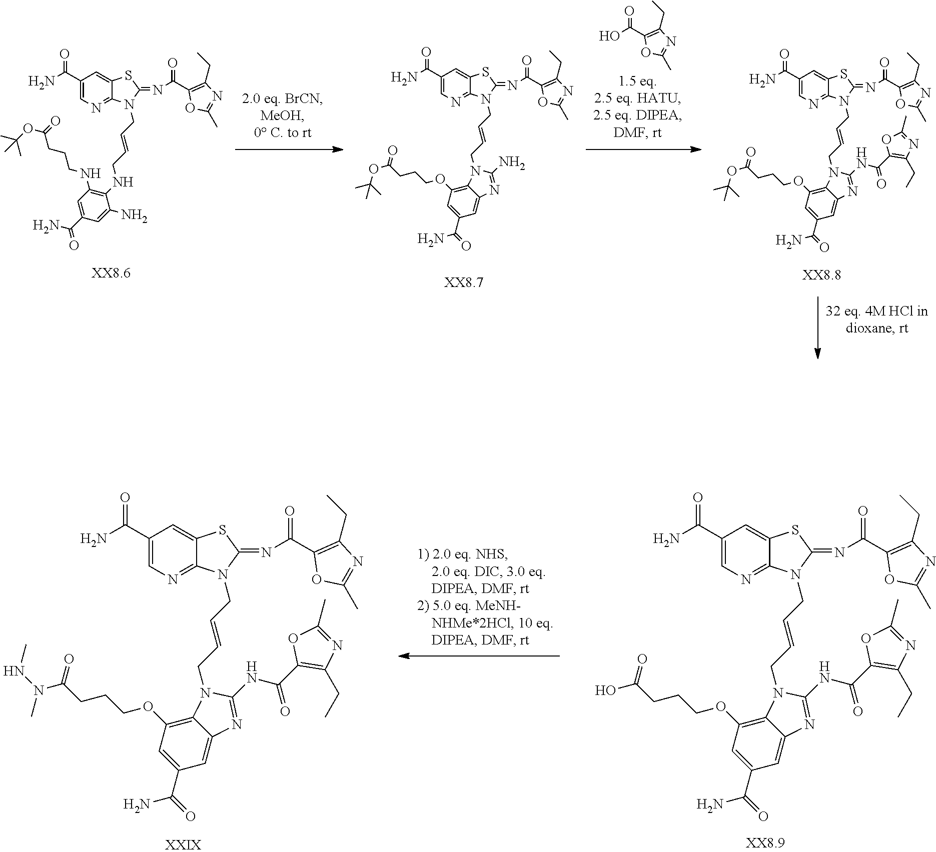

### Preparation of Methyl (Z)-3-((E)-4-((tert-butoxycarbonyl)amino)but-2-en-1-yl)-2-((4-ethyl-2-methyloxazole-5-carbonyl)imino)-2,3-dihydrothiazolo[4,5-b]pyridine-6-carboxylate (XX8.1)

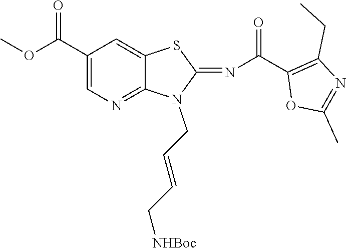 [SMILES: CCc1nc(C)oc1C(=O)/N=c1\sc2cc(C(=O)OC)cnc2n1C/C=C/CNC(=O)OC(C)(C)C]

Compound XX7.4 (2.7 g, 7.1 mmol) and 4-ethyl-2-methyl-1,3-oxazole-5-carboxylic acid (1.3 g, 8.5 mmol) were dissolved in DMF (20 mL). Afterwards, HATU (4.1 g, 11 mmol) and DIPEA (6.2 mL, 35 mmol) were added and the reaction mixture was stirred at room temperature for 30 min. The mixture was quenched with water (40 mL), the solid was filtered off and washed with water. The product was dried in vacuo to yield 3.5 g (6.7 mmol, 97%) as an off-white solid, which was used without further purification. LCMS (Method A) retention time 3.57 min, [M+H]⁺=516.1. ¹H-NMR (300 MHz, CDCl₃) δ [ppm]=9.11-9.01 (m, 1H), 8.59-8.51 (m, 1H), 5.86 (t, J=3.5 Hz, 2H), 5.19-5.11 (m, 2H), 4.50 (brs, 1H), 3.98 (s, 3H), 3.73 (d, J=5.6 Hz, 2H), 3.10 (q, J=7.5 Hz, 2H), 2.55 (s, 3H), 1.39 (s, 9H), 1.32 (t, J=7.5 Hz, 3H).

### Preparation of (Z)-3-((E)-4-((tert-Butoxycarbonyl)amino)but-2-en-1-yl)-2-((4-ethyl-2-methyloxazole-5-carbonyl)imino)-2,3-dihydrothiazolo[4,5-b]pyridine-6-carboxylic acid (XX8.2)

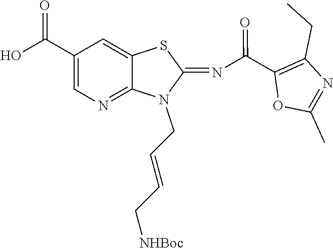 [SMILES: CCc1nc(C)oc1C(=O)/N=c1\sc2cc(C(=O)O)cnc2n1C/C=C/CNC(=O)OC(C)(C)C]

Compound XX8.1 (3.5 g, 6.7 mmol) was dissolved in CH₃OH/THF/H₂O (2:2:1; 50 mL). Afterwards, LiOH (487 mg, 20.3 mmol) was added and the resulting reaction mixture was stirred at room temperature for 18 h. The organic solvents were removed in vacuo (rotary evaporator). The aqueous phase was cooled to 0° C. and neutralized with 1 M aq. HCl. The precipitate was filtered and washed with water. The solid was dissolved/suspended in CH₃CN/H₂O (1:1, 25 mL) and freeze-dried to yield 2.16 g (4.31 μmol, 63%) of a yellowish solid, which was used without further purification. LCMS (Method A) retention time 2.27 min, [M+H]⁺=502.0. ¹H-NMR (300 MHz, DMSO-d₆) δ [ppm]=9.01-8.93 (m, 1H), 8.88-8.78 (m, 1H), 6.89 (brs, 1H), 5.84-5.55 (m, 2H), 5.05 (d, J=5.2 Hz, 2H), 3.49 (d, J=5.6 Hz, 2H), 3.01 (q, J=7.5 Hz, 2H), 2.48 (s, 3H), 1.27 (s, 9H), 1.22 (t, J=7.5 Hz, 3H).

### Preparation of tert-Butyl ((E)-4-((Z)-6-carbamoyl-2-((4-ethyl-2-methyloxazole-5-carbonyl)imino)thiazolo[4,5-b]pyridin-3(2H)-yl)but-2-en-1-yl)carbamate (XX8.3)

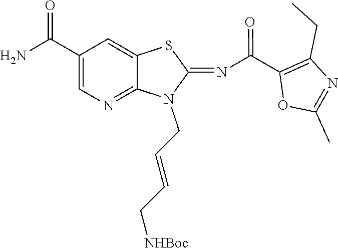 [SMILES: CCc1nc(C)oc1C(=O)/N=c1\sc2cc(C(N)=O)cnc2n1C/C=C/CNC(=O)OC(C)(C)C]

Compound XX8.2 (2.0 g, 4.0 mmol) was dissolved in DMF (8 mL) and, afterwards, NH₄Cl (639 mg, 12.0 mmol), HATU (2.2 g, 6.0 mmol), as well as DIPEA (2.0 mL, 12 mmol) were added. The reaction mixture was stirred at room temperature for 30 min. The mixture was diluted with water (30 mL) and the resulting suspension was filtered. The solid was washed with water and dried in vacuo to yield 1.59 g (3.18 mmol, 79%) of a white solid, which was used without further purification. LCMS (Method A) retention time 2.11 min, [M+H]⁺=501.0. ¹H-NMR (300 MHz, DMSO-d₆) d: 9.02-8.92 (m, 1H), 8.78-8.72 (m, 1H), 8.17 (brs, 1H), 7.62 (brs, 1H), 6.89 (t, J=6.0 Hz, 1H), 5.84-5.60 (m, 2H), 5.05 (d, J=5.2 Hz, 2H), 3.50 (t, J=5.5 Hz, 2H), 3.01 (q, J=7.5 Hz, 2H), 2.48 (s, 3H), 1.30 (s, 9H), 1.22 (t, J=7.5 Hz, 3H).

### Preparation of N-((Z)-3-((E)-4-Aminobut-2-en-1-yl)-6-carbamoylthiazolo[4,5-b]pyridin-2(3H)-ylidene)-4-ethyl-2-methyloxazole-5-carboxamide (XX8.4)

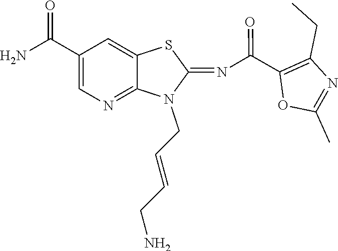 [SMILES: CCc1nc(C)oc1C(=O)/N=c1\sc2cc(C(N)=O)cnc2n1C/C=C/CN]

Compound XX8.3 (1.7 g, 3.4 mmol) was suspended and 4 M HCl in dioxane (25.4 mL, 102 mmol) was added. The resulting suspension was stirred at room temperature for 1 h. The formed solid was filtered off, washed with Et₂O and dried in vacuo. The residue was purified by automated flash column chromatography on Büchi C-850 (XBridge® Prep OBD™ C18 0.5 μM, 50 mm×250 mm, 100 mL/min; 5-100% CH₃CN in H₂O (50 M NH₄HCO₃) in 30 min). Product containing fractions were combined to yield 1.10 g (2.75 mmol, 81%) of a yellowish solid. LCMS (Method A) retention time 1.30 min, [M+H]⁺=401.0. ¹H-NMR (300 MHz, DMSO-d₆) δ [ppm]=8.95 (d, J=2.0 Hz, 1H), 8.74 (d, J=1.9 Hz, 1H), 8.17 (brs, 1H), 7.62 (brs, 1H), 5.83-5.75 (m, 2H), 5.09-5.02 (m, 2H), 3.15-3.08 (m, 2H), 3.02 (q, J=7.5 Hz, 2H), 2.48 (s, 3H), 1.22 (t, J=7.5 Hz, 3H).

### Preparation of tert-Butyl 4-(5-carbamoyl-2-(((E)-4-((Z)-6-carbamoyl-2-((4-ethyl-2-methyloxazole-5-carbonyl)imino)thiazolo[4,5-b]pyridin-3(2H)-yl)but-2-en-1-yl)amino)-3-nitrophenoxy)butanoate (XX8.5)

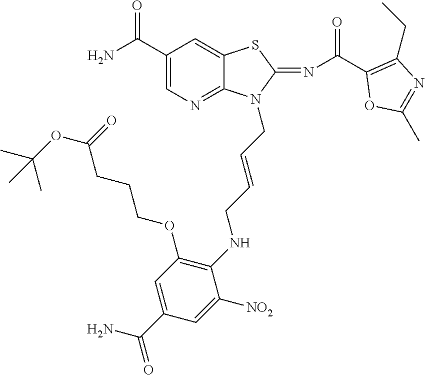 [SMILES: CCc1nc(C)oc1C(=O)/N=c1\sc2cc(C(N)=O)cnc2n1C/C=C/CNc1c(OCCCC(=O)OC(C)(C)C)cc(C(N)=O)cc1[N+](=O)[O-]]

Compound XX8.4 (220 mg, 549 μmol) was suspended in n-butanol (6 mL). Afterwards, DIPEA (483 μL, 2.74 mmol) and sodium bicarbonate (92 mg, 1.1 mmol) were added and the mixture was stirred at room temperature for 10 min. Then, tert-butyl 4-(5-carbamoyl-2-chloro-3-nitrophenoxy)butanoate (197 mg, 549 μmol) was added and the reaction mixture was stirred at 130° C. for 18 h. The reaction was cooled to room temperature and quenched with water (30 mL). The aqueous layer was extracted with CH₂Cl₂/CH₃OH (9:1, 70 mL). The organic layer was dried over sodium sulfate and filtered. The solvent was removed under reduced pressure and the resulting residue was purified on silica by automated flash column chromatography on a Biotage Selekt (Biotage Sfär HC 10 g, 40 mL/min, 0-20% CH₃OH in CH₂Cl₂ in 30 min). Product containing fractions were combined and the solvent was evaporated to obtain 230 mg (318 μmol, 57%) of an orange solid. LCMS (Method B) retention time 4.53 min, [M+H]⁺=723.1. ¹H-NMR (300 MHz, DMSO-d₆) δ [ppm]=8.91 (d, J=2.0 Hz, 1H), 8.73 (d, J=1.9 Hz, 1H), 8.15 (s, 1H), 8.02 (d, J=1.9 Hz, 1H), 7.89 (s, 1H), 7.67-7.57 (m, 2H), 7.40 (d, J=1.9 Hz, 1H), 7.26 (s, 1H), 5.81-5.73 (m, 2H), 5.04-4.98 (m, 2H), 4.12-4.01 (m, 2H), 3.90 (t, J=6.3 Hz, 2H), 2.90 (q, J=7.5 Hz, 2H), 2.46 (s, 3H), 2.28 (t, J=7.3 Hz, 2H), 1.85 (p, J=6.8 Hz, 2H), 1.36 (s, 9H), 1.13 (t, J=7.5 Hz, 3H).

### Preparation of tert-Butyl 4-(3-amino-5-carbamoyl-2-(((E)-4-((Z)-6-carbamoyl-2-((4-ethyl-2-methyloxazole-5-carbonyl)imino)thiazolo[4,5-b]pyridin-3(2H)-yl)but-2-en-1-yl)amino)phenoxy)butanoate (XX8.6)

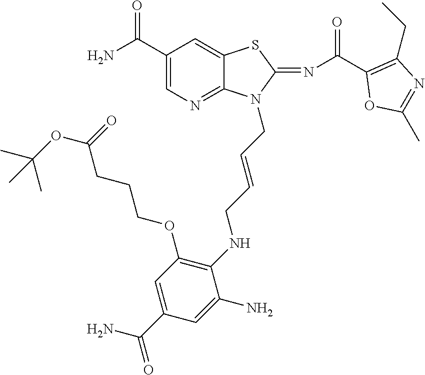 [SMILES: CCc1nc(C)oc1C(=O)/N=c1\sc2cc(C(N)=O)cnc2n1C/C=C/CNc1c(N)cc(C(N)=O)cc1OCCCC(=O)OC(C)(C)C]

Compound XX8.5 (735 mg, 1.02 mmol) was dissolved in MeOH (20 mL) and cooled to 0° C. Afterwards, sodium dithionite (1.7 g, 10.2 mmol) in water (5 mL) and aqueous NH₃ (30% aqueous solution, 1.0 mL, 7.6 mmol) were added. The reaction mixture was allowed to warm to room temperature and stirred for 1 h. The reaction mixture was diluted with H₂O (75 mL) and afterwards CH₂Cl₂ (100 mL) was added. The aqueous layer was extracted with CH₂Cl₂/MeOH (8:2, 2×50 mL). The organic layer was dried over sodium sulfate and filtered. The solvent was removed under reduced pressure to yield 591 mg (853 μmol, 84%) of an orange solid, which was used without further purification. LCMS (Method A) retention time 1.93 min, [M+H]⁺=693.3. ¹H-NMR (300 MHz, DMSO-d₆): δ [ppm]=8.93 (d, J=2.0 Hz, 1H), 8.74 (d, J=2.0 Hz, 1H), 8.17 (brs, 1H), 7.71-7.46 (m, 2H), 6.92 (brs, 1H), 6.80 (d, J=1.8 Hz, 1H), 6.68 (d, J=1.8 Hz, 1H), 5.93-5.70 (m, 2H), 5.03 (d, 2H), 4.60 (s, 2H), 3.83 (t, J=6.3 Hz, 2H), 3.54 (d, J=4.8 Hz, 2H), 2.98 (q, J=7.5 Hz, 2H), 2.47 (s, 3H), 2.28 (t, J=7.3 Hz, 2H), 1.81 (p, J=6.8 Hz, 2H), 1.34 (s, 9H), 1.18 (t, J=7.5 Hz, 3H).

### Preparation of tert-Butyl 4-((2-amino-5-carbamoyl-1-((E)-4-((Z)-6-carbamoyl-2-((4-ethyl-2-methyloxazole-5-carbonyl)imino)thiazolo[4,5-b]pyridin-3(2H)-yl)but-2-en-1-yl)-1H-benzo[d]imidazol-7-yl)oxy)butanoate (XX8.7)

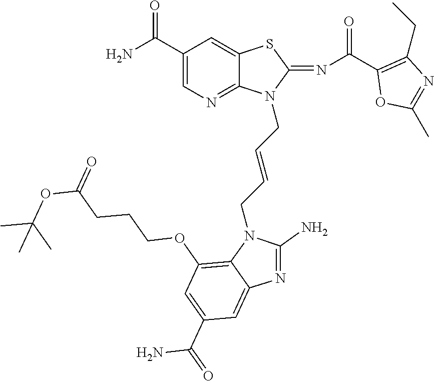 [SMILES: CCc1nc(C)oc1C(=O)/N=c1\sc2cc(C(N)=O)cnc2n1C/C=C/Cn1c(N)nc2cc(C(N)=O)cc(OCCCC(=O)OC(C)(C)C)c21]

Compound XX8.6 (575 mg, 830 μmol) was dissolved in MeOH (15 mL) and cooled to 0° C. Then, BrCN (175 mg, 1.66 mmol) was added and the reaction mixture was stirred at room temperature for 5 h. The solvent was removed under reduced pressure. The residue was suspended in petroleum ether and filtered off. The product was dried in vacuo to yield 540 mg (752 μmol, 91%) of a yellowish solid, which was used without further purification. LCMS (Method A) retention time 1.72 min, [M+H]⁺=718.2. ¹H-NMR (300 MHz, DMSO-d₆) δ [ppm]=8.93 (d, J=1.9 Hz, 1H), 8.77 (d, J=1.9 Hz, 1H), 8.38 (brs, 2H), 8.17 (brs, 1H), 8.00 (brs, 1H), 7.63 (brs, 1H), 7.45 (d, J=1.2 Hz, 1H), 7.37 (brs, 1H), 7.30 (d, J=1.3 Hz, 1H), 5.93-5.77 (m, 2H), 5.09-5.01 (m, 2H), 4.86-4.80 (m, 2H), 3.99 (t, J=6.3 Hz, 2H), 2.76 (q, J=7.5 Hz, 2H), 2.44 (s, 3H), 2.22 (t, J=7.3 Hz, 2H), 1.77 (p, J=6.9 Hz, 2H), 1.34 (s, 9H), 1.01 (t, J=7.5 Hz, 3H).

### Preparation of tert-Butyl 4-((5-carbamoyl-1-((E)-4-((Z)-6-carbamoyl-2-((4-ethyl-2-methyloxazole-5-carbonyl)imino)thiazolo[4,5-b]pyridin-3(2H)-yl)but-2-en-1-yl)-2-(4-ethyl-2-methyloxazole-5-carboxamido)-1H-benzo[d]imidazol-7-yl)oxy)butanoate (XX8.8)

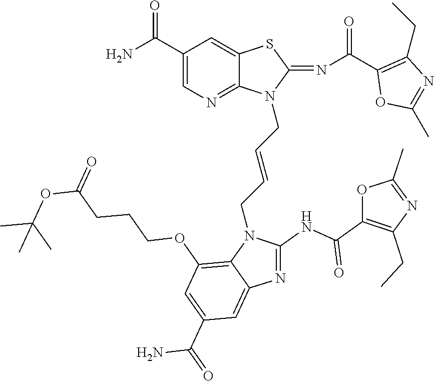 [SMILES: CCc1nc(C)oc1C(=O)/N=c1\sc2cc(C(N)=O)cnc2n1C/C=C/Cn1c(NC(=O)c2oc(C)nc2CC)nc2cc(C(N)=O)cc(OCCCC(=O)OC(C)(C)C)c21]

Compound XX8.7 (520 mg, 724 μmol) and 4-ethyl-2-methyl-1,3-oxazole-5-carboxylic acid (112 mg, 724 μmol) were dissolved in DMF (6 mL). Afterwards, HATU (417 mg, 1.09 mmol) and DIPEA (191 μL, 1.09 mmol) were added and the reaction mixture was stirred at room temperature for 22 h. Again, 4-ethyl-2-methyl-1,3-oxazole-5-carboxylic acid (56.2 mg, 362 μmol), HATU (278 mg, 724 μmol) and DIPEA (127 UL, 724 μmol) were added and the reaction was stirred at room temperature for 20 h. The product formation was controlled by LC/MS. The mixture was quenched with water (10 mL), and the solid was filtered off and washed with water. The product was dried in vacuo and the residue was suspended in acetonitrile, filtered off and dried. The solid was purified on preparative TLC (2 mm plate, CH₂Cl₂/CH₃OH=9:1). The product containing filtrate was purified on flash column chromatography on Büchi C-850 (XSelect® CSH™ Prep OBD™ C18 0.5 μM, 50 mm×150 mm, 100 mL/min; 5-100% CH₃CN in H₂O (+0.15% TFA) in 30 min) to yield 115 mg (134 μmol, 18%) of a yellowish solid. LCMS (Method A) retention time 2.20 min, [M+H]⁺=855.3. ¹H-NMR (300 MHz, DMSO-d₆) δ [ppm]=12.69 (brs, 1H), 8.91 (d, J=2.0 Hz, 1H), 8.74 (d, J=1.9 Hz, 1H), 8.14 (brs, 1H), 7.92 (brs, 1H), 7.65-7.59 (m, 2H), 7.31-7.25 (m, 2H), 5.98-5.83 (m, 1H), 5.82-5.67 (m, 1H), 5.07-4.99 (m, 2H), 4.90 (d, J=5.0 Hz, 2H), 4.02 (t, J=6.3 Hz, 2H), 2.85-2.68 (m, 4H), 2.42 (s, 3H), 2.39 (s, 3H), 2.26 (t, J=7.3 Hz, 2H), 1.82 (p, J=6.9 Hz, 2H), 1.35 (s, 9H), 1.05-0.91 (m, 6H).

### Preparation of 4-((5-Carbamoyl-1-((E)-4-((Z)-6-carbamoyl-2-((4-ethyl-2-methyloxazole-5-carbonyl)imino)thiazolo[4,5-b]pyridin-3(2H)-yl)but-2-en-1-yl)-2-(4-ethyl-2-methyloxazole-5-carboxamido)-1H-benzo[d]imidazol-7-yl)oxy) butanoic acid (XX8.9)

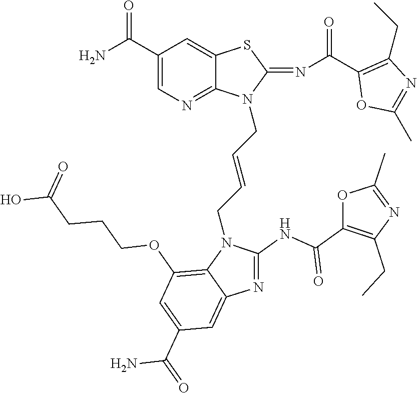 [SMILES: CCc1nc(C)oc1C(=O)/N=c1\sc2cc(C(N)=O)cnc2n1C/C=C/Cn1c(NC(=O)c2oc(C)nc2CC)nc2cc(C(N)=O)cc(OCCCC(=O)O)c21]

Compound XX8.8 (60.0 mg, 70.2 μmol) was suspended in dioxane (2 mL), and 4 M HCl in dioxane (560 μL, 2.25 mmol) was added. The resulting suspension was stirred at room temperature for 20 min. The product formation was controlled by LC/MS. The formed solid was filtered off, washed with diethyl ether and dried in vacuo to give 42 mg (50 μmol, 72%) of an off-white solid, which was used without further purification. LCMS (Method A) retention time 1.89 min, [M+H]⁺=799.5. ¹H-NMR (300 MHz, DMSO-d₆) δ [ppm]=12.70 (brs, 1H), 8.92 (d, J=2.0 Hz, 1H), 8.81-8.71 (m, 1H), 8.15 (s, 1H), 7.93 (s, 1H), 7.66-7.59 (m, 2H), 7.33-7.27 (m, 2H), 5.99-5.70 (m, 2H), 5.04 (d, J=5.4 Hz, 2H), 4.91 (d, J=5.1 Hz, 2H), 4.05 (t, J=6.3 Hz, 2H), 2.85-2.69 (m, 4H), 2.43 (s, 3H), 2.41 (s, 3H), 2.31 (t, J=7.2 Hz, 2H), 1.92-1.77 (m, 2H), 1.05-0.92 (m, 6H).

### Preparation of N-(5-Carbamoyl-1-((E)-4-((Z)-6-carbamoyl-2-((4-ethyl-2-methyloxazole-5-carbonyl)imino)thiazolo[4,5-b]pyridin-3(2H)-yl)but-2-en-1-yl)-7-(4-(1,2-dimethylhydrazineyl)-4-oxobutoxy)-1H-benzo[d]imidazol-2-yl)-4-ethyl-2-methyloxazole-5-carboxamide (Compound XXIX)

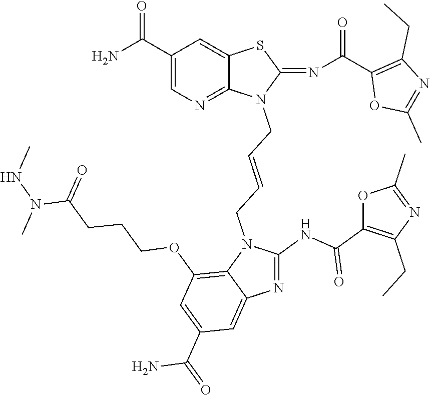 [SMILES: CCc1nc(C)oc1C(=O)/N=c1\sc2cc(C(N)=O)cnc2n1C/C=C/Cn1c(NC(=O)c2oc(C)nc2CC)nc2cc(C(N)=O)cc(OCCCC(=O)N(C)NC)c21]

Compound XX8.9 (50.0 mg, 59.9 μmol) was suspended in DMF (3 mL). Afterwards, DIPEA (31.2 μL, 179 μmol), NHS (14.1 mg, 119 μmol) and DIC (18.5 μL, 119 μmol) were added and the resulting reaction mixture was stirred at 45° C. for 24 h. The reaction progress was controlled by LC/MS. Then, 1,2-dimethylhydrazine dihydrochloride (39.8 mg, 299 μmol) and DIPEA (104 μL, 598 μmol) were added and the resulting reaction mixture was stirred at room temperature for 1 h. The reaction progress was controlled by LC/MS. The solvent was removed under reduced pressure and the residue was purified on RP18 silica by prepHPLC (Method I). Product containing fractions were freeze-dried to obtain 19 mg (20 μmol, 33%, purity 95.9% by HPLC Method A) of a white solid. LCMS (Method D) retention time 4.24 min, [M+H]⁺=842.2.

### Example 35: Synthetic Scheme for Compound XXX

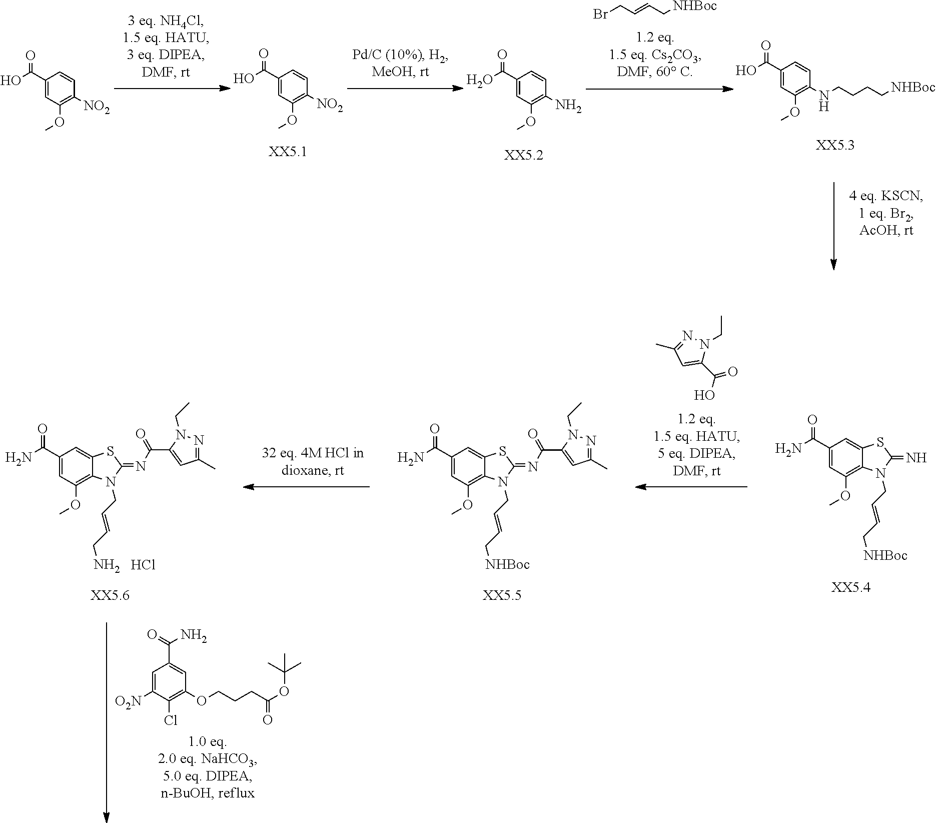

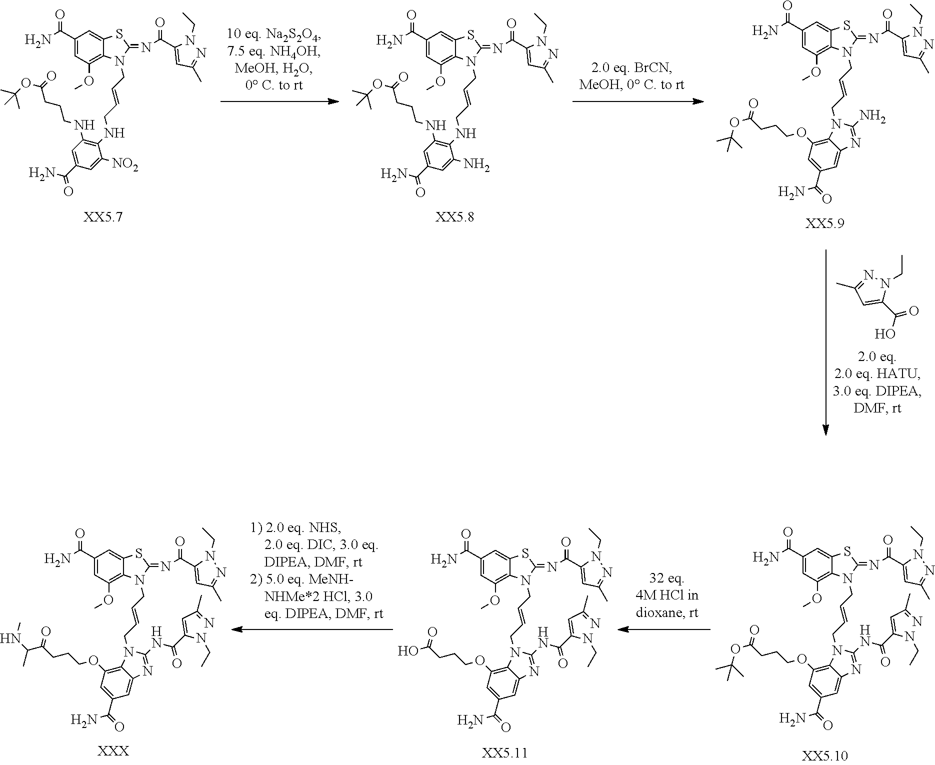

### Preparation of 3-Methoxy-4-nitrobenzamide (XX5.1)

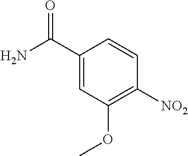

To a stirred solution of 3-methoxy-4-nitrobenzoic acid (11.3 g, 57.3 mmol) in DMF (100 mL) was added NH₄Cl (9.2 g, 172.0 mmol), HATU (32.7 g, 86.0 mmol) and DIPEA (29.4 mL, 172.0 mmol). The mixture was stirred at room temperature for 1 h. The mixture was diluted with water. A precipitate was formed, filtered off and washed with water. The solid was dried in vacuo to yield 10.4 g (53.0 mmol, 93%) of a white solid. LCMS (Method A) retention time 1.85 min, [M+H]⁺=197.0. ¹H-NMR (300 MHz, DMSO-d₆) δ [ppm]=8.22 (s, 1H), 7.94 (d, J=8.3 Hz, 1H), 7.75 (d, J=1.6 Hz, 1H), 7.68 (s, 1H), 7.57 (dd, J=8.3, 1.7 Hz, 1H), 3.98 (s, 3H).

### Preparation of 4-Amino-3-methoxybenzamide (XX5.2)

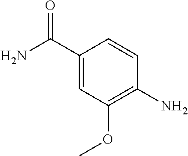

3-Methoxy-4-nitrobenzamide (10.4 g, 53.0 mmol) was dissolved in CH₃OH (200 mL) and 10% Pd—C(1.9 g, 1.8 mmol) was added under inert atmosphere. The resulting mixture was stirred under a H₂ atmosphere at room temperature for 18 h. The catalyst was removed by filtration through a small pad of CELITE® Hyflo Supercel. The filter cake was washed with CH₃OH. The solvent was removed under reduced pressure. The product was dried in vacuo to yield 8.5 g (51.1 mmol, 96%) of an off-white solid, which was used without further purification. LCMS (Method A) retention time 1.06 min, [M+H]⁺=167.0. ¹H-NMR (300 MHz, DMSO-d₆) δ [ppm]: 7.57 (brs, 1H), 7.37-7.25 (m, 2H), 6.87 (brs, 1H), 6.59 (d, J=8.0 Hz, 1H), 5.21 (brs, 2H), 3.79 (s, 3H).

### Preparation of tert-Butyl (E)-(4-((4-carbamoyl-2-methoxyphenyl)amino)but-2-en-1-yl)carbamate (XX5.3)

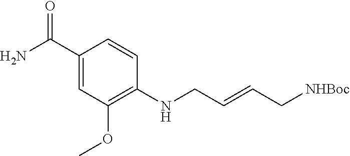 [SMILES: COc1cc(C(N)=O)ccc1NC/C=C/CNC(=O)OC(C)(C)C]

4-Amino-3-methoxybenzamide (2.5 g, 15 mmol) was dissolved in DMF (15 mL). Then, Cs₂CO₃ (7.4 g, 22 mmol) and tert-butyl N-[(2E)-4-bromobut-2-en-1-yl]carbamate (3.7 g, 15 mmol) were added and the mixture was stirred at 60° C. for 1 h. The mixture was diluted with water (30 mL) and the product was extracted with ethyl acetate (50 mL). The organic solvent was removed under reduced pressure. The residue was suspended in diethyl ether and afterwards filtered. The solid was washed with Et₂O and dried in vacuo to yield 2.81 g (8.38 mmol, 56%) of a white solid, which was used without further purification. LCMS (Method A) retention time 2.33 min, [M+H]⁺=336.0. ¹H-NMR (300 MHz, DMSO-d₆) δ [ppm]: 7.59 (brs, 1H), 7.42-7.34 (m, 1H), 7.32 (d, J=1.9 Hz, 1H), 6.91 (brs, 2H), 6.46 (d, J=8.3 Hz, 1H), 5.59-5.54 (m, 2H), 5.50 (t, J=6.1 Hz, 1H), 3.82 (s, 3H), 3.78-3.71 (m, 2H), 3.58-3.47 (m, 2H), 1.36 (s, 9H).

### Preparation of tert-Butyl (E)-(4-(6-carbamoyl-2-imino-4-methoxybenzo[d]thiazol-3(2H)-yl)but-2-en-1-yl)carbamate (XX5.4)

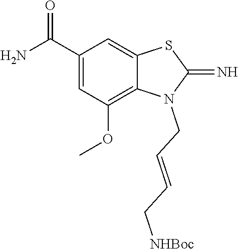 [SMILES: COc1cc(C(N)=O)cc2sc(=N)n(C/C=C/CNC(=O)OC(C)(C)C)c12]

Compound XX5.3 (4.25 g, 12.7 mmol) was dissolved in acetic acid (14.0 mL) and KSCN (4.93 g, 50.7 mmol) was added. The reaction mixture was stirred at room temperature for 1 h. Then, Br₂ (649 μL, 12.7 mmol) was dissolved in acetic acid (4.5 mL) and was added to the mixture. The reaction was stirred at room temperature for 30 min. The mixture was quenched with water (35 mL). The solid was filtered off. The filtrate was adjusted to pH 9 with aq. ammonia solution (33%) and the aqueous layer was extracted with ethyl acetate (2×100 mL). The combined organic layers were dried over sodium sulfate and filtered. The solvent was removed under reduced pressure. The residue was purified by flash column chromatography on a Biotage Selekt (DCM/[DCM/MeOH (85:15)]; 80:20 to 50:50; linear gradient; 120 mL/min, 30 min, Biotage Sfär Select HC 50 g) The product containing fractions were combined and the solvent was evaporated to obtain 2.02 g (5.15 mmol, 41%) of a beige solid. LCMS (Method A) retention time 1.35 min, [M+H]⁺=393.2. ¹H-NMR: (300 MHz, DMSO-d₆) δ [ppm]: 8.39 (brs, 1H), 7.84 (brs, 1H), 7.53 (d, J=1.5 Hz), 7.41 (d, J=1.6 Hz), 7.25 (brs, 1H), 6.91 (t, J=5.9 Hz, 1H), 5.68-5.44 (m, 2H), 4.77 (d, J=5.1 Hz, 2H), 3.86 (s, 3H), 3.53-3.44 (m, 2H), 1.34 (s, 9H).

### Preparation of tert-Butyl ((E)-4-((Z)-6-carbamoyl-2-((1-ethyl-3-methyl-1H-pyrazole-5-carbonyl)imino)-4-methoxybenzo[d]thiazol-3(2H)-yl)but-2-en-1-yl)carbamate (XX5.5)

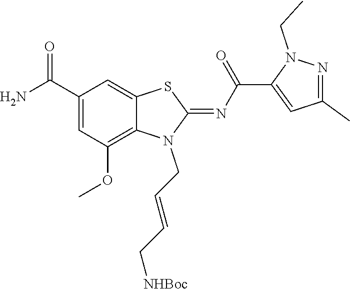 [SMILES: CCn1nc(C)cc1C(=O)/N=c1\sc2cc(C(N)=O)cc(OC)c2n1C/C=C/CNC(=O)OC(C)(C)C]

Compound XX5.4 (100 mg, 254 μmol) and 1-ethyl-3-methyl-1H-pyrazole-5-carboxylic acid (47 mg, 0.30 mmol) were dissolved in DMF (2 mL). Afterwards, HATU (147 mg, 382 μmol) and DIPEA (224 μL, 1.27 mmol) were added and the reaction mixture was stirred at room temperature for 2.5 h. The mixture was quenched with water (15 mL), the solid was filtered off and washed with water. The product was dried in vacuo to obtain 100 mg (189 μmol, 75%) of an off-white solid, which was used without further purification. LCMS (Method A) retention time 2.76 min, [M+H]⁺=529.1. ¹H-NMR (300 MHz, DMSO-d₆) δ [ppm]: 8.08 (brs, 1H), 8.00 (d, J=1.4 Hz, 1H), 7.62 (d, J=1.5 Hz, 1H), 7.47 (brs, 1H), 6.96-6.85 (m, 1H), 6.78 (s, 1H), 5.84-5.61 (m, 2H), 5.39-5.31 (m, 2H), 4.58 (q, J=7.1 Hz, 2H), 4.00 (s, 3H), 3.55-3.46 (m, 2H), 2.20 (s, 3H), 1.42-1.27 (m, 12H).

### Preparation of (Z)-3-((E)-4-aminobut-2-en-1-yl)-2-((1-ethyl-3-methyl-1H-pyrazole-5-carbonyl)imino)-4-methoxy-2,3-dihydrobenzo[d]thiazole-6-carboxamide hydrochloride (XX5.6)

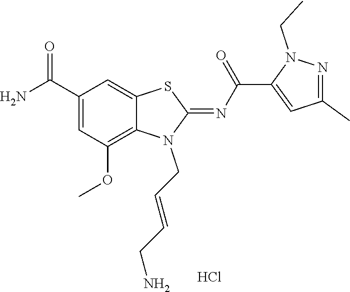 [SMILES: CCn1nc(C)cc1C(=O)/N=c1\sc2cc(C(N)=O)cc(OC)c2n1C/C=C/CN ; Cl]

Compound XX5.5 (1.0 g, 1.9 mmol) was suspended and 4 M HCl dioxane (15 mL, 61 mmol) was added. The resulting suspension was stirred at room temperature for 2 h. The formed solid was filtered off, washed with diethyl ether and dried in vacuo to obtain 860 mg (1.85 mmol, 98%) of an off-white solid. LCMS (Method A) retention time 1.38 min, [M+H]⁺=429.0. ¹H-NMR (300 MHz, DMSO-d₆) δ [ppm]: 8.16 (s, 4H), 8.06-8.00 (m, 1H), 7.70-7.64 (m, 1H), 6.16-6.00 (m, 1H), 5.85-5.69 (m, 1H), 5.38 (d, J=5.8 Hz, 2H), 4.57 (q, J=7.1 Hz, 2H), 4.03 (s, 3H), 3.49-3.31 (m, 2H), 2.20 (s, 3H), 1.34 (t, J=7.1 Hz, 3H).

### Preparation of tert-Butyl 4-(5-carbamoyl-2-(((E)-4-((Z)-6-carbamoyl-2-((1-ethyl-3-methyl-1H-pyrazole-5-carbonyl)imino)-4-methoxybenzo[d]thiazol-3 (2i)-yl)but-2-en-1-yl)amino)-3-nitrophenoxy)butanoate (XX5.7)

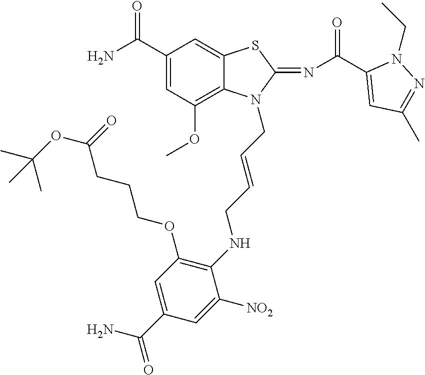 [SMILES: CCn1nc(C)cc1C(=O)/N=c1\sc2cc(C(N)=O)cc(OC)c2n1C/C=C/CNc1c(OCCCC(=O)OC(C)(C)C)cc(C(N)=O)cc1[N+](=O)[O-]]

Compound XX5.6 (500 mg, 1.07 mmol) was suspended in n-butanol (15 mL) and afterwards DIPEA (945 μL, 5.37 mmol) as well as sodium bicarbonate (180 mg, 2.15 mmol) were added. The resulting mixture was stirred at room temperature for 10 min. Then, tert-butyl 4-(5-carbamoyl-2-chloro-3-nitrophenoxy)butanoate (385 mg, 1.07 mmol) was added to the mixture and the reaction was stirred at 130° C. for 18 h. The reaction was cooled to room temperature and quenched with water (50 mL). The aqueous layer was extracted with CH₂Cl₂/CH₃OH (9/1, 75 mL). The organic layer was dried over sodium sulfate and filtered. The solvent was removed under reduced pressure. The residue was purified on silica by automated flash column chromatography on a Biotage Selekt (Biotage Sfär HC 25 g, 80 mL/min, 0-20% CH₃OH in CH₂Cl₂ in 30 min). Product containing fractions were pooled and the solvent was evaporated to yield 299 mg (398 μmol, 37%) of a reddish solid. LCMS (Method A) retention time 3.72 min, [M+H]⁺=751.3. ¹H-NMR (300 MHz, DMSO-d₆) δ [ppm]: 8.08-8.02 (m, 2H), 8.00-7.95 (m, 1H), 7.93 (brs, 1H), 7.64 (t, J=6.3 Hz, 1H), 7.59-7.54 (m, 1H), 7.46 (s, 1H), 7.43-7.37 (m, 1H), 7.26 (s, 1H), 6.72-6.66 (m, 1H), 5.82-5.72 (m, 2H), 5.29 (d, J=4.4 Hz, 2H), 4.55 (q, J=7.1 Hz, 2H), 4.10 (t, J=4.9 Hz, 2H), 3.92-3.82 (m, 5H), 2.25 (t, J=7.4 Hz, 2H), 2.17 (s, 3H), 1.91-1.76 (m, 2H), 1.44-1.27 (m, 12H).

### Preparation of tert-Butyl 4-(3-amino-5-carbamoyl-2-(((E)-4-((Z)-6-carbamoyl-2-((1-ethyl-3-methyl-1H-pyrazole-5-carbonyl)imino)-4-methoxybenzo[d]thiazol-3(2H)-yl)but-2-en-1-yl)amino)phenoxy)butanoate (XX5.8)

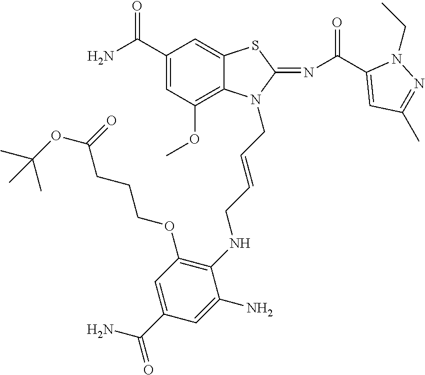 [SMILES: CCn1nc(C)cc1C(=O)/N=c1\sc2cc(C(N)=O)cc(OC)c2n1C/C=C/CNc1c(N)cc(C(N)=O)cc1OCCCC(=O)OC(C)(C)C]

To a stirred solution of compound XX5.7 (900 mg, 1.20 mmol) in CH₃OH (20 mL) was added sodium dithionite (2.1 g, 12 mmol) dissolved in water (5 mL) at 0° C. Then, NH₃ (30% aqueous solution, 1.19 mL, 8.99 mmol) was added to the mixture at 0° C. The reaction was allowed to warm to room temperature and stirred at room temperature for 3.5 h. The reaction was diluted with H₂O (75 mL) and CH₂Cl₂ (100 mL) was added. The aqueous layer was extracted with CH₂CL/CH₃OH (8:2, 5×50 mL). The organic layer was dried over sodium sulfate and filtered. The solvent was removed under reduced pressure to obtain 695 mg of yellowish solid (964 μmol, 80%). The residue was used without further purification. LCMS (Method A) retention time 2.07 min, [M+H]⁺=721.2. ¹H-NMR (300 MHz, DMSO-d₆): δ [ppm]=8.08 (s, 1H), 7.98 (d, J=1.4 Hz, 1H), 7.59 (d, J=1.5 Hz, 1H), 7.47 (s, 1H), 6.93 (s, 1H), 6.82 (d, J=1.8 Hz, 1H), 6.74 (d, J=0.6 Hz, 1H), 6.69 (d, J=1.9 Hz, 1H), 5.91-5.66 (m, 2H), 5.32 (d, J=5.2 Hz, 2H), 4.63-4.50 (m, 4H), 3.90 (s, 4H), 3.79 (t, J=6.3 Hz, 2H), 3.62-3.52 (m, 3H), 2.30-2.14 (m, 5H), 1.86-1.71 (m, 2H), 1.37-1.31 (m, 12H).

### Preparation of tert-Butyl 4-((2-amino-5-carbamoyl-1-((E)-4-((Z)-6-carbamoyl-2-((1-ethyl-3-methyl-1H-pyrazole-5-carbonyl)imino)-4-methoxybenzo[d]thiazol-3(2H)-yl)but-2-en-1-yl)-1H-benzo[d]imidazol-7-yl)oxy)butanoate (XX5.9)

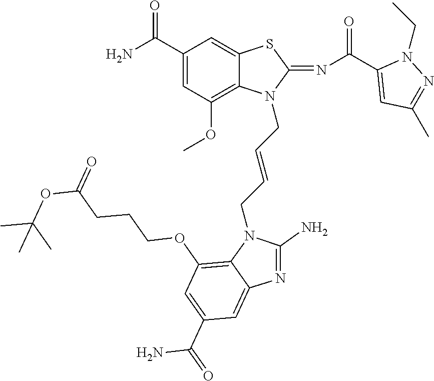 [SMILES: CCn1nc(C)cc1C(=O)/N=c1\sc2cc(C(N)=O)cc(OC)c2n1C/C=C/Cn1c(N)nc2cc(C(N)=O)cc(OCCCC(=O)OC(C)(C)C)c21]

Compound XX5.8 (660 mg, 915 μmol) was dissolved in CH₃OH (15 mL), cooled to 0° C. and afterwards BrCN (193 mg, 1.81 mmol) was added. The reaction mixture was stirred at room temperature for 3 h. The product formation was controlled by LC/MS. The solvent was removed under reduced pressure. The residue was suspended in petroleum ether and the solid was filtered off. The product was dried in vacuo to yield 737 mg of a yellowish solid, which was used without further purification. LCMS (Method A) retention time 1.83 min, [M+H]⁺=746.3. ¹H-NMR (300 MHz, DMSO-d₆) δ [ppm]=8.55 (brs, 2H), 8.10-7.97 (m, 3H), 7.60-7.54 (m, 1H), 7.53-7.44 (m, 2H), 7.44-7.29 (m, 2H), 6.67-6.57 (m, 1H), 5.97-5.73 (m, 2H), 5.33 (d, J=5.0 Hz, 2H), 4.86 (d, J=4.9 Hz, 2H), 4.51 (q, J=7.1 Hz, 2H), 3.97 (t, J=6.4 Hz, 2H), 3.80 (s, 3H, CH₃), 2.21 (t, J=7.3 Hz, 2H), 2.15-2.10 (m, 3H), 1.77 (p, J=6.8 Hz, 2H), 1.34 (s, 9H), 1.29 (t, J=7.1 Hz, 3H).

### Preparation of tert-Butyl 4-((5-carbamoyl-1-((E)-4-((Z)-6-carbamoyl-2-((1-ethyl-3-methyl-1H-pyrazole-5-carbonyl)imino)-4-methoxybenzo[d]thiazol-3(2H)-yl)but-2-en-1-yl)-2-(1-ethyl-3-methyl-1H-pyrazole-5-carboxamido)-1H-benzo[d]imidazol-7-yl)oxy)butanoate (XX5.10)

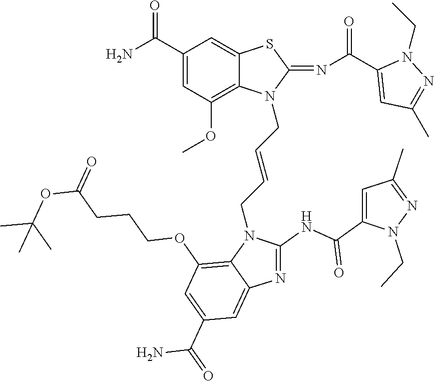 [SMILES: CCn1nc(C)cc1C(=O)/N=c1\sc2cc(C(N)=O)cc(OC)c2n1C/C=C/Cn1c(NC(=O)c2cc(C)nn2CC)nc2cc(C(N)=O)cc(OCCCC(=O)OC(C)(C)C)c21]

Compound XX5.9 (430 mg, 576 μmol) and 1-ethyl-3-methyl-1H-pyrazole-5-carboxylic acid (133 mg, 865 μmol) were dissolved in DMF (5 mL). Afterwards, HATU (332 mg, 865 μmol) and DIPEA (152 μL, 865 μmol) were added and the reaction mixture was stirred at room temperature for 19 h. Again, HATU (110 mg, 288 μmol) and DIPEA (50 μL, 288 μmol) were added and the mixture was stirred at room temperature for 4 h. Then, DIPEA (100 μL, 576 μmol) was added and the reaction was stirred at room temperature for 2 h. Again, 1-ethyl-3-methyl-1H-pyrazole-5-carboxylic acid (44 mg, 288 μmol) was added and the reaction was stirred at room temperature for 18 h. The product formation was controlled by LC/MS. The mixture was quenched with water (15 mL), the solid was filtered off and washed with water. The product was dried in vacuo. The residue was purified on silica by automated flash column chromatography on a Biotage Selekt (Biotage Sfär HC 10 g, 40 mL/min, 0-20% CH₃OH in CH₂Cl₂ in 30 min) followed by a purification on preparative TLC (CH₂Cl₂/CH₃OH=9:1). The resulting product was suspended in acetonitrile, the solid was filtered off and dried in vacuo. The solid was suspended in methanol, the solid was filtered off and dried in vacuo to yield 89.0 mg (101 μmol, 17%) of an off-white solid. LCMS (Method A) retention time 2.50 min, [M+H]⁺=882.3. ¹H-NMR (300 MHz, DMSO-d₆) δ [ppm]=12.80 (brs, 1H), 8.04 (brs, 1H), 8.00-7.91 (m, 2H), 7.67-7.61 (m, 1H), 7.56-7.50 (m, 1H), 7.46 (brs, 1H), 7.34-7.24 (m, 2H), 6.63-6.54 (m, 1H), 6.53-6.47 (m, 1H), 5.96-5.81 (m, 2H), 5.31 (d, J=4.7 Hz, 2H), 4.93 (d, J=4.6 Hz, 2H), 4.50 (q, J=7.0 Hz, 4H), 3.95 (t, J=6.3 Hz, 2H), 3.75 (s, 3H), 2.20 (t, J=7.3 Hz, 2H), 2.10 (s, 6H), 1.81-1.71 (m, 2H), 1.34 (s, 9H), 1.30-1.15 (m, 6H).

### Preparation of 4-((5-carbamoyl-1-((E)-4-((Z)-6-carbamoyl-2-((1-ethyl-3-methyl-1H-pyrazole-5-carbonyl)imino)-4-methoxybenzo[d]thiazol-3(2H)-yl)but-2-en-1-yl)-2-(1-ethyl-3-methyl-1H-pyrazole-5-carboxamido)-1H-benzo[d]imidazol-7-yl)oxy) butanoic acid (XX5.11)

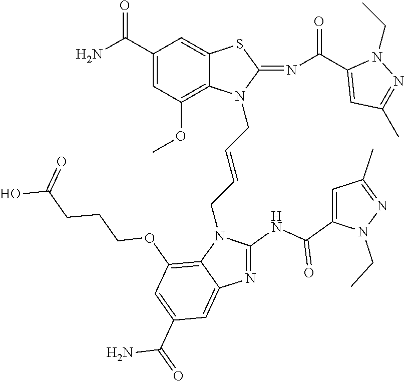 [SMILES: CCn1nc(C)cc1C(=O)/N=c1\sc2cc(C(N)=O)cc(OC)c2n1C/C=C/Cn1c(NC(=O)c2cc(C)nn2CC)nc2cc(C(N)=O)cc(OCCCC(=O)O)c21]

Compound XX5.10 (220 mg, 221 μmol) was suspended in dioxane (1 mL) and 4 M HCl in dioxane (1.77 mL, 7.07 mmol) was added. The resulting suspension was stirred at room temperature for 1 h. The product formation was controlled by LC/MS. The formed solid was filtered off, washed with diethyl ether and dried in vacuo to yield 118 mg (136 μmol, 62%) as a white solid. LCMS (Method A) retention time 2.05 min, [M+H]⁺=826.3. ¹H-NMR (300 MHz, DMSO-d₆) δ [ppm]=8.05 (brs, 1H), 7.99-7.93 (m, 1H), 7.67-7.61 (m, 1H), 7.56-7.50 (m, 1H), 7.45 (s, 1H), 7.33-7.27 (m, 2H), 6.59 (s, 1H), 6.48 (s, 1H), 5.98-5.74 (m, 2H), 5.30 (d, J=5.0 Hz, 2H), 4.93 (d, J=4.8 Hz, 2H), 4.57-4.42 (m, 4H), 3.98 (t, J=6.5 Hz, 2H), 3.76 (s, 3H), 2.26 (t, J=7.2 Hz, 2H), 2.09 (d, J=1.6 Hz, 6H), 1.79 (p, J=6.8 Hz, 2H), 1.37-1.19 (m, 6H).

### Preparation of (Z)-3-((E)-4-(5-carbamoyl-7-(4-(1,2-dimethylhydrazineyl)-4-oxobutoxy)-2-(1-ethyl-3-methyl-1H-pyrazole-5-carboxamido)-1H-benzo[d]imidazol-1-yl)but-2-en-1-yl)-2-((1-ethyl-3-methyl-1H-pyrazole-5-carbonyl)imino)-4-methoxy-2,3-dihydrobenzo[d]thiazole-6-carboxamide (Compound XXX)

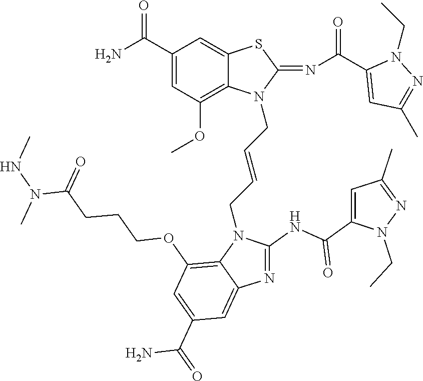 [SMILES: CCn1nc(C)cc1C(=O)/N=c1\sc2cc(C(N)=O)cc(OC)c2n1C/C=C/Cn1c(NC(=O)c2cc(C)nn2CC)nc2cc(C(N)=O)cc(OCCCC(=O)N(C)NC)c21]

Compound XX5.11 (109 mg, 126 μmol) was suspended in DMF (2 mL). Afterwards, DIPEA (66 UL, 0.37 mmol), NHS (30 mg, 0.25 mmol) and DIC (39 UL, 0.25 mmol) were added and the resulting reaction mixture was stirred at room temperature for 2.5 h. Again, NHS (30 mg, 0.25 mmol) was added and the mixture was stirred at 40° C. for 4 h and at room temperature for 13 h. The reaction progress was controlled by LC/MS. Afterwards, 1,2-dimethylhydrazine dihydrochloride (84.1 mg, 632 μmol) and DIPEA (66.0 μL, 379 μmol) were added and the resulting reaction mixture was stirred at room temperature for 18 h and at 40° C. for 2 h. The reaction progress was controlled by LC/MS. The solvent was removed under reduced pressure and the residue was purified on RP18 silica by prepHPLC (Method I). Product containing fractions were freeze-dried to obtain 10.3 mg (10.5 μmol, 8%, purity 96.5% by HPLC Method B) of a white solid. LCMS (Method A) retention time 2.86 min, [M+H]⁺=868.2.

### Example 36: Synthetic Scheme for Compound XXXI

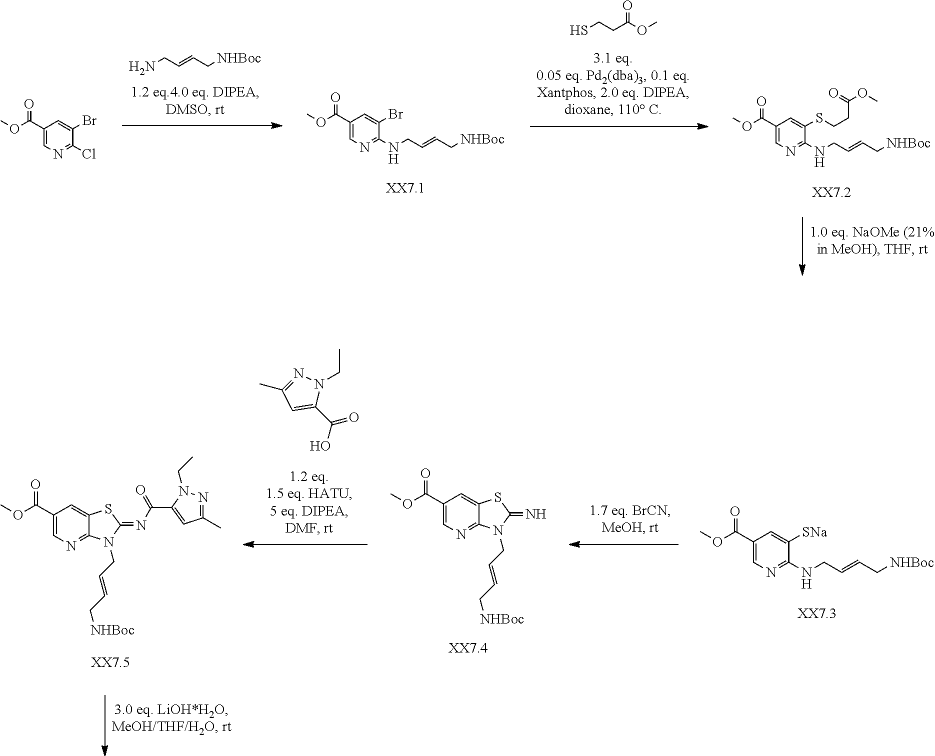

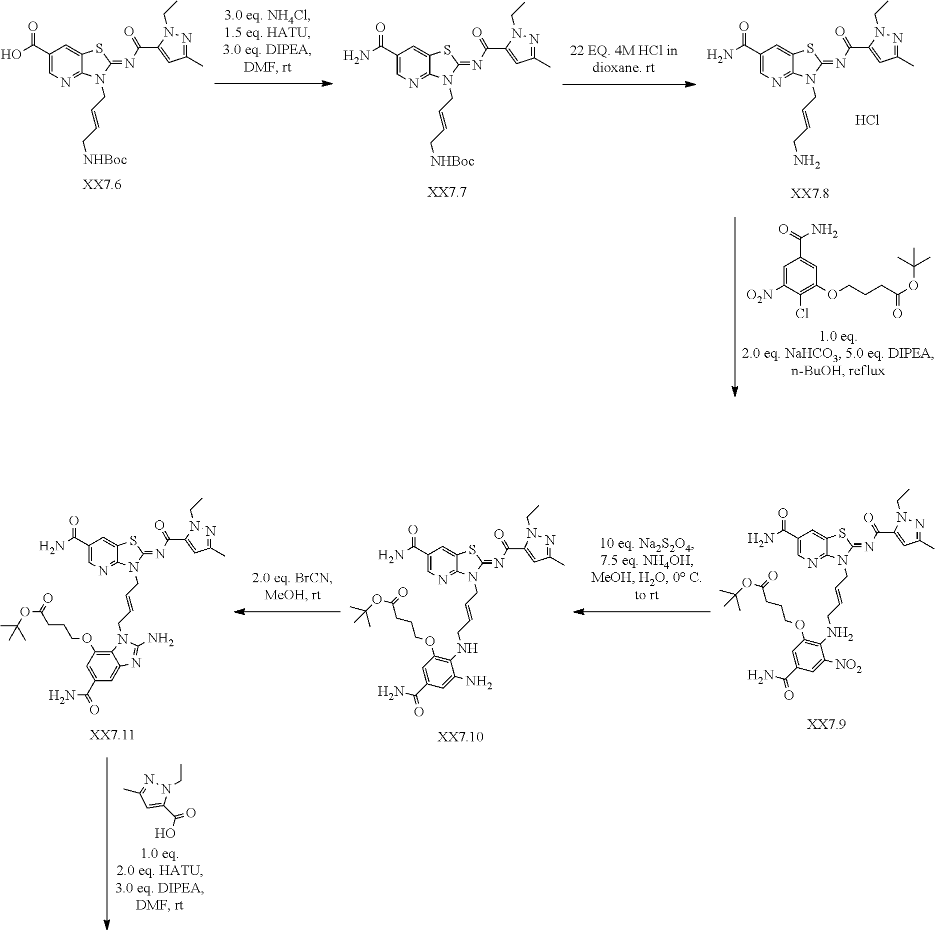

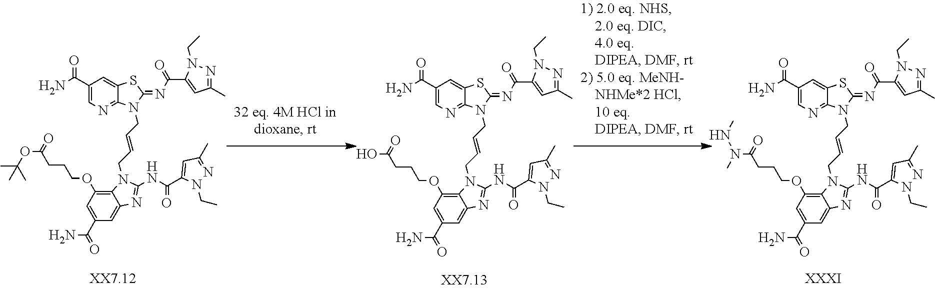

### Preparation of Methyl (E)-5-bromo-6-((4-((tert-butoxycarbonyl)amino)but-2-en-1-yl)amino)nicotinate (XX7.1)

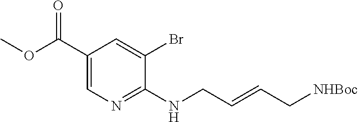 [SMILES: COC(=O)c1cnc(NC/C=C/CNC(=O)OC(C)(C)C)c(Br)c1]

Methyl 5-bromo-6-chloronicotinate (15.0 g, 59.8 mmol) and (E)-tert-butyl (4-aminobut-2-en-1-yl)carbamate (13.4 g, 71.8 mmol) were dissolved in DMSO (200 mL). DIPEA (42.1 mL, 239 mmol) was added and the reaction mixture was stirred at room temperature for 67 h. Again, (E)-tert-butyl (4-aminobut-2-en-1-yl)carbamate (1.04 g, 5.98 mmol) was added and the reaction was stirred at room temperature for 2 h. The reaction mixture was diluted with ice-water (200 mL) and extracted with ethyl acetate (2×250 mL). The organic layer was dried over Na₂SO₄ and filtered. The solvent was removed under reduced pressure. The residue was purified on silica by automated flash column chromatography on a Biotage Selekt (Biotage Sfär HC 200 g, 200 mL/min, 0-80% ethyl acetate in n-hexane in 30 min) to yield 18.1 g (45.2 mmol, 75%) of a colorless oil. LCMS (Method A) retention time 2.82 min, [M+H]⁺=399.9. ¹H NMR (300 MHz, DMSO-d₆) δ [ppm]=8.59-8.51 (m, 1H), 8.12-8.04 (m, 1H), 7.30 (t, J=5.8 Hz, 1H), 6.90 (t, J=6.0 Hz, 1H), 5.69-5.43 (m, 2H), 4.11-3.96 (m, 2H), 3.78 (s, 3H), 3.51 (t, J=5.0 Hz, 2H), 1.35 (s, 9H).

### Preparation of Methyl (E)-6-((4-((tert-butoxycarbonyl)amino)but-2-en-1-yl)amino)-5-((3-methoxy-3-oxopropyl)thio) nicotinate (XX7.2)

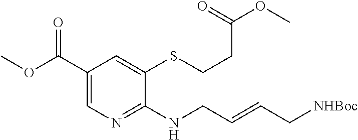 [SMILES: COC(=O)CCSc1cc(C(=O)OC)cnc1NC/C=C/CNC(=O)OC(C)(C)C]

Methyl (E)-5-bromo-6-((4-((tert-butoxycarbonyl)amino)but-2-en-1-yl)amino)nicotinate (18.1 g, 45.2 mmol) was co-evaporated with anhydrous 1,4-dioxane (30 mL). The residue was dissolved in anhydrous 1,4-dioxane (200 mL) and DIPEA (15.5 mL, 90.4 mmol) as well as methyl 3-mercaptopropanoate (15 mL, 0.14 mol) were added. Afterwards, Xantphos (2.6 g, 4.5 mmol) and Pd₂(dba)₃ (1.0 g, 2.2 mmol) were added and the resulting mixture was heated to 110° C. for 1.5 h. Water was added and the aqueous layer was extracted with ethyl acetate. The organic layer was concentrated (~150 mL) under reduced pressure and filtered through a small pad of Celite® Hyflo Supercel. The solvent was removed under reduced pressure. The residue was purified on silica by automated flash column chromatography on a Biotage Selekt (Biotage Sfär HC 200 g, 200 mL/min, 0-100% ethyl acetate in n-hexane in 30 min) to yield 17.0 g (38.6 mmol, 85%) of a colorless oil. LCMS (Method A) retention time 3.67 min, [M+H]⁺=440.1. ¹H-NMR (300 MHz, DMSO-d₆) δ [ppm]=8.58-8.52 (m, 1H), 7.98-7.91 (m, 1H), 7.27 (t, J=5.9 Hz, 1H), 6.89 (t, J=5.8 Hz, 1H), 5.70-5.43 (m, 2H), 4.09-3.96 (m, 2H), 3.78 (s, 3H), 3.57 (s, 3H), 3.49 (d, J=5.8 Hz, 2H), 2.97 (t, J=6.9 Hz, 2H), 2.56 (t, J=6.9 Hz, 2H), 1.35 (s, 9H).

### Preparation of Sodium (E)-2-((4-((tert-butoxycarbonyl)amino)but-2-en-1-yl)amino)-5-(methoxycarbonyl)pyridine-3-thiolate (XX7.3)

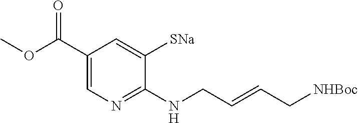

Compound XX7.2 (6.0 g, 13 mmol) was dissolved in THF (40 mL). Then, sodium methylate (3.1 mL, 13 mmol) was added and the mixture was stirred at room temperature for 30 min. The mixture was diluted with CH₂Cl₂. The resulting solid was filtered off, washed with CH₂Cl₂, and dried in vacuo to obtain 4.5 g (12 mmol, 88%) of a yellowish solid, which was used without further purification. LCMS (Method A) retention time 1.73 min, [M+H]⁺=354.0. ¹H-NMR (300 MHz, DMSO-d₆) δ [ppm]=7.96-7.89 (m, 1H), 7.58-7.51 (m, 1H), 7.38 (t, 1H), 6.95 (t, 1H), 5.78-5.48 (m, 2H), 4.01-3.91 (m, 2H), 3.70 (s, 3H), 3.61-3.47 (m, 2H), 1.37 (s, 9H).

### Preparation of Methyl (E)-3-(4-((tert-butoxycarbonyl)amino)but-2-en-1-yl)-2-imino-2,3-dihydrothiazolo[4,5-b]pyridine-6-carboxylate (XX7.4)

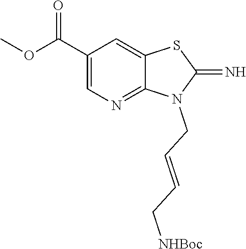 [SMILES: COC(=O)c1cnc2c(c1)sc(=N)n2C/C=C/CNC(=O)OC(C)(C)C]

Compound XX7.3 (4.5 g, 12 mmol) was dissolved in CH₃OH (50 mL) and cooled to 0° C. Then, BrCN (2.1 g, 20 mmol) was added and the reaction was stirred at room temperature for 15 min. The solvent was removed under reduced pressure. The residue was dissolved in CH₂Cl₂ and washed with water. The organic layer was dried over sodium sulfate and filtered. The solvent was removed under reduced pressure and the residue was purified on silica by automated flash column chromatography on a Biotage Selekt (Biotage Sfär HC 50 g, 120 mL/min, 0-100% ethyl acetate in petroleum ether in 30 min) to yield 2.7 g (7.1 mmol, 60%) of a yellow solid. LCMS (Method A) retention time 2.13 min, [M+H]⁺=379.0. ¹H-NMR (300-MHz, DMSO-d6) δ ppm=8.99 (s, 1H), 8.67-8.58 (m, 1H), 8.30-8.24 (m, 1H), 6.89 (t, J=6.1 Hz, 1H), 5.70-5.48 (m, 2H), 4.55 (d, J=4.5 Hz, 2H), 3.84 (s, 3H), 3.49 (t, J=5.0 Hz, 2H), 1.34 (s, 9H).

### Preparation of Methyl (Z)-3-((E)-4-((tert-butoxycarbonyl)amino)but-2-en-1-yl)-2-((1-ethyl-3-methyl-1H-pyrazole-5-carbonyl)imino)-2,3-dihydrothiazolo[4,5-b]pyridine-6-carboxylate (XX7.5)

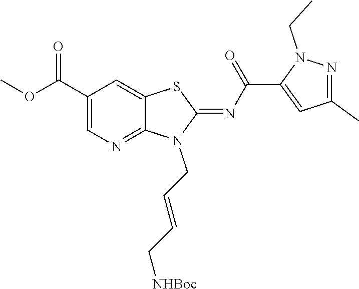

Compound XX7.4 (1.20 g, 3.17 mmol) and 1-ethyl-3-methyl-1H-pyrazole-5-carboxylic acid (586 mg, 3.81 mmol) was dissolved in anhydrous DMF (10 mL). Afterwards, HATU (1.8 g, 4.7 mmol) and DIPEA (2.8 mL, 16 mmol) were added and the reaction mixture was stirred at room temperature for 15 min. The mixture was quenched with water (20 mL), and the solid was filtered off and washed with water. The product was dried in vacuo to yield 1.55 g (3.01 mmol, 95%) of an off-white solid, which was used without further purification. LCMS (Method A) retention time 2.88 min, [M+H]⁺=515.1. ¹H-NMR (300 MHz, DMSO-d₆) δ [ppm]=9.10-9.03 (m, 1H), 8.56-8.48 (m, 1H), 6.87-6.80 (m, 1H), 5.93-5.84 (m, 2H), 5.20-5.12 (m, 2H), 4.69 (q, J=7.1 Hz, 2H), 4.54 (s, 1H), 3.98 (s, 3H), 3.81-3.67 (m, 2H), 2.32 (s, 3H), 1.51-1.41 (m, 3H), 1.39 (s, 9H).

### Preparation of (Z)-3-((E)-4-((tert-butoxycarbonyl)amino)but-2-en-1-yl)-2-((1-ethyl-3-methyl-1H-pyrazole-5-carbonyl)imino)-2,3-dihydrothiazolo[4,5-b]pyridine-6-carboxylic acid (XX7.6)

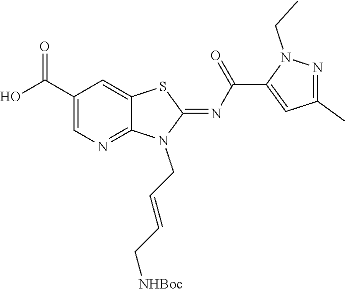 [SMILES: CCn1nc(C)cc1C(=O)/N=c1\sc2cc(C(=O)O)cnc2n1C/C=C/CNC(=O)OC(C)(C)C]

Compound XX7.5 (1.5 g, 2.9 mmol) was dissolved in CH₃OH/THF/H₂O (2:2:1; 25 mL). Afterwards, LiOH (209 mg, 8.74 mmol) was added and the resulting reaction mixture was stirred at room temperature for 2.5 h. The organic solvents were removed under reduced pressure. The aqueous phase was cooled to 0° C. and neutralized with 1 M aq. HCl. The precipitate was filtered and washed with water. The solid was dissolved/suspended in CH₃CN/H₂O (1:1, 15 mL) and freeze-dried to obtain 1.18 g (2.36 mmol, 81%) of an off-white solid, which was used without further purification. LCMS (Method A) retention time 2.49 min, [M+H]⁺=501.1. ¹H-NMR (300 MHz, DMSO-d₆) δ [ppm]=13.42 (brs, 1H), 9.01-8.94 (m, 1H), 8.90-8.82 (m, 1H), 6.90 (t, J=5.8 Hz, 1H), 6.84-6.78 (m, 1H), 5.80-5.70 (m, 2H), 5.08 (d, J=4.5 Hz, 2H), 4.57 (q, J=7.1 Hz, 2H), 3.50 (t, J=4.8 Hz, 2H), 2.20 (s, 3H), 1.35 (t, J=7.1 Hz, 3H), 1.30 (s, 9H).

### Preparation of tert-Butyl ((E)-4-((Z)-6-carbamoyl-2-((1-ethyl-3-methyl-1H-pyrazole-5-carbonyl)imino)thiazolo[4,5-b]pyridin-3(2H)-yl)but-2-en-1-yl)carbamate (XX7.7)

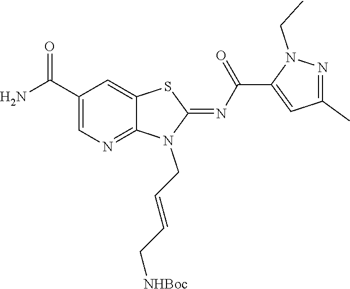 [SMILES: CCn1nc(C)cc1C(=O)/N=c1\sc2cc(C(N)=O)cnc2n1C/C=C/CNC(=O)OC(C)(C)C]

Compound XX7.6 (1.18 g, 2.36 mmol) was dissolved in DMF (8 mL) and afterwards NH₄Cl (378 mg, 7.07 mmol), HATU (1.34 g, 3.54 mmol) as well as DIPEA (1.21 mL, 7.07 mmol) were added followed by stirring at room temperature for 25 min. The reaction mixture was diluted with water (30 mL). The precipitate was filtered off and washed with water. The solid was dried in vacuo to obtain 1.1 g (2.2 mmol, 93%) as an off-white solid, which was used without further purification. LCMS (Method A) retention time 2.23 min, [M+H]⁺=500.1. ¹H-NMR (300 MHz, DMSO-d6) δ [ppm]=9.00-8.94 (m, 1H), 8.80-8.74 (m, 1H), 8.18 (brs, 1H), 7.62 (brs, 1H), 6.90 (t, J=5.9 Hz, 1H), 6.85-6.79 (m, 1H), 5.83-5.65 (m, 2H), 5.09 (d, J=4.5 Hz, 2H), 4.59 (q, J=7.1 Hz, 2H), 3.51 (t, J=4.7 Hz, 2H), 2.21 (s, 3H), 1.35 (t, J=7.1 Hz, 3H), 1.30 (s, 9H).

### Preparation of (Z)-3-((E)-4-Aminobut-2-en-1-yl)-2-((1-ethyl-3-methyl-1H-pyrazole-5-carbonyl)imino)-2,3-dihydrothiazolo[4,5-b]pyridine-6-carboxamide hydrochloride (XX7.8)

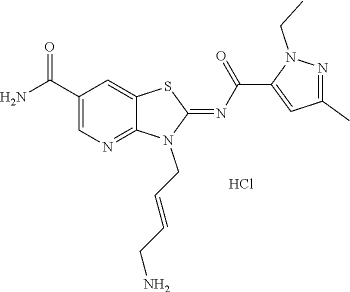 [SMILES: CCn1nc(C)cc1C(=O)/N=c1\sc2cc(C(N)=O)cnc2n1C/C=C/CN ; Cl]

Compound XX7.7 (1.80 g, 3.60 mmol) was suspended in 4 M HCl in dioxane (20.3 mL, 81.1 mmol). The resulting suspension was stirred at room temperature for 30 min. The formed solid was filtered off, washed with diethyl ether and dried in vacuo to yield 1.58 g (3.60 mmol, 100%) as an off-white solid, which was used without further purification. LCMS (Method A) retention time 1.36 min, [M+H]⁺=400.1. ¹H-NMR (300 MHz, DMSO-d₆) δ [ppm]=9.05-8.96 (m, 1H), 8.91-8.58 (m, 1H), 8.30 (brs, 1H), 8.12 (brs, 2H), 7.64 (brs, 1H), 6.89-6.83 (m, 1H), 6.17-6.01 (m, 1H), 5.87-5.70 (m, 1H), 5.18-5.09 (m, 2H), 4.58 (q, J=7.1 Hz, 2H), 3.48-3.34 (m, 2H), 2.21 (s, 3H), 1.36 (t, J=7.1 Hz, 3H).

### Preparation of tert-Butyl 4-(5-carbamoyl-2-(((E)-4-((Z)-6-carbamoyl-2-((1-ethyl-3-methyl-1H-pyrazole-5-carbonyl)imino)thiazolo[4,5-b]pyridin-3(2H)-yl)but-2-en-1-yl)amino)-3-nitrophenoxy)butanoate (XX7.9)

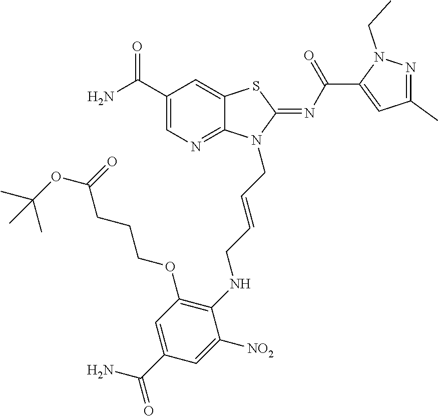 [SMILES: CCn1nc(C)cc1C(=O)/N=c1\sc2cc(C(N)=O)cnc2n1C/C=C/CNc1c(OCCCC(=O)OC(C)(C)C)cc(C(N)=O)cc1[N+](=O)[O-]]

Compound XX7.8 (1.55 g, 3.56 mmol) was suspended in n-butanol (25 mL) and afterwards DIPEA (3.1 mL, 18 mmol) and sodium bicarbonate (597 mg, 7.11 mmol) were added. The mixture was stirred at room temperature for 10 min followed by the addition of tert-butyl 4-(5-carbamoyl-2-chloro-3-nitrophenoxy)butanoate (1.28 g, 3.56 mmol). Then, the reaction mixture was stirred at 130° C. for 29 h. The reaction was cooled to room temperature and quenched with water (50 mL). The aqueous layer was extracted with CH₂Cl₂/CH₃OH (9:1, 100 mL). The organic layer was dried over sodium sulfate and filtered. The solvent was removed under reduced pressure and the resulting residue was purified on silica by automated flash column chromatography on a Biotage Selekt (Biotage Sfär HC 50 g, 120 mL/min, 0-20% CH₃OH in CH₂Cl₂ in 30 min) to obtain 1.25 g, (1.73 mmol, 49%) of an orange solid. LCMS (Method A) retention time 3.61 min, [M+H]⁺=722.2. ¹H-NMR (300 MHz, DMSO-d₆) δ [ppm]=8.95-8.87 (m, 1H), 8.78-8.70 (m, 1H), 8.16 (s, 1H), 8.05-7.99 (m, 1H), 7.91 (brs, 1H), 7.68-7.58 (m, 2H), 7.43-7.36 (m, 1H), 7.26 (brs, 1H), 6.75-6.69 (m, 1H), 5.92-5.76 (m, 2H), 5.04 (d, J=4.6 Hz, 2H), 4.55 (q, J=7.1 Hz, 2H), 4.09 (t, J=5.3 Hz, 2H), 3.88 (t, J=6.3 Hz, 2H), 2.26 (t, J=7.3 Hz, 2H), 2.17 (s, 3H), 1.84 (p, J=6.8 Hz, 2H), 1.43-1.25 (m, 12H).

### Preparation of tert-Butyl 4-(3-amino-5-carbamoyl-2-(((E)-4-((Z)-6-carbamoyl-2-((1-ethyl-3-methyl-1H-pyrazole-5-carbonyl)imino)thiazolo[4,5-b]pyridin-3(2H)-yl)but-2-en-1-yl)amino)phenoxy)butanoate (XX7.10)

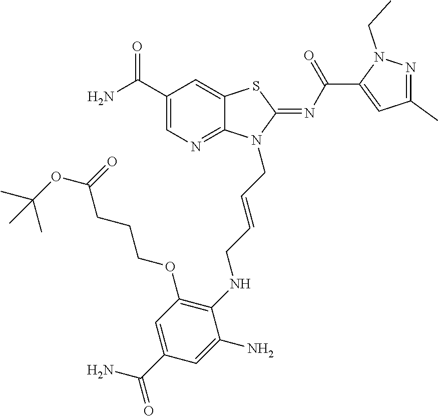 [SMILES: CCn1nc(C)cc1C(=O)/N=c1\sc2cc(C(N)=O)cnc2n1C/C=C/CNc1c(N)cc(C(N)=O)cc1OCCCC(=O)OC(C)(C)C]

Compound XX7.9 (1.25 g, 1.73 mmol) was dissolved in CH₃OH (20 mL) and cooled to 0° C. Then, sodium dithionite (3.0 g, 17 mmol) in water (5 mL) and aqueous NH₃ (30% aqueous solution, 1.7 mL, 13 mmol) were added. The reaction was allowed to warm to room temperature and stirred at room temperature for 1 h. The reaction was diluted with H₂O (75 mL) followed by the addition of CH₂Cl₂ (100 mL). The aqueous layer was extracted with CH₂Cl₂/CH₃OH (8:2, 2×50 mL). The organic layer was dried over sodium sulfate and filtered. The solvent was removed under reduced pressure to obtain 1.07 g (1.55 mmol, 89%) of an orange solid, which was used without further purification. LCMS (Method A) retention time 2.02 min, [M+H]⁺=692.3. ¹H-NMR (300 MHz, DMSO-d₆) δ [ppm]=9.00-8.91 (m, 1H), 8.80-8.70 (m, 1H), 8.18 (s, 1H), 7.67-7.49 (m, 2H), 6.98-6.86 (m, 1H), 6.84-6.78 (m, 2H), 6.74-6.65 (m, 1H), 5.92-5.81 (m, 2H), 5.10-5.03 (m, 2H), 4.64-4.51 (m, 4H), 3.91-3.78 (m, 3H), 3.58-3.52 (m, 2H), 2.34-2.23 (m, 2H), 2.20 (s, 3H), 1.88-1.73 (m, 2H), 1.39-1.26 (m, 12H).

### Preparation of tert-butyl 4-((2-amino-5-carbamoyl-1-((E)-4-((Z)-6-carbamoyl-2-((1-ethyl-3-methyl-1H-pyrazole-5-carbonyl)imino)thiazolo[4,5-b]pyridin-3(2H)-yl)but-2-en-1-yl)-1H-benzo[d]imidazol-7-yl)oxy)butanoate (XX7.11)

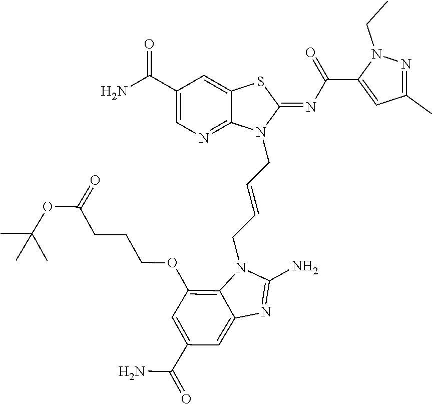 [SMILES: CCn1nc(C)cc1C(=O)/N=c1\sc2cc(C(N)=O)cnc2n1C/C=C/Cn1c(N)nc2cc(C(N)=O)cc(OCCCC(=O)OC(C)(C)C)c21]

Compound XX7.10 (1.07 g, 1.55 mmol) was dissolved in CH₃OH (20 mL), cooled to 0° C., and BrCN (327 mg, 3.09 mmol) was added. The reaction mixture was stirred at room temperature for 3.5 h. The solvent was removed under reduced pressure. The residue was suspended in petroleum ether and the solid was filtered off as well as washed with petroleum ether. The product was dried in vacuo to yield 961 mg (1.34 mmol, 86%) of a yellowish solid, which was used without further purification. LCMS (Method A) retention time 1.76 min, [M+H]⁺=717.2. ¹H-NMR (300 MHz, DMSO-d₆) δ [ppm]=8.97-8.91 (m, 1H), 8.82-8.72 (m, 1H), 8.53 (brs, 2H), 8.18 (brs, 1H), 8.02 (brs, 1H), 7.64 (brs, 1H), 7.52-7.43 (m, 1H), 7.40 (brs, 1H), 7.36-7.27 (m, 1H), 6.64-6.58 (m, 1H), 5.91 (q, J=2.9 Hz, 2H), 5.13-5.05 (m, 2H), 4.89-4.82 (m, 2H), 4.50 (q, J=7.1 Hz, 2H), 3.99 (t, J=6.3 Hz, 2H), 2.21 (t, J=7.3 Hz, 2H), 2.13 (s, 3H), 1.78 (q, J=6.8 Hz, 2H), 1.43-1.20 (m, 12H).

### Preparation of tert-Butyl 4-((5-carbamoyl-1-((E)-4-((Z)-6-carbamoyl-2-((1-ethyl-3-methyl-1H-pyrazole-5-carbonyl)imino)thiazolo[4,5-b]pyridin-3(2H)-yl)but-2-en-1-yl)-2-(1-ethyl-3-methyl-1H-pyrazole-5-carboxamido)-1H-benzo[d]imidazol-7-yl)oxy)butanoate (XX7.12)

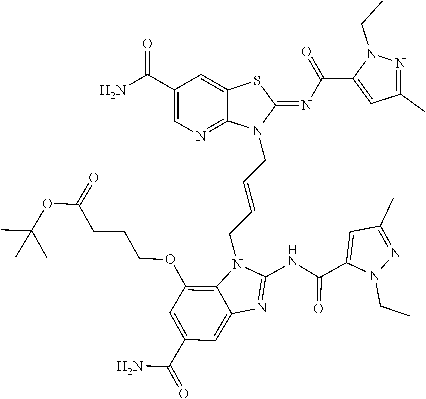 [SMILES: CCn1nc(C)cc1C(=O)/N=c1\sc2cc(C(N)=O)cnc2n1C/C=C/Cn1c(NC(=O)c2cc(C)nn2CC)nc2cc(C(N)=O)cc(OCCCC(=O)OC(C)(C)C)c21]

Compound XX7.11 (900 mg, 1.26 mmol) and 1-ethyl-3-methyl-1H-pyrazole-5-carboxylic acid (193 mg, 1.26 mmol) were dissolved in DMF (10 mL). Afterwards, HATU (723 mg, 1.88 mmol) and DIPEA (331 μL, 1.88 mmol) were added and the reaction mixture was stirred at room temperature for 4 h. Again, HATU (241 mg, 634 μmol) and DIPEA (331 μL, 1.88 mmol) were added and the mixture was stirred at room temperature for 14 h. The product formation was controlled by LC/MS. The mixture was quenched with water (20 mL), the solid was filtered off and washed with water. The residue was suspended in acetonitrile, the solid was filtered off and dried in vacuo. The resulting residue was suspended in MeOH and the solid was filtered off. The product was dried in vacuo to give 327 mg (383 μmol, 31%) of an off-white solid, which was used without further purification. LCMS (Method A) retention time 2.39 min, [M+H]⁺=853.3. ¹H-NMR (300 MHz, DMSO-d₆) δ [ppm]=12.78 (br s, 1H), 8.95-8.88 (m, 1H), 8.78-8.71 (m, 1H), 8.14 (brs, 1H), 7.92 (brs, 1H), 7.66-7.58 (m, 2H), 7.31-7.25 (m, 2H), 6.63-6.57 (m, 1H), 6.51-6.45 (m, 1H), 6.09-5.73 (m, 2H), 5.08 (d, J=5.7 Hz, 2H), 4.93 (d, J=5.2 Hz, 2H), 4.50 (p, J=7.4 Hz, 4H), 4.00 (t, J=6.3 Hz, 2H), 2.25 (t, J=7.2 Hz, 2H), 2.09 (s, 6H), 1.88-1.73 (m, 2H), 1.34 (s, 9H), 1.30-1.20 (m, 6H).

### Preparation of 4-((5-Carbamoyl-1-((E)-4-((Z)-6-carbamoyl-2-((1-ethyl-3-methyl-1H-pyrazole-5-carbonyl)imino)thiazolo[4,5-b]pyridin-3(2H)-yl)but-2-en-1-yl)-2-(1-ethyl-3-methyl-1H-pyrazole-5-carboxamido)-1H-benzo[d]imidazol-7-yl)oxy) butanoic acid (XX7.13)

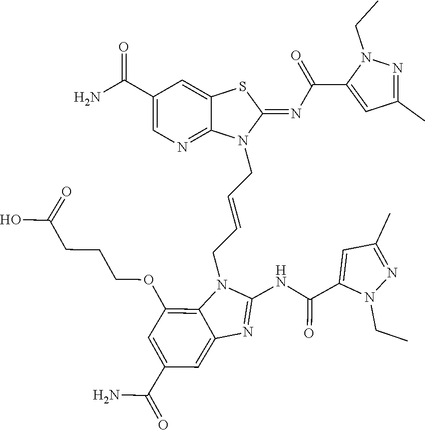 [SMILES: CCn1nc(C)cc1C(=O)/N=c1\sc2cc(C(N)=O)cnc2n1C/C=C/Cn1c(NC(=O)c2cc(C)nn2CC)nc2cc(C(N)=O)cc(OCCCC(=O)O)c21]

Compound XX7.12 (230 mg, 270 μmol) was suspended in dioxane (2 mL) and 4 M HCl in dioxane (2.16 mL, 8.63 mmol) was added. The resulting suspension was stirred at room temperature for 1 h. The product formation was controlled by LC/MS. The formed solid was filtered off, washed with diethyl ether and dried in vacuo to obtain 217 mg (260 μmol, 96%) of an off-white solid, which was used without further purification. LCMS (Method A) retention time 1.84 min, [M+H]⁺=797.5. ¹H-NMR (300 MHz, DMSO-d₆) δ [ppm]=8.92 (d, J=2.0 Hz, 1H), 8.74 (d, J=2.0 Hz, 1H), 8.16 (s, 1H), 7.94 (s, 1H), 7.66-7.57 (m, 2H), 7.34-7.28 (m, 2H), 6.59 (s, 1H), 6.49 (s, 1H), 6.07-5.93 (m, 1H), 5.89-5.74 (m, 1H), 5.07 (d, J=5.7 Hz, 2H), 4.95 (d, J=5.3 Hz, 2H), 4.49 (p, J=7.2 Hz, 4H), 4.03 (t, J=6.4 Hz, 2H), 2.28 (t, J=7.1 Hz, 2H), 2.09 (s, 6H), 1.83 (p, J=6.7 Hz, 2H), 1.32-1.20 (m, 6H).

### Preparation of (Z)-3-((E)-4-(5-Carbamoyl-7-(4-(1,2-dimethylhydrazineyl)-4-oxobutoxy)-2-(1-ethyl-3-methyl-1H-pyrazole-5-carboxamido)-1H-benzo[d]imidazol-1-yl)but-2-en-1-yl)-2-((1-ethyl-3-methyl-1H-pyrazole-5-carbonyl)imino)-2,3-dihydrothiazolo[4,5-b]pyridine-6-carboxamide (Compound XXXI)

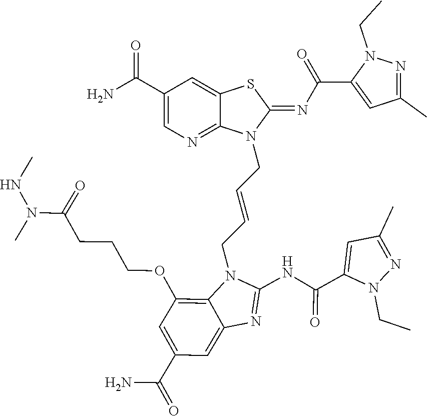

Compound XX7.13 (50.0 mg, 60.0 μmol) was suspended in DMF (3 mL). Afterwards, DIPEA (31.3 μL, 180 μmol), NHS (14.1 mg, 120 μmol) and DIC (18.6 UL, 120 μmol) were added and the resulting reaction mixture was stirred at 40° C. for 6.6 h. Again, NHS (7.0 mg, 60 μmol) and DIPEA (10 μL, 60 μL) were added and the reaction mixture was stirred at 45° C. for 15 h. Afterwards, the reaction mixture was stirred at room temperature for 15 h. The reaction progress was controlled by LC/MS. Then, 1,2-dimethylhydrazine dihydrochloride (39.9 mg, 300 μmol) and DIPEA (104 μL, 600 μmol) were added and the resulting reaction mixture was stirred at room temperature for 1 h.

The reaction progress was controlled by LC/MS. The solvent was removed under reduced pressure and the residue was purified on RP18 silica by prepHPLC (Method I). Product containing fractions were freeze-dried to obtain 8.0 mg (8.4 μmol, 14%, purity 95.6% by HPLC Method A) of a white solid. LCMS (Method D) retention time 4.70 min, [M+H]+=839.4.

### Example 37: Synthetic Scheme for Compound XXXII

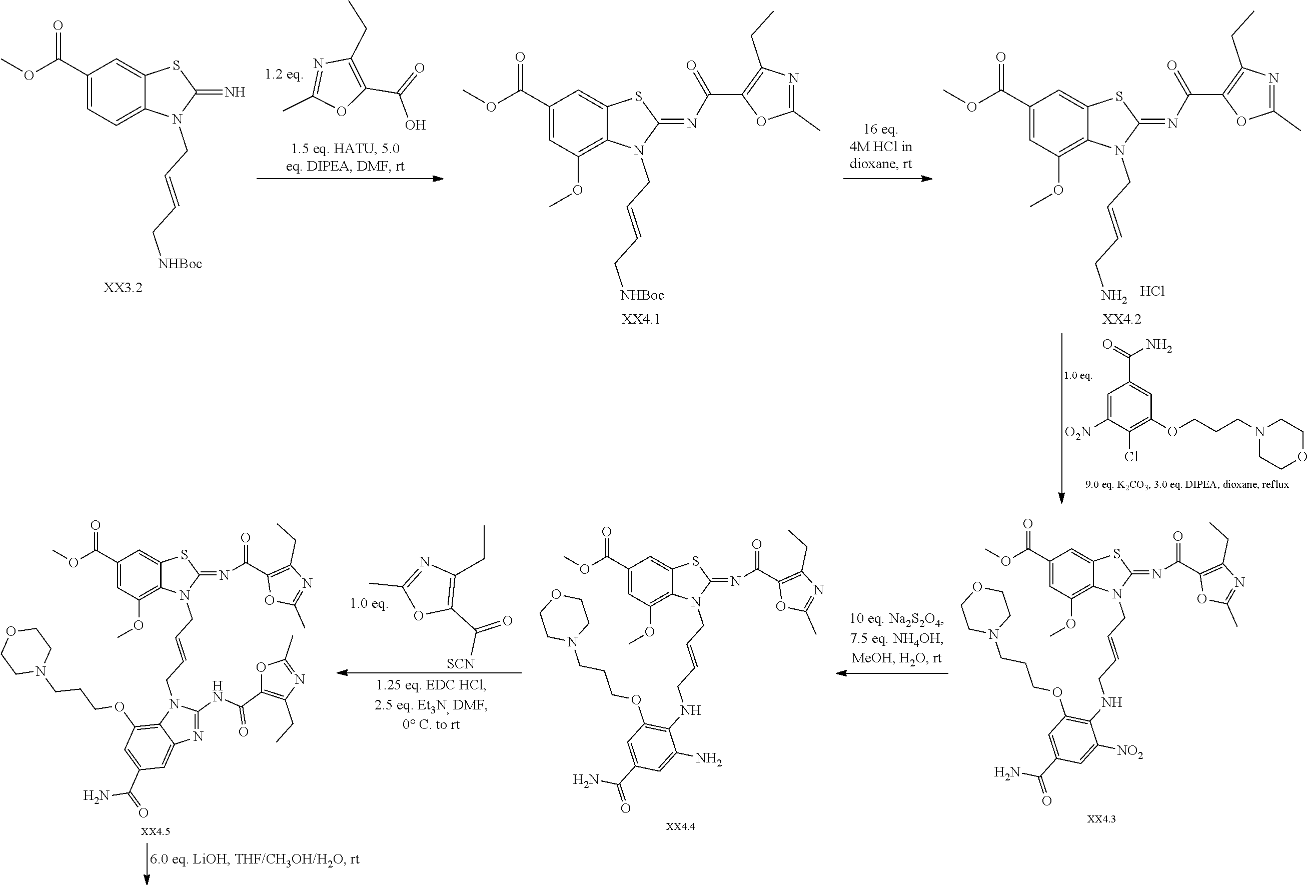

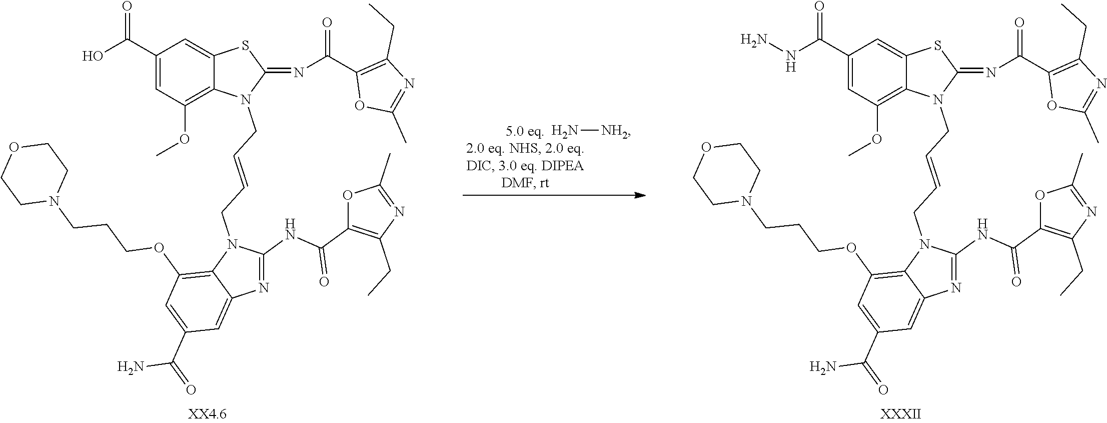

### Preparation of Methyl (Z)-3-((E)-4-((tert-butoxycarbonyl)amino)but-2-en-1-yl)-2-((4-ethyl-2-methyloxazole-5-carbonyl)imino)-4-methoxy-2,3-dihydrobenzo[d]thiazole-6-carboxylate (XX4.1)

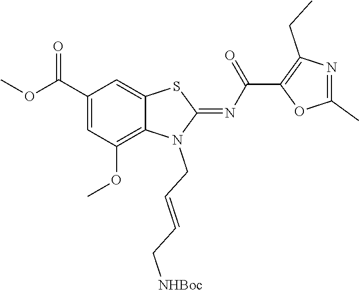

Compound XX3.2 (1.00 g, 2.45 mmol) and 4-ethyl-2-methyl-1,3-oxazole-5-carboxylic acid (450 mg, 2.95 mmol) were dissolved in DMF (10 mL). Afterwards, HATU (1.41 g, 3.68 mmol) and DIPEA (2.16 mL, 12.3 mmol) were added and the reaction mixture was stirred at room temperature for 1 h. The product formation was controlled by LC/MS. Then, water was added and the resulting solid was filtered off and washed with water. The product was dried in vacuo to give 1.40 g of a yellowish solid, which was used without further purification. LCMS (Method C) retention time 2.91 min, [M+Na]+=567.3. ¹H-NMR: (300 MHz, CDCl₃) δ [ppm]=7.96 (d, J=1.4 Hz, 1H), 7.61 (d, J=1.4 Hz, 1H), 5.91-5.63 (m, 2H), 5.40 (dd, J=5.5, 1.3 Hz, 2H), 4.50 (brs, 1H), 4.02 (s, 3H), 3.95 (s, 3H), 3.76-3.66 (m, 2H), 3.07 (q, J=7.6 Hz, 2H), 2.53 (s, 3H), 1.39 (s, 9H), 1.29 (t, J=7.6 Hz, 3H).

### Preparation of Methyl (Z)-3-((E)-4-aminobut-2-en-1-yl)-2-((4-ethyl-2-methyloxazole-5-carbonyl)imino)-4-methoxy-2,3-dihydrobenzo[d]thiazole-6-carboxylate (XX4.2)

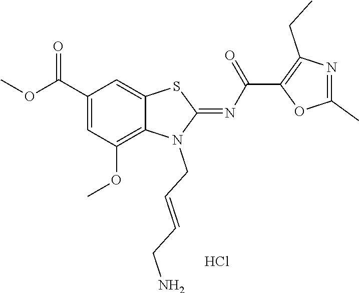

Compound XX4.1 (1.40 g, 2.57 mmol) was suspended and 4 M HCl in dioxane (10.3 mL, 41.1 mmol) was added. The resulting suspension was stirred at room temperature for 1 h. The product formation was controlled by LC/MS. The formed solid was filtered off, washed with diethyl ether and dried in vacuo to give 1.07 g (2.22 mmol, 94%) of a yellowish solid. LCMS (Method A) retention time 1.87 min, [M+H]+=445.1. ¹H-NMR: (300 MHz, DMSO-d6) δ [ppm]=8.34-7.93 (m, 2H), 7.57-7.49 (m, 1H), 7.12 (brs, 1H), 6.12-5.96 (m, 1H), 5.77-5.62 (m, 1H), 5.31 (d, J=5.7 Hz, 2H), 4.01 (s, 3H), 3.87 (s, 3H), 3.47-3.35 (m, 2H), 2.96 (q, J=7.5 Hz, 2H), 2.47 (s, 3H), 1.20 (t, J=7.5 Hz, 3H).

### Preparation of Methyl (Z)-3-((E)-4-((4-carbamoyl-2-(3-morpholinopropoxy)-6-nitrophenyl)amino)but-2-en-1-yl)-2-((4-ethyl-2-methyloxazole-5-carbonyl)imino)-4-methoxy-2,3-dihydrobenzo[d]thiazole-6-carboxylate (XX4.3)

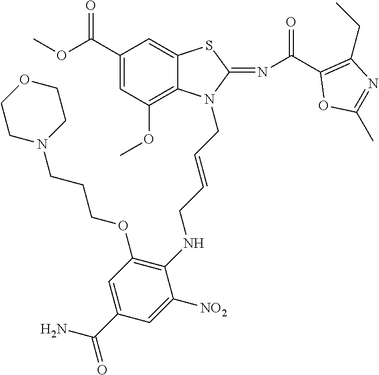 [SMILES: CCc1nc(C)oc1C(=O)/N=c1\sc2cc(C(=O)OC)cc(OC)c2n1C/C=C/CNc1c(OCCCN2CCOCC2)cc(C(N)=O)cc1[N+](=O)[O-]]

Methyl (Z)-3-((E)-4-aminobut-2-en-1-yl)-2-((4-ethyl-2-methyloxazole-5-carbonyl)imino)-4-methoxy-2,3-dihydrobenzo[d]thiazole-6-carboxylate (100 mg, 208 μmol) was suspended in dioxane (4 mL), 4-chloro-3-(3-morpholinopropoxy)-5-nitrobenzamide (71.5 mg, 208 μmol), DIPEA (73.2 μL, 415 μmol) and K₂CO₃ (86.2 mg, 624 μmol) and the mixture was stirred at 120° C. for 24 h. Again, K₂CO₃ (57.5 mg, 415 μmol) was added and the reaction mixture was stirred at 120° C. for 4 days. Then, K₂CO₃ (57.5 mg, 415 μmol) was added and the reaction mixture was stirred at 120° C. for 23 h. Again, 4-chloro-3-(3-morpholinopropoxy)-5-nitrobenzamide (17.8 mg, 52.0 μmol), DIPEA (36.6 μL, 208 μmol) and K₂CO₃ (57.5 mg, 415 μmol) were added and the reaction mixture was stirred at 120° C. for 4 h. The product formation was controlled by LC/MS. The reaction was cooled to room temperature and quenched with water (10 mL). The product was extracted with CH₂Cl₂/CH₃OH (9/1, 2×20 mL). The organic layer was dried over Na₂SO4 and filtered. The solvent was removed under reduced pressure. The residue was purified by automated flash column chromatography on Büchi C-850 (XSelect® CSH™ Prep C18 0.5 μM, 50 mm×150 mm, 100 mL/min; 15-100% CH₃CN in H₂O (+0.15% TFA) in 30 min). Product containing fractions were combined to give 25 mg (29 μmol, 14%) of an orange solid. LCMS (Method B) retention time 2.71 min, [M+H]+=752.3. ¹H-NMR: (300 MHz, DMSO-d6) δ [ppm]=8.16-8.09 (m, 1H), 8.03-7.97 (m, 1H), 7.87 (brs, 1H), 7.61-7.46 (m, 2H), 7.44-7.37 (m, 1H), 7.25 (brs, 1H), 5.83-5.55 (m, 2H), 5.27 (d, J=5.5 Hz, 2H), 4.07 (d, J=6.3 Hz, 2H), 4.01-3.92 (m, 4H), 3.90 (s, 3H), 3.85 (s, 3H), 3.62 (t, J=12.1 Hz, 2H), 3.40 (d, J=12.4 Hz, 2H), 3.29-3.14 (m, 2H), 3.06-3.00 (m, 2H), 2.91 (q, J=7.5 Hz, 2H), 2.48 (s, 3H), 2.06 (d, J=9.5 Hz, 2H), 1.15 (t, J=7.6 Hz, 3H).

### Preparation of Methyl (Z)-3-((E)-4-((2-amino-4-carbamoyl-6-(3-morpholinopropoxy)phenyl)amino)but-2-en-1-yl)-2-((4-ethyl-2-methyloxazole-5-carbonyl)imino)-4-methoxy-2,3-dihydrobenzo[d]thiazole-6-carboxylate (XX4.4)

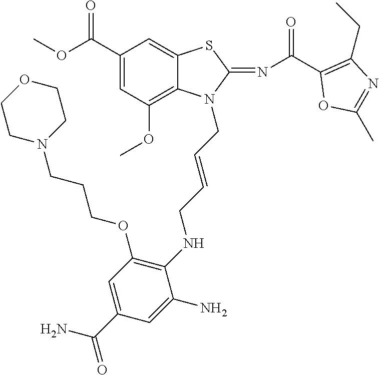 [SMILES: CCc1nc(C)oc1C(=O)/N=c1\sc2cc(C(=O)OC)cc(OC)c2n1C/C=C/CNc1c(N)cc(C(N)=O)cc1OCCCN1CCOCC1]

To a stirred solution of compound XX4.3 (1.15 g, 1.33 mmol) in CH₃OH (20 mL) was added sodium dithionite (2.31 g, 13.3 mmol) dissolved in water (5 mL) at 0° C. Then, NH₃ (30% aqueous solution, 1.32 mL, 9.96 mmol) was added to the mixture at 0° C. The reaction was allowed to warm to room temperature and stirred at room temperature for 30 min. The reaction progress was controlled by LC/MS. The reaction mixture was diluted with H₂O (75 mL) and CH₂Cl₂ (100 mL) was added. The aqueous layer was extracted with ethyl acetate (2×50 mL). The organic layer was dried over sodium sulfate and filtered. The solvent was removed under reduced pressure and dried in vacuo to yield 845 mg (1.17 mmol, 88%) of a yellowish solid. The residue was used without further purification. LCMS (Method A) retention time 1.76 min, [M+H]⁺=722.3. ¹H-NMR: (300 MHz, DMSO-d6) δ [ppm]=8.16-8.08 (m, 1H), 7.59-7.42 (m, 2H), 6.92 (s, 1H), 6.84-6.76 (m, 1H), 6.66-6.60 (m, 1H), 5.87-5.57 (m, 2H), 5.24 (d, J=5.4 Hz, 2H), 4.60 (d, 2H), 3.88 (d, J=2.8 Hz, 7H), 3.74 (t, J=6.3 Hz, 2H), 3.63-3.53 (m, 2H), 3.49 (t, J=4.6 Hz, 4H), 2.94 (q, J=7.5 Hz, 2H), 2.46 (s, 3H), 2.29-2.17 (m, 6H), 1.65 (p, J=6.6 Hz, 2H), 1.16 (t, J=7.5 Hz, 3H).

### Preparation of Methyl (Z)-3-((E)-4-(5-carbamoyl-2-(4-ethyl-2-methyloxazole-5-carboxamido)-7-(3-morpholinopropoxy)-1H-benzo[d]imidazol-1-yl)but-2-en-1-yl)-2-((4-ethyl-2-methyloxazole-5-carbonyl)imino)-4-methoxy-2,3-dihydrobenzo[d]thiazole-6-carboxylate trifluoroacetic acid salt (XX4.5)

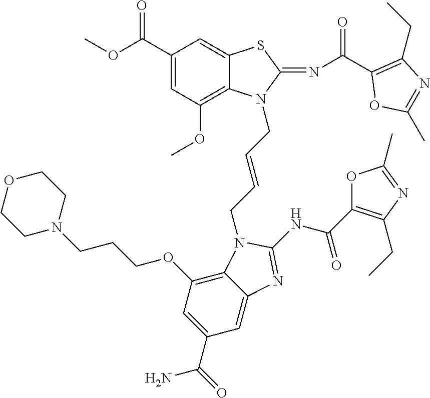 [SMILES: CCc1nc(C)oc1C(=O)/N=c1\sc2cc(C(=O)OC)cc(OC)c2n1C/C=C/Cn1c(NC(=O)c2oc(C)nc2CC)nc2cc(C(N)=O)cc(OCCCN3CCOCC3)c21]

Compound XX4.4 (250 mg, 346 μmol) was dissolved in anhydrous DMF (4 mL) and cooled to 0° C. Then, 4-ethyl-2-methyloxazole-5-carbonyl isothiocyanate (0.2 M in dioxane, 865 μL, 173 μmol) was added dropwise and the reaction was stirred at 0° C. for 30 min. Again, 4-ethyl-2-methyloxazole-5-carbonyl isothiocyanate (0.2 M in dioxane, 430 μL, 87.0 μmol) was added dropwise and the reaction mixture was stirred at 0° C. for 15 min. Then, 4-ethyl-2-methyloxazole-5-carbonyl isothiocyanate (0.2 M in dioxane, 430 μL, 87.0 μmol) was added dropwise and the reaction mixture was stirred at 0° C. for 1 h. Then, EDC·HCl (83.0 mg, 433 μmol) and Et₃N (121 μL, 866 μmol) were added and the reaction mixture was stirred at room temperature for 2 days. The product formation was controlled by LC/MS. The reaction mixture was diluted with water/sat. aqueous NH₄Cl (3:1, 20 mL) and extracted with CH₂Cl₂/CH₃OH (3:1, 3×25 mL). The combined organic layers were dried over sodium sulfate and filtered. The solvent was removed under reduced pressure. The residue was purified by flash column chromatography on Büchi C-850 (XSelect® CSH™ Prep OBD™ C18 0.5 μM, 50 mm×150 mm, 100 mL/min; 5-100% CH₃CN in H₂O (+0.15% TFA) in 30 min) to yield 240 mg (240 μmol, 69%) of an off-white solid. LCMS (Method C) retention time 3.31 min, [M+H]⁺=884.4. ¹H-NMR: (300 MHz, DMSO-d6) δ [ppm]=9.91 (s, 1H), 8.15-8.09 (m, 1H), 7.90 (s, 1H), 7.68-7.62 (m, 1H), 7.47-7.40 (m, 1H), 7.34 (s, 1H), 7.29-7.22 (m, 1H), 5.83-5.60 (m, 2H), 5.26 (d, J=4.6 Hz, 2H), 4.88 (d, J=4.5 Hz, 2H), 4.00 (t, J=5.8 Hz, 2H), 3.94-3.84 (m, 5H), 3.72 (s, 3H), 3.67-3.55 (m, 2H), 3.38-3.24 (m, 2H), 3.22-3.10 (m, 2H), 3.03-2.97 (m, 2H), 2.86-2.71 (m, 4H), 2.46-2.35 (m, 6H), 1.98-1.87 (m, 2H), 1.09-0.96 (m, 6H).

### Preparation of (Z)-3-((E)-4-(5-carbamoyl-2-(4-ethyl-2-methyloxazole-5-carboxamido)-7-(3-morpholinopropoxy)-1H-benzo[d]imidazol-1-yl)but-2-en-1-yl)-2-((4-ethyl-2-methyloxazole-5-carbonyl)imino)-4-methoxy-2,3-dihydrobenzo[d]thiazole-6-carboxylic acid (XX4.6)

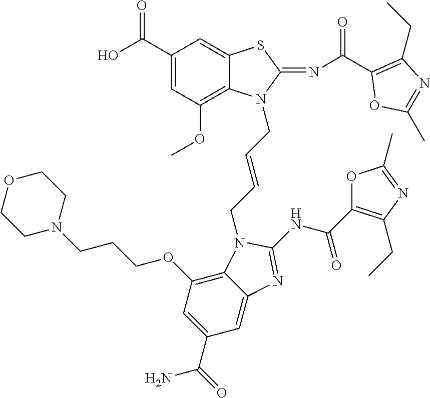 [SMILES: CCc1nc(C)oc1C(=O)/N=c1\sc2cc(C(=O)O)cc(OC)c2n1C/C=C/Cn1c(NC(=O)c2oc(C)nc2CC)nc2cc(C(N)=O)cc(OCCCN3CCOCC3)c21]

Compound XX4.5 (100 mg, 100 μmol) was dissolved in CH₃OH/THF/H₂O (2:2:1; 2.5 mL). Afterwards, LiOH (25.2 mg, 601 μmol) was added and the resulting reaction mixture was stirred at room temperature for 18 h. The reaction progress was controlled by LC/MS. The organic solvents were removed under reduced pressure. The aqueous phase was cooled to 0° C. and neutralized with 1 M aq. HCl. The precipitate was filtered and washed with water. The solid was dissolved/suspended in CH₃CN/H₂O (1:1, 15 mL) and freeze-dried to yield 64 mg (74 μmol, 73%) of a white solid. The product was used without further purification. LCMS (Method A) retention time 1.86 min, [M+H]⁺=870.5. ¹H-NMR: (300 MHz, DMSO-d6) δ [ppm]=13.16 (brs, 1H), 12.73 (brs, 1H), 10.94 (brs, 1H), 8.14-8.08 (m, 1H), 7.93 (s, 1H), 7.68-7.62 (m, 1H), 7.55-7.48 (m, 1H), 7.35-7.28 (m, 1H), 5.89-5.63 (m, 2H), 5.28 (d, J=4.9 Hz, 2H), 4.90 (d, J=4.8 Hz, 2H), 4.11-4.01 (m, 2H), 3.92-3.86 (m, 2H), 3.82-3.64 (m, 5H), 3.16-3.10 (m, 2H), 3.02-2.96 (m, 2H), 2.85-2.71 (m, 4H), 2.47-2.33 (m, 6H), 2.10-1.92 (m, 2H), 1.24 (s, 2H), 1.07-0.93 (m, 6H).

### Preparation of Compound XXXII

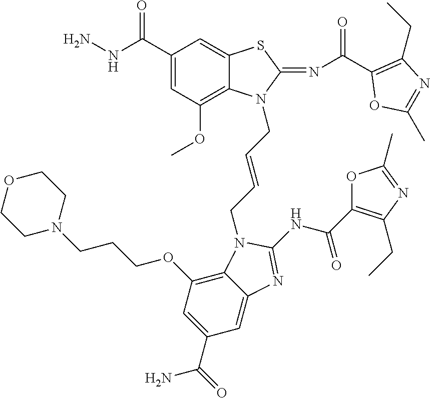 [SMILES: CCc1nc(C)oc1C(=O)/N=c1\sc2cc(C(=O)NN)cc(OC)c2n1C/C=C/Cn1c(NC(=O)c2oc(C)nc2CC)nc2cc(C(N)=O)cc(OCCCN3CCOCC3)c21]

Compound XX4.6 (62.0 mg, 71.3 μmol) was suspended in DMF (3 mL). Afterwards, DIPEA (37.2μ, 214 μmol), NHS (16.7 mg, 143 μmol) and DIC (22.1μ L, 143 μmol) were added and the resulting reaction mixture was stirred at 40° C. for 3 h. Afterwards, the reaction was stirred at room temperature for 17 h. The reaction progress was controlled by LC/MS. Then, 1 M hydrazine in THF (356 μL, 356 μmol) and DIPEA (62 μL, 356 μmol) were added and the resulting reaction mixture was stirred at room temperature for 1 h. The reaction progress was controlled by LC/MS. The solvent was removed under reduced pressure and the residue was purified on RP18 silica by prepHPLC (Method I). Product containing fractions were freeze-dried to obtain 43.0 mg (43.1 μmol, 60%, purity 95.9% by HPLC Method A) of a white solid. LCMS (Method C) retention time 2.72 min, [M+H]+=884.4.

### Example 38: Synthetic Scheme for Compound XXXIII

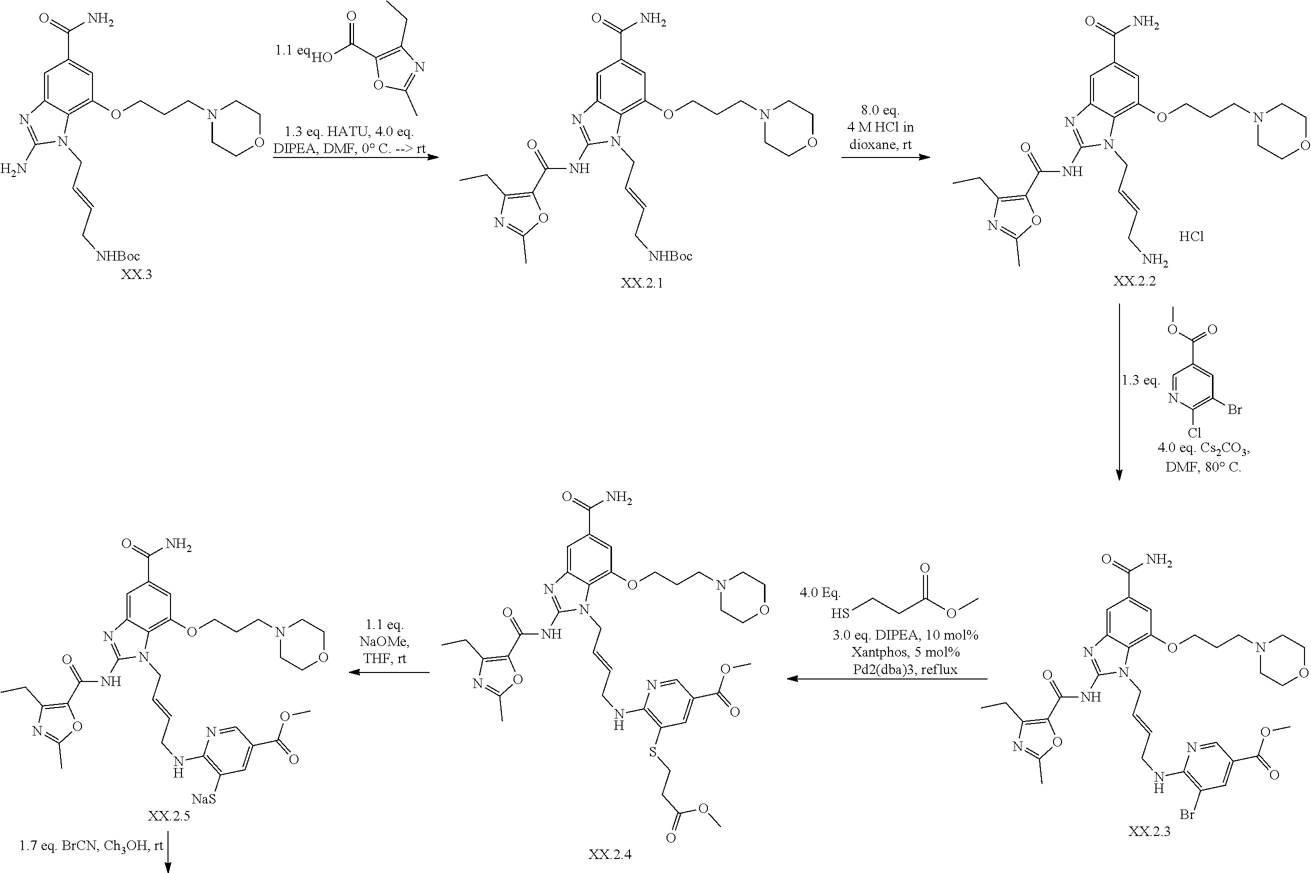

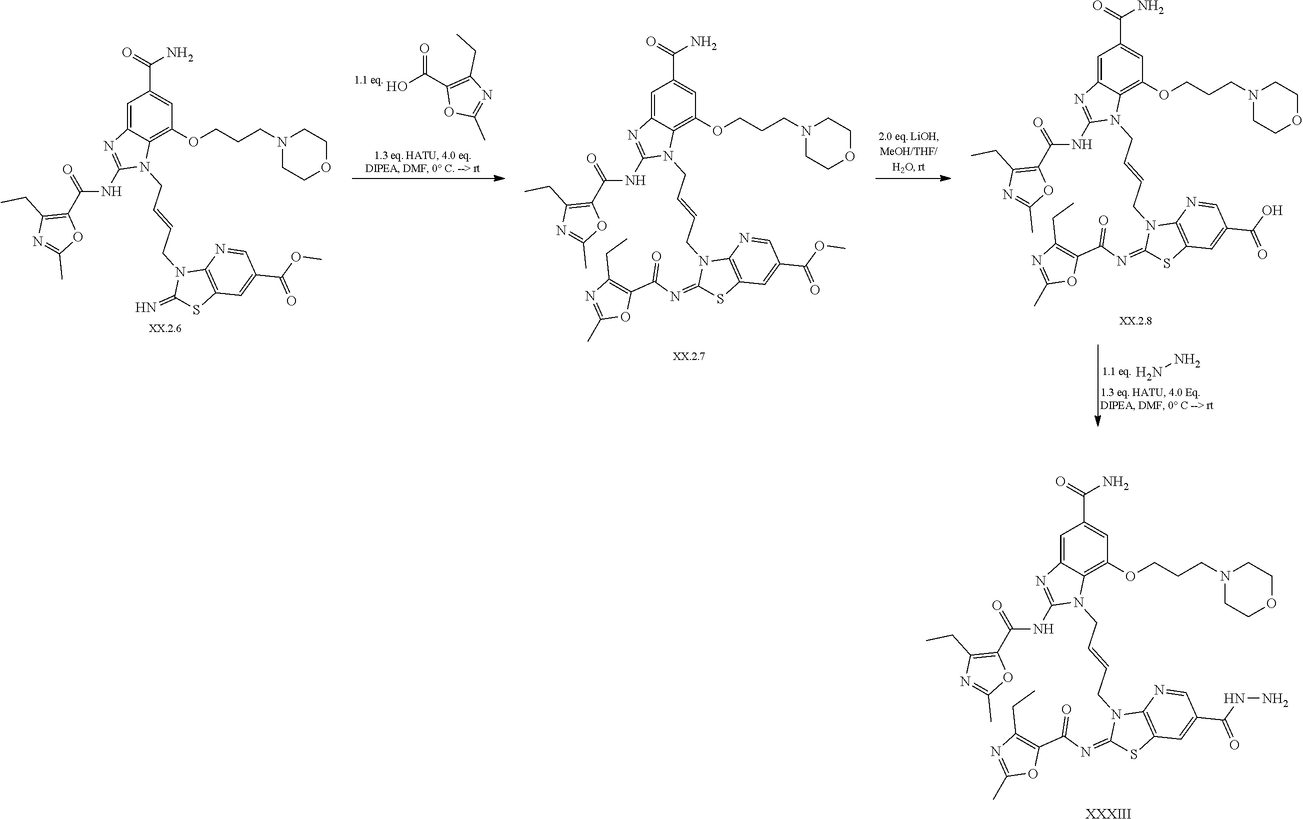

### Preparation of tert-Butyl (E)-(4-(5-carbamoyl-2-(4-ethyl-2-methyloxazole-5-carboxamido)-7-(3-morpholinopropoxy)-1H-benzo[d]imidazol-1-yl)but-2-en-1-yl)carbamate trifluoroacetic acid salt (XX2.1)

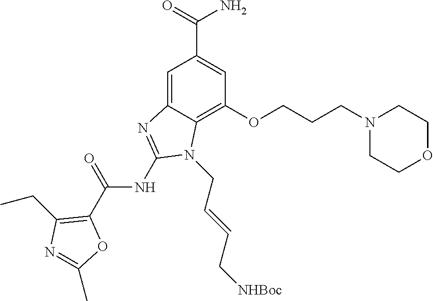 [SMILES: CCc1nc(C)oc1C(=O)Nc1nc2cc(C(N)=O)cc(OCCCN3CCOCC3)c2n1C/C=C/CNC(=O)OC(C)(C)C]

Compound XX.3 (1.96 g, 4.01 mmol) and 4-ethyl-2-methyl-1,3-oxazole-5-carboxylic acid (685.7 mg, 4.41 mmol) were dissolved in DMF (20 mL) and afterwards treated with DIPEA (2.65 mL, 16.1 mmol) for 15 min at 0° C. Then, HATU (1.98 g, 5.22 mmol) was added and the resulting yellowish reaction mixture was stirred at room temperature for 1 h. The product formation was controlled by LCMS. The solvent was removed in vacuo and the residue was purified on RP18 silica by automated flash column chromatography on Büchi C-850 (XSelect® CSH™ Prep OBD™ 0.5 UM, 50 mm×250 mm, 100 mL/min; 10-100% CH₃CN in H₂O (+0.15% TFA) in 47 min). Product containing fractions were combined and freeze-dried to obtain 1.87 g (2.53 mmol, 63%) of a reddish solid. LCMS (Method C) retention time 2.57 min, [M+H]⁺=626.2. ¹H-NMR (300 MHz, DMSO-d6): d [ppm]=9.80 (brs, 1H), 7.95 (brs, 1H), 7.68 (dd, J=2.5, 1.2 Hz, 1H), 7.42-7.31 (m, 2H), 6.97-6.85 (m, 1H), 5.84-5.67 (m, 1H), 5.61-5.39 (m, 1H), 5.03-4.90 (m, 2H), 4.26 (t, J=5.8 Hz, 2H), 4.03 (d, J=12.6 Hz, 2H), 3.68 (t, J=12.1 Hz, 2H), 3.60-3.40 (m, 4H), 3.37-3.26 (m, 2H), 3.25-3.06 (m, 2H), 2.99 (q, J=7.5 Hz, 2H), 2.44 (s, 3H), 2.31-2.15 (m, 2H), 1.39-1.06 (m, 12H).

### Preparation of (E)-N-(1-(4-aminobut-2-en-1-yl)-5-carbamoyl-7-(3-morpholinopropoxy)-1H-benzo[d]imidazol-2-yl)-4-ethyl-2-methyloxazole-5-carboxamide hydrochloric acid (XX2.2)

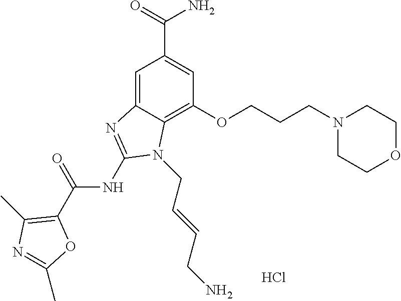 [SMILES: Cc1nc(C)c(C(=O)Nc2nc3cc(C(N)=O)cc(OCCCN4CCOCC4)c3n2C/C=C/CN)o1 ; Cl]

Compound XX2.1 (1.87 g, 2.53 mmol) was dissolved/suspended in CH₂Cl₂ (10 mL). Afterwards, HCl (4 M in dioxane, 5.06 mL) was added and the resulting mixture was stirred at room temperature for 24 h. The reaction progress was controlled by LCMS. HCl (0.15% in H₂O) was added and afterwards all volatiles were removed in vacuo. The residue was purified on RP18 silica by automated flash column chromatography on Büchi C-850 (XSelect® CSH™ Prep OBD™ 0.5 UM, 50 mm×150 mm, 100 ml/min; 10-100% CH₃CN in H₂O (+0.15% HCl) in 47 min). Product containing fractions were combined and freeze-dried to obtain 1.18 g (2.10 mmol, 83%) of a white solid. LCMS (Method C) retention time 1.67 min, [M+H]⁺=526.1. ¹H-NMR (300 MHz, DMSO-d6): d [ppm]=11.65 (s, 1H), 8.28-8.22 (m, 3H), 7.70 (d, J=1.2 Hz, 1H), 7.52-7.33 (m, 2H), 6.16-6.06 (m, 1H), 5.70-5.50 (m, 1H), 4.99 (d, J=5.2 Hz, 2H), 4.30 (t, J=6.0 Hz, 2H), 4.05-3.84 (m, 4H), 3.57-3.39 (m, 4H), 3.36-3.08 (m, 4H), 2.99 (q, J=7.5 Hz, 2H), 2.50-2.30 (m, 5H), 1.29-1.15 (m, 3H).

### Preparation of Methyl (E)-5-bromo-6-((4-(5-carbamoyl-2-(4-ethyl-2-methyloxazole-5-carboxamido)-7-(3-morpholinopropoxy)-1H-benzo[d]imidazol-1-yl)but-2-en-1-yl)amino)nicotinate trifluoroacetic acid salt (XX2.3)

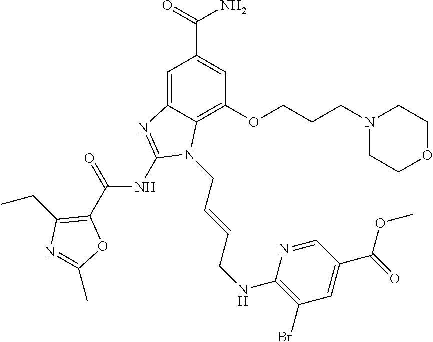 [SMILES: CCc1nc(C)oc1C(=O)Nc1nc2cc(C(N)=O)cc(OCCCN3CCOCC3)c2n1C/C=C/CNc1ncc(C(=O)OC)cc1Br]

Compound XX2.2 (1.07 g, 1.90 mmol) and methyl 5-bromo-6-chloronicotinate (572 mg, 2.28 mmol) were dissolved in DMF (25 mL). Afterwards, Et₃N (2.65 mL, 19.0 mmol) was added and the resulting reaction mixture was heated to 55° C. for 3 days. The product formation was controlled by LCMS. All volatiles were removed in vacuo and the residue was purified on RP18 silica by automated flash column chromatography on Büchi C-850 (XSelect® CSH™ Prep OBD™ 0.5 UM, 50 mm×150 mm, 100 mL/min; 5-100% CH₃CN in H₂O (+0.15% TFA) in 40 min). Product containing fractions were freeze-dried to obtain 759 mg (890 μmol, 47%) of a white solid. LCMS (Method C) retention time 1.63 min, [M+H]⁺=739.1. ¹H-NMR (300 MHz, DMSO-d6): d [ppm]=12.75 (brs, 1H), 10.10 (brs, 1H), 8.47 (d, J=2.0 Hz, 1H), 8.07 (d, J=2.0 Hz, 1H), 7.94 (brs, 1H), 7.67 (d, J=1.2 Hz, 1H), 7.39-7.25 (m, 3H), 5.88-5.73 (m, 1H), 5.73-5.58 (m, 1H), 4.92 (d, J=5.3 Hz, 2H), 4.21 (t, J=5.8 Hz, 2H), 4.06-3.96 (m, 4H), 3.79 (s, 3H), 3.70 (d, J=12.0 Hz, 1H), 3.47 (d, J=12.1 Hz, 2H), 3.29 (t, J=8.1 Hz, 2H), 3.12 (s, 2H), 2.89 (q, J=7.5 Hz, 2H), 2.42 (s, 3H), 2.22-2.10 (m, 2H), 1.09 (t, J=7.5 Hz, 3H).

### Preparation of Methyl (E)-6-((4-(5-carbamoyl-2-(4-ethyl-2-methyloxazole-5-carboxamido)-7-(3-morpholinopropoxy)-1H-benzo[d]imidazol-1-yl)but-2-en-1-yl)amino)-5-((3-methoxy-3-oxopropyl)thio) nicotinate trifluoroacetic acid salt (XX2.4)

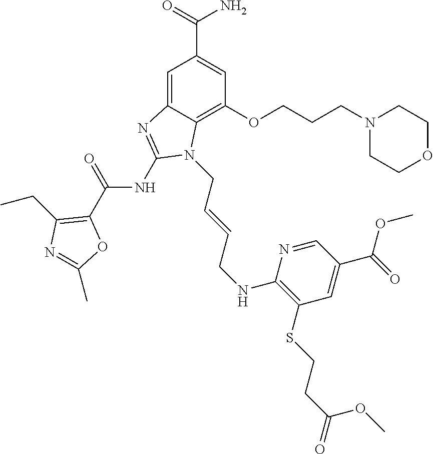 [SMILES: CCc1nc(C)oc1C(=O)Nc1nc2cc(C(N)=O)cc(OCCCN3CCOCC3)c2n1C/C=C/CNc1ncc(C(=O)OC)cc1SCCC(=O)OC]

Compound XX2.3 (750 mg, 879 μmol) was co-evaporated with anhydrous 1,4-dioxane (7.5 mL). The residue was dissolved in anhydrous 1,4-dioxane (7.5 mL) and DIPEA (451 μL, 2.65 mmol) and methyl 3-mercaptopropanoate (389 μL, 3.51 mmol) were added. Afterwards, Xantphos (50.8 mg, 87.9 μmol) and Pd₂(dba)₃ (40.2 mg, 43.9 μmol) were added under slight argon stream and the resulting mixture was heated to 130° C. for 30 min. The product formation was controlled by LCMS. Water was added and afterwards all solvents were removed in vacuo. The residue (filtered through syringe filter PVDF-45/25) was purified on RP18 silica by automated flash column chromatography on Büchi C-850 (XSelect® CSH™ Prep OBD™ 0.5 UM, 50 mm×150 mm, 100 mL/min; 10-100% CH₃CN in H₂O (+0.15% TFA) in 47 min). Product containing fractions were freeze dried to obtain 682 mg (764 μmol, 87%) of a colorless solid. LCMS (Method C) retention time 2.28 min, [M+H]⁺=779.2. ¹H-NMR (300 MHz, DMSO-d6): d [ppm]=12.77 (brs, 1H), 10.01 (brs, 1H), 8.48 (d, J=2.2 Hz, 1H), 7.97-7.88 (m, 2H), 7.67 (d, J=1.3 Hz, 1H), 7.39-7.33 (m, 2H), 7.26 (t, J=5.9 Hz, 1H), 5.88-5.58 (m, 2H), 4.93 (d, J=5.3 Hz, 2H), 4.20 (t, J=5.8 Hz, 2H), 4.09-3.96 (m, 4H), 3.79 (s, 3H), 3.68 (t, J=12.0 Hz, 2H), 3.51 (s, 3H), 3.45 (s, 1H), 3.37-3.23 (m, 2H), 3.15-3.07 (m, 2H), 2.95-2.81 (m, 4H), 2.48-2.39 (m, 5H), 2.22-2.10 (m, 2H), 1.08 (t, J=7.5 Hz, 3H).

### Preparation of Methyl (E)-6-((4-(5-carbamoyl-2-(4-ethyl-2-methyloxazole-5-carboxamido)-7-(3-morpholinopropoxy)-1H-benzo[d]imidazol-1-yl)but-2-en-1-yl)amino)-5-mercaptonicotinate trifluoroacetic acid salt (XX2.5)

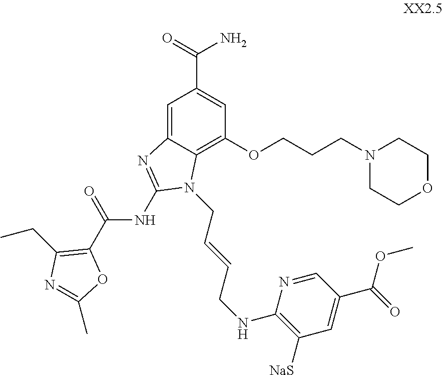

Compound XX2.4 (670 mg, 750 μmol) was dissolved in anhydrous THF (7.5 mL) and 25% NaOCH₃ in CH₃OH (686 μL, 3.00 mmol) was added under slight argon stream and the resulting mixture was stirred for 2 h. The product formation was controlled by LCMS. The mixture was diluted with CH₂Cl₂ (5 mL) and the resulting solid was filtered and washed with CH₂Cl₂ (3×2 mL). The residue was purified on RP18 silica by automated flash column chromatography on Buchi C-850 (XSelect® CSH™ Prep OBD™ 0.5 UM, 50 mm×150 mm, 100 mL/min; 10-100% CH₃CN in H₂O (+0.15% TFA) in 43 min). Product containing fractions were freeze-dried to obtain 396 mg (491 μmol, 65%) of a yellowish solid, which tends to form disulfide bonds. LCMS (Method C) retention time 1.87 min, [M+H]⁺=693.2.

### Preparation of Methyl (E)-3-(4-(5-carbamoyl-2-(4-ethyl-2-methyloxazole-5-carboxamido)-7-(3-morpholinopropoxy)-1H-benzo[d]imidazol-1-yl)but-2-en-1-yl)-2-imino-2,3-dihydrothiazolo[4,5-b]pyridine-6-carboxylate (XX2.6)

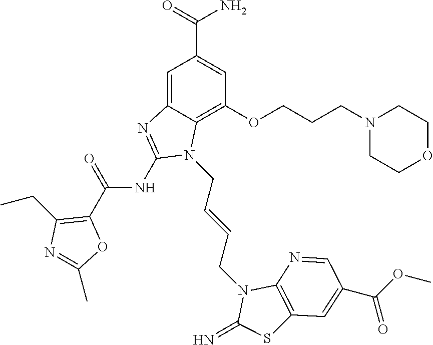 [SMILES: CCc1nc(C)oc1C(=O)Nc1nc2cc(C(N)=O)cc(OCCCN3CCOCC3)c2n1C/C=C/Cn1c(=N)sc2cc(C(=O)OC)cnc21]

Compound XX2.5 (390 mg, 483 μmol) was dissolved in CH₃OH (7.5 mL) and cooled to 0° C. Afterwards, cyanogen bromide (61.4 mg, 580 μmol) was added and the mixture was stirred at 0° C. for 1 h. Afterwards, the mixture was warmed to room temperature and stirred for 1 h. Afterwards, 0.5 M aq. TCEP (1 mL) was added and stirred at room temperature for 1 h. Then, 0.5 M aq. TCEP (2 mL) was added and stirred at room temperature for 2 h. All volatiles were removed in vacuo and the residue was dissolved in CH₃OH (7.5 mL) and stirred at room temperature for 1 h. The reaction progress was controlled by LCMS. The residue was purified on RP18 silica by automated flash column chromatography on Büchi C-850 (XSelect® CSH™ Prep OBD™ 0.5 UM, 50 mm×150 mm, 100 mL/min; 10-100% CH₃CN in H₂O (50 mM NH₄HCO₃) in 47 min). The product was obtained as a mixture with urea-side product. This material (153 mg) was used for further reaction. LCMS (Method C) retention time 1.62 min, [M+H]⁺=718.2.

### Preparation of Methyl (E)-3-((E)-4-(5-carbamoyl-2-(4-ethyl-2-methyloxazole-5-carboxamido)-7-(3-morpholinopropoxy)-1H-benzo[d]imidazol-1-yl)but-2-en-1-yl)-2-((4-ethyl-2-methyloxazole-5-carbonyl)imino)-2,3-dihydrothiazolo[4,5-b]pyridine-6-carboxylate trifluoroacetic acid salt (XX2.7)

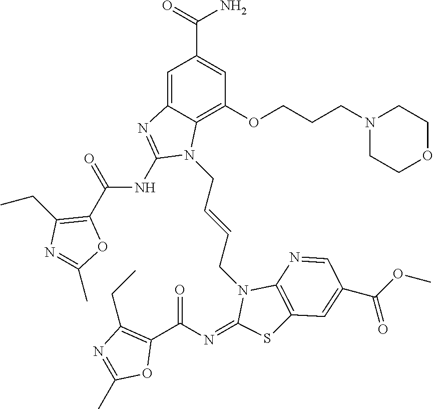 [SMILES: CCc1nc(C)oc1C(=O)/N=c1/sc2cc(C(=O)OC)cnc2n1C/C=C/Cn1c(NC(=O)c2oc(C)nc2CC)nc2cc(C(N)=O)cc(OCCCN3CCOCC3)c21]

Compound XX2.6 (153 mg, 213 μmol) and 4-ethyl-2-methyl-1,3-oxazole-5-carboxylic acid (36.4 mg, 234 μmol) were dissolved in DMF (4 mL) and afterwards treated with DIPEA (141 μL, 853 μmol) for 15 min at 0° C. Then, HATU (105 mg, 277 μmol) was added and the resulting yellowish reaction mixture was stirred at room temperature for 2 h. The product formation was controlled by LCMS. The solvent was removed in vacuo and the residue was purified on RP18 silica by automated flash column chromatography on Büchi C-850 (XSelect® CSH™ Prep OBD™ 0.5 μM, 50 mm×150 mm, 100 ml/min; 5-100% CH₃CN in H₂O (+0.15% TFA) in 40 min). Product containing fractions were freeze-dried (CH₃CN/H₂O=1:1) to give 116 mg (119 μmol, 56%) of a white solid. LCMS (Method C) retention time 3.22 min, [M+H]⁺=855.2. ¹H-NMR (300 MHz, DMSO-d6): d [ppm]=12.73 (brs, 1H), 9.84 (brs, 1H), 8.91 (dd, J=12.6, 2.0 Hz, 2H), 7.91 (brs, 1H), 7.64 (d, J=1.2 Hz, 1H), 7.37-7.27 (m, 2H), 5.97-5.83 (m, 1H), 5.77-5.62 (m, 1H), 5.02 (d, J=5.4 Hz, 2H), 4.93-4.85 (m, 2H), 4.11 (t, J=5.8 Hz, 2H), 4.02-3.84 (m, 5H), 3.64 (t, J=11.1 Hz, 2H), 3.47-3.29 (m, 2H), 3.29-3.15 (m, 2H), 3.15-2.96 (m, 2H), 2.83-2.66 (m, 4H), 2.43 (s, 3H), 2.39 (s, 3H), 2.07-1.98 (m, 2H), 1.03-0.91 (m, 5H).

### Preparation of (E)-3-((E)-4-(5-carbamoyl-2-(4-ethyl-2-methyloxazole-5-carboxamido)-7-(3-morpholinopropoxy)-1H-benzo[d]imidazol-1-yl)but-2-en-1-yl)-2-((4-ethyl-2-methyloxazole-5-carbonyl)imino)-2,3-dihydrothiazolo[4,5-b]pyridine-6-carboxylic acid (XX2.8)

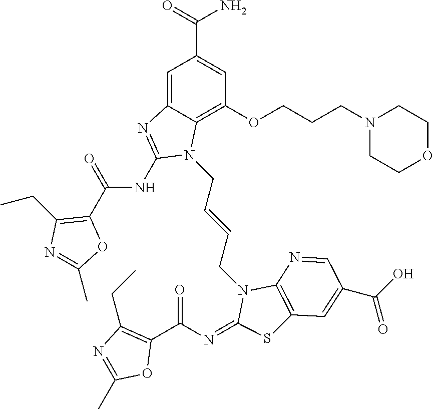 [SMILES: CCc1nc(C)oc1C(=O)/N=c1/sc2cc(C(=O)O)cnc2n1C/C=C/Cn1c(NC(=O)c2oc(C)nc2CC)nc2cc(C(N)=O)cc(OCCCN3CCOCC3)c21]

Compound XX2.7 (110 mg, 114 μmol) was dissolved in CH₃OH/THF/H₂O (2:2:1; 5 mL). Afterwards, LiOH (8.16 mg, 341 μmol) was added and the resulting reaction mixture was stirred at room temperature for 3 h. The reaction progress was controlled by LCMS. The organic solvents were removed in vacuo (rotary evaporator). The aqueous phase (additional water was added ~5 mL) was cooled to 0° C. and neutralized with 1 M aq. HCl. The suspension was transferred into a 50 mL Falcon tube and centrifugated with 7000 rpm for 10 min at 0° C. The pellet was washed with water (15 mL) and centrifugated with 7000 rpm for 10 min at 0° C. The solid was dissolved/suspended in CH₃CN/H₂O (1:1, 25 mL) and freeze-dried. The product (94.9 mg) was used without further purification. LCMS (Method C) retention time 2.89 min, [M+H]⁺=841.1. ¹H-NMR (300 MHz, DMSO-d6): d [ppm]=13.39 (s, 1H), 12.70 (s, 1H), 11.37 (s, 1H), 8.94 (d, J=2.0 Hz, 1H), 8.85 (d, J=1.9 Hz, 1H), 7.94 (s, 1H), 7.67-7.61 (m, 1H), 7.41-7.31 (m, 2H), 5.98-5.83 (m, 1H), 5.78-5.61 (m, 1H), 5.02 (d, J=5.3 Hz, 2H), 4.89 (d, J=4.9 Hz, 2H), 4.13 (t, J=5.8 Hz, 2H), 4.01-3.74 (m, 5H), 3.28-2.96 (m, 4H), 2.82-2.64 (m, 4H), 2.45-2.35 (m, 6H), 2.16-2.05 (m, 2H), 1.07-0.88 (m, 6H).

### Preparation of N-(5-carbamoyl-1-((E)-4-((E)-2-((4-ethyl-2-methyloxazole-5-carbonyl)imino)-6-(hydrazinecarbonyl)thiazolo[4,5-b]pyridin-3(2H)-yl)but-2-en-1-yl)-7-(3-morpholinopropoxy)-1H-benzo[d]imidazol-2-yl)-4-ethyl-2-methyloxazole-5-carboxamide (Compound XXXIII)

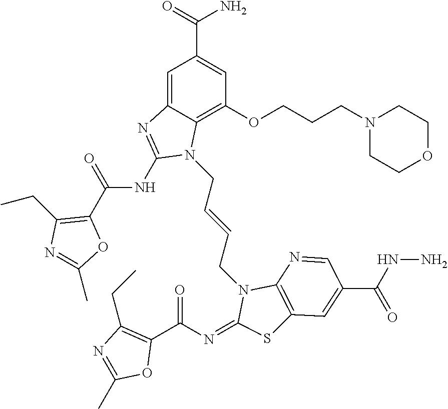 [SMILES: CCc1nc(C)oc1C(=O)/N=c1/sc2cc(C(=O)NN)cnc2n1C/C=C/Cn1c(NC(=O)c2oc(C)nc2CC)nc2cc(C(N)=O)cc(OCCCN3CCOCC3)c21]

Compound XX2.8 (95 mg, 0.13 mmol) was suspended in DMF (3 mL). Afterwards, DIPEA (59 UL, 0.34 mmol), NHS (26 mg, 0.23 mmol) and DIC (35 UL, 0.23 mmol) were added and the resulting reaction mixture was stirred at room temperature for 5.5 h. The reaction progress was controlled by LCMS. Afterwards, 1 M hydrazine in THF (564 μL, 564 μmol) and DIPEA (59 μL, 0.34 mmol) were added and the resulting reaction mixture was stirred at room temperature for 30 min. The reaction progress was controlled by LC/MS. The solvent was removed in vacuo (oil pump vacuo) and freeze-dried (CH₃CN/H₂O 1:1; 10 mL). The residue was purified on RP18 silica by prepHPLC (Method III). Product containing fractions were freeze-dried to obtain 52 mg (54%, purity 96% by HPLC Method A) of a white solid. LCMS (Method B) retention time 1.97 min, [M+H]⁺=855.2.

### Example 39: Synthetic Scheme for Compound XXXIV

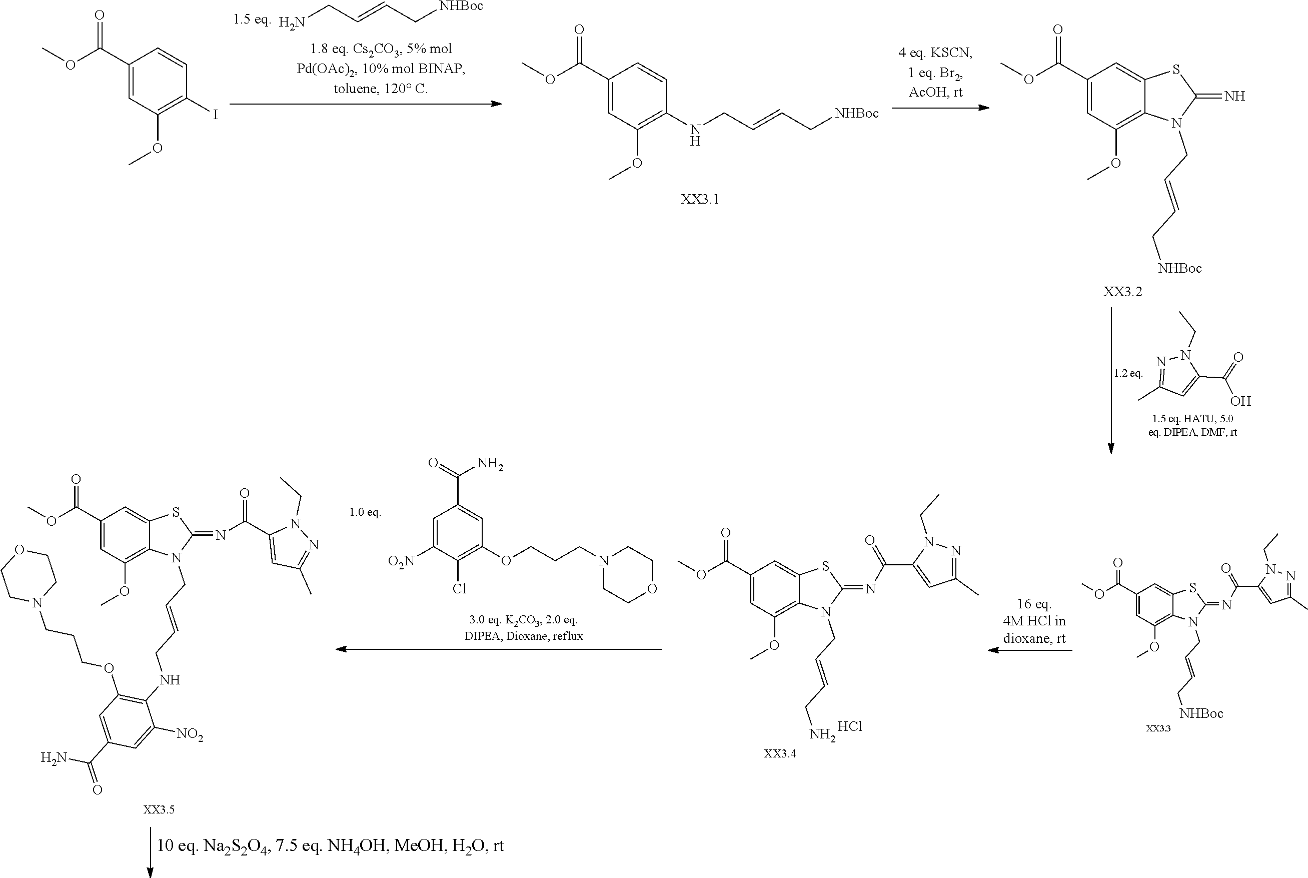

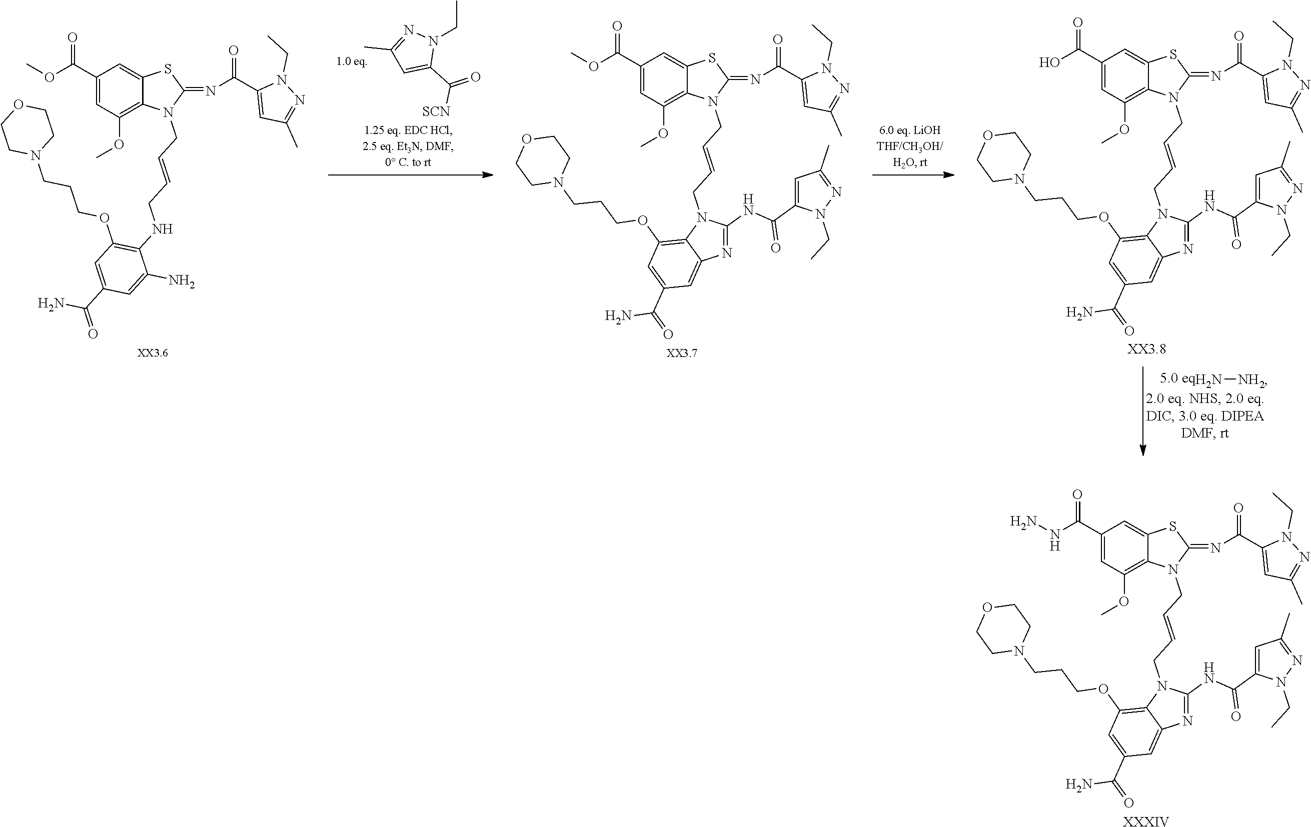

### Preparation of Methyl (E)-4-((4-((tert-butoxycarbonyl)amino)but-2-en-1-yl)amino)-3-methoxybenzoate (XX3.1)

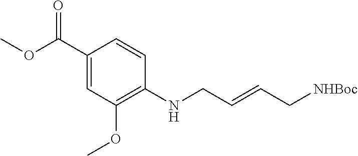 [SMILES: COC(=O)c1ccc(NC/C=C/CNC(=O)OC(C)(C)C)c(OC)c1]

Methyl 4-iodo-3-methoxybenzoate (1.00 g, 3.42 mmol), cesium carbonate (2.04 g, 6.16 mmol), palladium (II) acetate (20.2 mg, 171 μmol) and BINAP (213 mg, 342 μmol) were dissolved in toluene (10 mL). Afterwards, (E)-tert-butyl (4-aminobut-2-en-1-yl)carbamate (957 mg, 5.14 mmol) was added under slight argon stream and the resulting mixture was heated to 120° C. for 4 h. The mixture was diluted with water (50 mL) and the product was extracted with DCM (2×50 mL). The organic layers were combined, dried over Na₂SO₄, filtered and evaporated. The residue was purified by column chromatography using DCM/[DCM: MeOH (9:1)] (100:00->50:50; 120 mL/min, 35 min, Biotage Sfär HC 100 g) (linear gradient) as eluent. The product containing fractions were combined, evaporated and dried in high vacuo to yield 992 mg (2.83 mmol, 83%) of a light brown solid. LCMS (Method B) retention time 3.60 min, [M+H]⁺=351.2. ¹H-NMR: (300 MHz, DMSO-d6) d 7.50-7.41 (m, 1H), 7.28 (d, J=1.8 Hz, 1H), 6.91 (t, J=5.9 Hz, 1H), 6.51 (d, J=8.4 Hz, 1H), 5.91 (t, J=6.0 Hz, 1H), 5.59-5.51 (m, 2H), 3.83 (s, 3H, CH₃), 3.76 (s, 5H), 3.55-3.46 (m, 2H), 1.35 (s, 9H, Boc).

### Preparation of Methyl (E)-3-(4-((tert-butoxycarbonyl)amino)but-2-en-1-yl)-2-imino-4-methoxy-2,3-dihydrobenzo[d]thiazole-6-carboxylate (XX3.2)

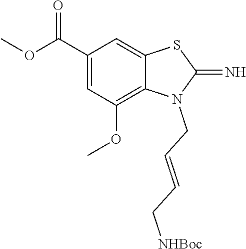 [SMILES: COC(=O)c1cc(OC)c2c(c1)sc(=N)n2C/C=C/CNC(=O)OC(C)(C)C]

Methyl (E)-4-((4-((tert-butoxycarbonyl)amino)but-2-en-1-yl)amino)-3-methoxybenzoate (1.90 g, 5.42 mmol) was dissolved in acetic acid (8 mL) and KSCN (2.11 g, 21.7 mmol) was added. The reaction mixture was stirred at room temperature for 30 min. Then, Br₂ (277 μL, 5.42 mmol) dissolved in acetic acid (2 mL) was added to the mixture and the reaction was stirred at room temperature for 3 h. The product formation was controlled by LC/MS. The mixture was quenched with water (50 mL). The solid was filtered off. The filtrate was adjusted to pH 9 with aq. ammonia solution (33%) and the product was extracted with ethyl acetate (2×80 mL). The combined organic layers were dried over sodium sulfate and filtered. The solvent was removed under reduced pressure. The residue was purified on silica by automated flash column chromatography on a Biotage Selekt (Biotage Sfär HC 50 g, 120 mL/min, 0-20% CH₃OH in CH₂Cl₂ in 30 min) to yield 407 mg (982 μmol, 18%) of a white solid. LCMS (Method A) retention time 1.56 min, [M+H]⁺=408.2. ¹H-NMR: (300 MHz, DMSO-d6) δ [ppm]=8.51 (brs, 1H), 7.70-7.62 (m, 1H), 7.43-7.35 (m, 1H), 6.90 (brs, 1H), 5.68-5.44 (m, 2H), 4.79 (d, J=5.1 Hz, 2H), 3.87 (s, 3H), 3.82 (s, 3H), 3.47 (d, J=5.6 Hz, 2H), 1.34 (s, 9H).

### Preparation of Methyl (Z)-3-((E)-4-((tert-butoxycarbonyl)amino)but-2-en-1-yl)-2-((1-ethyl-3-methyl-1H-pyrazole-5-carbonyl)imino)-4-methoxy-2,3-dihydrobenzo[d]thiazole-6-carboxylate (XX3.3)

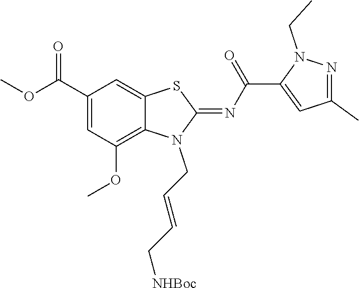

Compound XX3.2 (1.00 g, 2.54 mmol) and 1-ethyl-3-methyl-1H-pyrazole-5-carboxylic acid (450 mg, 2.95 mmol) were dissolved in DMF (10 mL). Afterwards, HATU (1.41 g, 3.68 mmol) and DIPEA (2.16 mL, 12.3 mmol) were added and the reaction mixture was stirred at room temperature for 30 min. The product formation was controlled by LC/MS. Then, water was added and the resulting solid was filtered off and washed with water. The product was dried in vacuo to give 1.60 g of a yellowish solid, which was used without further purification. LCMS (Method C) retention time 3.13 min, [M+Na]+=408.2. ¹H-NMR: (300 MHz, CDCl₃) δ [ppm]=8.04-7.91 (m, 1H), 7.64-7.58 (m, 1H), 6.83-6.76 (m, 1H), 5.93-5.67 (m, 2H), 5.45-5.37 (m, 2H), 4.70 (q, J=7.1 Hz, 2H), 4.53 (brs, 1H), 4.02 (s, 3H), 3.95 (s, 3H,), 3.77-3.67 (m, 2H), 2.31 (s, 3H), 1.47 (t, J=7.1 Hz, 3H), 1.39 (s, 9H).

### Preparation of Methyl (Z)-3-((E)-4-aminobut-2-en-1-yl)-2-((1-ethyl-3-methyl-1H-pyrazole-5-carbonyl)imino)-4-methoxy-2,3-dihydrobenzo[d]thiazole-6-carboxylate hydrochloride (XX3.4)

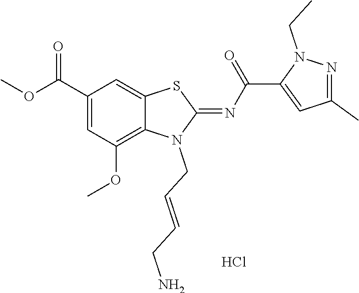

Compound XX3.3 (1.60 g, 2.94 mmol) was treated with 4 M HCl in dioxane (11.8 mL, 47.1 mmol). The resulting suspension was stirred at room temperature for 30 min. The product formation was controlled by LC/MS. The formed solid was filtered off, washed with diethyl ether and dried in vacuo to give 996 mg (2.08 mmol, 76%) of a yellowish solid. LCMS (Method A) retention time 1.87 min, [M+H]⁺=444.3. ¹H-NMR: (300 MHz, DMSO-d6) δ [ppm]=8.22-8.08 (m, 3H), 7.60-7.53 (m, 1H), 6.84-6.75 (m, 1H), 6.14-5.98 (m, 1H), 5.83-5.68 (m, 1H), 5.35 (d, J=5.8 Hz, 2H), 4.56 (q, J=7.1 Hz, 2H), 4.02 (s, 3H), 3.88 (s, 3H), 3.48-3.37 (m, 2H), 2.20 (s, 3H), 1.34 (t, J=7.1 Hz, 3H).

### Preparation of Methyl (Z)-3-((E)-4-((4-carbamoyl-2-(3-morpholinopropoxy)-6-nitrophenyl)amino)but-2-en-1-yl)-2-((1-ethyl-3-methyl-1H-pyrazole-5-carbonyl)imino)-4-methoxy-2,3-dihydrobenzo[d]thiazole-6-carboxylate trifluoroacetic acid salt (XX3.5)

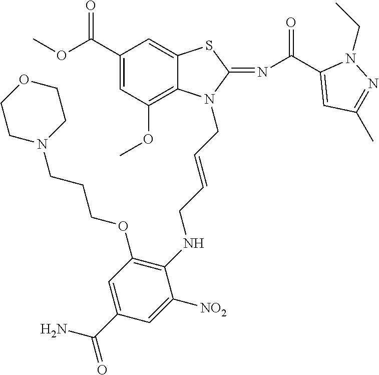 [SMILES: CCn1nc(C)cc1C(=O)/N=c1\sc2cc(C(=O)OC)cc(OC)c2n1C/C=C/CNc1c(OCCCN2CCOCC2)cc(C(N)=O)cc1[N+](=O)[O-]]

Compound XX3.4 (100 mg, 208 μmol) was suspended in dioxane (4 mL), 4-chloro-3-(3-morpholinopropoxy)-5-nitrobenzamide (71.5 mg, 208 μmol), DIPEA (73.2 UL, 415 μmol) and K₂CO₃ (86.2 mg, 624 μmol) and the mixture was stirred at 120° C. for 24 h. Again, K₂CO₃ (57.5 mg, 415 μmol) was added and the reaction mixture was stirred at 120° C. for 4 days. Then, K₂CO₃ (57.5 mg, 415 μmol) was added and the reaction mixture was stirred at 120° C. for 23 h. Again, 4-chloro-3-(3-morpholinopropoxy)-5-nitrobenzamide (17.8 mg, 52.0 μmol), DIPEA (36.6 μL, 208 μmol) and K₂CO₃ (57.5 mg, 415 μmol) were added and the reaction mixture was stirred at 120° C. for 4 h. The product formation was controlled by LC/MS. The reaction was cooled to room temperature and quenched with water (10 mL). The product was extracted with CH₂Cl₂/CH₃OH (9/1, 2×20 mL). The organic layer was dried over Na₂SO₄ and filtered. The solvent was removed under reduced pressure. The residue was purified by automated flash column chromatography on Büchi C-850 (XSelect® CSH™ Prep OBD™ C18 0.5 UM, 50 mm×150 mm, 100 mL/min; 15-100% CH₃CN in H₂O (+0.15% TFA) in 30 min). Product containing fractions were combined and freeze-dried to give 84 mg (97 μmol, 47%) of a yellowish solid. LCMS (Method B) retention time 2.84 min, [M+H]⁺=751.3. ¹H-NMR: (300 MHz, DMSO-d6) δ [ppm]=8.15-8.08 (m, 1H), 8.04-7.97 (m, 1H), 7.88 (s, 1H), 7.57 (s, 1H), 7.52-7.46 (m, 1H), 7.46-7.36 (m, 1H), 7.24 (s, 1H), 6.77-6.70 (m, 1H), 5.84-5.61 (m, 2H), 5.29 (d, J=5.0 Hz, 2H), 4.56 (q, J=7.1 Hz, 2H), 4.13-4.07 (m, 2H), 4.00-3.91 (m, 4H), 3.90 (s, 3H), 3.85 (s, 3H), 3.71-3.53 (m, 2H), 3.48-3.30 (m, 2H), 3.27-3.13 (m, 2H), 3.10-2.90 (m, 2H), 2.19 (s, 3H), 2.12-1.99 (m, 2H), 1.33 (t, J=7.1 Hz, 3H).

### Preparation of Methyl (Z)-3-((E)-4-((2-amino-4-carbamoyl-6-(3-morpholinopropoxy)phenyl)amino)but-2-en-1-yl)-2-((1-ethyl-3-methyl-1H-pyrazole-5-carbonyl)imino)-4-methoxy-2,3-dihydrobenzo[d]thiazole-6-carboxylate (XX3.6)

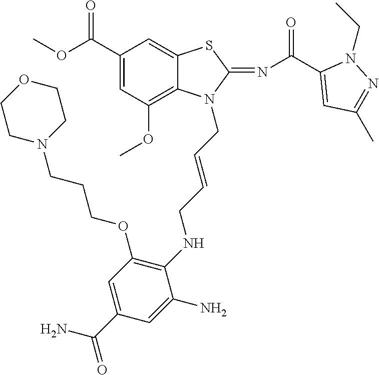 [SMILES: CCn1nc(C)cc1C(=O)/N=c1\sc2cc(C(=O)OC)cc(OC)c2n1C/C=C/CNc1c(N)cc(C(N)=O)cc1OCCCN1CCOCC1]

To a stirred solution of compound XX3.5 (1.73 g, 2.30 mmol) in CH₃OH (30 mL) was added sodium dithionite (4.01 g, 23.0 mmol) dissolved in water (10 mL) at 0° C. Then, NH₃ (30% aqueous solution, 2.29 mL, 17.3 mmol) was added to the mixture at 0° C. The reaction mixture was allowed to warm to room temperature and was stirred at room temperature for 1.5 h. The reaction progress was controlled by LC/MS. The reaction was diluted with H₂O (75 mL) and CH₂Cl₂ (100 mL) was added. The aqueous layer was extracted with CH₂Cl₂/CH₃OH (8:2, 5×50 mL). The organic layer was dried over sodium sulfate and filtered. The solvent was removed under reduced pressure to yield 1.31 g (1.82 mmol, 79%). The residue was used without further purification. LCMS (Method A) retention time 1.85 min, [M+H]⁺=721.4. ¹H-NMR: (300 MHz, DMSO-d6) δ [ppm]=8.16-8.09 (m, 1H), 7.57-7.47 (m, 2H), 6.92 (brs, 1H), 6.82-6.77 (m, 1H), 6.76-6.71 (m, 1H), 6.67-6.60 (m, 1H), 5.89-5.64 (m, 2H), 5.29 (d, J=5.2 Hz, 2H), 4.65-4.50 (m, 4H), 3.99-3.83 (m, 7H), 3.74 (t, J=6.2 Hz, 2H), 3.60 (t, J=5.9 Hz, 2H), 3.48 (t, J=4.6 Hz, 4H), 2.28-2.16 (m, 9H), 1.65 (p, J=6.6 Hz, 2H), 1.33 (t, J=7.1 Hz, 3H).

### Preparation of Methyl (Z)-3-((E)-4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-((1-ethyl-3-methyl-1H-pyrazole-5-carbonyl)imino)-4-methoxy-2,3-dihydrobenzo[d]thiazole-6-carboxylate trifluoroacetic acid salt (XX3.7)

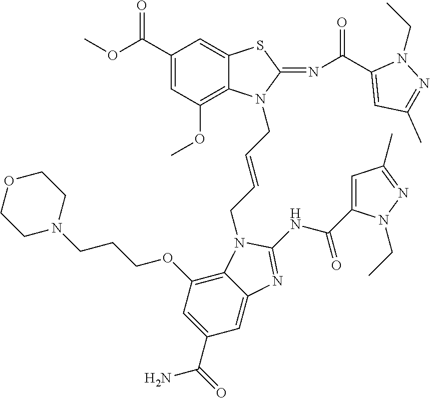 [SMILES: CCn1nc(C)cc1C(=O)/N=c1\sc2cc(C(=O)OC)cc(OC)c2n1C/C=C/Cn1c(NC(=O)c2cc(C)nn2CC)nc2cc(C(N)=O)cc(OCCCN3CCOCC3)c21]

Compound XX3.6 (250 mg, 347 μmol) was dissolved in anhydrous DMF (4 mL) and cooled to 0° C. Then, 1-ethyl-3-methyl-1H-pyrazole-5-carbonyl isothiocyanate (0.2 M in dioxane, 867 μL, 173 μmol) was added dropwise and the reaction was stirred at 0° C. for 1.5 h. Again, 1-ethyl-3-methyl-1H-pyrazole-5-carbonyl isothiocyanate (0.2 M in dioxane, 867 μL, 173 μmol) was added and the mixture was stirred at 0° C. for 30 min. Then, EDC·HCl (83.1 mg, 434 μmol) and Et₃N (122 μL, 867 μmol) were added and the reaction was stirred at room temperature for 18 h. Again, EDC·HCl (16.6 mg, 87.0 μmol) and Et₃N (24.3 μL, 173 μmol) were added and the reaction was stirred at room temperature for 72 h. The product formation was controlled by LC/MS. The reaction was diluted with water/sat. aqueous NH₄Cl (3:1, 20 mL) and extracted with CH₂C₁₂/CH₃OH (3:1, 3×25 mL). The combined organic layers were dried over sodium sulfate and filtered. The solvent was removed under reduced pressure and the residue was purified by flash column chromatography on Büchi C-850 (XSelect® CSH™ Prep OBD™ C18 0.5 μM, 50 mm×150 mm, 100 mL/min; 5-100% CH₃CN in H₂O (+0.15% TFA) in 30 min) to obtain 324 mg (325 μmol, 94%) of an off-white solid. LCMS (Method C) retention time 2.93 min, [M+H]⁺=882.6. ¹H-NMR: (300 MHz, DMSO-d6) δ [ppm]=9.83 (s, 1H), 8.15-8.07 (m, 1H), 7.91 (s, 1H), 7.69-7.63 (m, 1H), 7.45-7.38 (m, 1H), 7.34 (s, 1H), 7.29-7.23 (m, 1H), 6.69-6.63 (m, 1H), 6.49-6.43 (m, 1H), 5.91-5.67 (m, 2H), 5.31 (d, J=4.9 Hz, 2H), 4.91 (d, J=4.8 Hz, 2H), 4.58-4.44 (m, 4H), 3.98 (t, J=5.8 Hz, 2H), 3.92-3.86 (m, 5H), 3.68 (s, 3H), 3.65-3.51 (m, 2H), 3.40-3.23 (m, 2H), 3.16 (t, J=8.0 Hz, 2H), 3.02-2.96 (m, 2H), 2.16-2.07 (m, 6H), 1.98-1.86 (m, 2H), 1.35-1.20 (m, 6H).

### Preparation of (Z)-3-((E)-4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-((1-ethyl-3-methyl-1H-pyrazole-5-carbonyl)imino)-4-methoxy-2,3-dihydrobenzo[d]thiazole-6-carboxylic acid (XX3.8)

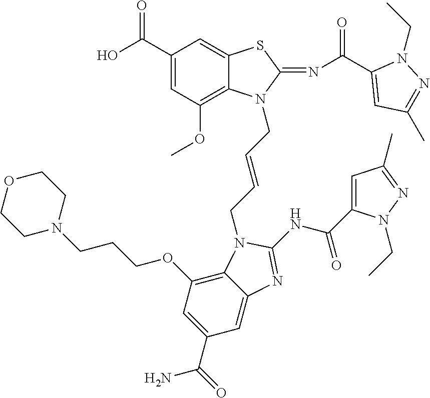 [SMILES: CCn1nc(C)cc1C(=O)/N=c1\sc2cc(C(=O)O)cc(OC)c2n1C/C=C/Cn1c(NC(=O)c2cc(C)nn2CC)nc2cc(C(N)=O)cc(OCCCN3CCOCC3)c21]

Compound XX3.7 (100 mg, 100 μmol) was dissolved in CH₃OH/THF/H₂O (2:2:1; 2.5 mL). Afterwards, LiOH (14 mg, 602 μmol) was added and the resulting reaction mixture was stirred at room temperature for 18 h. The reaction progress was controlled by LC/MS. The organic solvents were removed under reduced pressure. The aqueous phase was cooled to 0° C. and neutralized with 1 M aq. HCl. The precipitate was filtered and washed with water. The solid was dissolved/suspended in CH₃CN/H₂O (1:1, 15 mL) and freeze-dried to obtain 74 mg (85 μmol, 85%) of a white solid. The product was used without further purification. LCMS (Method A) retention time 1.96 min, [M+H]⁺=868.6. ¹H-NMR: (300 MHz, DMSO-d6) δ [ppm]=13.16 (brs, 1H), 12.84 (brs, 1H), 10.77 (brs, 1H), 8.09 (d, J=1.5 Hz, 1H), 7.93 (s, 1H), 7.66 (d, J=1.2 Hz, 1H), 7.50 (d, J=1.5 Hz, 1H), 7.32 (d, J=1.4 Hz, 2H), 6.63 (s, 1H), 6.46 (s, 1H), 5.97-5.83 (m, 1H), 5.84-5.69 (m, 1H), 5.31 (d, J=5.3 Hz, 2H), 4.94 (d, J=4.9 Hz, 2H), 4.51 (q, J=7.1 Hz, 4H), 4.04 (t, J=5.9 Hz, 2H), 3.94-3.83 (m, 2H), 3.79-3.66 (m, 5H), 3.31-3.21 (m, 2H), 3.21-3.05 (m, 2H), 3.01-2.91 (m, 2H), 2.14-2.06 (m, 5H), 2.05-1.92 (m, 2H), 1.34-1.20 (m, 6H).

### Preparation of 1-((E)-4-((Z)-2-((1-ethyl-3-methyl-1H-pyrazole-5-carbonyl)imino)-6-(hydrazinecarbonyl)-4-methoxybenzo[d]thiazol-3(2H)-yl)but-2-en-1-yl)-2-(1-ethyl-3-methyl-1H-pyrazole-5-carboxamido)-7-(3-morpholinopropoxy)-1H-benzo[d]imidazole-5-carboxamide trifluoroacetic acid salt (Compound XXXIV)

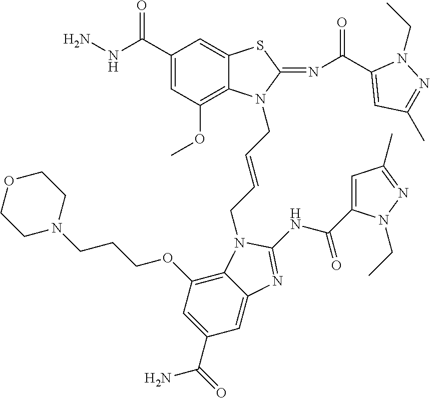 [SMILES: CCn1nc(C)cc1C(=O)/N=c1\sc2cc(C(=O)NN)cc(OC)c2n1C/C=C/Cn1c(NC(=O)c2cc(C)nn2CC)nc2cc(C(N)=O)cc(OCCCN3CCOCC3)c21]

BeSp-0708/1 (72.0 mg, 82.9 μmol) was suspended in DMF (3 mL). Afterwards, DIPEA (43.3 μL, 248 μmol), NHS (19.5 mg, 165 μmol) and DIC (25.7 μL, 165 μmol) were added and the resulting reaction mixture was stirred at 45° C. for 3 h. Afterwards, the reaction mixture was stirred at room temperature for 17 h. The reaction progress was controlled by LC/MS. Then, 1 M hydrazine in THF (414 μL, 414 μmol) and DIPEA (72.2 μL, 141 μmol) were added and the resulting reaction mixture was stirred (Start: 6 Oct. 2023; 08:00 am) at room temperature for 1 h. The reaction progress was controlled by LC/MS. The solvent was removed under reduced pressure. The residue was purified on RP18 silica by prepHPLC (Method I). Product containing fractions were freeze-dried to obtain 41.0 mg (41.2 μmol, 79%, purity 96.7% by HPLC Method A) of a white solid. LCMS (Method C) retention time 2.94 min, [M+H]⁺=882.5.

### Example 40: Synthetic Scheme for Compound XXXV

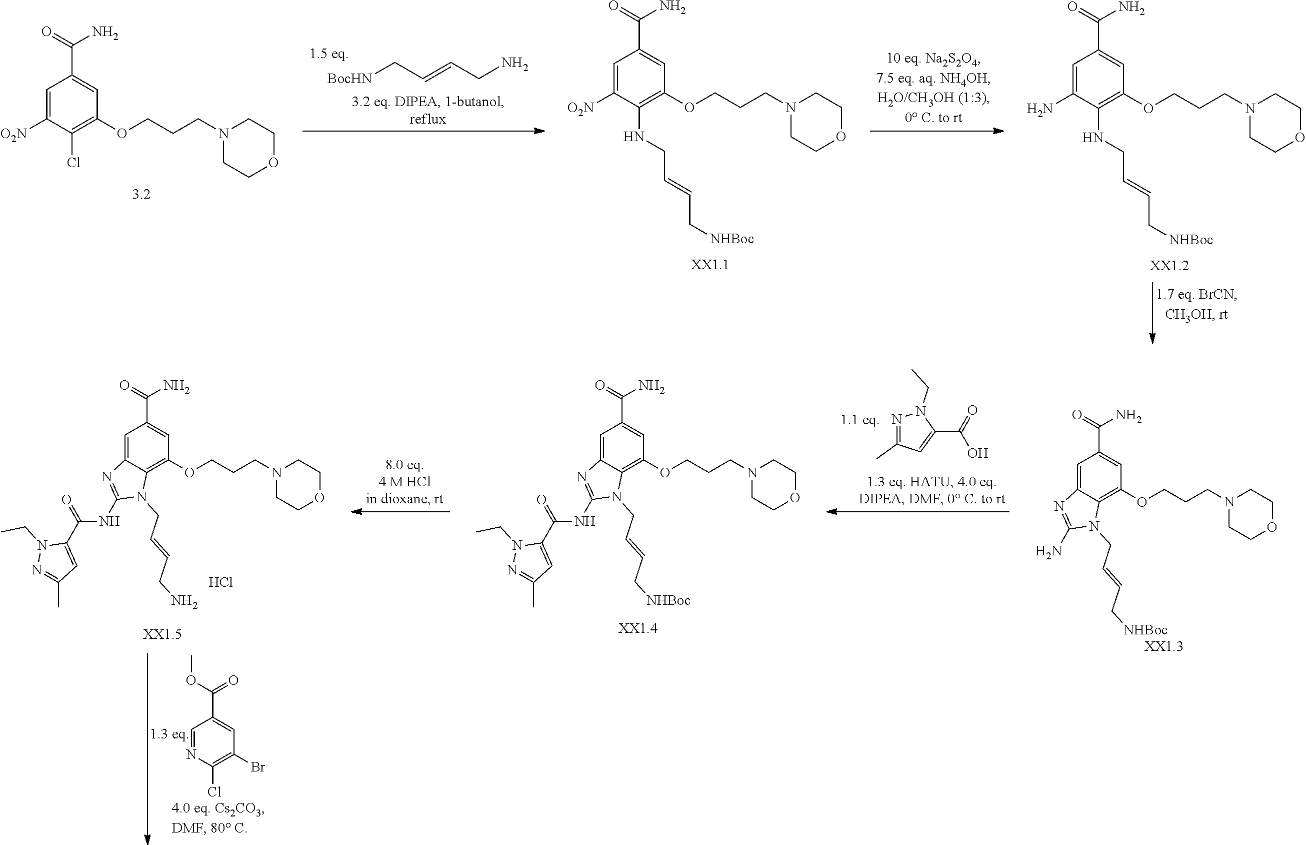

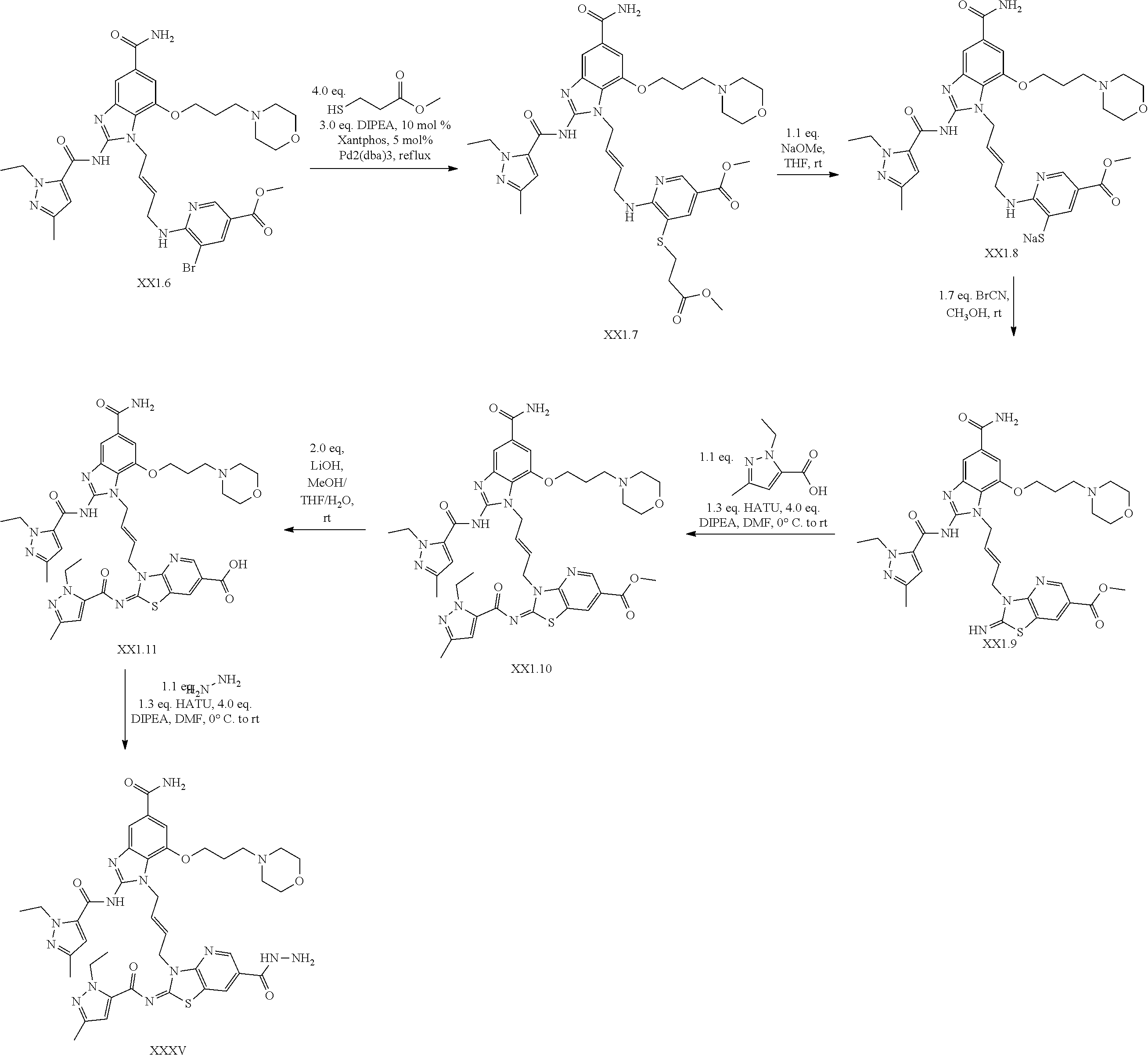

### Preparation of tert-Butyl (E)-(4-((4-carbamoyl-2-(3-morpholinopropoxy)-6-nitrophenyl)amino)but-2-en-1-yl)carbamate (XX1.1)

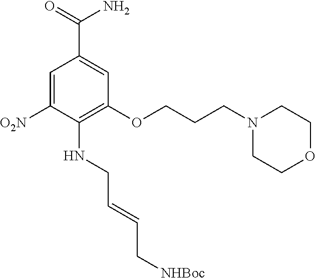 [SMILES: CC(C)(C)OC(=O)NC/C=C/CNc1c(OCCCN2CCOCC2)cc(C(N)=O)cc1[N+](=O)[O-]]

To a suspension of tert-butyl N-[(2E)-4-aminobut-2-en-1-yl]carbamate (4.27 g, 22.9 mmol) and 4-chloro-3-(3-morpholinopropoxy)-5-nitrobenzamide (5.25 g, 15.3 mmol) in n-butanol (100 mL) was added DIPEA (6.01 mL, 48.9 mmol) and the resulting reaction mixture was heated to 120° C. for 48 h. The solvent was removed in vacuo. The residue was purified on silica by automated flash column chromatography on Biotage Selekt (Biotage Sfär Silica HC, 20 μM, 100 g, 100 mL/min; 5-15% CH₃OH in CH₂Cl₂ in 30 min). Product containing fractions were combined to give 7.02 g (14.2 mmol, 93%) of a red solid. LCMS (Method C) retention time 2.97 min, [M+H]⁺=494.1. ¹H-NMR (300 MHz, DMSO-d6): d [ppm]=8.19 (d, J=1.9 Hz, 1H), 8.02 (brs, 1H), 7.76 (t, J=6.1 Hz, 1H), 7.56 (d, J=1.9 Hz, 1H), 7.29 (brs, 1H), 6.92 (t, J=5.9 Hz, 1H), 5.65-5.48 (m, 2H), 4.19-4.05 (m, 4H), 3.67-3.41 (m, 6H), 2.48-2.28 (m, 6H), 2.03-1.90 (m, 2H), 1.41-1.19 (m, 9H).

### Preparation of tert-Butyl (E)-(4-((2-amino-4-carbamoyl-6-(3-morpholinopropoxy)phenyl)amino)but-2-en-1-yl)carbamate (XX1.2)

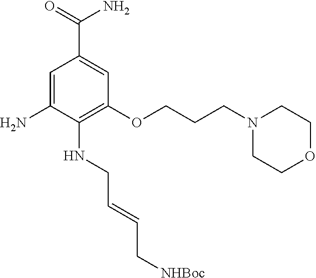 [SMILES: CC(C)(C)OC(=O)NC/C=C/CNc1c(N)cc(C(N)=O)cc1OCCCN1CCOCC1]

To a stirred solution of compound XX.1 (7.02 g, 14.2 mmol) in CH₃OH (210 mL) was added sodium dithionite (24.8 g, 142 mmol) dissolved in water (70 mL) at 0° C. Then, NH₃ (30% aqueous solution, 14.2 mL, 107 mmol) was added to the mixture at 0° C. The reaction was allowed to warm to room temperature and stirred at room temperature for 2 h. The reaction progress was controlled by LCMS. The reaction was diluted with H₂O (500 mL) and CH₂Cl₂ (700 mL) was added. The aqueous layer was extracted with CH₂Cl₂/CH₃OH (2×300 mL). The organic layer was dried over Na₂SO₄ and filtered. The solvent was removed under reduced pressure to obtain 5.00 g (10.8 mmol, 76%) of a yellowish foam. The compound was used without further purification. LCMS (Method C) retention time 1.78 min, [M+H]⁺=464.1. ¹H-NMR (300 MHz, DMSO-d6): d [ppm]=7.58 (brs, 1H), 7.04-6.81 (m, 3H), 6.78 (d, J=1.9 Hz, 1H), 5.69-5.47 (m, 2H), 4.65 (brs, 2H), 3.98 (t, J=6.2 Hz, 2H), 3.81 (t, J=7.0 Hz, 1H), 3.63-3.43 (m, 8H), 2.47-2.30 (m, 6H), 1.96-1.81 (m, 2H), 1.37 (s, 9H).

### Preparation of tert-Butyl (E)-(4-(2-amino-5-carbamoyl-7-(3-morpholinopropoxy)-1H-benzo[d]imidazol-1-yl)but-2-en-1-yl)carbamate (XX1.3)

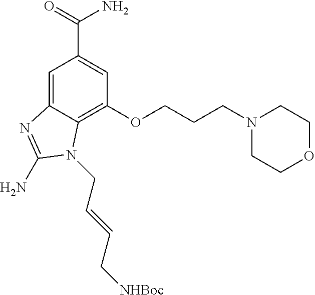 [SMILES: CC(C)(C)OC(=O)NC/C=C/Cn1c(N)nc2cc(C(N)=O)cc(OCCCN3CCOCC3)c21]

Compound XX1.2 (5.00 g, 10.8 mmol) was dissolved in CH₃OH (100 mL) and cooled to 0° C. Afterwards. cyanogen bromide (1.94 g, 18.3 mmol) was added and the mixture was warmed to room temperature. The resulting mixture was stirred at room temperature for 24 h. The reaction progress was controlled by LCMS. All volatiles were removed under reduced pressure. The residue was purified on silica by automated flash column chromatography on Biotage Selekt (Biotage Sfär Silica HC, 20 μM, 200 g, 120 mL/min; 5-15% CH₃OH (+10% aq. NH₄OH (35%)) in CH₂Cl₂ in 45 min) to yield 3.46 g (7.08 mmol, 66%). LCMS (Method C) retention time 1.86 min, [M+H]⁺=489.1. ¹H-NMR (300 MHz, DMSO-d6): d [ppm]=7.77 (brs, 1H), 7.39 (d, J=1.3 Hz, 1H), 7.13-6.97 (m, 2H), 6.96-6.85 (m, 1H), 6.38 (s, 2H), 5.71-5.42 (m, 2H), 4.78 (d, J=5.4 Hz, 2H), 4.11 (t, J=6.3 Hz, 2H), 3.63-3.54 (m, 4H), 3.53-3.43 (m, 2H), 2.49-2.32 (m, 6H), 2.01-1.86 (m, 2H), 1.34 (s, 9H).

### Preparation of tert-Butyl (E)-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)carbamate trifluoroacetic acid salt (XX1.4)

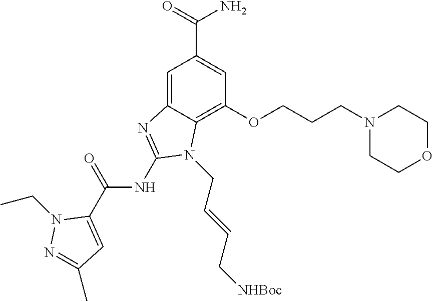 [SMILES: CCn1nc(C)cc1C(=O)Nc1nc2cc(C(N)=O)cc(OCCCN3CCOCC3)c2n1C/C=C/CNC(=O)OC(C)(C)C]

Compound XX1.3 (1.50 g, 3.07 mmol) and 1-ethyl-3-methyl-1H-pyrazole-5-carboxylic acid (521 mg, 3.38 mmol) were dissolved in DMF (20 mL) and afterwards treated with DIPEA (2.03 mL, 12.3 mmol) for 15 min at 0° C. Then, HATU (1.52 g, 3.99 mmol) was added and the resulting yellowish reaction mixture was stirred at room temperature for 1 h. The product formation was controlled by LCMS. The solvent was removed in vacuo and the residue was purified on RP18 silica by automated flash column chromatography on Büchi C-850 (XSelect® CSH™ Prep OBD™ 0.5 μM, 50 mm×150 mm, 100 mL/min; 10-100% CH₃CN in H₂O (+0.15% TFA) in 47 min). Product containing fractions were combined and freeze-dried to obtain 1.91 g (2.59 mmol, 84%) of a colorless solid. LCMS (Method C) retention time 3.15 min, [M+H]⁺=625.2. ¹H-NMR (300 MHz, DMSO-d6): d [ppm]=9.78 (brs, 1H), 7.97 (brs, 1H), 7.69 (d, J=1.2 Hz, 1H), 7.44-7.32 (m, 2H), 6.91 (brs, 1H), 6.66-6.60 (m, 1H), 5.84-5.69 (m, 1H), 5.58-5.43 (m, 1H), 5.03-4.89 (m, 2H), 4.68-4.54 (m, 2H), 4.27 (t, J=5.9 Hz, 2H), 4.11-3.96 (m, 2H), 3.76-3.60 (m, 2H), 3.60-3.42 (m, 4H), 3.38-3.26 (m, 2H), 3.25-3.06 (m, 2H), 2.33-2.11 (m, 5H), 1.43-1.08 (m, 12H).

### Preparation of (E)-1-(4-aminobut-2-en-1-yl)-2-(1-ethyl-3-methyl-1H-pyrazole-5-carboxamido)-7-(3-morpholinopropoxy)-1H-benzo[d]imidazole-5-carboxamide hydrochloric acid (XX1.5)

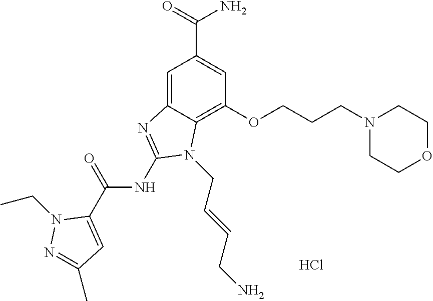 [SMILES: CCn1nc(C)cc1C(=O)Nc1nc2cc(C(N)=O)cc(OCCCN3CCOCC3)c2n1C/C=C/CN ; Cl]

Compound XX1.4 (1.90 g, 2.57 mmol) was dissolved/suspended in CH₂Cl₂ (5 mL). Afterwards, 4 M HCl in dioxane (5.14 mL) was added and the resulting mixture was stirred at room temperature for 2.5 h. The reaction progress was controlled by LCMS. Afterwards, aqueous HCl (0.15% in H₂O) was added and then all volatiles were removed in vacuo. The residue was purified on RP18 silica by automated flash column chromatography on Büchi C-850 (XSelect® CSH™ Prep OBD™ 0.5 M, 50 mm×150 mm, 100 ml/min; 10-100% CH₃CN in H₂O (+0.15% HCl) in 47 min). Product containing fractions were combined and freeze-dried to yield 1.41 g (2.51 mmol, 98%) of a white solid. LCMS (Method C) retention time 2.03 min, [M+H]⁺=525.1. ¹H-NMR (300 MHz, DMSO-d6): d [ppm]=11.65 (s, 1H), 8.26 (brs, 3H), 7.71 (d, J=1.2 Hz, 1H), 7.52-7.29 (m, 2H), 6.74 (s, 1H), 6.19-6.05 (m, 1H), 5.70-5.55 (m, 1H), 5.04 (d, J=5.4 Hz, 2H), 4.68-4.54 (m, 2H), 4.31 (t, J=6.1 Hz, 2H), 4.04-3.84 (m, 4H), 3.59-3.06 (m, 9H), 2.45-2.30 (m, 2H), 2.23-2.17 (m, 3H), 1.36 (t, J=7.1 Hz, 3H).

### Preparation of Methyl (E)-5-bromo-6-((4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)amino)nicotinate trifluoroacetic acid salt (XX1.6)

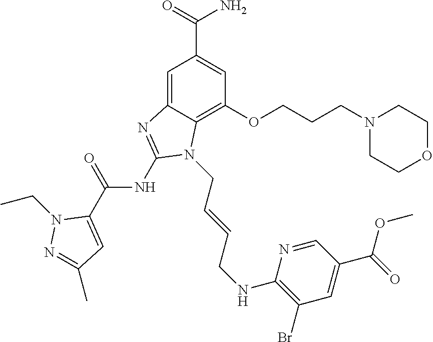 [SMILES: CCn1nc(C)cc1C(=O)Nc1nc2cc(C(N)=O)cc(OCCCN3CCOCC3)c2n1C/C=C/CNc1ncc(C(=O)OC)cc1Br]

Compound XX1.5 (1.29 g, 2.30 mmol) and methyl 5-bromo-6-chloronicotinate (748.7 mg, 2.99 mmol) were dissolved in DMF (40 mL). Afterwards, Cs₂CO₃ (3.05 g, 9.20 mmol) was added and the resulting reaction mixture was heated to 80° C. for 18 h. The product formation was controlled by LCMS. The suspension was filtered and the solvent was removed in vacuo. The residue was purified on RP18 silica by automated flash column chromatography on Büchi C-850 (XSelect® CSH™ Prep OBD™ 0.5 μM, 50 mm×250 mm, 100 mL/min; 5-100% CH₃CN in H₂O (+0.15% TFA) in 45 min). Product containing fractions were combined and freeze-dried to obtain 648 mg (33%, 759 μmol) of a white solid. LCMS (Method C) retention time 3.56 min, [M+2H]²⁺=369.5. ¹H-NMR (300 MHz, DMSO-d6): d [ppm]=12.76 (brs, 1H), 9.99 (brs, 1H), 8.47 (d, J=2.0 Hz, 1H), 8.07 (d, J=2.0 Hz, 1H), 7.95 (brs, 1H), 7.68 (d, J=1.2 Hz, 1H), 7.41-7.25 (m, 3H), 6.56 (d, J=0.6 Hz, 1H), 5.89-5.61 (m, 2H), 4.94 (d, J=5.3 Hz, 2H), 4.56 (q, J=7.1 Hz, 2H), 4.21 (t, J=5.9 Hz, 2H), 4.08-3.93 (m, 4H), 3.79 (s, 3H), 3.67 (t, J=12.1 Hz, 2H), 3.46 (d, J=12.2 Hz, 2H), 3.29 (t, J=8.0 Hz, 2H), 3.21-3.01 (m, 2H), 2.23-2.12 (m, 5H), 1.30 (t, J=7.1 Hz, 3H).

### Preparation of Methyl (E)-6-((4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)amino)-5-((3-methoxy-3-oxopropyl)thio) nicotinate trifluoroacetic acid salt (XX1.7)

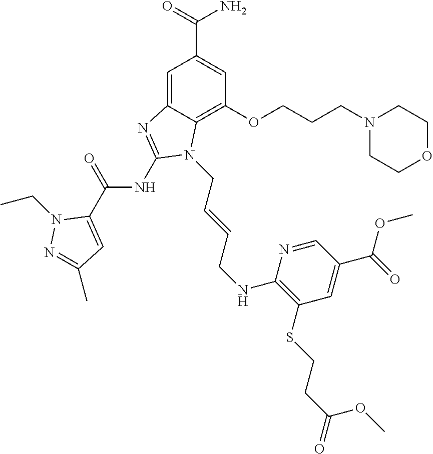 [SMILES: CCn1nc(C)cc1C(=O)Nc1nc2cc(C(N)=O)cc(OCCCN3CCOCC3)c2n1C/C=C/CNc1ncc(C(=O)OC)cc1SCCC(=O)OC]

Methyl (E)-5-bromo-6-((4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)amino)nicotinate (618 mg, 725 μmol) was co-evaporated with anhydrous 1,4-dioxane (5 mL). The residue was dissolved in anhydrous 1,4-dioxane (7.5 mL), and DIPEA (372 μL, 2.17 mmol) and methyl 3-mercaptopropanoate (321 μL, 2.90 mmol) were added. Afterwards, Xantphos (41.9 mg, 72.5 μmol) and Pd₂(dba)₃ (33.2 mg, 36.2 μmol) were added under slight argon stream and the resulting mixture was heated to 130° C. (temp. on heat plate) for 2 h. The product formation was controlled by LCMS. Water was added and afterwards all solvents were removed in vacuo. The residue (filtered through syringe filter PVDF-45/25) was purified on RP18 silica by automated flash column chromatography on Büchi C-850 (XSelect® CSH™ Prep OBD™ 0.5 M, 50 mm×250 mm, 100 ml/min; 10-100% CH₃CN in H₂O (+0.15% TFA) in 47 min). Product containing fractions were freeze dried (CH₃CN/H₂O; 1:1) to obtain 424 mg (475 μmol, 66%) of a white solid. LCMS (Method C) retention time 2.95 min, [M+H]⁺=778.2. ¹H-NMR (300 MHz, DMSO-d6): d [ppm]=12.82 (s, 1H), 9.98 (s, 1H), 8.49 (d, J=2.2 Hz, 1H), 7.98-7.88 (m, 2H), 7.67 (d, J=1.2 Hz, 1H), 7.42-7.21 (m, 3H), 6.55 (d, J=0.6 Hz, 1H), 5.89-5.61 (m, 2H), 4.94 (d, J=5.2 Hz, 2H), 4.56 (q, J=7.1 Hz, 2H), 4.21 (t, J=5.9 Hz, 2H), 4.11-3.91 (m, 4H), 3.79 (s, 3H), 3.67 (t, J=12.1 Hz, 2H), 3.53-3.40 (m, 5H), 3.29 (t, J=8.0 Hz, 2H), 3.21-3.01 (m, 2H), 2.87 (t, J=6.8 Hz, 2H), 2.45 (t, J=6.8 Hz, 2H), 2.22-2.11 (m, 5H), 1.30 (t, J=7.1 Hz, 3H).

### Preparation of Sodium (E)-2-((4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)amino)-5-(methoxycarbonyl)pyridine-3-thiolate (XX1.8)

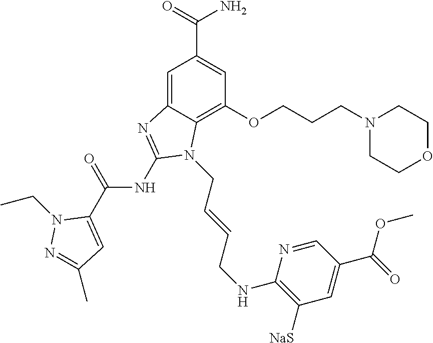

Methyl (E)-6-((4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)amino)-5-((3-methoxy-3-oxopropyl)thio) nicotinate (400 mg, 449 μmol) was dissolved in anhydrous THF (5 mL) and 25% NaOCH₃ in CH₃OH (410 μL, 1.79 mmol) was added under slight argon stream and the resulting mixture was stirred for 2 h. The product formation was controlled by LCMS. The mixture was diluted with CH₂Cl₂ (5 mL) and the resulting solid was filtered and washed with CH₂Cl₂ (3×2 mL). The solid was suspended in CH₃CN (5 mL) and stirred at room temperature for 1 h. The solid was filtered and washed with cold CH₃CN (2×2 mL). After drying in high vacuum the crude product (311 mg) was used without further purification. LCMS (Method C) retention time 2.22 min, [M+H]⁺=692.2.

### Preparation of Methyl (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-imino-2,3-dihydrothiazolo[4,5-b]pyridine-6-carboxylate (XX1.9)

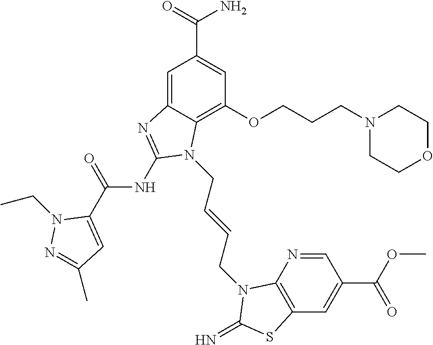 [SMILES: CCn1nc(C)cc1C(=O)Nc1nc2cc(C(N)=O)cc(OCCCN3CCOCC3)c2n1C/C=C/Cn1c(=N)sc2cc(C(=O)OC)cnc21]

Compound XX.8 (300 mg, 420 μmol) was dissolved in CH₃OH (5 mL) and cooled to 0° C. Afterwards, cyanogen bromide (53.4 mg, 504 μmol) was added and the mixture was stirred at 0° C. for 1 h. Afterwards, the mixture was warmed to room temperature and stirred for 1 h. The reaction progress was controlled by LCMS. The resulting solid was filtered and washed with ice-cold CH₃OH (3 mL). The solid (358 mg) was dried in vacuo and used without further purification. LCMS (Method C) retention time 1.99 min, [M+H]⁺=717.2.

### Preparation of Methyl (E)-3-((E)-4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-((1-ethyl-3-methyl-1H-pyrazole-5-carbonyl)imino)-2,3-dihydrothiazolo[4,5-b]pyridine-6-carboxylate trifluoroacetic acid salt (XX1.10)

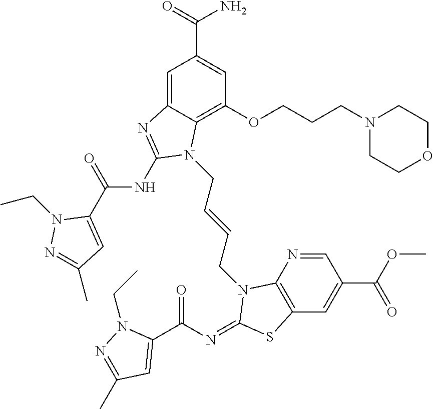 [SMILES: CCn1nc(C)cc1C(=O)/N=c1/sc2cc(C(=O)OC)cnc2n1C/C=C/Cn1c(NC(=O)c2cc(C)nn2CC)nc2cc(C(N)=O)cc(OCCCN3CCOCC3)c21]

Compound XX.9 (300 mg, 419 mmol) and 1-ethyl-3-methyl-1H-pyrazole-5-carboxylic acid (71.0 mg, 460 μmol) were dissolved in DMF (5 mL) and afterwards treated with DIPEA (277 μL, 1.67 mmol) for 15 min at 0° C. Then, HATU (207 mg, 544 μmol) was added and the resulting yellowish reaction mixture was stirred at room temperature for 1 h. The product formation was controlled by LCMS. The solvent was removed in vacuo and the residue was purified on RP18 silica by automated flash column chromatography on Büchi C-850 (XSelect® CSH™ Prep OBD™ 0.5 μM, 50 mm×150 mm, 100 mL/min; 10-100% CH₃CN in H₂O (+0.15% TFA) in 47 min). Product containing fractions were combined and freeze-dried (CH₃CN/H₂O=1:1) to give 123 mg (127.0 μmol, 34% over three steps) of a white solid. LCMS (Method C) retention time 3.32 min, [M+H]⁺=853.2.

### Preparation of (E)-3-((E)-4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-((1-ethyl-3-methyl-1H-pyrazole-5-carbonyl)imino)-2,3-dihydrothiazolo[4,5-b]pyridine-6-carboxylic acid (XX1.11)

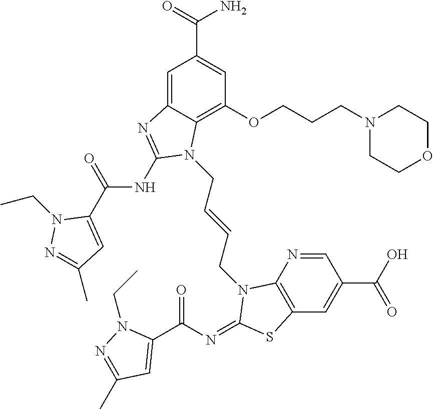 [SMILES: CCn1nc(C)cc1C(=O)/N=c1/sc2cc(C(=O)O)cnc2n1C/C=C/Cn1c(NC(=O)c2cc(C)nn2CC)nc2cc(C(N)=O)cc(OCCCN3CCOCC3)c21]

Methyl (E)-3-((E)-4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-((1-ethyl-3-methyl-1H-pyrazole-5-carbonyl)imino)-2,3-dihydrothiazolo[4,5-b]pyridine-6-carboxylate (122 mg, 126 μmol) was dissolved in CH₃OH/THF/H₂O (2:2:1; 5 mL). Afterwards, LiOH (9.06 mg, 378 μmol) was added and the resulting reaction mixture was stirred at room temperature for 4 h. The reaction progress was controlled by LCMS. The organic solvents were removed in vacuo (rotary evaporator). The aqueous phase was cooled to 0° C. and neutralized with 1 M aq. HCl. The precipitate was filtered and washed with water (2 mL). The solid was dissolved/suspended in CH₃CN/H₂O (1:1, 25 mL) and freeze-dried. The obtained product (90.8 mg) was used without further purification. LCMS (Method C) retention time 3.13 min, [M+2H]²⁺=420.2. ¹H-NMR (300 MHz, DMSO-d6): d [ppm]=12.80 (s, 1H), 8.93 (d, J=1.9 Hz, 1H), 8.85 (d, J=1.9 Hz, 1H), 7.92 (s, 1H), 7.63 (s, 1H), 7.34-7.28 (m, 2H), 6.61 (d, J=0.7 Hz, 1H), 6.45 (s, 1H), 6.02 (dt, J=15.5, 5.3 Hz, 1H), 5.79 (dd, J=15.2, 6.2 Hz, 1H), 5.07 (d, J=5.7 Hz, 2H), 4.94 (d, J=5.2 Hz, 2H), 4.58-4.42 (m, 4H), 4.08 (t, J=5.9 Hz, 2H), 3.73-3.65 (m, 5H), 3.05-2.63 (m, 4H), 2.19-2.03 (m, 6H), 1.96-1.90 (m, 2H), 1.33-1.17 (m, 6H).

### Preparation of 1-((E)-4-((E)-2-((1-ethyl-3-methyl-1H-pyrazole-5-carbonyl)imino)-6-(hydrazinecarbonyl)thiazolo[4,5-b]pyridin-3(2H)-yl)but-2-en-1-yl)-2-(1-ethyl-3-methyl-1H-pyrazole-5-carboxamido)-7-(3-morpholinopropoxy)-1H-benzo[d]imidazole-5-carboxamide trifluoroacetic acid salt (Compound XXXV)

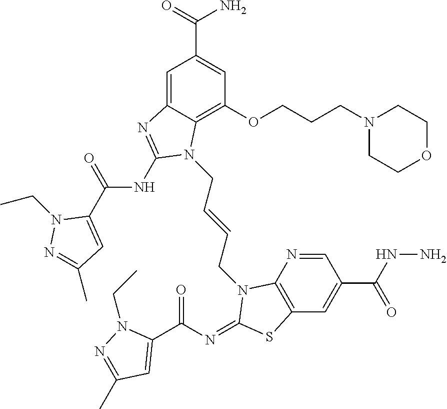 [SMILES: CCn1nc(C)cc1C(=O)/N=c1/sc2cc(C(=O)NN)cnc2n1C/C=C/Cn1c(NC(=O)c2cc(C)nn2CC)nc2cc(C(N)=O)cc(OCCCN3CCOCC3)c21]

Compound XX.11 (51.7 mg) was suspended in DMF (3 mL). Afterwards, DIPEA (32.2 L), NHS (14.5 mg) and DIC (19.1 μL) were added and the resulting reaction mixture was stirred at 40° C. for 3 h. Afterwards, the reaction was stirred at room temperature for 15 h. The reaction progress was controlled by LC/MS. Then, 1 M hydrazine in THF (308 μL) and DIPEA (32.2 μL) were added and the resulting reaction mixture was stirred at room temperature for 1 h. The reaction progress was controlled by LC/MS. The solvent was removed in vacuo (oil pump vacuo) and freeze-dried (CH₃CN/H₂O 1:1; 10 mL). The residue was purified on RP18 silica by prepHPLC (Method I). Product containing fractions were freeze-dried to obtain 46.9 mg (79%, purity 95.9% by HPLC Method A) of a white solid.

### Example 41: PBMC and THP-1 Assays of Compounds XXVIII-XXXV

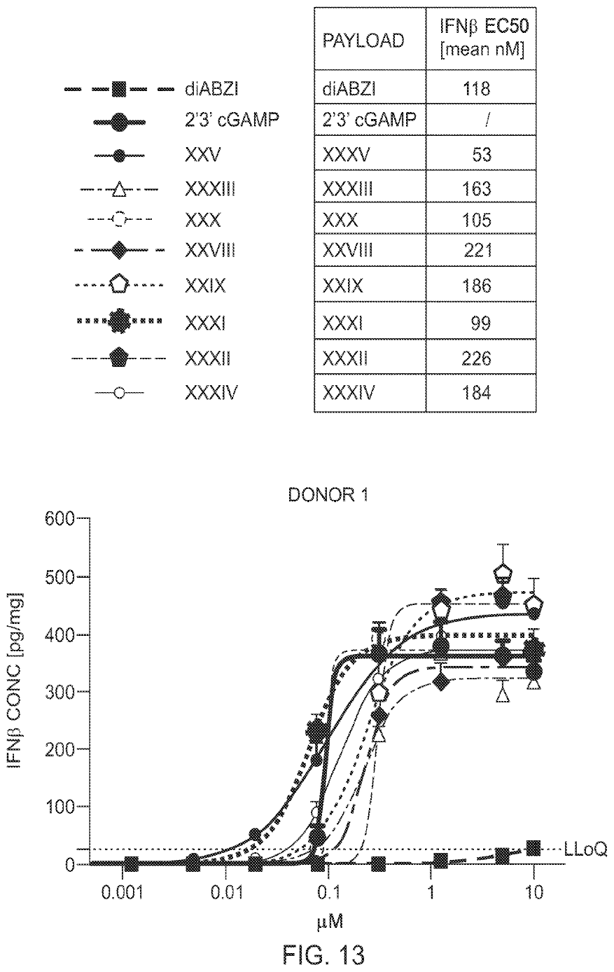

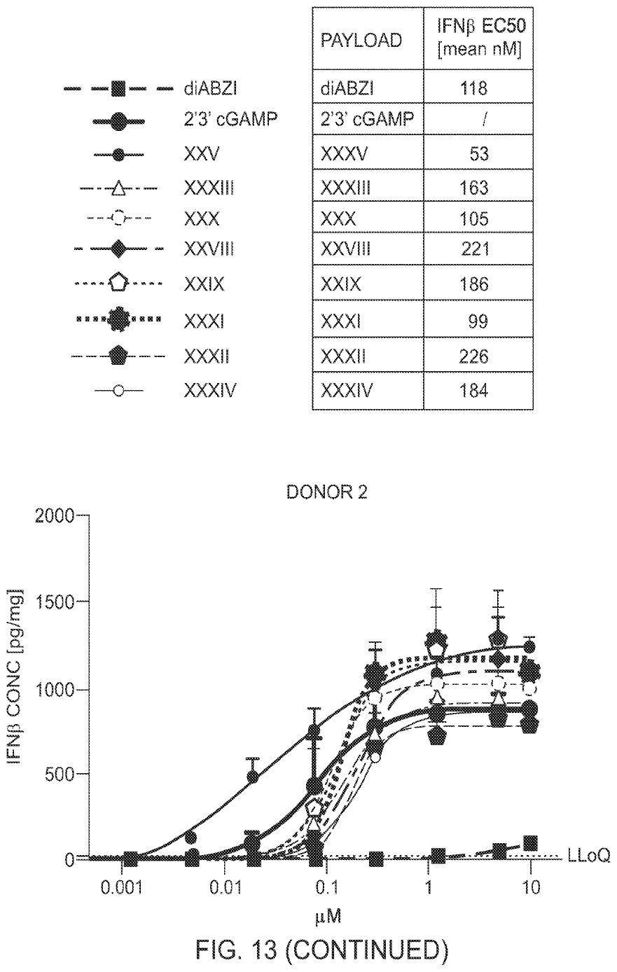

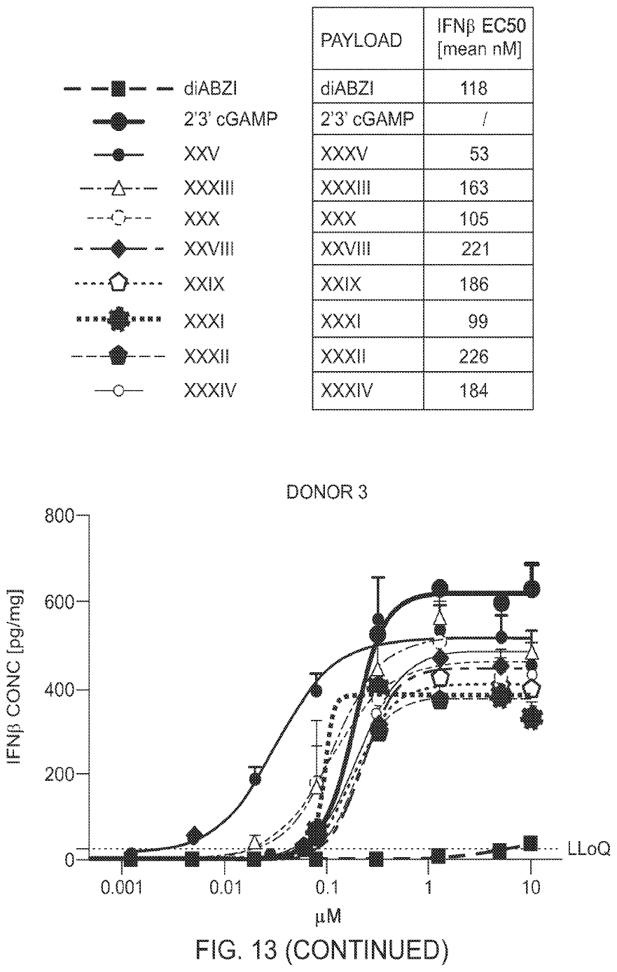

PBMC assays were conducted per the methods set forth in Example 23 above. The activity of compounds XXVIII-XXXV are shown in FIG. 13.

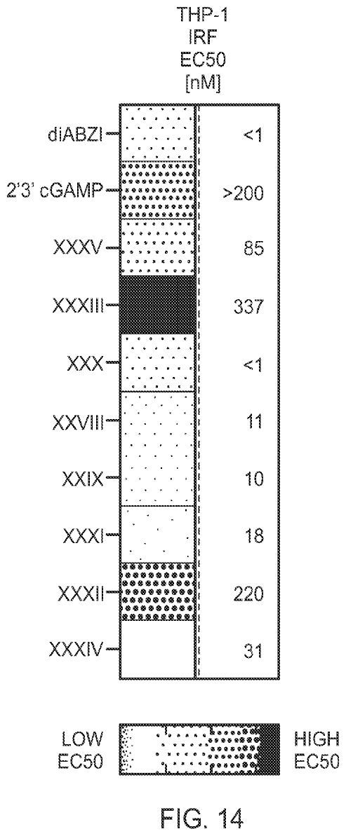

THP-1 assays were further conducted per the methods set forth in Example 20 above, to evaluate membrane permeability of the compounds. The results are shown below in FIG. 14.

### Example 42: Synthesis of Compound XL

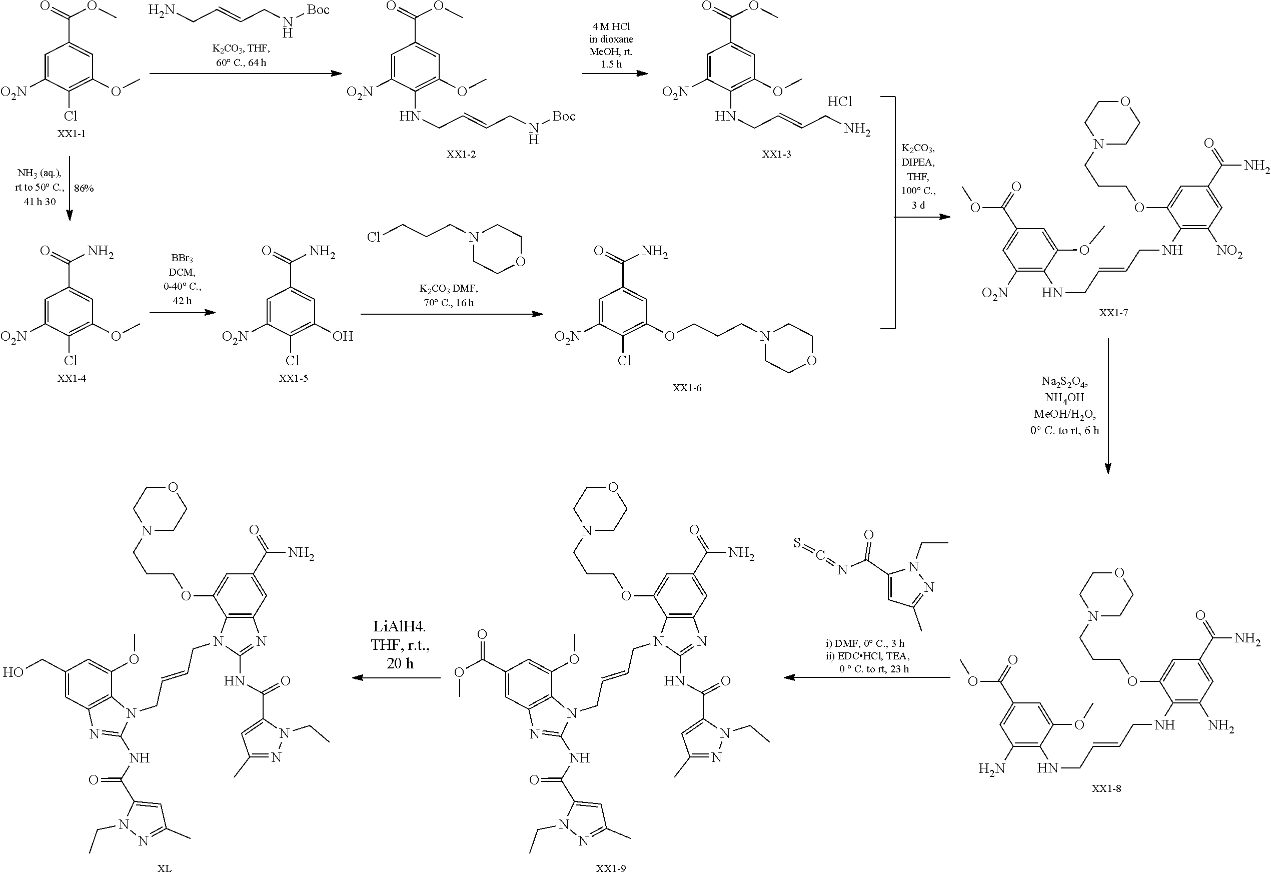

### Preparation of Methyl 4-{[(2E)-4-{[(tert-butoxy)carbonyl]amino}but-2-en-1-yl]amino}-3-methoxy-5-nitrobenzoate (XX1-2)

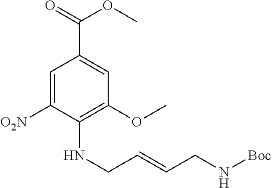 [SMILES: COC(=O)c1cc(OC)c(NC/C=C/CNC(=O)OC(C)(C)C)c([N+](=O)[O-])c1]

To a solution of methyl 4-chloro-3-methoxy-5-nitrobenzoate XX1-1 (1.00 g; 4.07 mmol; 1.00 eq.) and tert-butyl N-[(2E)-4-aminobut-2-en-1-yl]carbamate (834 mg; 4.48 mmol; 1.10 eq.) in tetrahydrofuran (15 mL) was added potassium carbonate (1.13 g; 8.14 mmol; 2.00 eq.) at room temperature. The reaction mixture was stirred at 60° C. for 64 hours. The resulting mixture was concentrated in vacuo. The residue was re-dissolved in ethyl acetate (100 mL), and washed with water (2×50 mL) and saturated aqueous sodium chloride (100 mL). The organic extracts were dried over anhydrous sodium sulfate and concentrated in vacuo to afford pure compound XX1-2 (1.61 g; 4.07 mmol; quantitative) as an orange solid. ¹H NMR (DMSO-d₆, 400 MHz): 1.34 (s, 9H), 3.42-3.50 (m, 2H), 3.83 (s, 3H), 3.89 (s, 3H), 4.11-4.13 (m, 2H), 5.52-5.53 (m, 2H), 6.56 (br s, 0.1H), 6.92 (t, 0.9H, J=5.4 Hz), 7.43 (d, 1H, J=1.8 Hz), 8.01 (t, 1H, J=6.1 Hz), 8.16 (t, 1H, J=1.8 Hz). LCMS (2-100 ACN/H₂O+0.1% FA): retention time=3.23 min; [M+Na⁺]=418.4.

### Preparation of Methyl 4-{[(2E)-4-aminobut-2-en-1-yl]amino}-3-methoxy-5-nitrobenzoate hydrochloride (XX1-3)

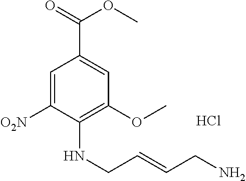 [SMILES: COC(=O)c1cc(OC)c(NC/C=C/CN)c([N+](=O)[O-])c1 ; Cl]

To a suspension of compound XX1-2 (3.25 g; 7.23 mmol; 1.00 eq.) in MeOH (7.15 mL) was added HCl (4N in dioxane) (14.3 mL; 57.2 mmol; 7.90 eq.) dropwise at room temperature. The reaction mixture was stirred at room temperature for 1.5 hours. The precipitate was filtered, and washed with diethyl ether (3×20 mL). The filtrate was re-filtered and washed with diethyl ether (10 mL). The solids were combined and dried under reduced pressure to afford pure crude compound XX1-3 (2.38 g; 7.11 mmol; 98%) as an orange solid. ¹H NMR (DMSO-d₆, 400 MHz): 3.38-3.42 (m, 2H), 3.84 (s, 3H), 3.90 (s, 3H), 4.21 (t, 2H, J=5.8 Hz), 5.59-5.66 (m, 1H), 5.83-5.90 (m, 1H), 7.46 (d, 1H, J=1.8 Hz), 7.99 (br s, 3H), 8.13 (t, 1H, J=6.4 Hz), 8.19 (d, 1H, J=1.8 Hz). LCMS (2-100 ACN/H₂O+0.1% FA): retention time=2.43 min; [M−HCl+H⁺]=296.4.

### Preparation of 4-Chloro-3-methoxy-5-nitrobenzamide (XX1-4)

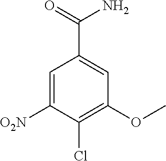

A suspension of methyl 4-chloro-3-methoxy-5-nitrobenzoate XX1-1 (50.0 g; 204 mmol; 1.00 eq.) in 30% aqueous ammonium hydroxide solution (733 mL) was stirred at 50° C. for 1.5 hours (sealed vessel) then at room temperature for 16 hours. Then the mixture was stirred at 50° C. for 24 hours (open vessel). The reaction mixture was allowed to cool to room temperature and filtered. The solid was washed with water (500 mL), diethyl ether (400 mL) and dried (50° C., 61 h) to afford pure compound XX1-4 (40.6 g; 176 mmol; 86%) as a yellow solid. ¹H NMR (DMSO-d₆, 400 MHz): δ 4.02 (s, 3H), 7.78 (br s, 1H), 7.88 (d, 1H, J=1.7 Hz), 8.05 (d, 1H, J=1.7 Hz), 8.29 (br s, 1H). LCMS (2-100 ACN/H₂O+0.1% FA): retention time=2.66 min; [M+H⁺]=231.2.

### Preparation of 4-Chloro-3-hydroxy-5-nitrobenzamide (XX1-5)

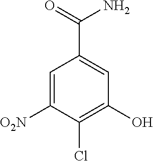 [SMILES: NC(=O)c1cc(O)c(Cl)c([N+](=O)[O-])c1]

To a solution of compound XX1-4 (31.5 g; 136 mmol; 1.00 eq.) in dichloromethane (400 mL), cooled to 0° C. was added over 20 minutes boron tribromide (1 M in DCM; 545 mL; 545 mmol; 4.00 eq.) and this mixture was stirred at 40° C. for 42 hours. The reaction mixture was allowed to reach room temperature and was poured into 500 ml of ice/water and stirred for 10 minutes (until ice melts). The precipitate was filtered, washed two times with water, two times with n-pentane, dried under reduced pressure (50° C., 16 hours) to afford a white solid (41.4 g). The residue was crushed and triturated in water (400 mL) for 1 hour and filtered. The filter cake was washed with water (500 mL) and n-pentane (2×500 mL), dried under vacuum to afford a white solid (25.3 g). The residue was crushed and triturated in water (400 mL) for 2 hours, then filtered. The filter cake was washed with water (500 mL) and n-pentane (2×500 mL), and dried under vacuum to afford crude compound XX1-5 (23.5 g; 109 mmol; 80%) as a white solid. ¹H NMR (DMSO-d₆, 400 MHz): δ 7.67 (br s, 1H), 7.72 (d, 1H, J=1.8 Hz), 7.93 (d, 1H, J=1.9 Hz), 8.18 (br s, 1H), 11.53 (br s, 1H). LCMS (2-100 ACN+0.1% AF/H₂O+0.1% AF): retention time=2.49 min; [M+H⁺]=217.2.

### Preparation of 4-Chloro-3-[3-(morpholin-4-yl) propoxy]-5-nitrobenzamide (XX1-6)

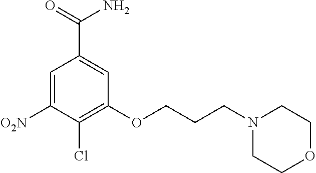 [SMILES: NC(=O)c1cc(OCCCN2CCOCC2)c(Cl)c([N+](=O)[O-])c1]

A mixture of compound XX1-5 (1.00 g; 4.62 mmol; 1.00 eq.), 4-(3-chloropropyl) morpholine (907 mg; 5.54 mmol; 1.20 eq.), potassium carbonate (830 mg; 6.00 mmol; 1.30 eq.) in N,N-dimethylformamide (6.00 mL) was stirred at 70° C. for 16 hours. The solvent was removed in vacuo to give a yellow solid (2.71 g). The residue was purified by flash chromatography on silica gel (50 μm irregular, 80 g) using dichloromethane/methanol (100:0 to 85:15 in 30 minutes and then 85:15 for 10 minutes) to afford pure compound XX1-6 (1.17 g; 3.40 mmol; 74%) as a pale yellow solid. ¹H NMR (DMSO-d₆, 400 MHz): δ 1.92-1.98 (m, 2H), 2.35-2.37 (m, 4H), 2.45 (t, 2H, J=7.1 Hz), 3.56 (t, 4H, J=4.6 Hz), 4.28 (t, 2H, J=6.3 Hz), 7.77 (br s, 1H), 7.87 (d, 1H, J=1.7 Hz), 8.04 (d, 1H, J=1.7 Hz), 8.27 (br s, 1H). LCMS (2-100 ACN/H₂O+0.1% AF): retention time=2.37 min; [M+H⁺]=344.3.

### Preparation of 4-{[(2E)-4-({4-Carbamoyl-2-[3-(morpholin-4-yl) propoxy]-6-nitrophenyl}amino)but-2-en-1-yl]amino}-3-methoxy-5-nitrobenzamide (XX1-7)

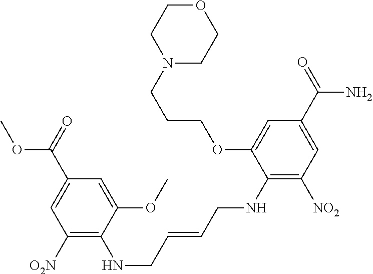 [SMILES: COC(=O)c1cc(OC)c(NC/C=C/CNc2c(OCCCN3CCOCC3)cc(C(N)=O)cc2[N+](=O)[O-])c([N+](=O)[O-])c1]

To a stirred solution of compound XX1-3 (1.06 g; 3.20 mmol; 1.10 eq.) and compound XX1-6 (1.00 g; 2.91 mmol; 1.00 eq.) in tetrahydrofuran (10.7 mL) was added potassium carbonate (804 mg; 5.82 mmol; 2.00 eq.) and DIPEA (1.68 mL; 10.2 mmol; 3.49 eq.). The reaction mixture was stirred at 100° C. for 3 days in a sealed vessel. The reaction mixture was cooled down to room temperature, diluted with water (40 mL) and extracted with a mixture of dichloromethane/methanol (8/2, 3×40 mL). The combined organic layers were dried over anhydrous sodium sulfate, filtered and concentrated to afford an orange solid (2.17 g). The residue was purified by flash chromatography on silica gel (50 μm irregular, 80 g) using dichloromethane/methanol (98:2 for 10 minutes, to 92:8 in 20 minutes and then 92:8 for 10 minutes) to afford pure compound XX1-7 (1.17 g; 1.95 mmol; 67%) as an orange solid. ¹H NMR (DMSO-d₆, 400 MHz): δ 1.85-1.91 (m, 2H), 2.32-2.34 (m, 4H), 2.38 (t, 2H, J=7.1 Hz), 3.55 (t, 4H, J=4.5 Hz), 3.80 (s, 3H), 3.84 (s, 3H), 4.00 (t, 2H, J=6.3 Hz), 4.07-4.11 (m, 4H), 5.52-5.62 (m, 2H), 7.30 (br s, 1H), 7.35 (d, 1H, J=1.8 Hz), 7.47 (d, 1H, J=1.8 Hz), 7.73 (t, 1H, J=6.2 Hz), 7.95-7.98 (m, 2H), 8.10 (d, 1H, J=1.8 Hz), 8.14 (d, 1H, J=1.8 Hz). LCMS (2-100 ACN/H₂O+0.1% AF): retention time=2.59 min; [M+H⁺]=603.5.

### Preparation of Methyl 3-amino-4-{[(2E)-4-({2-amino-4-carbamoyl-6-[3-(morpholin-4-yl) propoxy]phenyl}amino)but-2-en-1-yl]amino}-5-methoxybenzoate (XX1-8)

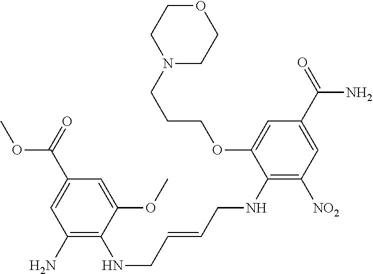 [SMILES: COC(=O)c1cc(N)c(NC/C=C/CNc2c(OCCCN3CCOCC3)cc(C(N)=O)cc2[N+](=O)[O-])c(OC)c1]

To a stirred solution of compound XX1-7 (11.7 g; 19.5 mmol; 1.00 eq.) in MeOH (305 mL) at 0° C. was added sodium dithionite (33.9 g; 195 mmol; 10.0 eq.) dissolved in water (105 mL). To this stirred mixture was added a 30% aqueous ammonia solution (19.4 mL; 146 mmol; 7.50 eq.) at 0° C. The mixture was warmed to room temperature and stirred for 6 hours. The reaction mixture was quenched with water (500 mL). Dichloromethane was added to the mixture (700 mL). The layers were separated and the aqueous layer was extracted with a mixture dichloromethane/methanol (9:1, 2×300 mL). The combined organic layers were washed with brine (500 mL), dried over anhydrous sodium sulfate, filtered and concentrated in vacuo to afford compound XX1-8 (7.34 g; 13.5 mmol; 69%) as a brown foam. The crude product was used in the next step. ¹H NMR (DMSO-d₆, 400 MHz): δ 1.82-1.88 (m, 2H), 2.32-2.34 (m, 4H), 2.40 (t, 2H, J=7.0 Hz), 3.51-3.56 (m, 6H), 3.60-3.62 (m, 2H), 3.73 (s, 3H), 3.76 (s, 3H), 3.80 (t, 1H, J=7.1 Hz), 3.95 (t, 2H, J=6.2 Hz), 4.07 (t, 1H, J=7.0 Hz), 4.65 (s, 2H), 4.79 (s, 2H), 5.59-5.70 (m, 2H), 6.76 (d, 1H, J=1.8 Hz), 6.81 (d, 1H, J=1.8 Hz), 6.84 (d, 1H, J=1.8 Hz), 6.95 (br s, 1H), 7.01 (d, 1H, J=1.8 Hz), 7.60 (br s, 1H). LCMS (2-100 ACN/H₂O+0.1% AF): retention time=2.22 min; [M+H⁺]=543.5.

### Preparation of Methyl 1-[(2E)-4-[5-carbamoyl-2-(1-ethyl-3-methyl-1H-pyrazole-5-amido)-7-[3-(morpholin-4-yl) propoxy]-1H-1,3-benzodiazol-1-yl]but-2-en-1-yl]-2-(1-ethyl-3-methyl-1H-pyrazole-5-amido)-7-methoxy-1H-1,3-benzodiazole-5-carboxylate (XX1-9)

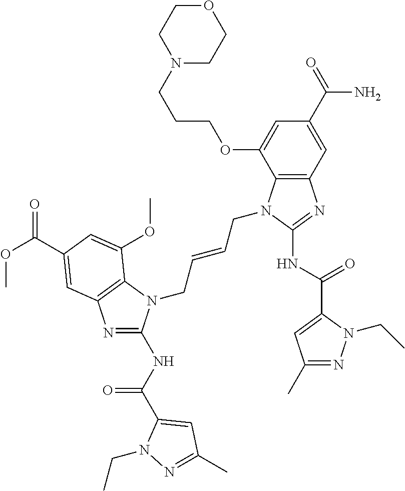 [SMILES: CCn1nc(C)cc1C(=O)Nc1nc2cc(C(=O)OC)cc(OC)c2n1C/C=C/Cn1c(NC(=O)c2cc(C)nn2CC)nc2cc(C(N)=O)cc(OCCCN3CCOCC3)c21]

To a solution of compound XX1-8 (2.00 g; 3.69 mmol; 1.00 eq.) in N,N-dimethylformamide (40 mL) at 0° C. was added dropwise 1-ethyl-3-methyl-1H-pyrazole-5-carbonyl isothiocyanate (0.2 M in dioxane; 18.4 mL; 3.69 mmol; 1.00 eq.) and the reaction mixture was stirred for 30 minutes. 1-ethyl-3-methyl-1H-pyrazole-5-carbonyl isothiocyanate (0.2 M in dioxane; 7.37 mL; 1.47 mmol; 0.40 eq.) was added dropwise at 0° C. and the reaction mixture was stirred for 15 minutes. Compound 1-ethyl-3-methyl-1H-pyrazole-5-carbonyl isothiocyanate (0.2 M in dioxane; 3.69 mL; 0.74 mmol; 0.20 eq.) was added dropwise at 0° C. and the reaction mixture was stirred for 15 minutes. Compound 1-ethyl-3-methyl-1H-pyrazole-5-carbonyl isothiocyanate (0.2 M in dioxane; 7.37 mL; 1.47 mmol; 0.40 eq.) was added dropwise at 0° C. and the reaction mixture was stirred for 2 hours. EDC·HCl (1.77 g; 9.21 mmol; 2.50 eq.) followed by triethylamine (2.56 mL; 18.4 mmol; 5.00 eq.) were added to the reaction at 0° C. The reaction mixture was stirred at room temperature for 17 hours. EDC·HCl (0.18 g; 0.92 mmol; 0.25 eq.) and triethylamine (0.064 mL; 0.46 mmol; 0.13 eq.) were added at room temperature. The reaction mixture was stirred at room temperature for 6 hours. The reaction was quenched with a mixture of water/saturated aqueous ammonium chloride (3:1, 80 mL) and extracted with a mixture of dichloromethane/methanol (3:1, 3×80 mL). The combined organic phases were washed with water (100 mL), dried over anhydrous magnesium sulfate, filtered and concentrated under reduced pressure to afford an orange oil. The residue was purified by flash chromatography on silica gel (50 μm irregular, 220 g) using dichloromethane/methanol (96:4 during 5 minutes, to 80:20 in 30 minutes and then to 80:20 in 25 minutes) as eluent to afford impure XX1-9 (1.83 g) as a beige solid. The residue was triturated in dichloromethane (40 mL) to afford pure compound XX1-9 (1.74 g; 2.01 mmol; 55%) as a white solid. ¹H NMR (DMSO-d₆, 400 MHz): δ 1.27-1.32 (m, 6H), 1.60-1.66 (m, 2H), 2.11 (s, 3H), 2.13 (s, 3H), 2.17-2.19 (m, 4H), 2.23 (t, 2H, J=7.2 Hz), 3.45 (t, 4H, J=4.5 Hz), 3.65 (s, 3H), 3.67-3.90 (m, 5H), 4.51-4.58 (m, 4H), 4.90-4.92 (m, 4H), 5.75-5.87 (m, 2H), 6.54 (m, 2H), 7.23 (d, 2H, J=5.2 Hz), 7.31 (br s, 1H), 7.63 (s, 1H), 7.77 (d, 1H, J=1.2 Hz), 7.92 (br s, 1H), 12.82 (br s, 1H), 12.86 (br s, 1H). LCMS (2-100 ACN/H₂O+0.1% AF): retention time=7.06 min; [M+H⁺]=865.75.

### Preparation of (E)-2-(1-ethyl-3-methyl-1H-pyrazole-5-carboxamido)-1-(4-(2-(1-ethyl-3-methyl-1H-pyrazole-5-carboxamido)-5-(hydroxymethyl)-7-methoxy-1H-benzo[d]imidazol-1-yl)but-2-en-1-yl)-7-(3-morpholinopropoxy)-1H-benzo[d]imidazole-5-carboxamide (Compound XL)

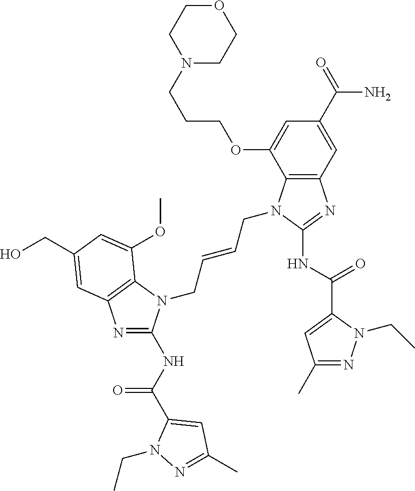 [SMILES: CCn1nc(C)cc1C(=O)Nc1nc2cc(CO)cc(OC)c2n1C/C=C/Cn1c(NC(=O)c2cc(C)nn2CC)nc2cc(C(N)=O)cc(OCCCN3CCOCC3)c21]

Compound XX1-9 (74 mg, 86 μmol) was dissolved in THF (2.0 mL). Then, LiAlH₄ (6.5 mg, 0.17 mmol) was added, and the mixture was stirred at room temperature for 2.5 h. Again, LiAlH₄ (6.5 mg, 0.17 mmol) was added, and the mixture was stirred at room temperature for 18 h. The mixture was diluted with water (10 mL) and filtered. The residue was freeze-dried and then purified by automated flash column chromatography on Büchi C-850 (XSelect® CSH™ Prep OBD™ C18 0.5 μM, 50 mm×150 mm, 100 mL/min; 5-100% CH₃CN in H₂O (50 mM NH₄HCO₃) in 30 min) to afford compound XL (35.0 mg; 42.0 μmol; 79%) as white solid. ¹H NMR (400 MHz, DMSO-d6) δ 12.65 (br s, 2H), 7.87 (br s, 1H), 7.63 (d, J=1.2 Hz, 1H), 7.23 (br s, 1H), 7.16 (br s, 1H), 7.11 (s, 1H), 6.68 (s, 1H), 6.50 (s, 2H), 5.89-5.68 (m, 2H), 5.21 (t, J=5.9 Hz, 1H), 4.94-4.81 (m, 4H), 4.60-4.48 (m, 6H), 3.89 (t, J=6.2 Hz, 2H), 3.59 (s, 3H), 3.46 (t, J=4.6 Hz, 4H), 2.25 (t, J=7.2 Hz, 2H), 2.22-2.16 (m, 4H), 2.13 (s, 3H), 2.11 (s, 3H), 1.64 (p, 2H), 1.28 (t, J=7.0 Hz, 6H). LCMS (Method B): retention time=2.33 min; [M+H⁺]=837.28.

### Activity of Compound XL

The activity of Compound XL with and without permeabilization is shown below in Table 16.

TABLE 16

| | Background | | | | | |
|---|---|---|---|---|---|---|
| | Activity w/o | | | | Activity with | |
| | permeabilization | | | | permeabilization | |
| | [nM] | | | | [nM] | |
| | Payload | | | | | |
| | HEK | HEK | THP-1 | THP-1 | HEK | HEK |
| | IFNβ | IRF | IRF | NFkB | IFNβ | IRF |
| | MEC | EC50 | MEC | MEC | MEC | EC50 |
| XL | 0.8 | 0.53 | 78 | 78 | 4.0 | 1.11 |

### Example 43: Synthesis of Compound XLI

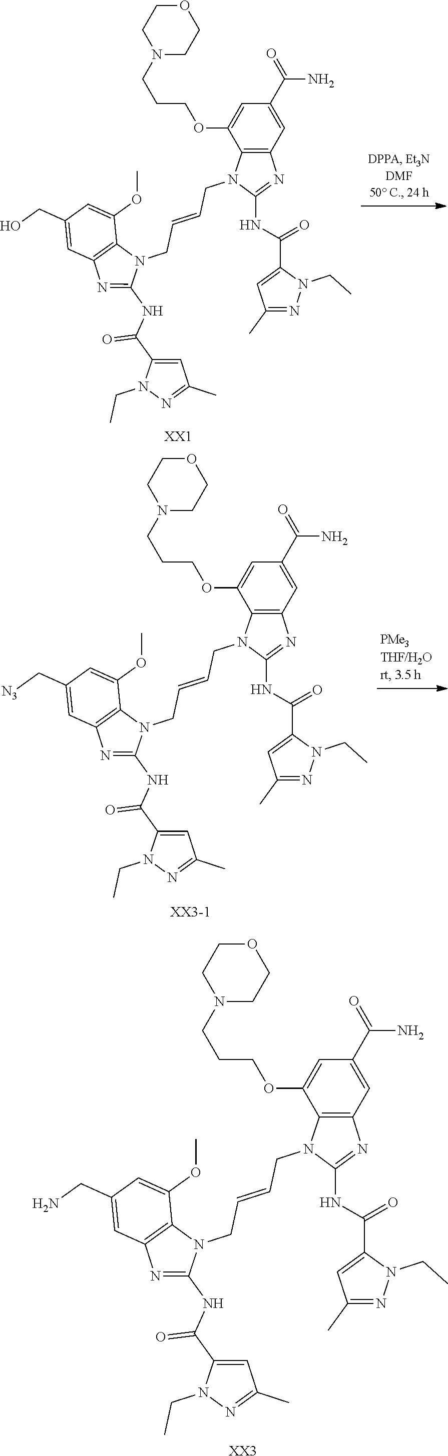

### Preparation of 1-[(2E)-4-[5-(Azidomethyl)-2-(1-ethyl-3-methyl-1H-pyrazole-5-amido)-7-methoxy-1H-1,3-benzodiazol-1-yl]but-2-en-1-yl]-2-(1-ethyl-3-methyl-1H-pyrazole-5-amido)-7-[3-(morpholin-4-yl) propoxy]-1H-1,3-benzodiazole-5-carboxamide (XX3-1)

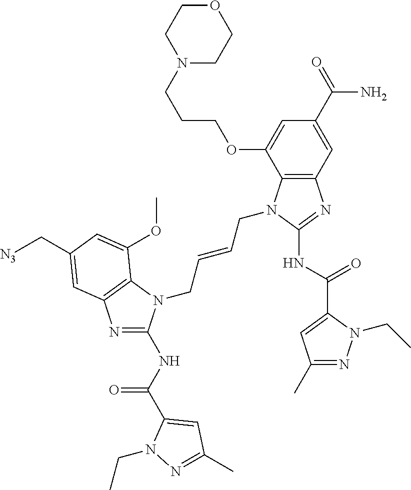 [SMILES: CCn1nc(C)cc1C(=O)Nc1nc2cc(CN=[N+]=[N-])cc(OC)c2n1C/C=C/Cn1c(NC(=O)c2cc(C)nn2CC)nc2cc(C(N)=O)cc(OCCCN3CCOCC3)c21]

In a flask under argon, compound XL (1.00 eq., 550 mg, 0.66 mmol) and anhydrous triethylamine (3.00 eq., 0.27 mL, 1.97 mmol) were dissolved in anhydrous dimethylformamide (4.66 mL) at room temperature. Then, diphenylphosphoryl azide (3.00 eq., 0.43 mL, 1.97 mmol) was added and the reaction mixture was stirred at 50° C. for 24 hours. The reaction mixture was diluted with dichloromethane (25 mL) and a saturated aqueous solution of NaHCO₃ (20 mL). The organic layer was washed with a saturated aqueous solution of NaHCO₃ (2×20 mL), dried over anhydrous sodium sulfate, filtered, and concentrated under vacuum to afford the crude product XX3-1 (352 mg) as a clear oil. The residue was purified by reverse phase chromatography (Column: Interchim C18 50 μm 40 g; Loading: Solid (C18 Silica, 5 g); Eluents: Water/MeCN; 98:2 for 5 minutes, to 30:70 over 40 minutes, and to 0:100 over 5 minutes). The fractions containing compound were combined, evaporated in vacuo and co-evaporated with acetonitrile to afford impure XX3-1 (352 mg) as a white solid. The residue was purified by reverse phase chromatography (Column: Interchim C18 50 μm 24 g; Loading: Solid (C18 Silica, 1 g); Eluents: Water/MeCN (+0.1% HCO₂H); 95:5 for 10 minutes, to 70:30 over 15 minutes, 70:30 for 11 minutes and to 50:50 over 15 minutes). The fractions containing compound were combined, evaporated in vacuo and co-evaporated with acetonitrile to afford impure XX3-1 (237 mg) as a white solid. The residue was diluted with a mixture DCM/MeOH (8:2, 15 mL). Then, it was washed with a saturated aqueous solution of NaHCO₃ (10×10 mL). The organic layer was dried over anhydrous sodium sulfate, filtered and concentrated under vacuum to afford compound XX3-1 (163 mg, 0.18 mmol, 28%) as an off-white solid. ¹H NMR (400 MHz, DMSO-d6) δ 12.76 (s, 2H), 7.92 (s, 1H), 7.63 (d, J=1.3 Hz, 1H), 7.30 (s, 1H), 7.21 (s, 1H), 7.14 (d, J=1.3 Hz, 1H), 6.76 (d, J=1.4 Hz, 1H), 6.57 (s, 1H), 6.51 (s, 1H), 5.89-5.76 (m, 2H), 4.97-4.81 (m, 4H), 4.55 (dq, J=11.1, 7.0 Hz, 4H), 4.46 (s, 2H), 3.89 (t, J=6.2 Hz, 2H), 3.61 (s, 3H), 3.45 (t, J=4.6 Hz, 4H), 2.24 (t, J=7.2 Hz, 2H), 2.18 (t, J=4.6 Hz, 4H), 2.14 (s, 3H), 2.11 (s, 3H), 1.64 (p, J=6.5 Hz, 2H), 1.29 (q, J=7.2 Hz, 6H). LC/MS (2-100 ACN/H₂O+0.1% AF): retention time=1.27 min, [M+H]⁺=862.9.

### Preparation of 1-[(2E)-4-[5-(Aminomethyl)-2-(1-ethyl-3-methyl-1H-pyrazole-5-amido)-7-methoxy-1H-1,3-benzodiazol-1-yl]but-2-en-1-yl]-2-(1-ethyl-3-methyl-1H-pyrazole-5-amido)-7-[3-(morpholin-4-yl) propoxy]-1H-1,3-benzodiazole-5-carboxamide (Compound XLI)

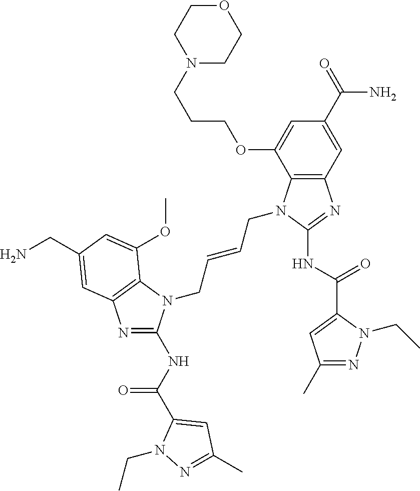 [SMILES: CCn1nc(C)cc1C(=O)Nc1nc2cc(CN)cc(OC)c2n1C/C=C/Cn1c(NC(=O)c2cc(C)nn2CC)nc2cc(C(N)=O)cc(OCCCN3CCOCC3)c21]

Under argon, compound XX3-1 (1.00 eq., 155 mg, 0.18 mmol) was diluted in tetrehydrofuran (2.0 mL) and water (0.18 mL). Then, PMe₃ (1M in THF) (2.00 eq., 0.36 mL, 0.36 mmol) was added dropwise over 2 minutes at room temperature. The reaction mixture was stirred for 3.5 hours. The reaction mixture was concentrated under vacuum to afford the crude product (168 mg) as an off-white solid. The residue was purified by reverse phase chromatography (Column: Interchim C18 12 g; Loading: solid (C18 Silica, 500 mg); Eluents: water/MeCN+ (0.1% NH₄HCO₃), 90:10 for 5 minutes, to 0:100 over 90 minutes). The fractions containing compound were combined, evaporated in vacuo and co-evaporated with acetonitrile to afford impure XLI (85 mg) as an off-white solid. The residue was triturated in acetonitrile (2.00 mL, sonicated for 10 minutes) and centrifugated (4000 rpm, 4 minutes). The supernatant was removed (operation repeated twice). The solid was dried under vacuum to afford compound XLI (25 mg, 0.030 mmol, 25%) as an off-white solid. ¹H NMR (400 MHz, DMSO-d6) δ 7.93 (s, 1H), 7.63 (d, J=1.3 Hz, 1H), 7.28 (s, 1H), 7.19 (s, 1H), 7.07 (s, 1H), 6.74 (s, 1H), 6.55 (s, 1H), 6.51 (s, 1H), 5.81 (tt, J=10.6, 5.1 Hz, 2H), 4.94-4.79 (m, 4H), 4.55 (p, J=7.2 Hz, 4H), 3.88 (t, J=6.2 Hz, 2H), 3.72 (s, 2H), 3.57 (s, 3H), 3.46 (t, J=4.6 Hz, 4H), 2.25 (t, J=7.3 Hz, 2H), 2.19 (t, J=4.3 Hz, 4H), 2.14 (s, 3H), 2.11 (s, 3H), 1.65 (q, J=6.9 Hz, 2H), 1.29 (td, J=7.1, 5.5 Hz, 6H). LC/MS (Method C): retention time=2.27 min, [M+H]⁺=836.5.

### Example 44: Synthesis of Compound XLII

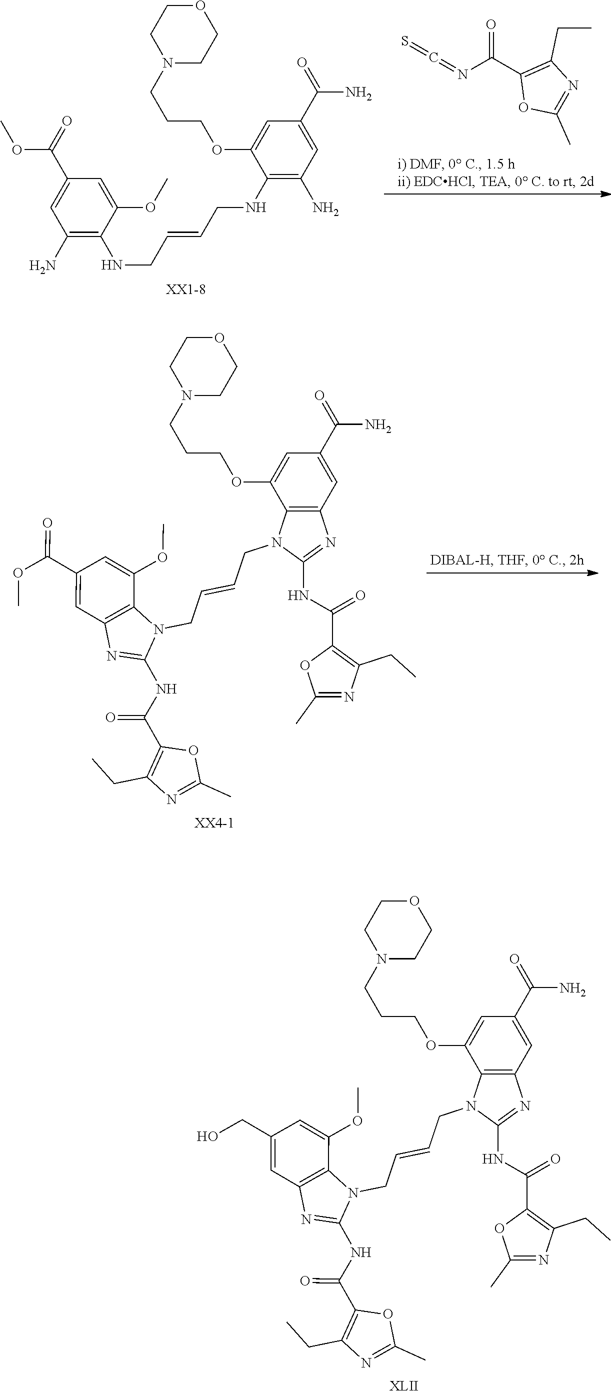 [SMILES: COC(=O)c1cc(N)c(NC/C=C/CNc2c(N)cc(C(N)=O)cc2OCCCN2CCOCC2)c(OC)c1 ; CCc1nc(C)oc1C(=O)N=C=S ; CCc1nc(C)oc1C(=O)Nc1nc2cc(C(=O)OC)cc(OC)c2n1C/C=C/Cn1c(NC(=O)c2oc(C)nc2CC)nc2cc(C(N)=O)cc(OCCCN3CCOCC3)c21 ; CCc1nc(C)oc1C(=O)Nc1nc2cc(CO)cc(OC)c2n1C/C=C/Cn1c(NC(=O)c2oc(C)nc2CC)nc2cc(C(N)=O)cc(OCCCN3CCOCC3)c21]

### Preparation of methyl (E)-1-(4-(5-carbamoyl-2-(4-ethyl-2-methyloxazole-5-carboxamido)-7-(3-morpholinopropoxy)-1H-benzo[d]imidazol-1-yl)but-2-en-1-yl)-2-(4-ethyl-2-methyloxazole-5-carboxamido)-7-methoxy-1H-benzo[d]imidazole-5-carboxylate trifluoroacetic acid salt (XX4-1)

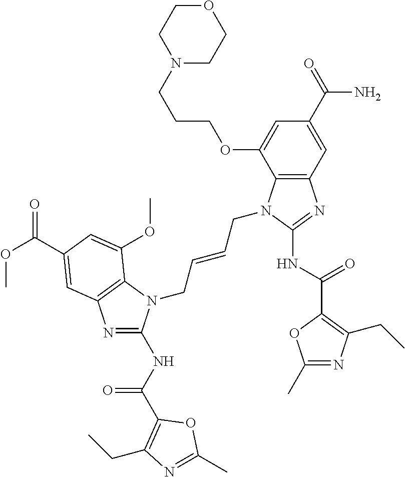 [SMILES: CCc1nc(C)oc1C(=O)Nc1nc2cc(C(=O)OC)cc(OC)c2n1C/C=C/Cn1c(NC(=O)c2oc(C)nc2CC)nc2cc(C(N)=O)cc(OCCCN3CCOCC3)c21]

Compound XX1-8 (400 mg, 737 μmol) was dissolved in anhydrous DMF (4.00 mL) and cooled to 0° C. Then, 4-ethyl-2-methyloxazole-5-carbonyl isothiocyanate (0.2 M in dioxane, 3.68 mL, 737 μmol) was added dropwise and the reaction mixture was stirred at 0° C. for 30 min. Again, 4-ethyl-2-methyloxazole-5-carbonyl isothiocyanate (0.2 M in dioxane, 1.84 mL, 369 μmol) was added dropwise and the reaction mixture was stirred at 0° C. for 20 min. Again, 4-ethyl-2-methyloxazole-5-carbonyl isothiocyanate (0.2 M in dioxane, 369 μL, 73.7 μmol) was added dropwise and the reaction mixture was stirred at 0° C. for 30 min. Then, EDC·HCl (353 mg, 1.84 mmol) and Et₃N (517 UL, 3.69 mmol) were added and the reaction mixture was stirred at room temperature for 2 days. The product formation was controlled by LC/MS. The reaction mixture was diluted with water/sat. aqueous NH₄Cl (3:1, 20 mL) and extracted with CH₂Cl₂/CH₃OH (3:1, 3×25 mL). The combined organic layers were dried over Na₂SO₄ and filtered. The solvent was removed under reduced pressure. The residue was purified by flash column chromatography on Büchi C-850 (XSelect® CSH™ Prep OBD™ C18 0.5 μM, 50 mm×150 mm, 100 mL/min; 5-100% CH₃CN in H₂O (+0.15% TFA) in 30 min) to afford compound XX4-1 (490 mg, 500 μmol, 68%) as an off-white solid. ¹H-NMR (300 MHz, DMSO-d₆): δ [ppm]=1.12-0.97 (m, 6H), 1.65 (p, J=6.5 Hz, 2H), 2.31-2.15 (m, 6H), 2.39 (s, 3H), 2.40 (s, 3H), 2.92-2.73 (m, 4H), 3.47 (t, J=4.6 Hz, 4H), 3.68 (s, 3H), 3.87 (s, 3H), 3.91 (t, 2H, J=6.3 Hz), 4.88 (m, 4H), 5.88-5.71 (m, 2H), 7.22 (d, 1H, J=1.4 Hz), 7.24 (d, 1H, J=1.4 Hz), 7.29 (br s, 1H), 7.63 (d, 1H, J=1.2 Hz), 7.77 (d, 1H, J=1.3 Hz), 7.90 (br s, 1H), 12.71 (br s, 1H), 12.77 (br s, 1H). LC/MS (Method C): retention time=3.10 min, [M+H]⁺=867.8.

### Preparation of (E)-N-(5-carbamoyl-1-(4-(2-(4-ethyl-2-methyloxazole-5-carboxamido)-5-(hydroxymethyl)-7-methoxy-1H-benzo[d]imidazol-1-yl)but-2-en-1-yl)-7-(3-morpholinopropoxy)-1H-benzo[d]imidazol-2-yl)-4-ethyl-2-methyloxazole-5-carboxamide (Compound XLII)

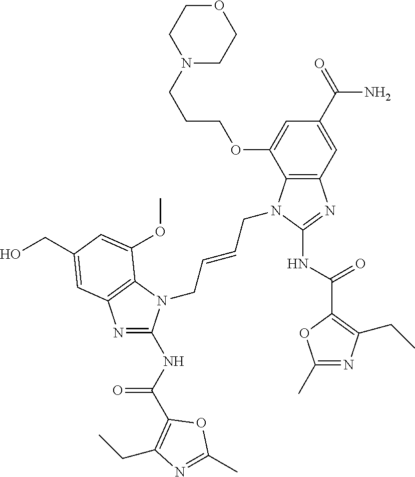 [SMILES: CCc1nc(C)oc1C(=O)Nc1nc2cc(CO)cc(OC)c2n1C/C=C/Cn1c(NC(=O)c2oc(C)nc2CC)nc2cc(C(N)=O)cc(OCCCN3CCOCC3)c21]

Compound XX4-1 (150 mg, 0.153 mmol) was dissolved under argon in dry THF (4.00 mL). The reaction mixture was cooled to 0° C. in an ice bath, then DIBALH (0.92 mL, 1.00 M in THF, 0.920 mmol) was added slowly. The resulting mixture was then stirred at 0° C. for 45 min. Control by LC/MS showed incomplete conversion. Additional DIBALH (0.45 mL, 1.00 M in THF, 0.450 mmol) was added and stirring was continued at 0° C. for 1 h. The reaction mixture was quenched at 0° C. with 1N HCl (10 mL) and then stirred at r.t. for 30 min. The reaction mixture was poured into saturated aqueous NaHCO₃ (40 mL) and then extracted with dichloromethane/methanol=8:2 (7×15 mL). The combined organic layers were washed with saturated aqueous NaHCO₃ (30 mL), dried over Na₂SO₄ and concentrated in vacuo, redissolved in DCM/MeOH=95:5 (3.00 mL) and filtered through a syringe filter (0.45 M). The solvent was removed in vacuo to afford 101 mg of the crude product as beige solid. The material was purified by automated flash chromatography on Büchi C650 (XSelect® CSH™ Prep OBD™ C18 0.5 μM, 50 mm×150 mm, 100 mL/min; 5-100% CH₃CN in aqueous 50 mM ammonium bicarbonate buffer in 30 min) to give compound XLII (41.9 mg, 0.050 mmol, 33%) as white solid. ¹H-NMR (300 MHz, DMSO-d₆): δ [ppm]=1.03 (t, 3H, J=7.5 Hz), 1.09 (t, 3H, J=7.5 Hz), 1.68 (p, 2H, J=6.4 Hz), 2.17-2.24 (m, 4H), 2.28 (t, 2H, J=7.3 Hz), 2.39 (s, 3H), 2.42 (s, 3H), 2.85 (dq, 4H, J=15.2 Hz, 7.5 Hz), 3.46-3.51 (m, 4H), 3.63 (s, 3H), 3.94 (t, 2H, 6.2 Hz), 4.51 (d, 2H, J=5.5 Hz), 4.88 (dd, 4H, J=16.8 Hz, 4.1 Hz), 5.21 (t, 1H, J=5.7 Hz), 5.69-5.87 (m, 2H), 6.70 (d, 1H, J=1.2 Hz), 7.12 (d, 1H, J=1.2 Hz), 7.24 (d, 1H, J=1.4 Hz), 7.32 (br s, 1H), 7.63 (d, 1H, J=1.2 Hz), 7.93 (br s, 1H), 12.59 (br s, 1H). LC/MS (Method A): retention time=1.65 min, [M+H]⁺=839.5.

### Example 45: Synthesis of Compound XLIV

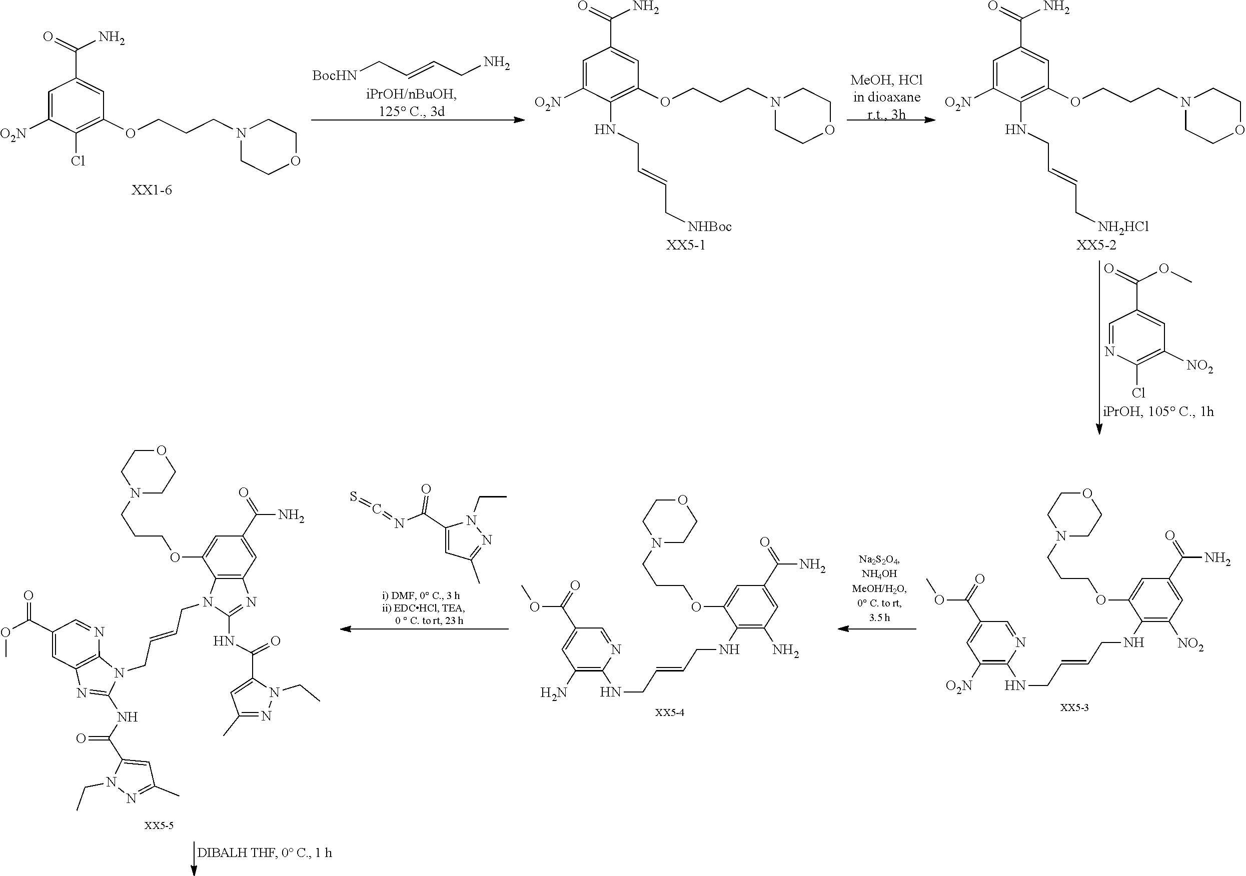

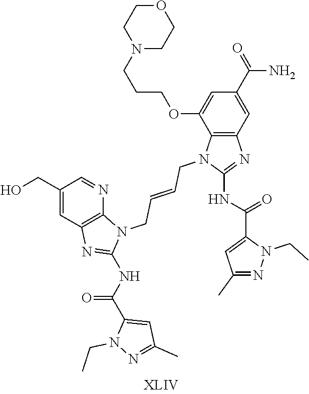 [SMILES: CCn1nc(C)cc1C(=O)Nc1nc2cc(CO)cnc2n1C/C=C/Cn1c(NC(=O)c2cc(C)nn2CC)nc2cc(C(N)=O)cc(OCCCN3CCOCC3)c21]

### Preparation of tert-butyl (E)-(4-((4-carbamoyl-2-(3-morpholinopropoxy)-6-nitrophenyl)amino)but-2-en-1-yl)carbamate (XX5-1)

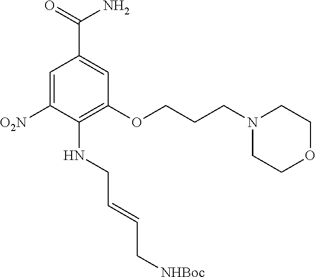 [SMILES: CC(C)(C)OC(=O)NC/C=C/CNc1c(OCCCN2CCOCC2)cc(C(N)=O)cc1[N+](=O)[O-]]

Compound XX1-6 (1000 mg, 2.91 mmol) and (E)-tert-Butyl (4-aminobut-2-en-1-yl)carbamate (813 mg, 4.36 mmol) were suspended in iPrOH (12 mL) and n-BuOH (30 mL). DIPEA (3.04 mL, 17.9 mmol) was added, and the mixture was heated to 125° C. under reflux for 3d. The reaction mixture was evaporated. The residue was purified on silica by automated flash column chromatography on Biotage Selekt (Biotage Sfär Silica HC, 20 μM, 25 g, 80 mL/min; 5-15% CH₃OH in CH₂Cl₂ in 30 min) to afford compound XX5-1 (1.21 g, 2.45 mmol, 84%) as bright orange solid. ¹H-NMR (300 MHz, DMSO-d₆): δ [ppm]=1.22-1.32 (m, 4H), 1.36 (s, 9H), 1.91-2.06 (m, 2H), 2.32-2.45 (m, 2H), 3.44-3.53 (m, 2H), 3.55-3.71 (m, 4H), 4.06-4.20 (m, 4H), 5.48-5.66 (m, 2H), 6.88-6.99 (m, 1H), 7.30 (br s, 1H), 7.56 (d, 1H, J=1.9 Hz), 7.76 (t, 1H, J=6.1 Hz), 8.02 (br s, 1H), 8.20 (d, 1H, J=1.9 Hz). LC/MS (Method A): retention time=1.49 min, [M+H]⁺=494.2.

### Preparation of (E)-4-((4-aminobut-2-en-1-yl)amino)-3-(3-morpholinopropoxy)-5-nitrobenzamide hydrochloride (XX5-2)

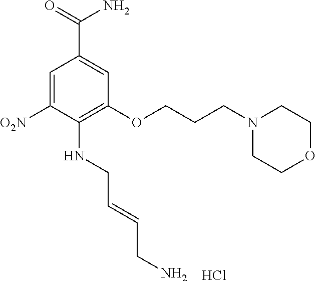 [SMILES: NC/C=C/CNc1c(OCCCN2CCOCC2)cc(C(N)=O)cc1[N+](=O)[O-] ; Cl]

Compound XX5-1 (1.21 g, 2.45 mmol) was dissolved in methanol (5.0 mL). HCl (4.0 M in dioxane, 1.5 mL, 6.0 mmol) was added and the resulting mixture was stirred at r.t. for 1.5 h. Additional HCl (4.0 M in dioxane, 2.0 mL, 8.0 mmol) was added and stirring was continued at r.t. for 1.5 h. The reaction mixture was evaporated to give 1.3 g of the crude product as a red solid, which was used in the following step as such. LC/MS (Method A): retention time=0.39 min+0.45 min, [M+H]⁺=394.2.

### Preparation of methyl (E)-6-((4-((4-carbamoyl-2-(3-morpholinopropoxy)-6-nitrophenyl)amino)but-2-en-1-yl)amino)-5-nitronicotinate (XX5-3)

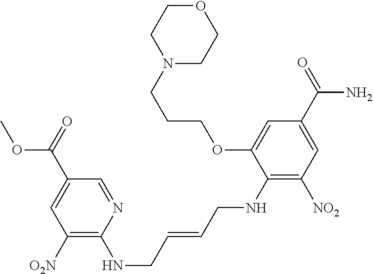 [SMILES: COC(=O)c1cnc(NC/C=C/CNc2c(OCCCN3CCOCC3)cc(C(N)=O)cc2[N+](=O)[O-])c([N+](=O)[O-])c1]

Crude compound XX5-2 (1.30 g) and methyl 6-chloro-5-nitronicotinate (528 mg, 2.44 mmol) were suspended in iPrOH (25 mL). N,N-Diisopropylethylamine (2.50 mL, 14.6 mmol) was added, and the resulting mixture was heated under reflux to 105° C. for 1 h. The reaction mixture was cooled to r.t., then the majority of the iPrOH was removed in vacuo. The residue was partitioned between DCM (40 mL) and water (30 mL). The layers were separated, and the aqueous layer was extracted with DCM (2×20 mL). The combined organic layers were dried over Na₂SO₄ and concentrated in vacuo to afford 1.45 g crude product as red solid. Purification by automated flash column chromatography on Silica: Biotage Selekt (Biotage Sfär Silica HC, 20 μM, 25 g, 80 mL/min; 5CV DCM->15 CV gradient from DCM to DCM/MeOH=9:1->10 CV DCM/MeOH=9:1) to afford compound XX5-3 (1.04 g, 1.81 mmol, 74% over two steps) as red solid. ¹H-NMR (300 MHz, DMSO-d₆): δ [ppm]=1.90 (p, 2H, J=6.7 Hz), 2.08-2.11 (m, 2H), 2.29-2.36 (m, 4H), 2.39 (t, 2H, J=7.0 Hz), 3.52-3.60 (m, 4H), 3.86 (s, 3H), 4.05 (t, 2H, J=6.4 Hz), 4.10-4.22 (m, 4H), 5.61-5.76 (m, 2H), 7.27 (br s, 1H), 7.49 (d, 1H, J=2.0 Hz), 7.69 (t, 1H, 6.3 Hz), 8.11 (d, 1H, 1.9 Hz), 8.73 (d, 1H, J=2.1 Hz), 8.79 (d, 1H, J=2.1 Hz), 9.03 (t, 1H, J=5.9 Hz). LC/MS (Method A): retention time=1.57 min, [M+H]⁺=574.2.

### Preparation of methyl (E)-5-amino-6-((4-((2-amino-4-carbamoyl-6-(3-morpholinopropoxy)phenyl)amino)but-2-en-1-yl)amino)nicotinate (XX5-4)

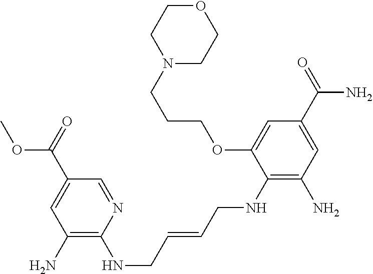 [SMILES: COC(=O)c1cnc(NC/C=C/CNc2c(N)cc(C(N)=O)cc2OCCCN2CCOCC2)c(N)c1]

Compound XX5-3 (1.04 g, 1.81 mmol) was suspended in MeOH (25 mL) and water (5.0 mL) and subsequently cooled in an ice bath to 0° C. Na₂S₂O₄ (2.74 g) and NH₃ (1.57 mL, 35% in water) was added, and the resulting suspension was stirred at 0° C. for 15 min. The mixture was then allowed to come to r.t. and stirred for 3.5 h. The solids were filtered off and washed with DCM/MeOH=8:2 (2×20 mL). The filtrate was evaporated to dryness. The residue was suspended in MeOH with sonication and filtered. The filtrate was reduced to a volume of approximately 10 mL, diluted with DCM (40 mL) and again filtered. The filtrate was evaporated and dried in vacuo to afford 930 mg of crude XX5-4 as beige foam. The material was used for further transformation as such. LC/MS (Method A): retention time=0.32 min, [M+H]⁺=514.3.

### Preparation of methyl (E)-3-(4-(5-carbamoyl-2-(1-ethyl-3-methyl-1H-pyrazole-5-carboxamido)-7-(3-morpholinopropoxy)-1H-benzo[d]imidazol-1-yl)but-2-en-1-yl)-2-(1-ethyl-3-methyl-1H-pyrazole-5-carboxamido)-3H-imidazo[4,5-b]pyridine-6-carboxylate (XX5-5)

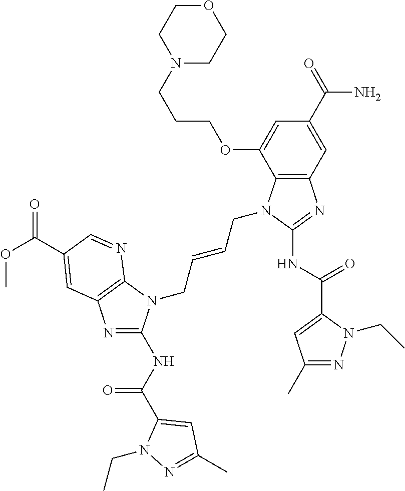 [SMILES: CCn1nc(C)cc1C(=O)Nc1nc2cc(C(=O)OC)cnc2n1C/C=C/Cn1c(NC(=O)c2cc(C)nn2CC)nc2cc(C(N)=O)cc(OCCCN3CCOCC3)c21]

Crude XX5-4 (500 mg, 0.97 mmol) was dissolved under argon in dry DMF (8.00 mL) and cooled to 0° C. in an ice bath. 1-Ethyl-3-methyl-1H-pyrazole-5-carbonyl isothiocyanate (0.2 M in dioxane, 5.00 mL, 1.00 mmol) was slowly added and the resulting mixture was stirred at 0° C. for 1.5 h. The ice bath was then removed and EDCl (560 mg, 2.92 mmol) and NEt₃ (820 μL, 5.84 mmol) was added and the resulting mixture was stirred at r.t. for 20 h. The reaction mixture was diluted with water (40 mL) and extracted with DCM (3×30 mL). The combined organic layers were dried over Na₂SO₄, and the solvent removed in vacuo. The residue was purified by preparative TLC (CH₂Cl₂/MeOH=8:2) to afford compound XX5-5 (52.0 mg, 62.2 μmol, 6%) as a yellow oil. ¹H-NMR (300 MHz, DMSO-d₆): δ [ppm]=1.17-1.26 (m, 6H), 1.65 (p, 2H, J=6.7 Hz), 2.04 (s, 3H), 2.05 (s, 3H), 2.10-2.17 (m, 4H), 2.20 (t, 2H, J=7.1 Hz), 3.37-3.44 (m, 4H), 3.82 (s, 3H), 3.92 (t, 2H, J=6.3 Hz), 4.39-4.52 (m, 4H), 4.71 (d, 2H, J=5.4 Hz), 4.86 (d, 2H, J=5.1 Hz), 5.68-5.79 (m, 1H), 5.87 (dt, 1H, J=15.7 Hz, 5.2 Hz), 6.47 (d, 2H, J=2.7 Hz), 7.19 (d, 1H, J=1.4 Hz), 7.23 (br s, 1H), 7.56 (s, 1H), 7.84 (br s, 1H), 8.07 (d, 1H, J=1.9 Hz), 8.60 (d, 1H, J=1.9 Hz), 12.73 (br s, 1H). LC/MS (Method C): retention time=3.07 min, [M+H]⁺=836.4.

### Preparation of (E)-2-(1-ethyl-3-methyl-1H-pyrazole-5-carboxamido)-1-(4-(2-(1-ethyl-3-methyl-1H-pyrazole-5-carboxamido)-6-(hydroxymethyl)-3H-imidazo[4,5-b]pyridin-3-yl)but-2-en-1-yl)-7-(3-morpholinopropoxy)-1H-benzo[d]imidazole-5-carboxamide trifluoroacetic acid salt (Compound XLIV)

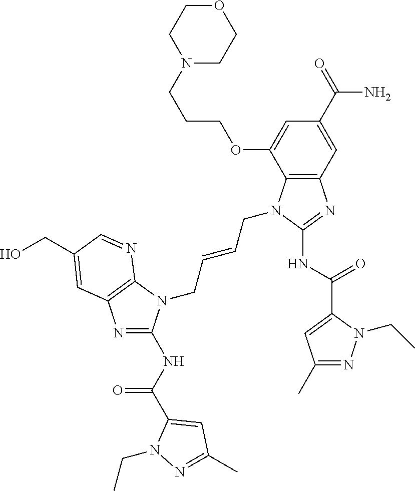 [SMILES: CCn1nc(C)cc1C(=O)Nc1nc2cc(CO)cnc2n1C/C=C/Cn1c(NC(=O)c2cc(C)nn2CC)nc2cc(C(N)=O)cc(OCCCN3CCOCC3)c21]

Compound XX5-5 (52 mg, 62.2 μmol) was dissolved under argon in dry THF (4.00 mL). The reaction mixture was cooled to 0° C. in an ice bath, then DIBALH (0.37 mL, 1.00 M in THF, 0.37 mmol) was added slowly. The resulting mixture was then stirred at 0° C. for 15 min. DIBALH (0.20 mL, 1.00 M in THF, 0.20 mmol) was added again and stirring was continued at 0° C. for 15 min. DIBALH (0.20 mL, 1.00 M in THF, 0.20 mmol) was added again and stirring was continued at 0° C. for 20 min. The reaction mixture was quenched at 0° C. with 1N HCl (10 mL) and then stirred at r.t. for 30 min. The reaction mixture was poured into saturated aqueous NaHCO₃ (40 mL) and then extracted with DCM/MeOH=8:2 (4×15 mL). The combined organic layers were dried over Na₂SO₄ and concentrated in vacuo, redissolved in DCM/MeOH=95:5 (3 mL) and filtered through a syringe filter (0.45 μM). The solvent was removed in vacuo to afford 50 mg of the crude product as yellow oil. The material was purified by automated flash chromatography on Büchi-C-850 (XSelect® CSH™ Prep OBD™ C18 0.5 μM, 30 mm×150 mm, 65 mL/min; 5-100% CH₃CN in H₂O (+0.15% TFA) in 30 min) to afford compound XLIV (7.4 mg, 8.0 μmol, 13%) as white solid. ¹H-NMR (300 MHz, DMSO-d₆): δ [ppm]=1.27 (t, 6H, J=7.1 Hz), 1.96-2.09 (m, 2H), 2.11 (s, 3H), 2.12 (s, 3H), 2.95-3.14 (m, 2H), 3.15-3.28 (m, 2H), 3.29-3.43 (m, 2H), 3.65 (t, 2H, J=12.2 Hz), 3.91-4.01 (m, 2H), 4.08-4.14 (m, 2H), 4.46-4.57 (m, 4H), 4.58 (s, 2H), 4.77 (d, 2H, J=5.4 Hz), 4.94 (d, 2H, J=5.1 Hz), 5.68-5.81 (m, 1H), 5.93 (dt, 1H, J=15.7 Hz, 5.2 Hz), 6.52 (d, 2H, J=3.1 Hz), 7.34 (d, 1H, J=1.1 Hz), 7.36 (br s, 1H), 7.67 (d, 1H, J=1.2 Hz), 7.74 (d, 1H, J=1.8 Hz), 7.94 (br s, 1H), 8.11 (d, 1H, J=1.8 Hz), 9.74 (br s, 1H), 12.76 (br s, 2H). LC/MS (Method C): retention time=2.58 min, [M+H]⁺=808.5.

### Example 46: THP-1 and HEK Assays of Compounds XL, XLI, XLII and XLIV

THP-1 assays were conducted per the methods set forth in Example 20 above, to monitor immune activation upon binding of compounds XL, XLI, XLII and XLIV. HEK assays were further conducted per the methods set forth in Example 21 above, to evaluate membrane permeability of the compounds. The results are shown below in Table 17 alongside results for diABZI (structure shown above in Example 37) and 2′3′ cGAMP benchmarks.

TABLE 17

| | | 2′3′ | Compound | Compound | Compound | Compound |
|---|---|---|---|---|---|---|
| Payload | diABZI | cGAMP | XL | XLI | XLII | XLIV |
| THP-1 NFkB | 14 | | 149 | 364 | 290 | 320 |
| EC50 [nM] | | | | | | |
| THP-1 IRF | 16 | | 221 | 811 | 333 | 315 |
| EC50 [nM] | | | | | | |
| HEK STING | 1.6 | | 2.7 | 38 | 26 | 23 |
| EC50 no | | | | | | |
| PFO [nM] | | | | | | |
| HEK STING | 1.2 | 19 | 2.7 | 25 | 14 | 16 |
| EC50 with | | | | | | |
| PFO [nM] | | | | | | |

Although the foregoing invention has been described in some detail by way of illustration and example for purposes of clarity of understanding, it is apparent to those skilled in the art that certain minor changes and modifications will be practiced.

Therefore, the description and examples should not be construed as limiting the scope of the invention.

1. A compound of Formula I: 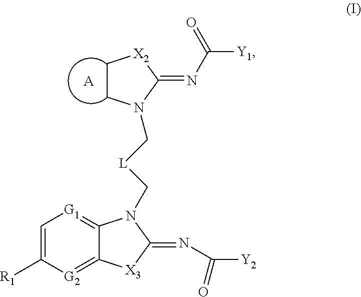
or a solvate, pharmaceutically acceptable salt, or tautomer thereof, wherein:
Ring A is selected from the group consisting of 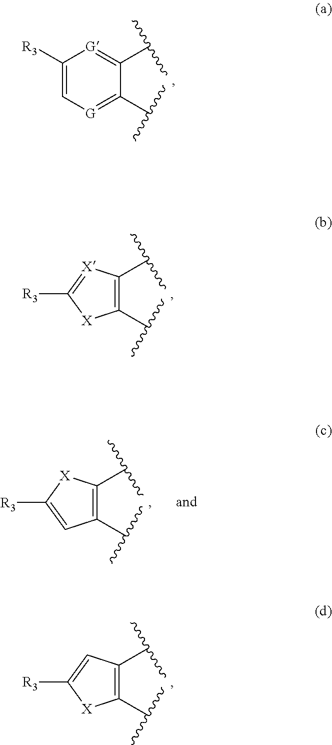
wherein
G and G₁ are independently N, CH, or C—X₁—R₂;
G′ and G₂ are independently N or CH;
X is N—R, O, or S;
X′ is N or CH;
X₁ is CH₂, O or S;
R is hydrogen or a C₁₋₄ alkyl, and
wherein when G and G₁ are each C—X₁—R₂, the R₂ groups are optionally linked to form L₁;
L and L₁ are each independently C₂₋₄ alkylene or C₂₋₄ alkenylene;
R₂ is selected from the group consisting of
hydrogen, a C₂₋₄ cyclic ether, a C₃₋₄ cyclic alkyl, C₁₋₄ alkyl, 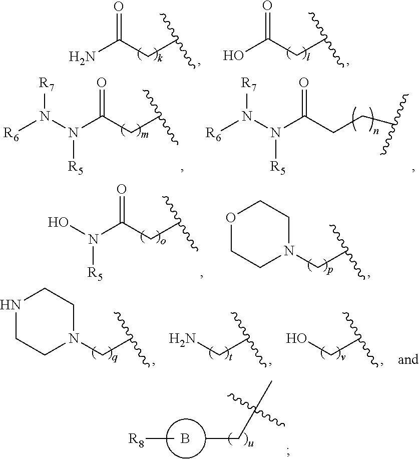
R₁ and R₅ are independently selected from the group consisting of 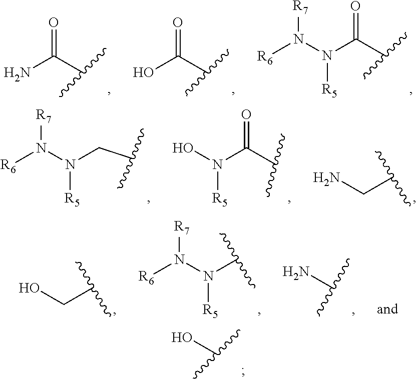
Ring B is a 6-membered aromatic ring or a 5- or 6-membered heteroaromatic ring comprising 1 to 2 heteroatoms selected from N, O, and S;
R₈ is —OH or —NR₉R₁₀;
R₉ and R₁₀ are independently selected from hydrogen and C₁-C₆ alkyl;
X₂ and X₃ are independently NH or S;
Y₁ and Y₂ are independently 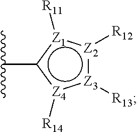
Z₁, Z₂, Z₃, and Z₄ are each independently C, N, O, or S;
R₅, R₆, and R₇ are independently selected from hydrogen, C₁-C₆ alkyl, and C₂-C₆ alkenyl, 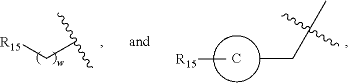
wherein R₅ and Rₑ are optionally connected to form a 5- or 6-membered heterocyclic ring;
R₁₅ is —OH or —NR₉R₁₀;
Ring C is a 6-membered aromatic ring or a 5- or 6-membered heteroaromatic ring comprising 1 to 2 heteroatoms selected from N, O, and S;
R₁₁, R₁₂, R₁₃, and R₁₄ are independently absent, hydrogen, or C₁₋₄ alkyl;
n, p, q, t, and v are independently an integer from 2 to 6; and
k, l, m, o, u, and w are independently an integer from 1 to 6, and
provided that at least one of G and G₁ is C—X₁—R₂, wherein R₂ is 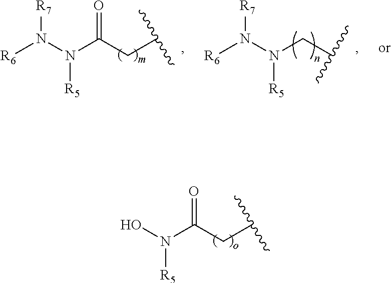
and/or
at least one of R₁ and R₃ is

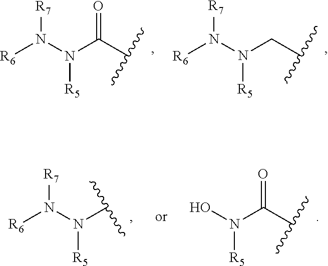

2. The compound of claim 1, wherein L is a C₂-alkenylene.

3. The compound of claim 1, wherein:
L is a C₂-alkenylene; and
X₂ and X₃ are NH.

4. The compound of claim 1 selected from the group consisting of

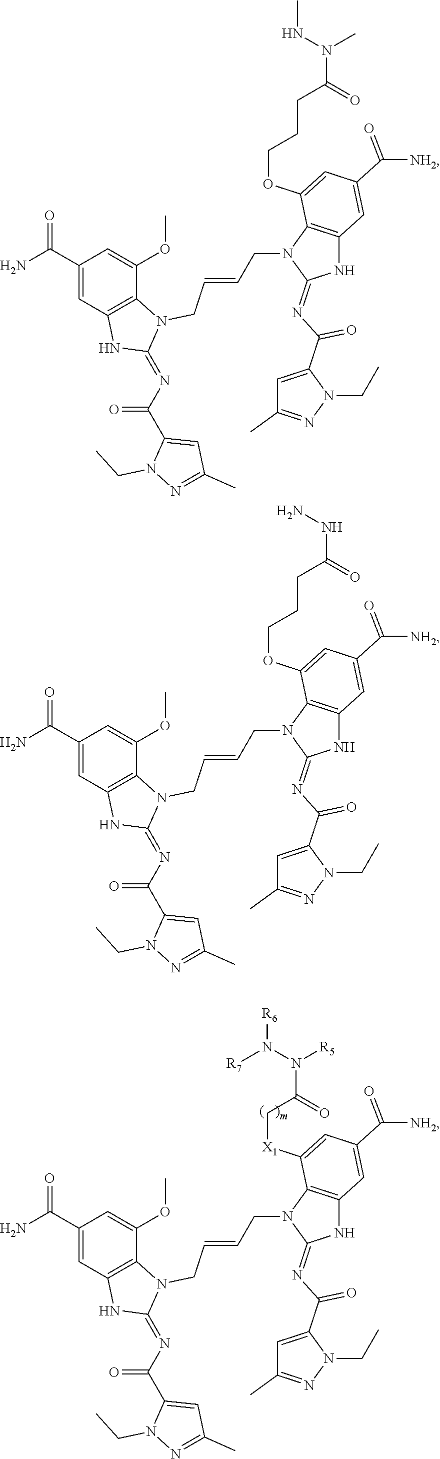

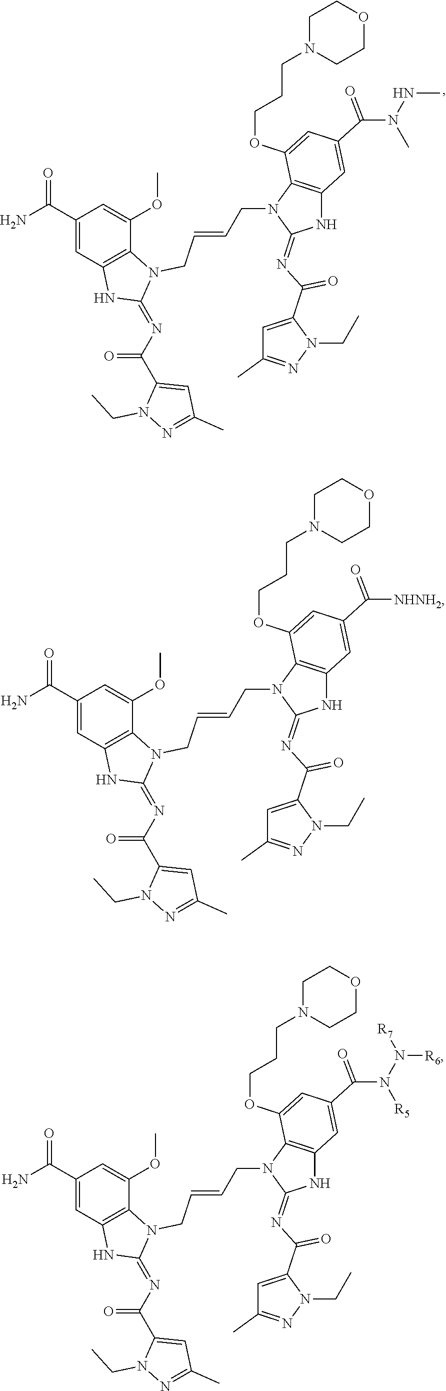

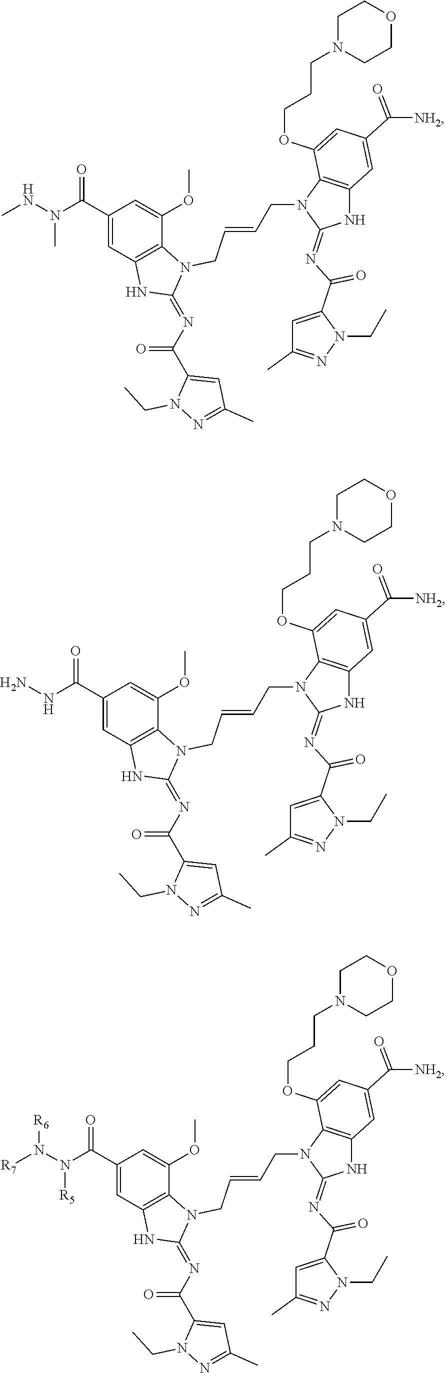

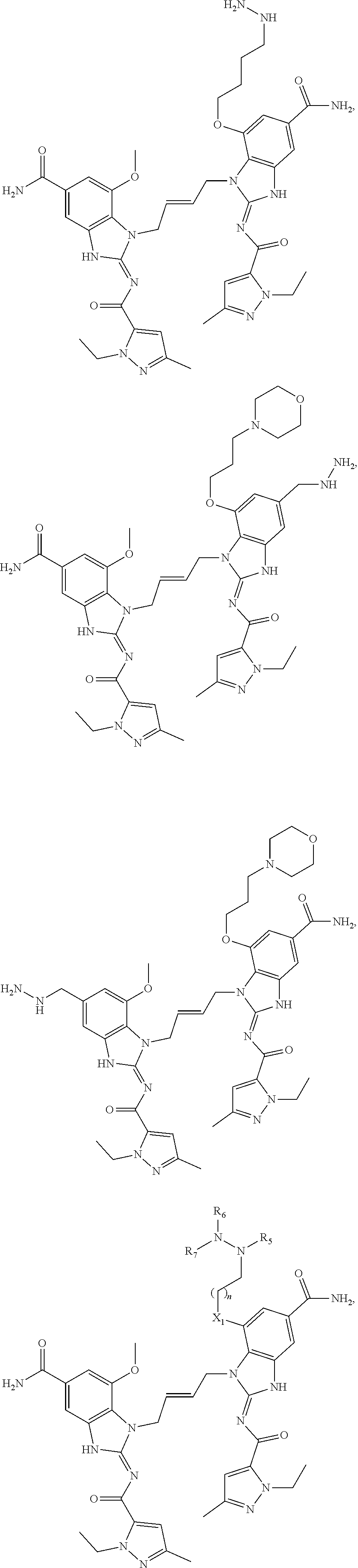

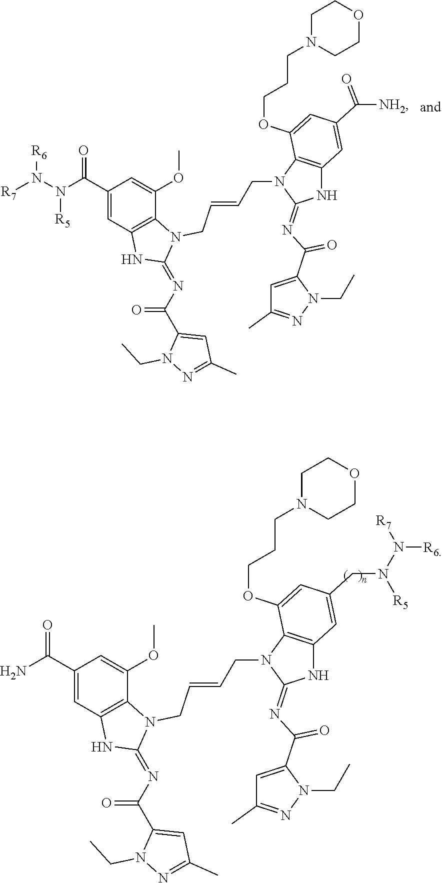

5. The compound of claim 1 selected from the group consisting of

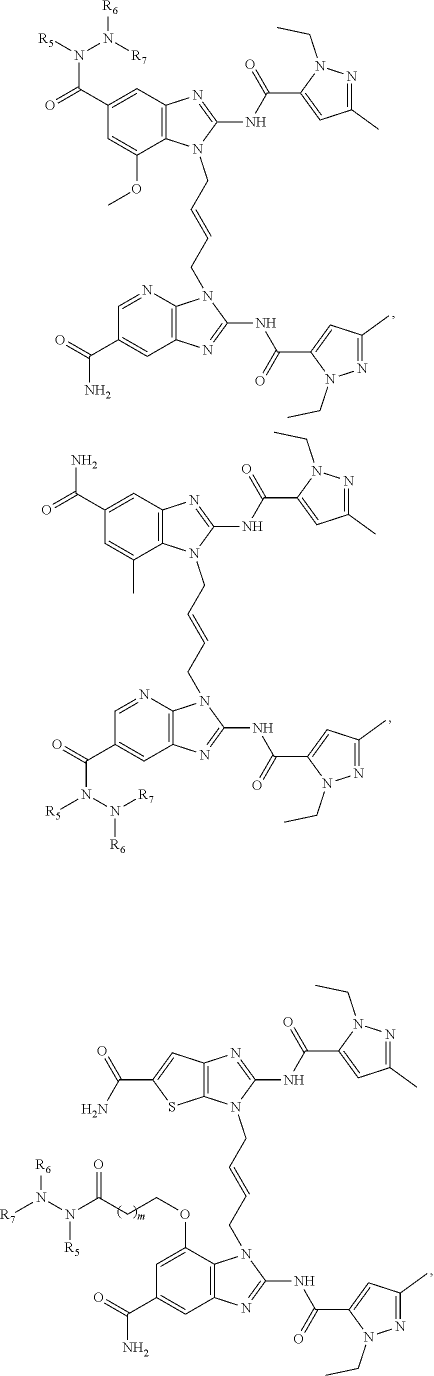

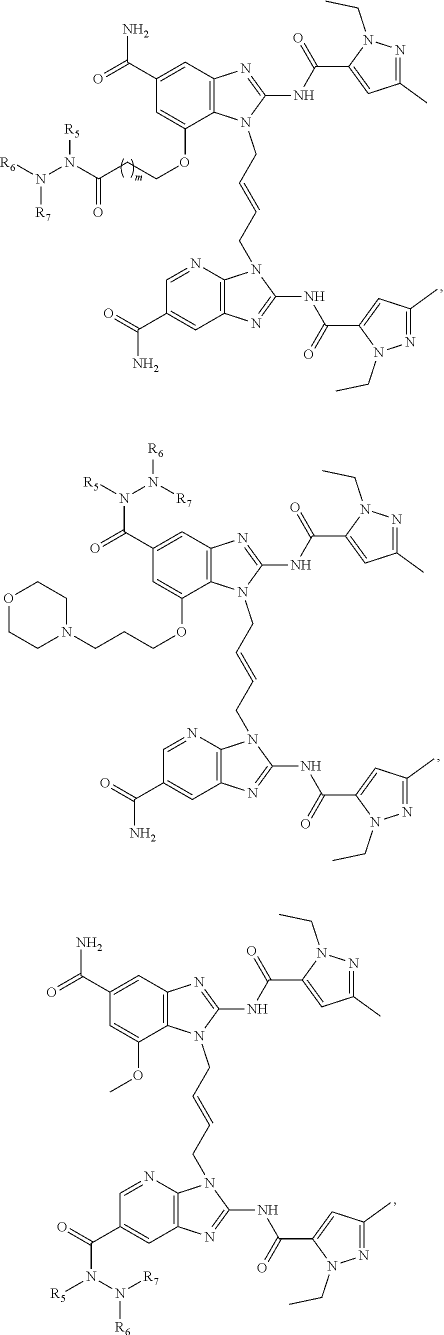

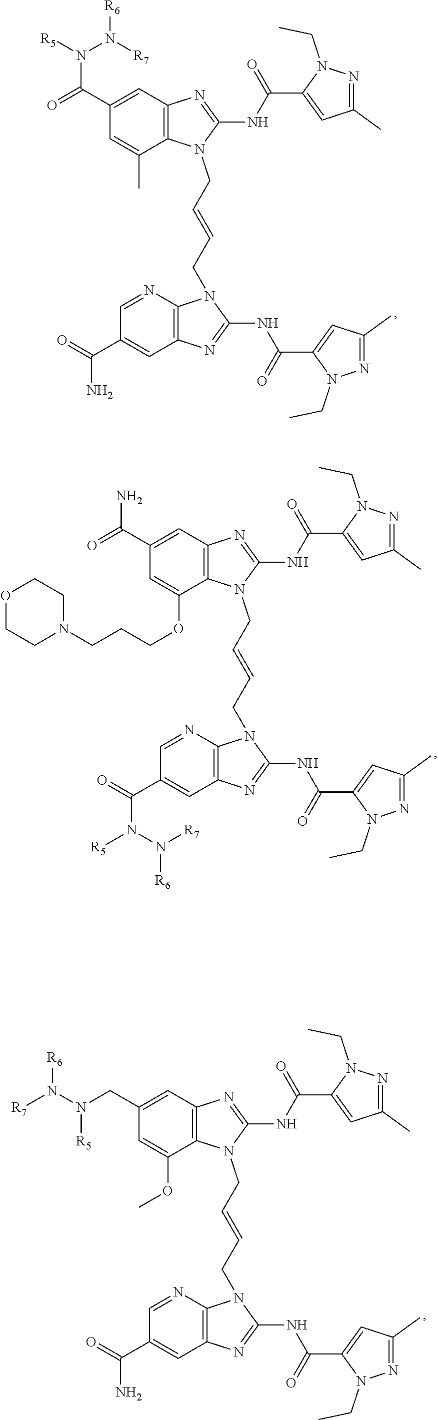

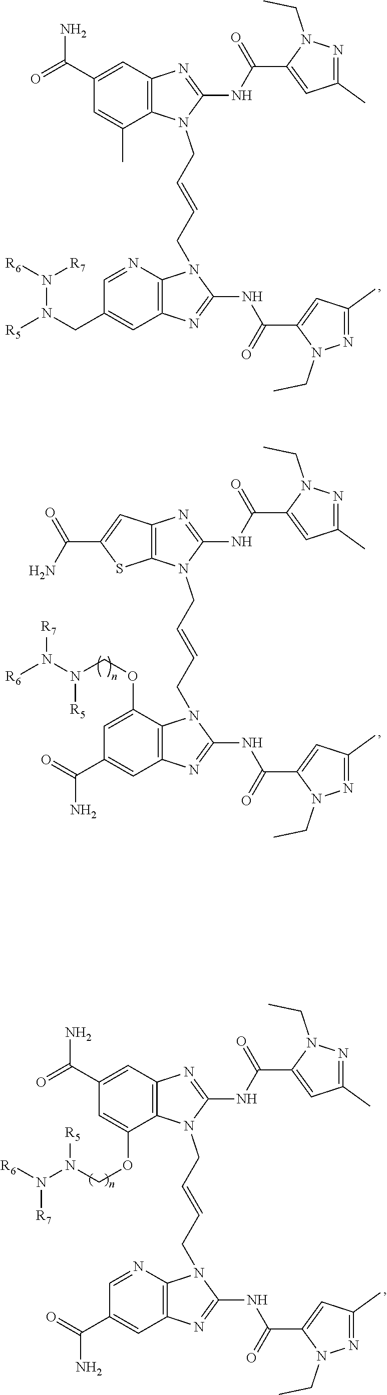

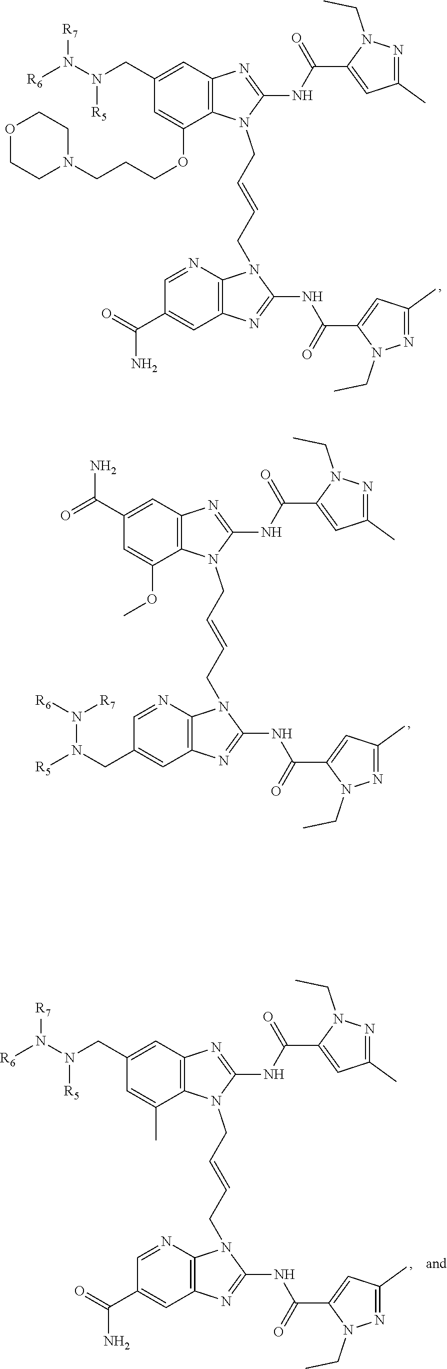

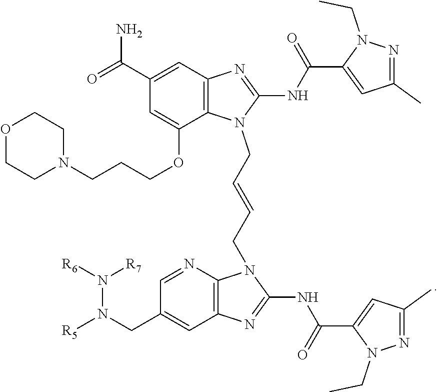

6. A compound of Formula IA: 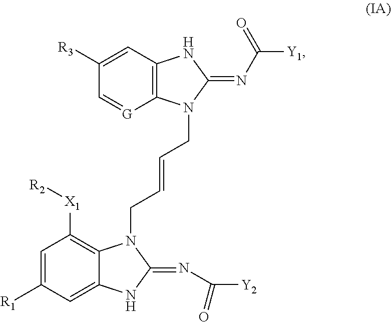
or a solvate, pharmaceutically acceptable salt, or tautomer thereof, wherein:
X₁ is CH₂, O or S;
G is CH, C—SCH₃, C—OCH₃, or N;
R₂ is selected from the group consisting of
hydrogen, a C₂₋₄ cyclic ether, a C₃₋₄ cyclic alkyl, C₁₋₄ alkyl, 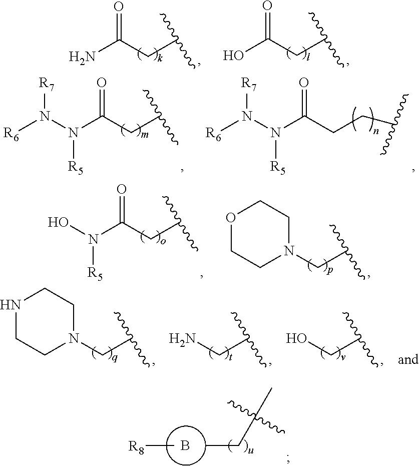
R₁ and R₃ are independently selected from the group consisting of 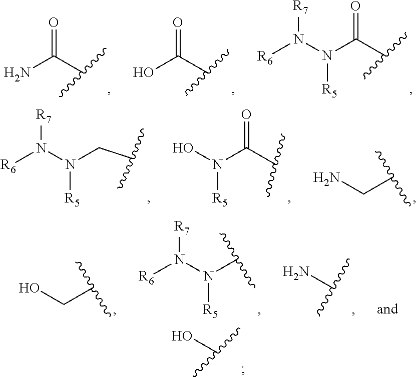
Ring B is a 6-membered aromatic ring or a 5- or 6-membered heteroaromatic ring comprising 1 to 2 heteroatoms selected from N, O, and S;
R₈ is —OH or —NH₂;
Y₁ and Y₂ are independently 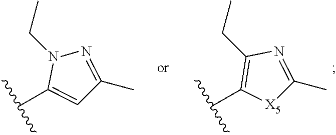
X₅ is S, O, or NR₇;
R₅, R₆, and R₇ are independently selected from hydrogen, C₁-C₆ alkyl, and C₂-C₆ alkenyl, 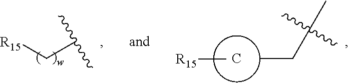
wherein R₅ and R₆ are optionally connected to form a 5- or 6-membered heterocyclic ring;
R₁₅ is —OH or —NR R₁₀;
R₉ and R₁₀ are independently selected from hydrogen and C₁-C₆ alkyl;
Ring C is a 6-membered aromatic ring or a 5- or 6-membered heteroaromatic ring comprising 1 to 2 heteroatoms selected from N, O, and S;
n, p, q, t, and v are independently an integer from 2 to 6; and
k, l, m, o, u, and w are independently an integer from 1 to 6, and
provided that R₂ is 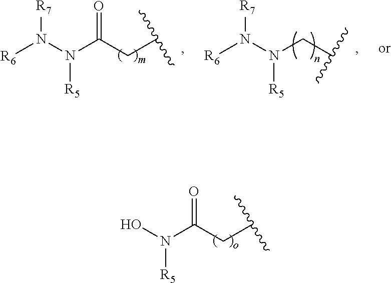
and/or
at least one of R₁ and R₃ is

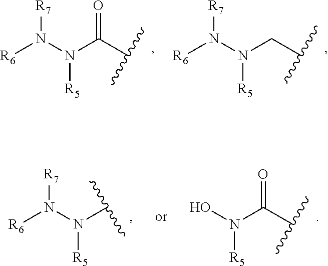

7. The compound of claim 6, wherein X₁ is O.

8. The compound of claim 6 selected from the group consisting of

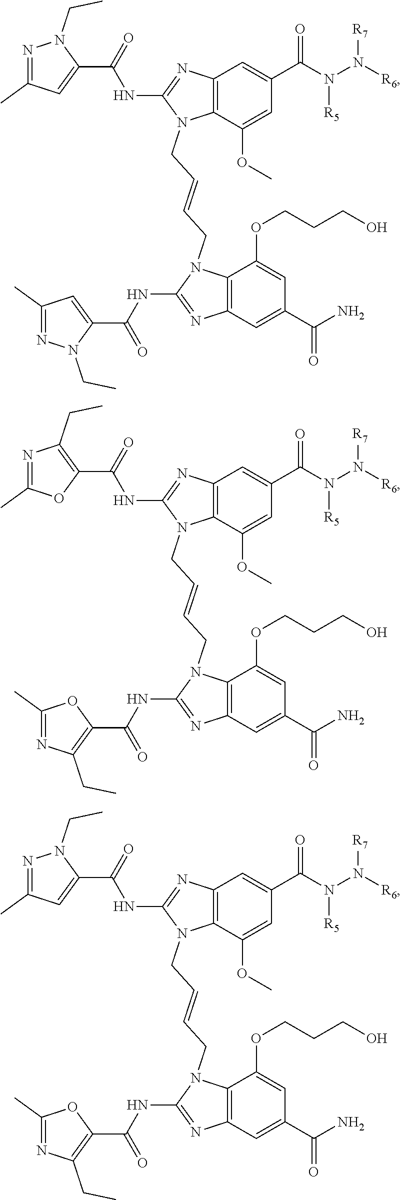

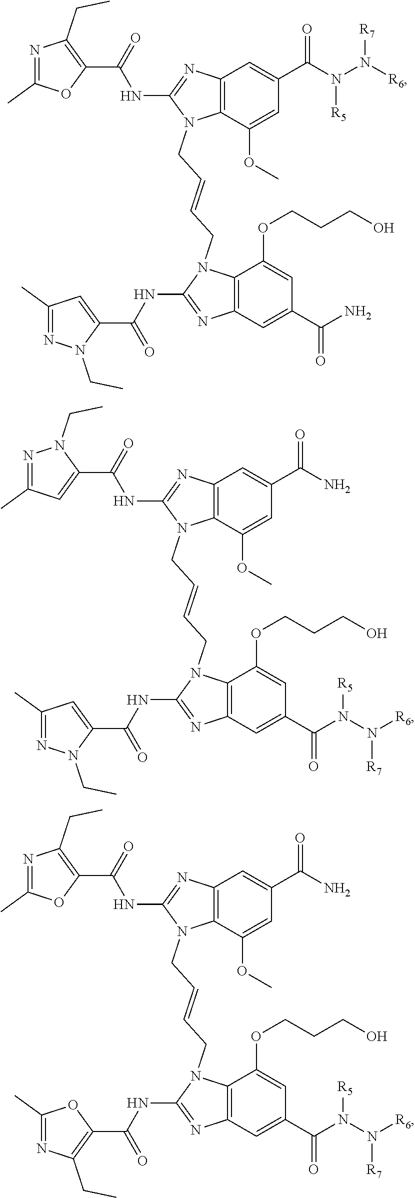

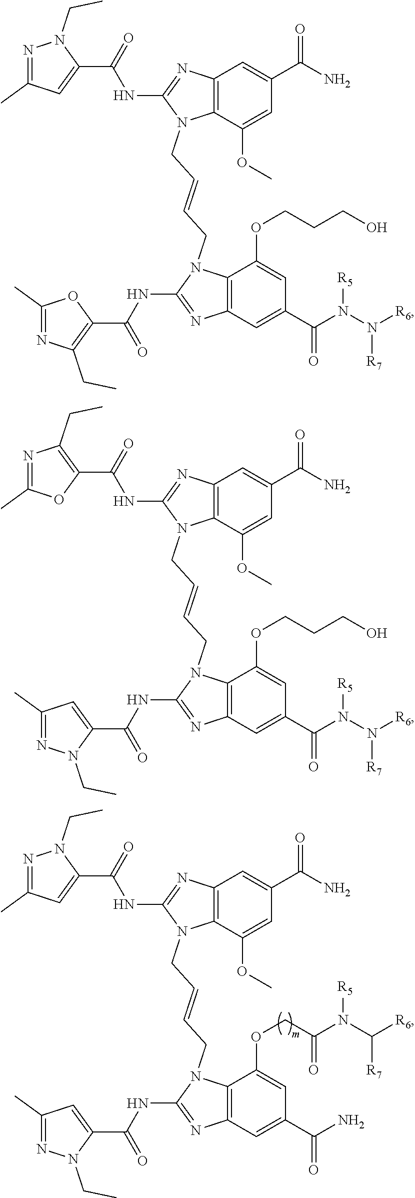

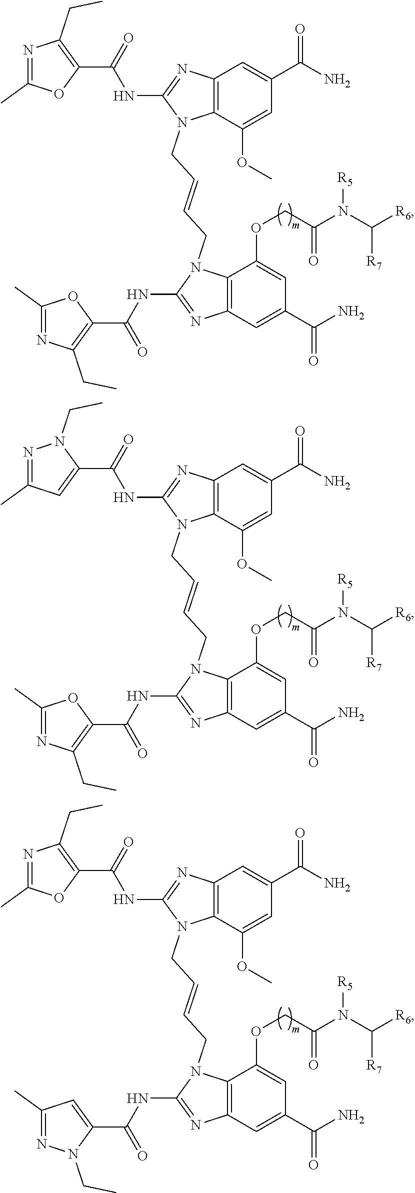

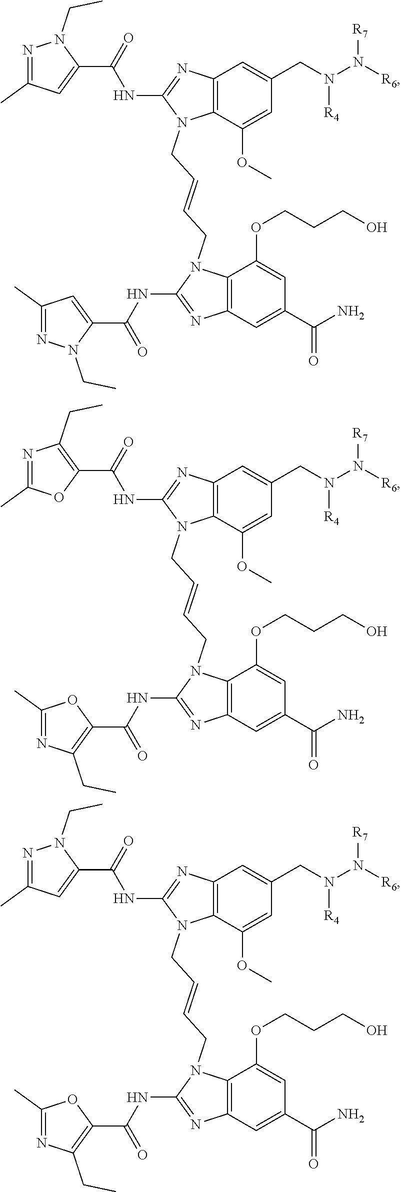

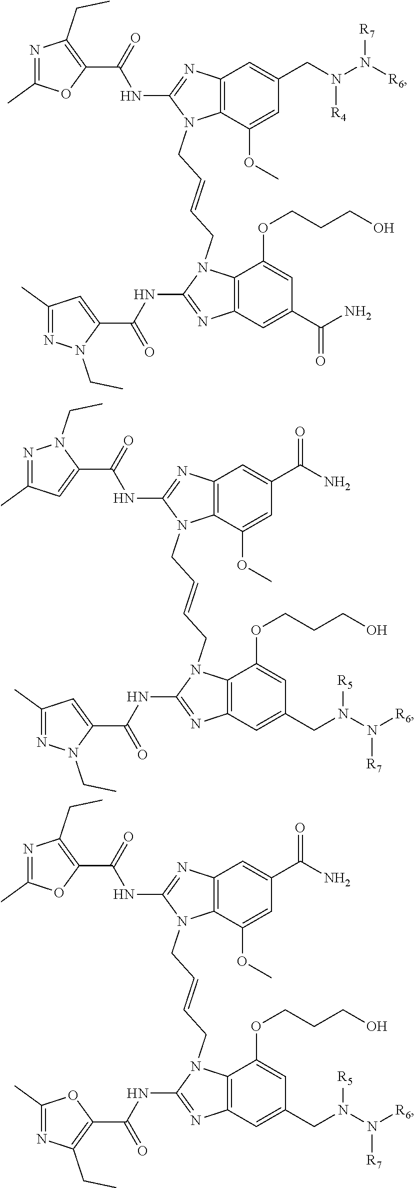

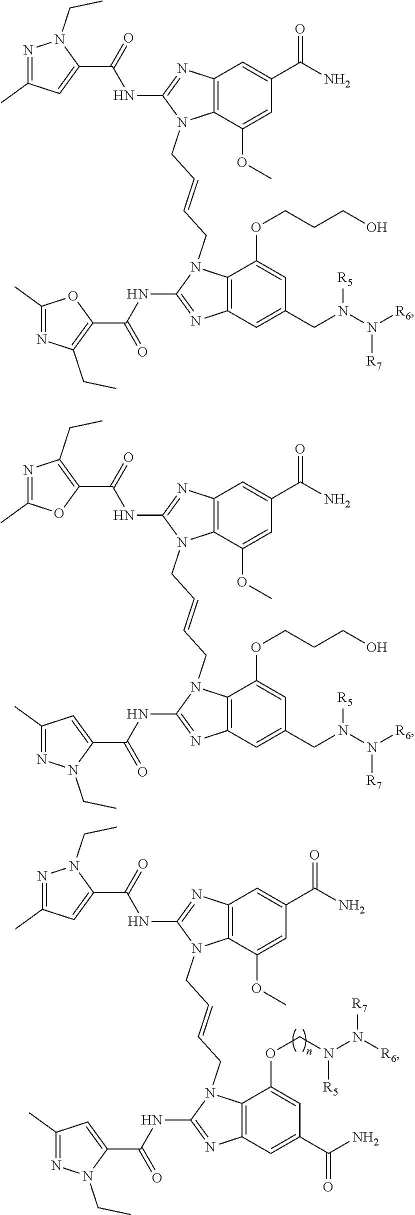

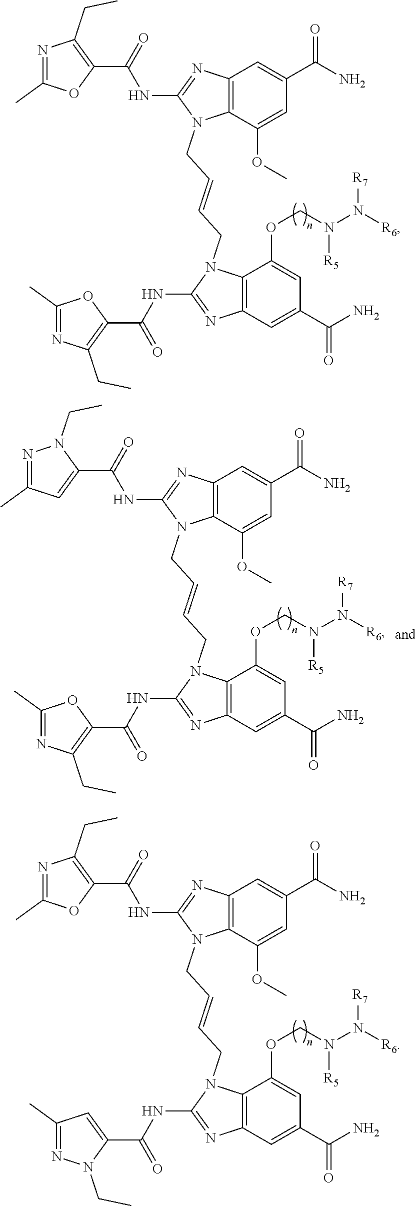

9. The compound of claim 6 selected from the group consisting of

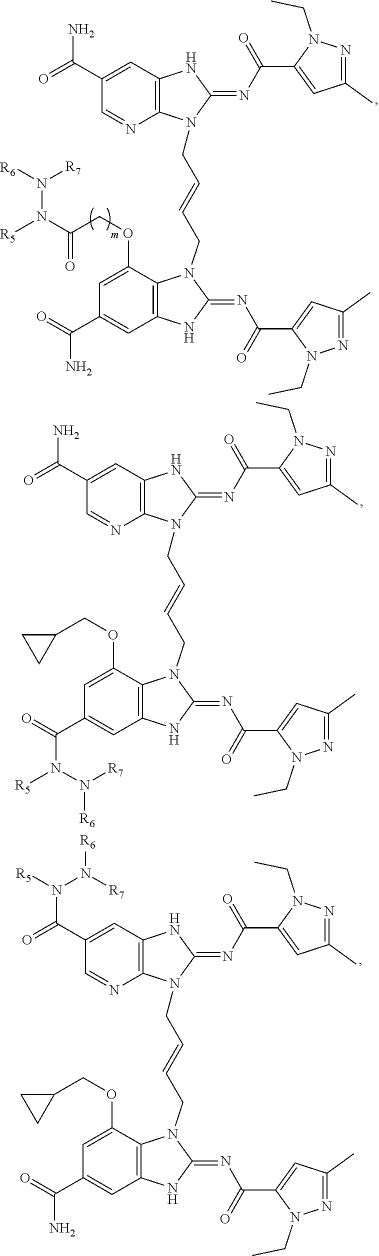

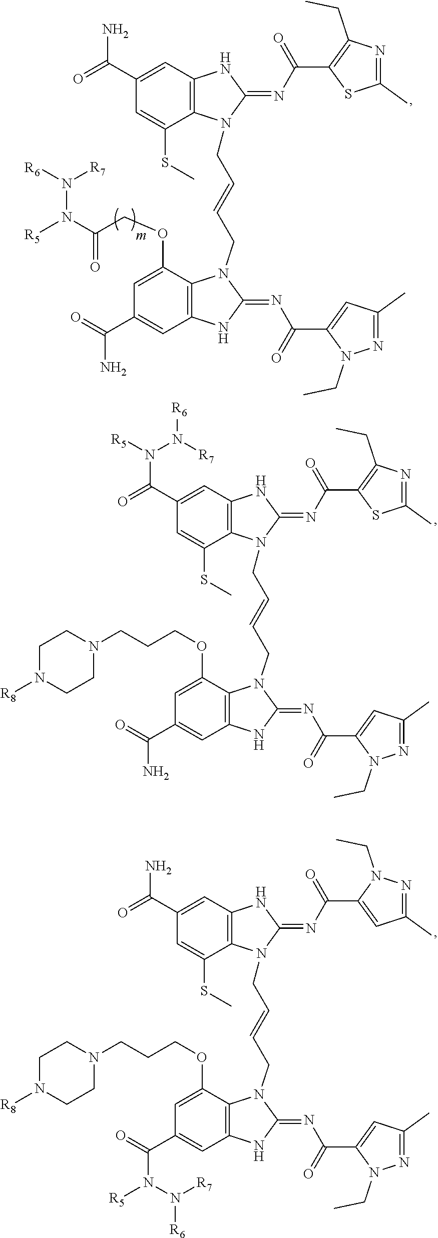

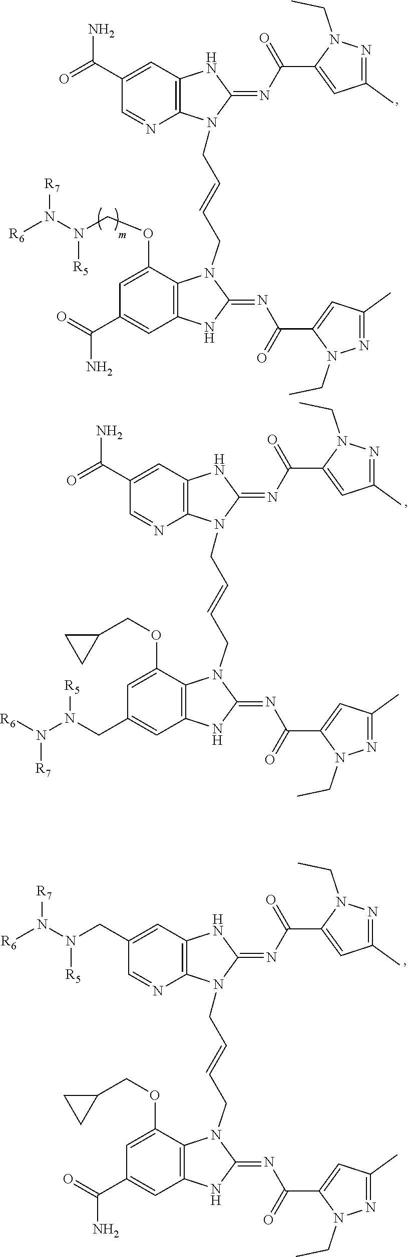

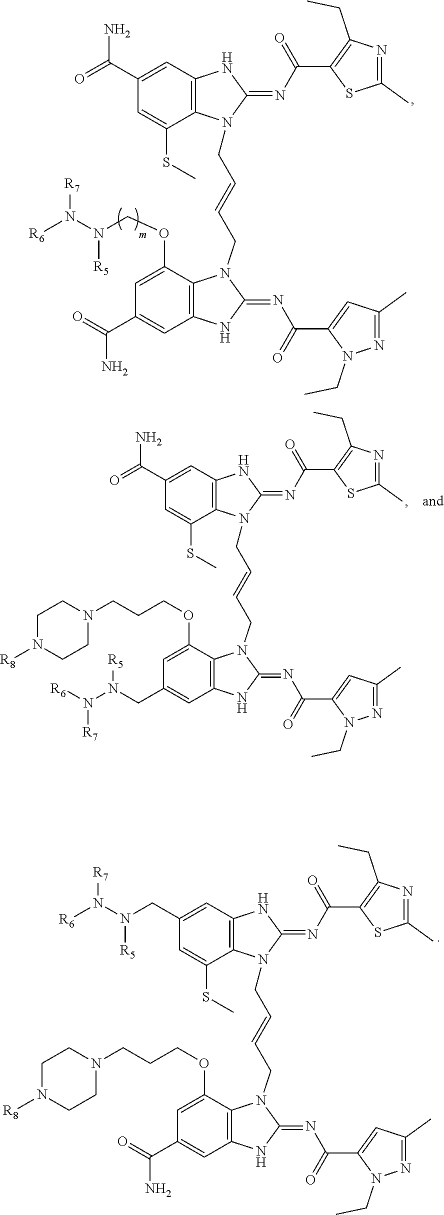

10. A compound of Formula IA′: 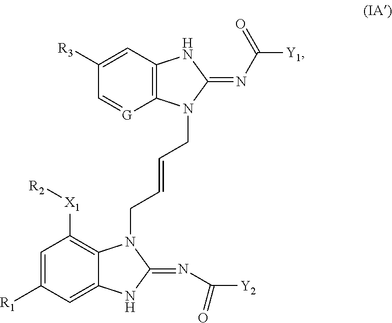
or a solvate, pharmaceutically acceptable salt, or tautomer thereof, wherein:
X₁ is CH₂, O or S;
G is G is CH, C—SCH₃, C—OCH₃, or N;
R₂ is selected from the group consisting of
hydrogen, a C₂₋₄ cyclic ether, a C₃₋₄ cyclic alkyl, C₁₋₄ alkyl, 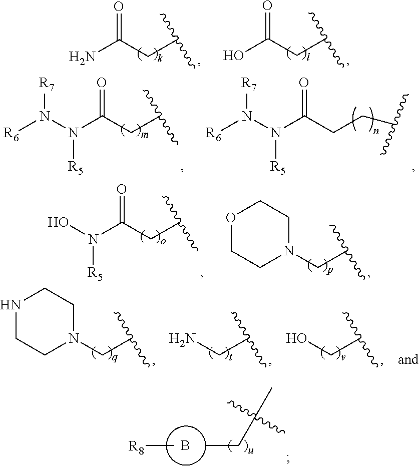
R₁ and R₅ are independently selected from the group consisting of 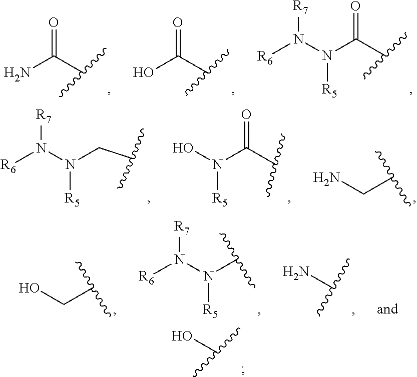
Ring B is a 6-membered aromatic ring or a 5- or 6-membered heteroaromatic ring comprising 1 to 2 heteroatoms selected from N, O, and S;
R₈ is —OH or —NH₂;
Y₁ and Y₂ are independently 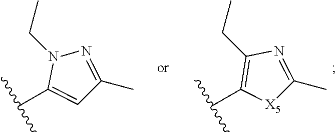
X₅ is S, O, or NR₇;
R₅, R₆, and R₇ are independently selected from hydrogen, —OH, C₁-C₆ alkyl, 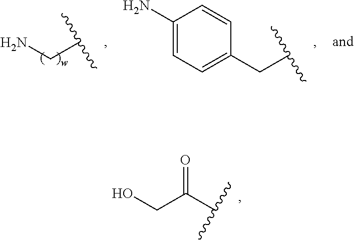
wherein R₅ and R₆ are optionally connected to form a 5- or 6-membered heterocyclic ring;
n, p, q, t, and v are independently an integer from 2 to 6; and
k, l, m, o, u, and w are independently an integer from 1 to 6, and
provided that R₂ is 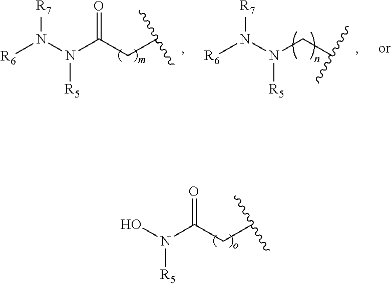
and/or
at least one of R₁ and Ra is

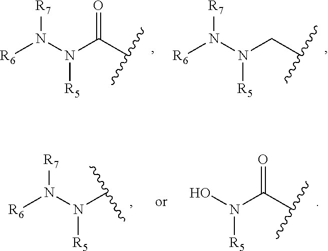

11. The compound of claim 10 selected from the group consisting of

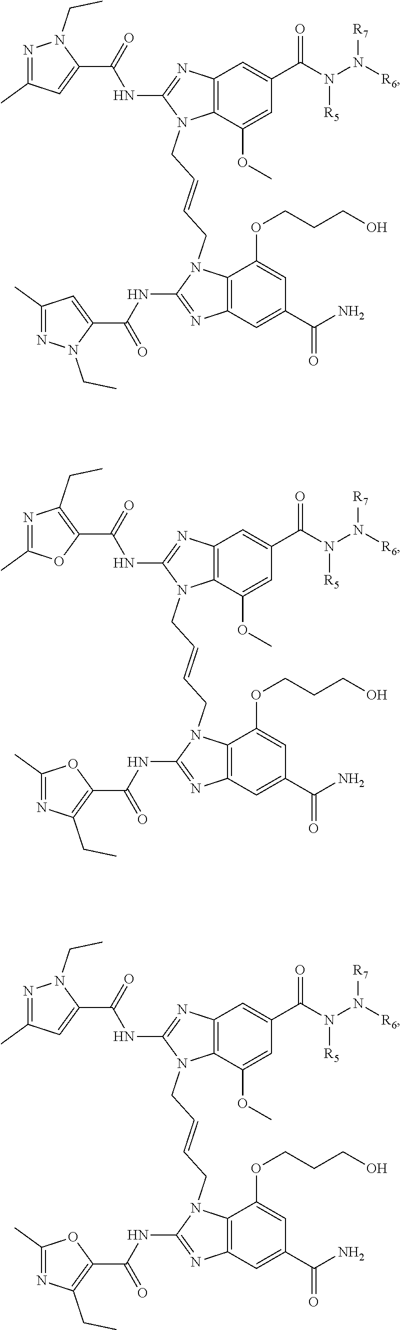

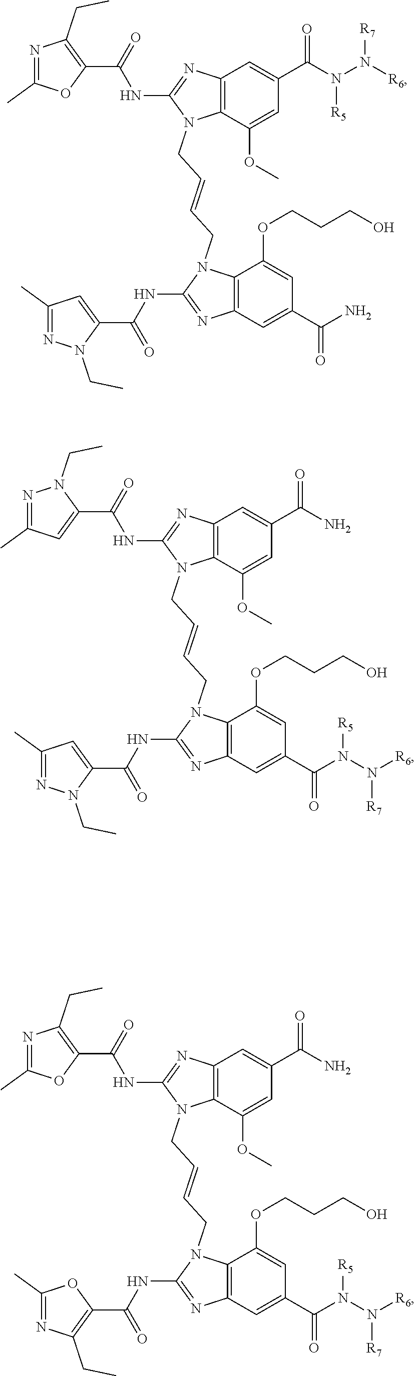

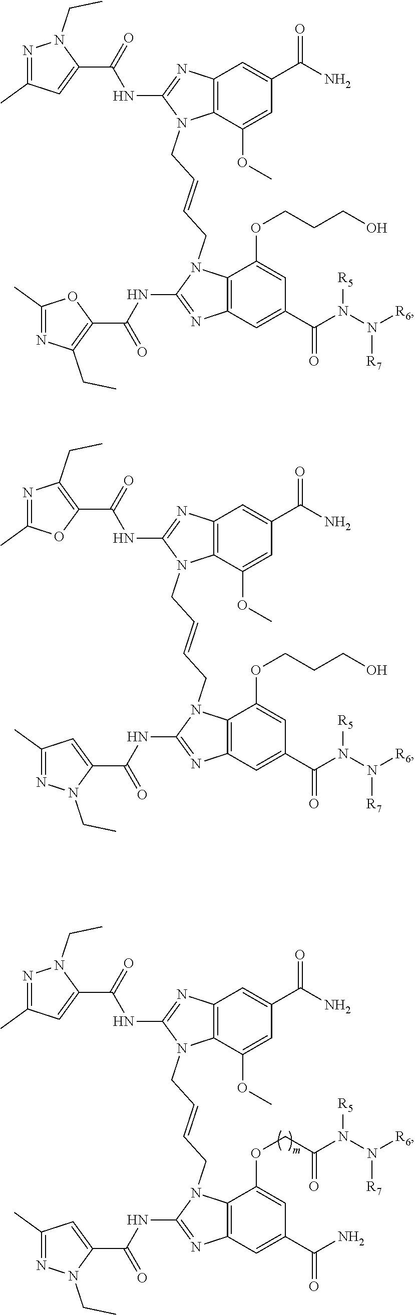

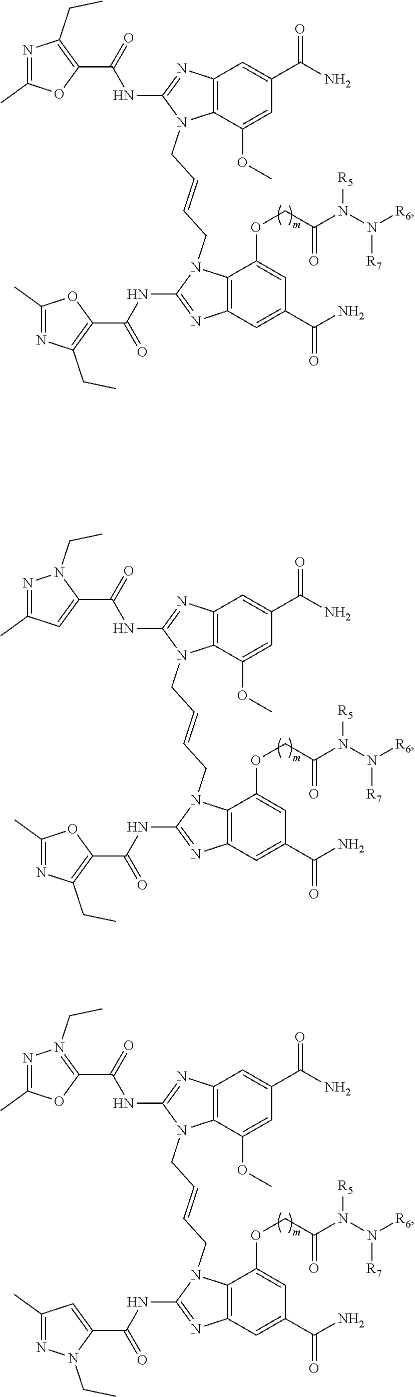

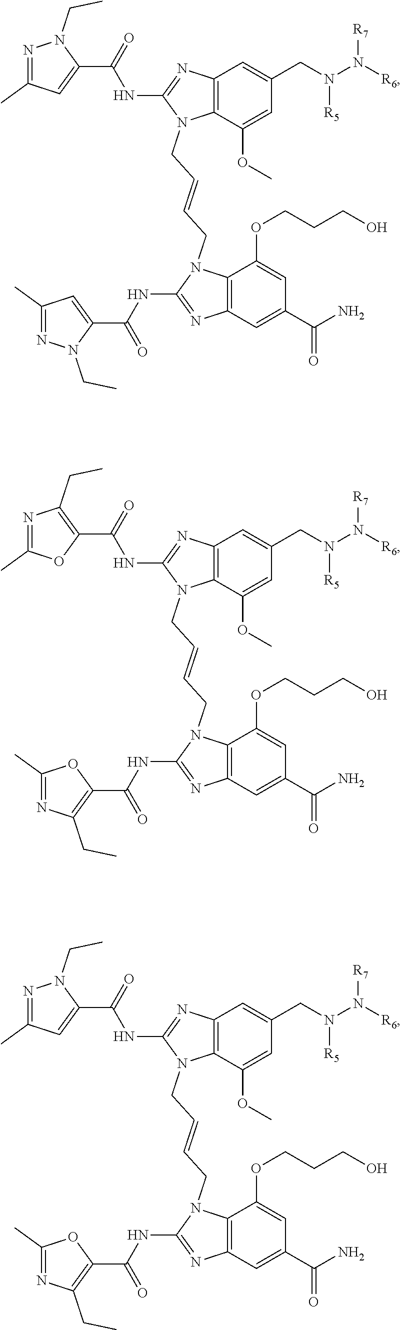

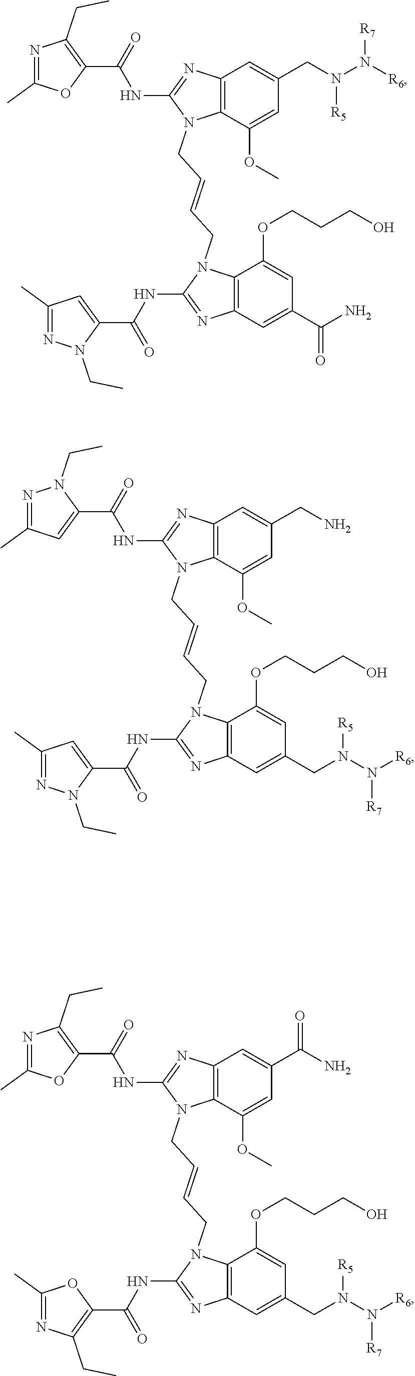

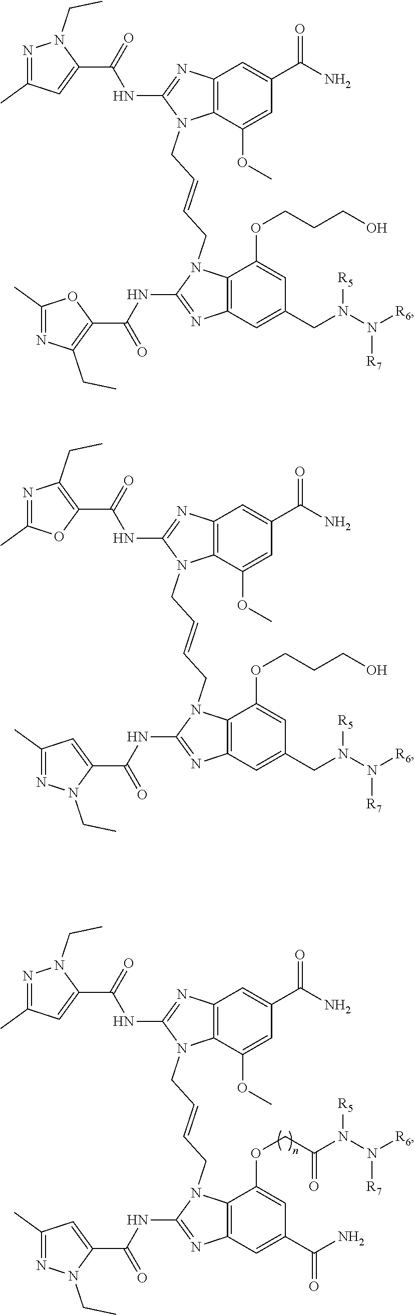

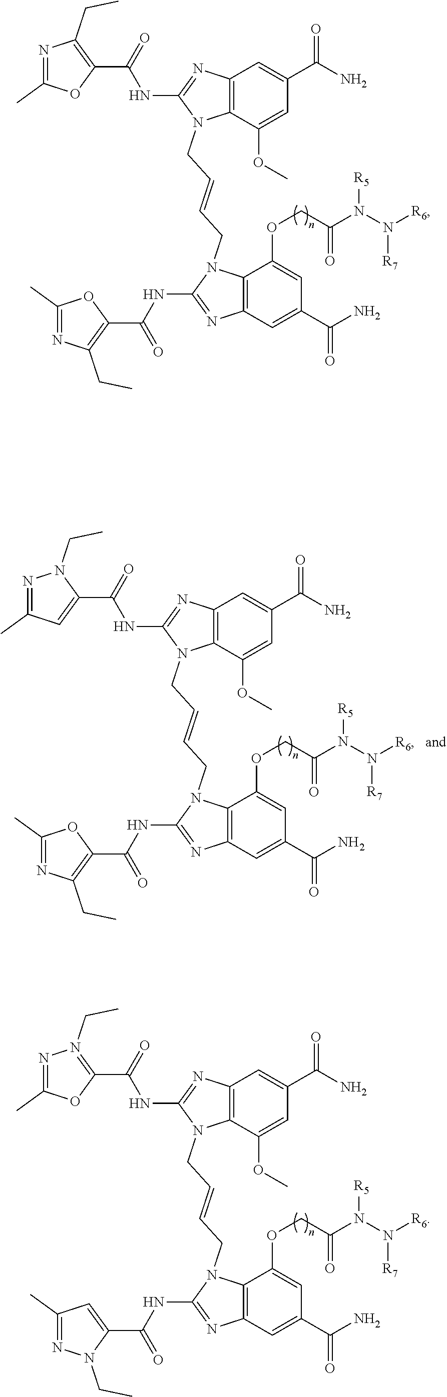

12. The compound of claim 10 selected from the group consisting of

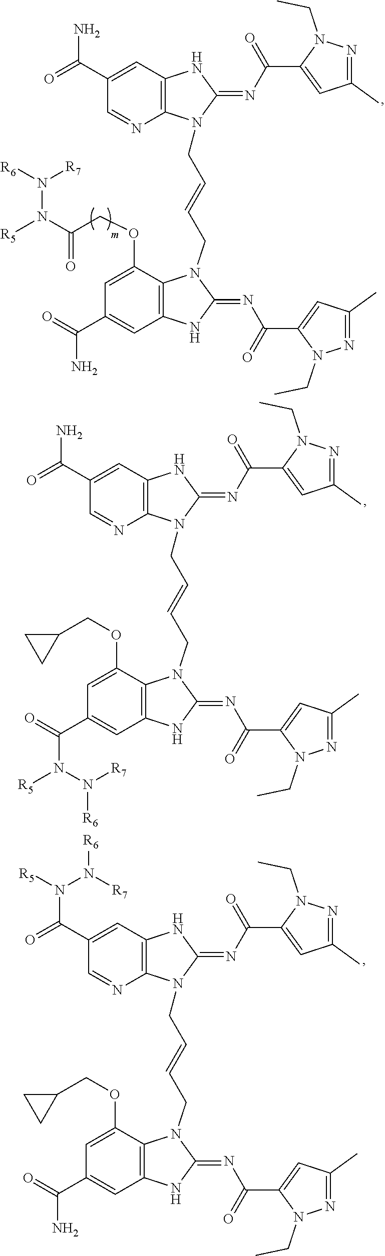

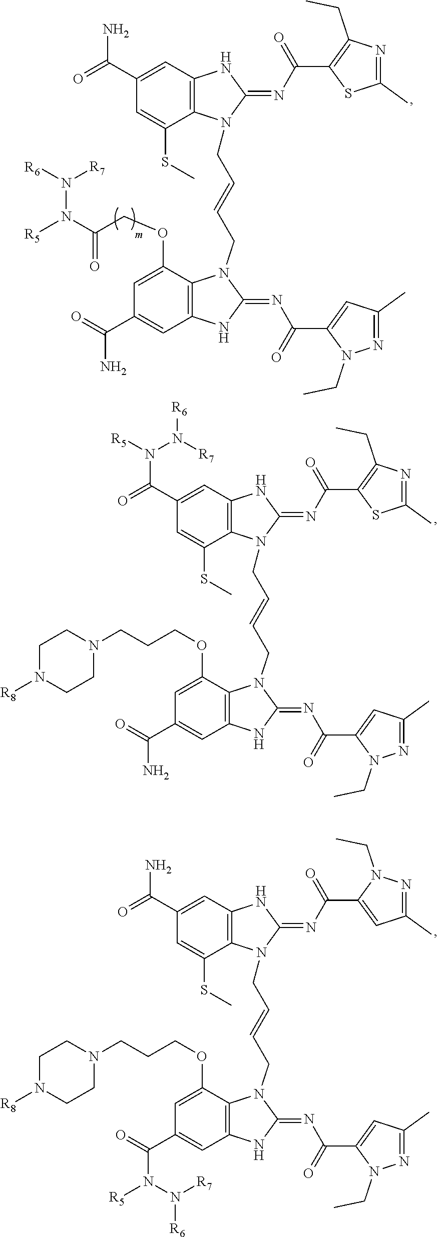

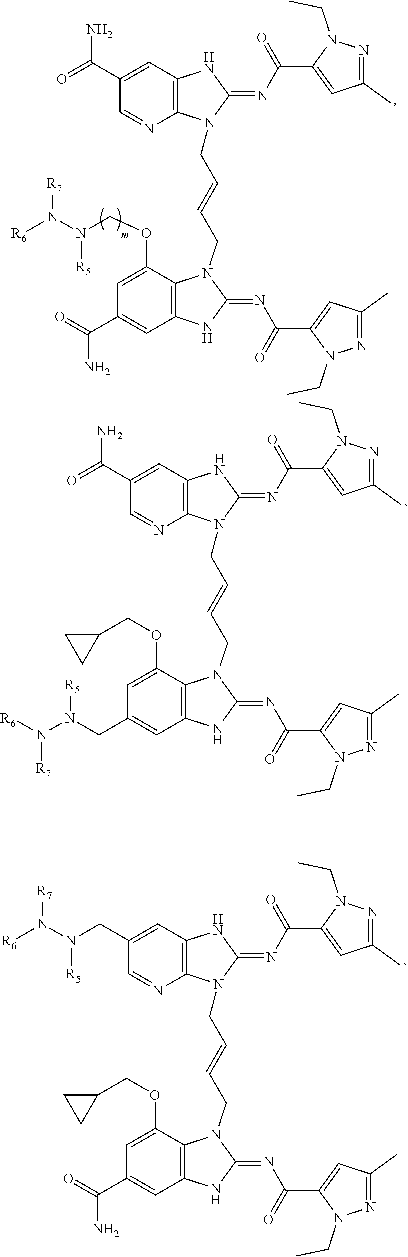

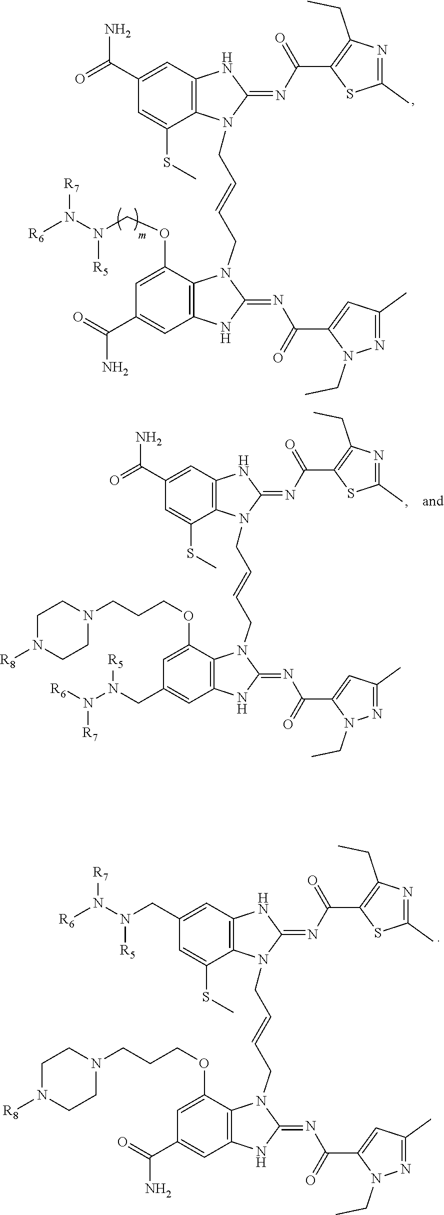

13. A compound of Formula IB: 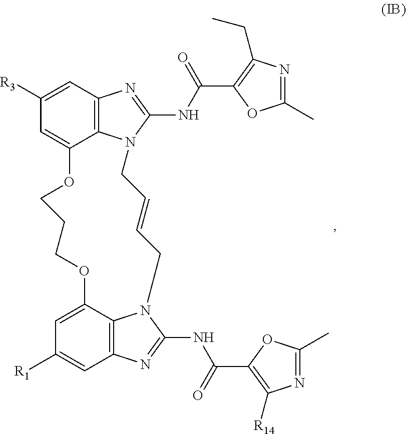
or a solvate, pharmaceutically acceptable salt, or tautomer thereof, wherein:
R₁ and R₃ are independently selected from the group consisting of 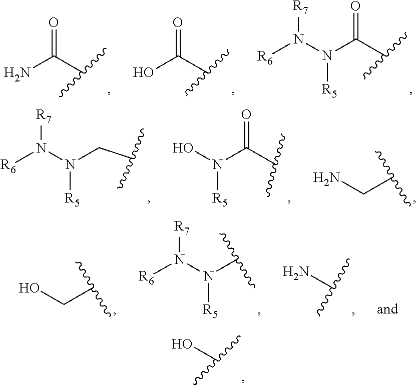
wherein optionally R₅ and R₆ are connected to form a 5- or 6-membered ring;
R₁₄ is hydrogen, or C₁₋₄ alkyl;
R₅, R₆, and R₇ are independently selected from hydrogen, —OH, C₁-C₆ alkyl, 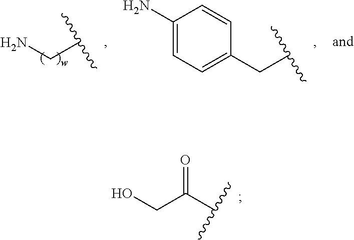
and
w is an integer from 1 to 6 and provided that at least one of R₁ and R₃ is

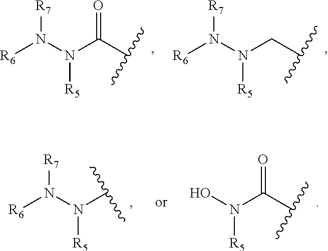

14. The compound of claim 13 selected from the group consisting of:

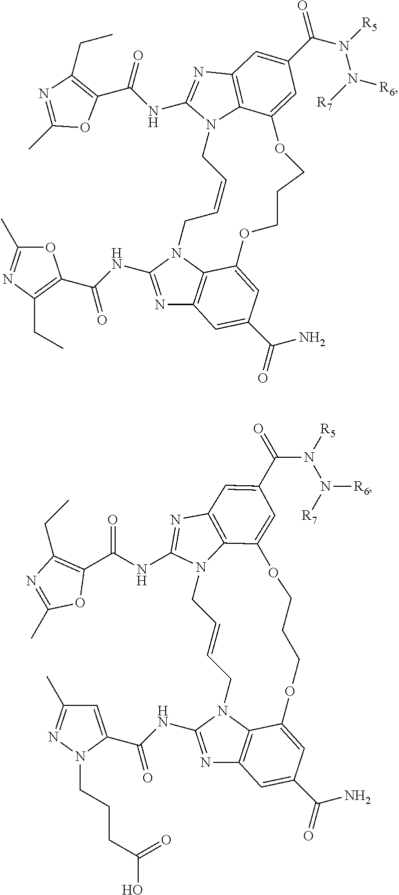

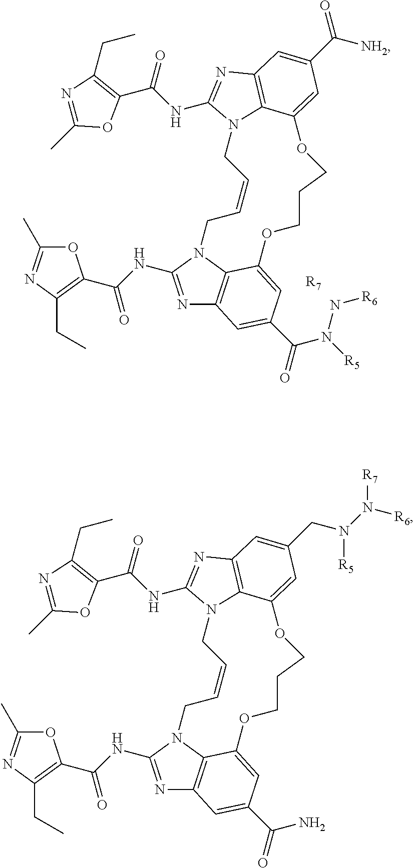

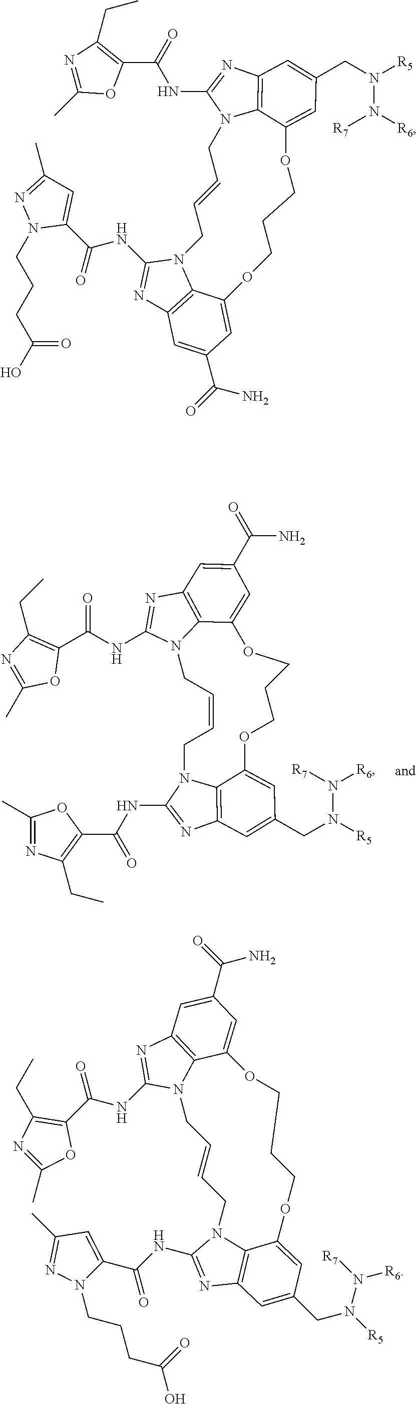

15. The compound of claim 1 wherein the compound is 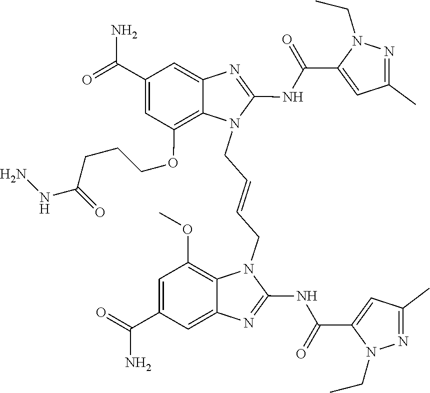 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NN)c21]
or a solvate, pharmaceutically acceptable salt, or tautomer thereof.

16. The compound of claim 1 wherein the compound is 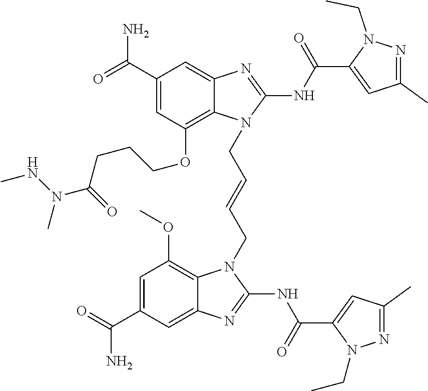
or a solvate, pharmaceutically acceptable salt, or tautomer thereof.

17. The compound of claim 1 wherein the compound is 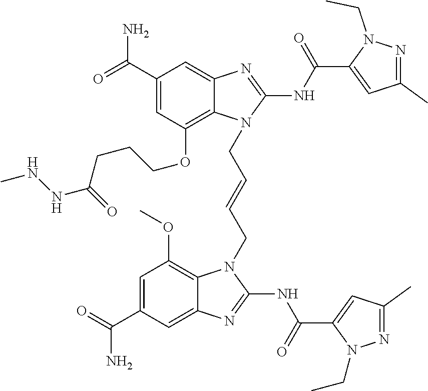
or a solvate, pharmaceutically acceptable salt, or tautomer thereof.

18. The compound of claim 1 wherein the compound is 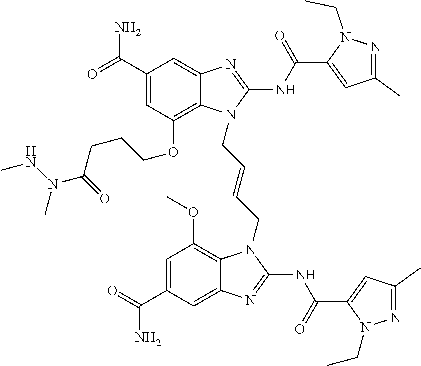
or a solvate, pharmaceutically acceptable salt, or tautomer thereof.

19. The compound of claim 1 wherein the compound is 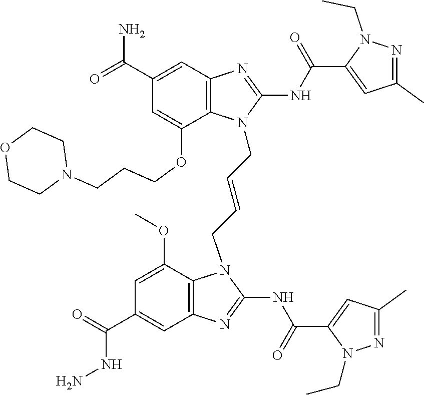 [SMILES: CCn1nc(C)cc1C(=O)Nc1nc2cc(C(=O)NN)cc(OC)c2n1C/C=C/Cn1c(NC(=O)c2cc(C)nn2CC)nc2cc(C(N)=O)cc(OCCCN3CCOCC3)c21]
or a solvate, pharmaceutically acceptable salt, or tautomer thereof.

20. The compound of claim 1 wherein the compound is 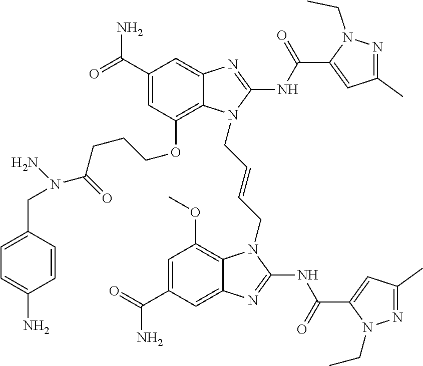 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(N)Cc3ccc(N)cc3)c21]
or a solvate, pharmaceutically acceptable salt, or tautomer thereof.

21. The compound of claim 1 wherein the compound is 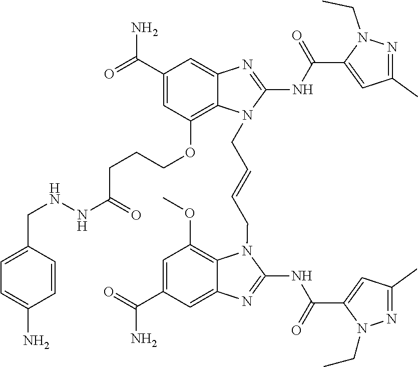 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NNCc3ccc(N)cc3)c21]
or a solvate, pharmaceutically acceptable salt, or tautomer thereof.

22. The compound of claim 1 wherein the compound is 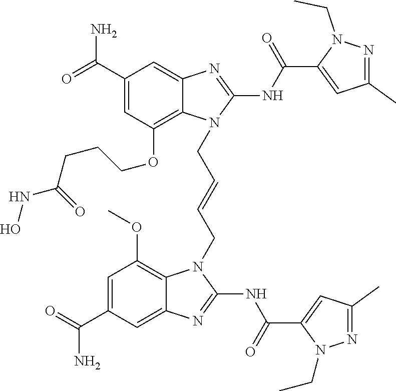 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)NO)c21]
or a solvate, pharmaceutically acceptable salt, or tautomer thereof.

23. The compound of claim 1 wherein the compound is 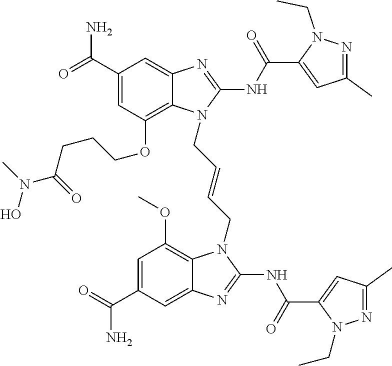
or a solvate, pharmaceutically acceptable salt, or tautomer thereof.

24. The compound of claim 1 wherein the compound is 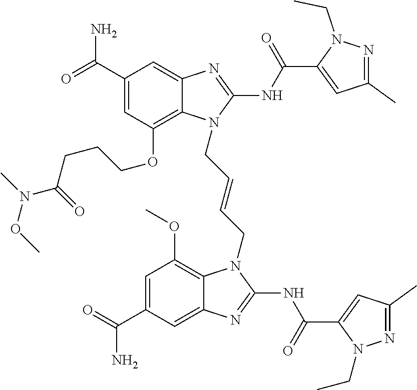
or a solvate, pharmaceutically acceptable salt, or tautomer thereof.

25. The compound of claim 1 wherein the compound is 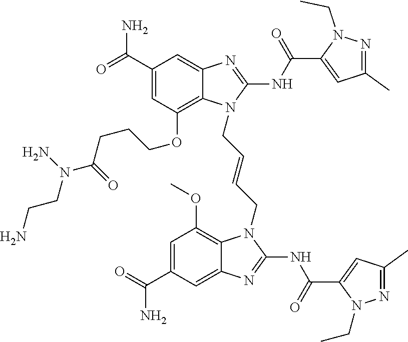 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(N)CCN)c21]
or a solvate, pharmaceutically acceptable salt, or tautomer thereof.

26. The compound of claim 1 wherein the compound is 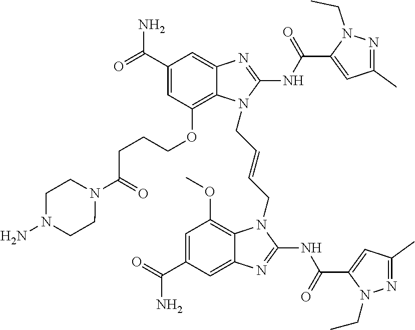 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N3CCN(N)CC3)c21]
or a solvate, pharmaceutically acceptable salt, or tautomer thereof.

27. The compound of claim 1 wherein the compound is 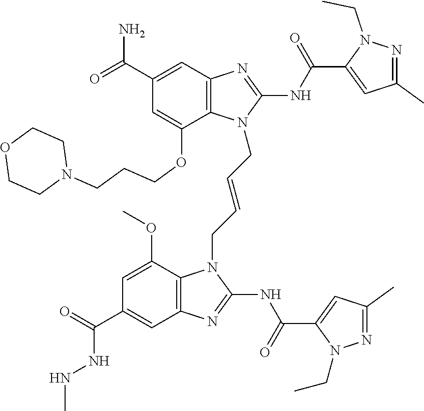 [SMILES: CCn1nc(C)cc1C(=O)Nc1nc2cc(C(=O)NNC)cc(OC)c2n1C/C=C/Cn1c(NC(=O)c2cc(C)nn2CC)nc2cc(C(N)=O)cc(OCCCN3CCOCC3)c21]
or a solvate, pharmaceutically acceptable salt, or tautomer thereof.

28. The compound of claim 1 wherein the compound is 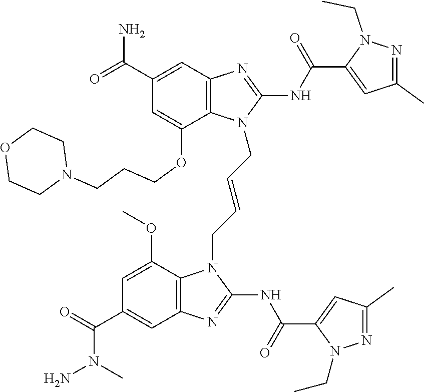 [SMILES: CCn1nc(C)cc1C(=O)Nc1nc2cc(C(=O)N(C)N)cc(OC)c2n1C/C=C/Cn1c(NC(=O)c2cc(C)nn2CC)nc2cc(C(N)=O)cc(OCCCN3CCOCC3)c21]
or a solvate, pharmaceutically acceptable salt, or tautomer thereof.

29. The compound of claim 1 wherein the compound is 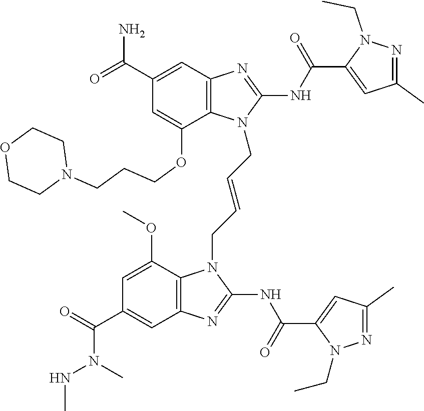 [SMILES: CCn1nc(C)cc1C(=O)Nc1nc2cc(C(=O)N(C)NC)cc(OC)c2n1C/C=C/Cn1c(NC(=O)c2cc(C)nn2CC)nc2cc(C(N)=O)cc(OCCCN3CCOCC3)c21]
or a solvate, pharmaceutically acceptable salt, or tautomer thereof.

30. The compound of claim 1 wherein the compound is 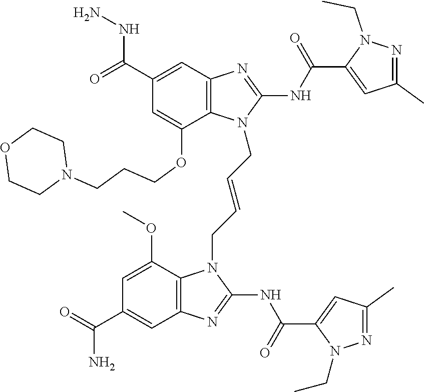 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NN)cc(OCCCN3CCOCC3)c21]
or a solvate, pharmaceutically acceptable salt, or tautomer thereof.

31. The compound of claim 1 wherein the compound is 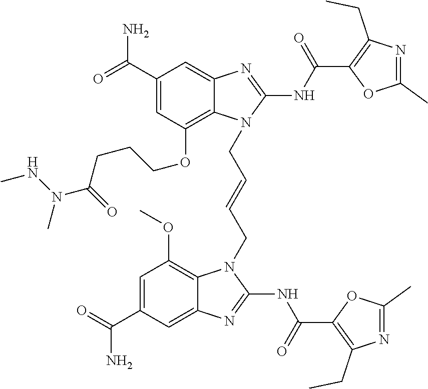
or a solvate, pharmaceutically acceptable salt, or tautomer thereof.

32. The compound of claim 1 wherein the compound is 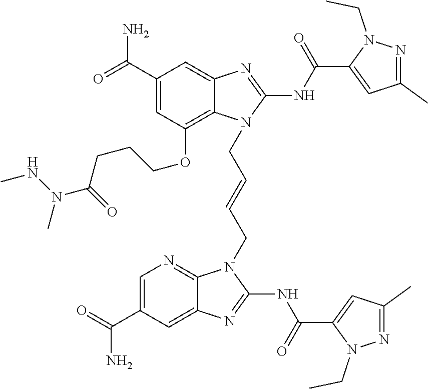
or a solvate, pharmaceutically acceptable salt, or tautomer thereof.

33. The compound of claim 1 wherein the compound is 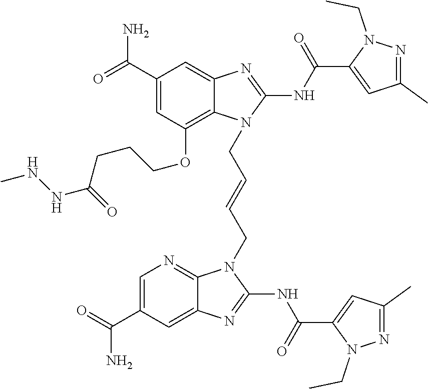
or a solvate, pharmaceutically acceptable salt, or tautomer thereof.

34. The compound of claim 1 wherein the compound is 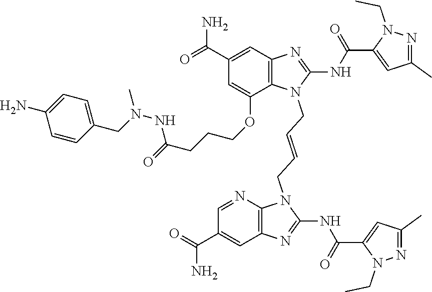 [SMILES: CCn1nc(C)cc1C(=O)Nc1nc2cc(C(N)=O)cnc2n1C/C=C/Cn1c(NC(=O)c2cc(C)nn2CC)nc2cc(C(N)=O)cc(OCCCC(=O)NN(C)Cc3ccc(N)cc3)c21]
or a solvate, pharmaceutically acceptable salt, or tautomer thereof.

35. The compound of claim 1 wherein the compound is 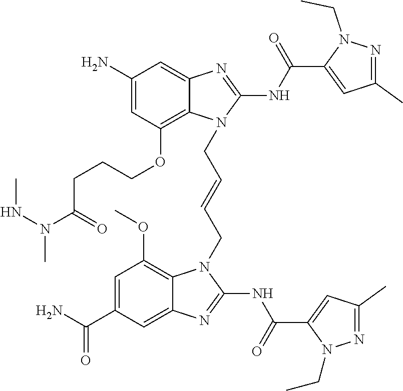 [SMILES: CCn1nc(C)cc1C(=O)Nc1nc2cc(C(N)=O)cc(OC)c2n1C/C=C/Cn1c(NC(=O)c2cc(C)nn2CC)nc2cc(N)cc(OCCCC(=O)N(C)NC)c21]
or a solvate, pharmaceutically acceptable salt, or tautomer thereof.

36. The compound of claim 1 wherein the compound is 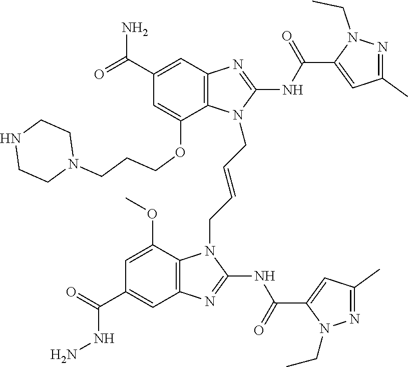 [SMILES: CCn1nc(C)cc1C(=O)Nc1nc2cc(C(=O)NN)cc(OC)c2n1C/C=C/Cn1c(NC(=O)c2cc(C)nn2CC)nc2cc(C(N)=O)cc(OCCCN3CCNCC3)c21]
or a solvate, pharmaceutically acceptable salt, or tautomer thereof.

37. The compound of claim 1 wherein the compound is 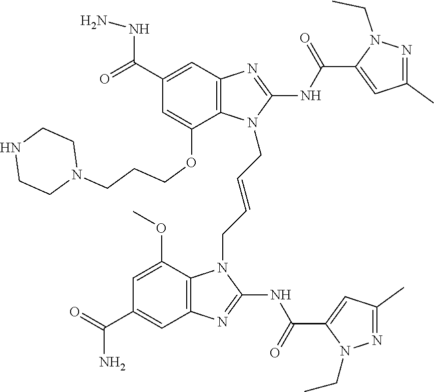 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NN)cc(OCCCN3CCNCC3)c21]
or a solvate, pharmaceutically acceptable salt, or tautomer thereof.

38. The compound of claim 1 wherein the compound is 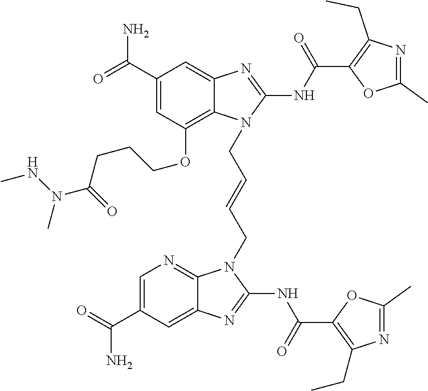
or a solvate, pharmaceutically acceptable salt, or tautomer thereof.

39. The compound of claim 1 wherein the compound is 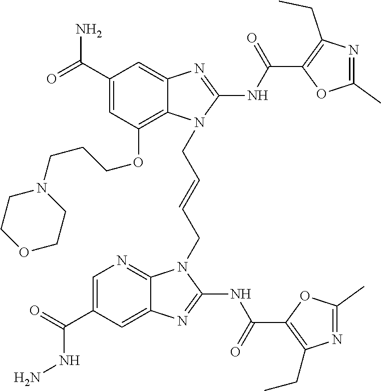 [SMILES: CCc1nc(C)oc1C(=O)Nc1nc2cc(C(=O)NN)cnc2n1C/C=C/Cn1c(NC(=O)c2oc(C)nc2CC)nc2cc(C(N)=O)cc(OCCCN3CCOCC3)c21]
or a solvate, pharmaceutically acceptable salt, or tautomer thereof.

40. The compound of claim 1 wherein the compound is 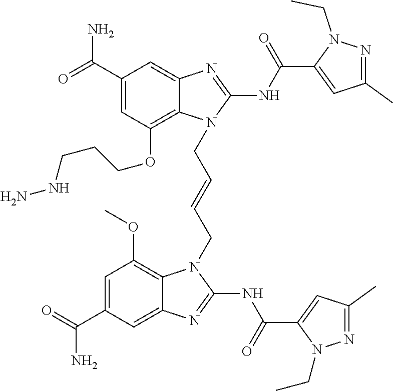 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCNN)c21]
or a solvate, pharmaceutically acceptable salt, or tautomer thereof.

41. The compound of claim 1 wherein the compound is 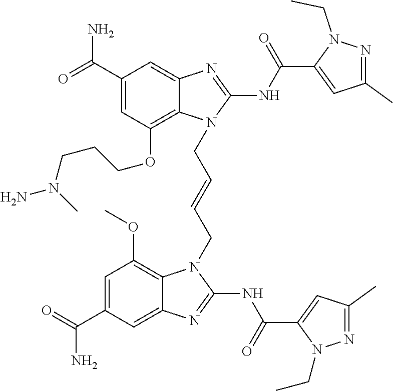 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(C)N)c21]
or a solvate, pharmaceutically acceptable salt, or tautomer thereof.

42. The compound of claim 1 wherein the compound is 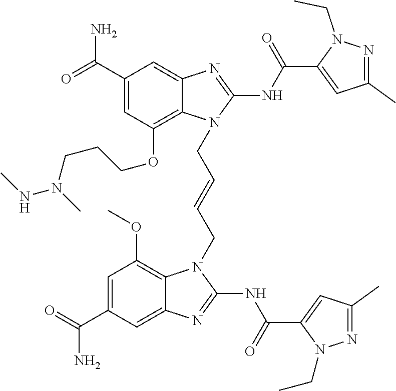
or a solvate, pharmaceutically acceptable salt, or tautomer thereof.

43. The compound of claim 1 wherein the compound is 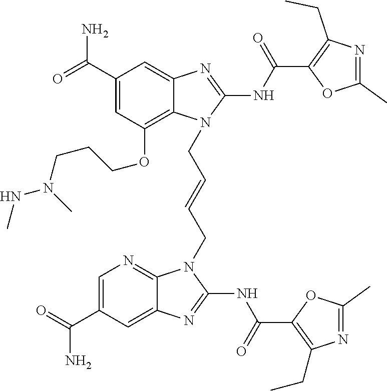
or a solvate, pharmaceutically acceptable salt, or tautomer thereof.

44. The compound of claim 1 wherein the compound is 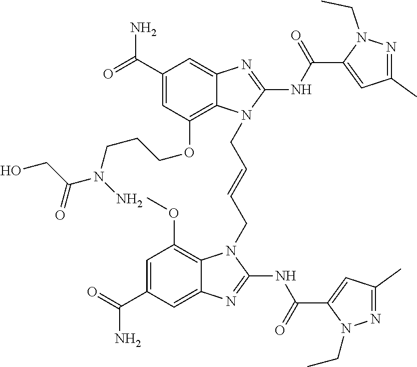 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCN(N)C(=O)CO)c21]
or a solvate, pharmaceutically acceptable salt, or tautomer thereof.

45. The compound of claim 1 wherein the compound is 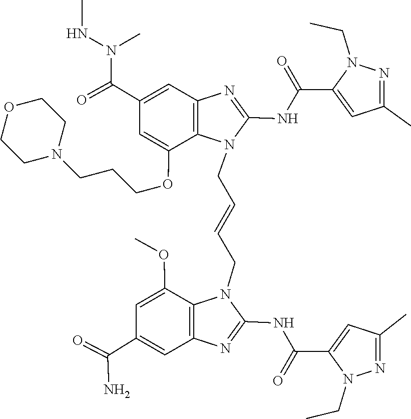 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)N(C)NC)cc(OCCCN3CCOCC3)c21]
or a solvate, pharmaceutically acceptable salt, or tautomer thereof.

46. The compound of claim 1 wherein the compound is 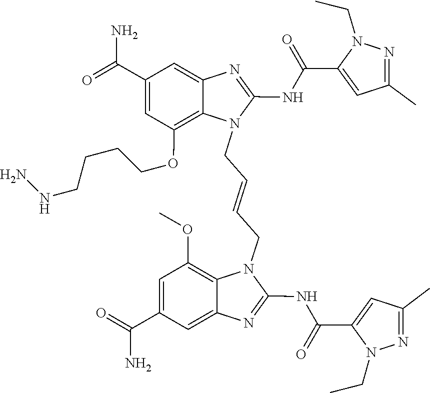 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCCNN)c21]
or a solvate, pharmaceutically acceptable salt, or tautomer thereof.

47. The compound of claim 1 wherein the compound is 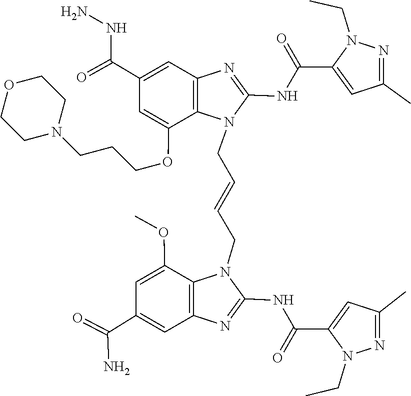 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(=O)NN)cc(OCCCN3CCOCC3)c21]
or a solvate, pharmaceutically acceptable salt, or tautomer thereof.

48. The compound of claim 1 wherein the compound is 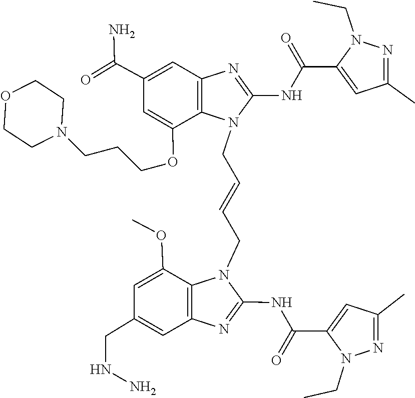 [SMILES: CCn1nc(C)cc1C(=O)Nc1nc2cc(CNN)cc(OC)c2n1C/C=C/Cn1c(NC(=O)c2cc(C)nn2CC)nc2cc(C(N)=O)cc(OCCCN3CCOCC3)c21]
or a solvate, pharmaceutically acceptable salt, or tautomer thereof.

49. The compound of claim 1 wherein the compound is 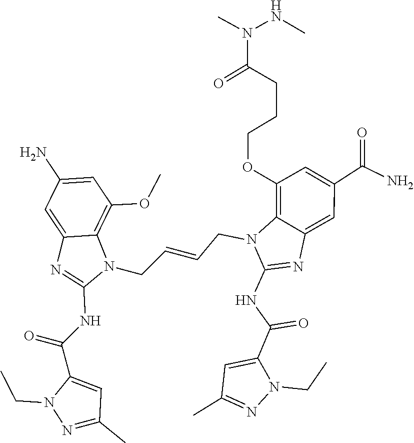 [SMILES: CCn1nc(C)cc1C(=O)Nc1nc2cc(N)cc(OC)c2n1C/C=C/Cn1c(NC(=O)c2cc(C)nn2CC)nc2cc(C(N)=O)cc(OCCCC(=O)N(C)NC)c21]
or a solvate, pharmaceutically acceptable salt, or tautomer thereof.

50. The compound of claim 1 wherein the compound is 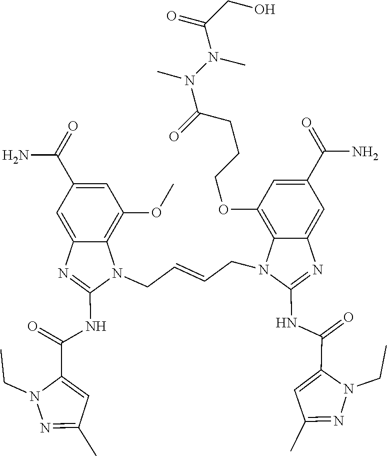 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N(C)N(C)C(=O)CO)c21]
or a solvate, pharmaceutically acceptable salt, or tautomer thereof.

51. The compound of claim 1 wherein the compound is 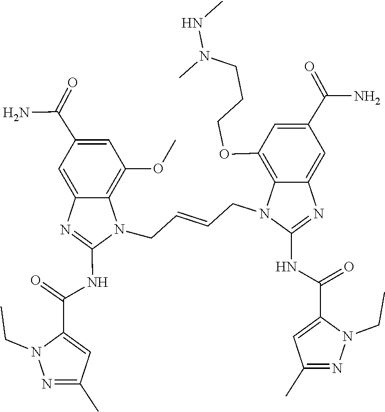
or a solvate, pharmaceutically acceptable salt, or tautomer thereof.

52. The compound of claim 1 wherein the compound is 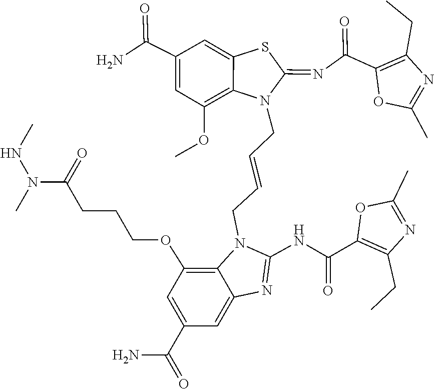 [SMILES: CCc1nc(C)oc1C(=O)/N=c1\sc2cc(C(N)=O)cc(OC)c2n1C/C=C/Cn1c(NC(=O)c2oc(C)nc2CC)nc2cc(C(N)=O)cc(OCCCC(=O)N(C)NC)c21]
or a solvate, pharmaceutically acceptable salt, or tautomer thereof.

53. The compound of claim 1 wherein the compound is 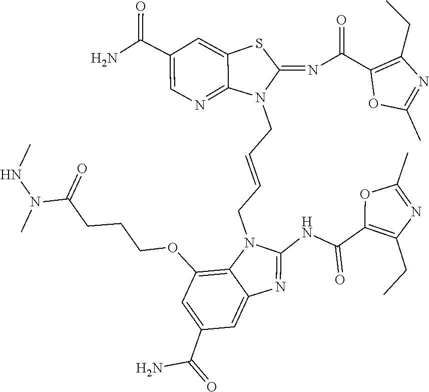 [SMILES: CCc1nc(C)oc1C(=O)/N=c1\sc2cc(C(N)=O)cnc2n1C/C=C/Cn1c(NC(=O)c2oc(C)nc2CC)nc2cc(C(N)=O)cc(OCCCC(=O)N(C)NC)c21]
or a solvate, pharmaceutically acceptable salt, or tautomer thereof.

54. The compound of claim 1 wherein the compound is 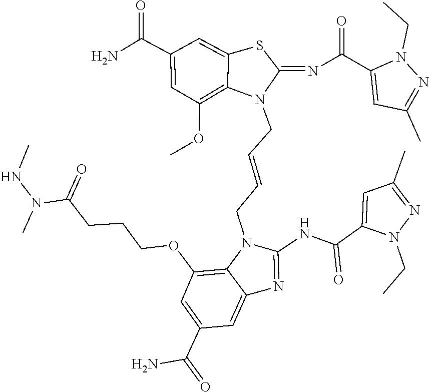 [SMILES: CCn1nc(C)cc1C(=O)/N=c1\sc2cc(C(N)=O)cc(OC)c2n1C/C=C/Cn1c(NC(=O)c2cc(C)nn2CC)nc2cc(C(N)=O)cc(OCCCC(=O)N(C)NC)c21]
or a solvate, pharmaceutically acceptable salt, or tautomer thereof.

55. The compound of claim 1 wherein the compound is 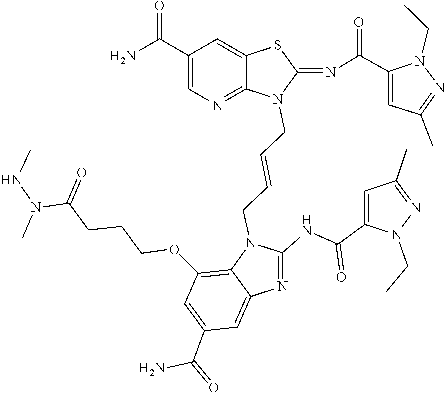 [SMILES: CCn1nc(C)cc1C(=O)/N=c1\sc2cc(C(N)=O)cnc2n1C/C=C/Cn1c(NC(=O)c2cc(C)nn2CC)nc2cc(C(N)=O)cc(OCCCC(=O)N(C)NC)c21]
or a solvate, pharmaceutically acceptable salt, or tautomer thereof.

56. The compound of claim 1 wherein the compound is 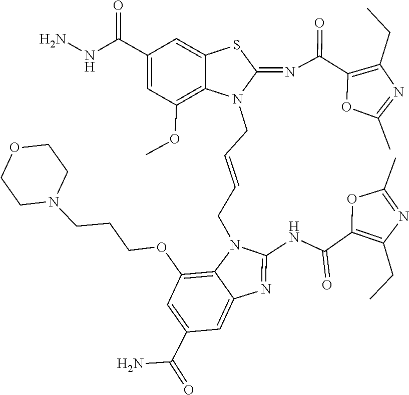 [SMILES: CCc1nc(C)oc1C(=O)/N=c1\sc2cc(C(=O)NN)cc(OC)c2n1C/C=C/Cn1c(NC(=O)c2oc(C)nc2CC)nc2cc(C(N)=O)cc(OCCCN3CCOCC3)c21]
or a solvate, pharmaceutically acceptable salt, or tautomer thereof.

57. The compound of claim 1 wherein the compound is 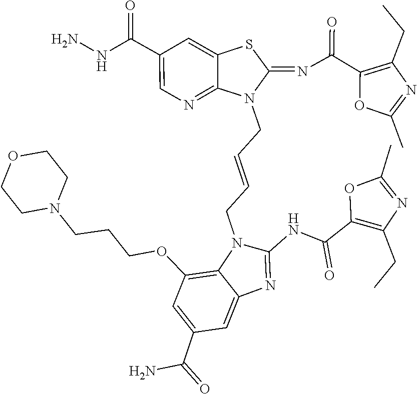 [SMILES: CCc1nc(C)oc1C(=O)/N=c1\sc2cc(C(=O)NN)cnc2n1C/C=C/Cn1c(NC(=O)c2oc(C)nc2CC)nc2cc(C(N)=O)cc(OCCCN3CCOCC3)c21]
or a solvate, pharmaceutically acceptable salt, or tautomer thereof.

58. The compound of claim 1 wherein the compound is 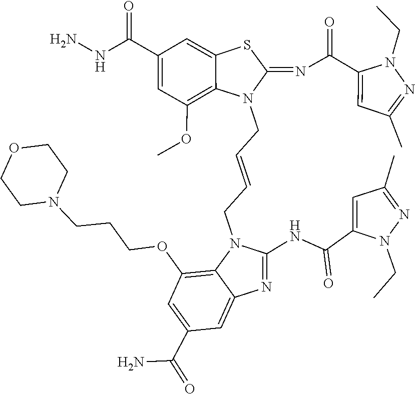 [SMILES: CCn1nc(C)cc1C(=O)/N=c1\sc2cc(C(=O)NN)cc(OC)c2n1C/C=C/Cn1c(NC(=O)c2cc(C)nn2CC)nc2cc(C(N)=O)cc(OCCCN3CCOCC3)c21]
or a solvate, pharmaceutically acceptable salt, or tautomer thereof.

59. The compound of claim 1 wherein the compound is 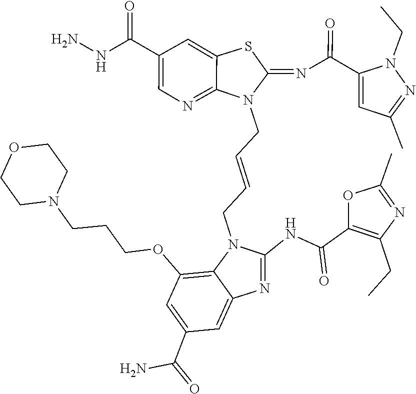 [SMILES: CCc1nc(C)oc1C(=O)Nc1nc2cc(C(N)=O)cc(OCCCN3CCOCC3)c2n1C/C=C/Cn1/c(=N/C(=O)c2cc(C)nn2CC)sc2cc(C(=O)NN)cnc21]
or a solvate, pharmaceutically acceptable salt, or tautomer thereof.

60. The compound of claim 1 wherein the compound is 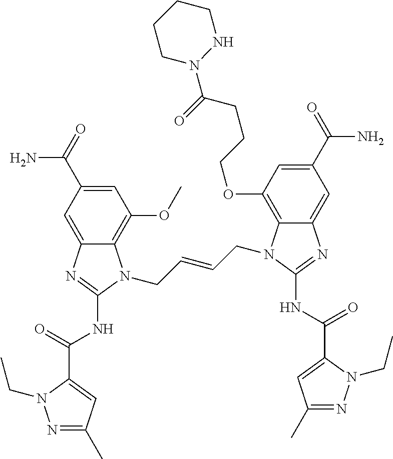 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N3CCCCN3)c21]
or a solvate, pharmaceutically acceptable salt, or tautomer thereof.

61. The compound of claim 1 wherein the compound is 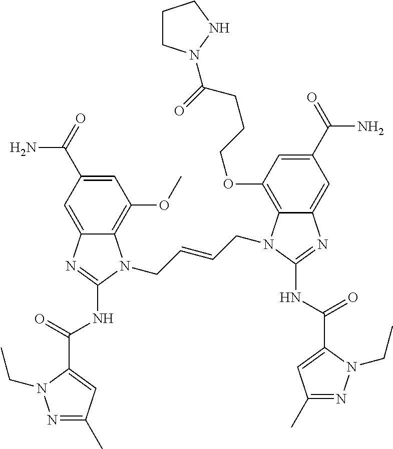 [SMILES: CCn1nc(C)cc1C(=O)Nc1nc2cc(C(N)=O)cc(OC)c2n1C/C=C/Cn1c(NC(=O)c2cc(C)nn2CC)nc2cc(C(N)=O)cc(OCCCC(=O)N3CCCN3)c21]
or a solvate, pharmaceutically acceptable salt, or tautomer thereof.

62. The compound of claim 1 wherein the compound is 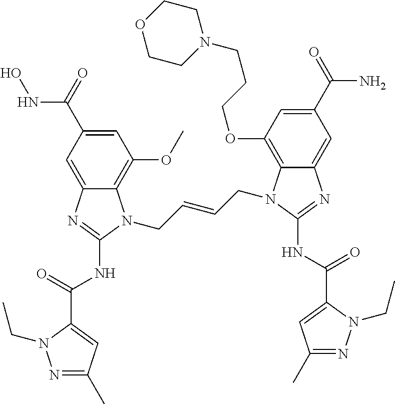 [SMILES: CCn1nc(C)cc1C(=O)Nc1nc2cc(C(=O)NO)cc(OC)c2n1C/C=C/Cn1c(NC(=O)c2cc(C)nn2CC)nc2cc(C(N)=O)cc(OCCCN3CCOCC3)c21]
or a solvate, pharmaceutically acceptable salt, or tautomer thereof.

63. The compound of claim 1 wherein the compound is

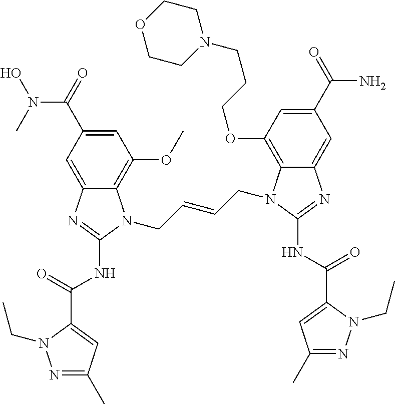 [SMILES: CCn1nc(C)cc1C(=O)Nc1nc2cc(C(=O)N(C)O)cc(OC)c2n1C/C=C/Cn1c(NC(=O)c2cc(C)nn2CC)nc2cc(C(N)=O)cc(OCCCN3CCOCC3)c21]

64. The compound of claim 1 wherein at least one of R₅, R₆, and R₇ is hydrogen.

65. The compound of claim 1 wherein both R₁ and R₃ are independently

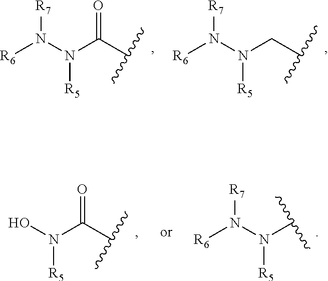

66. The compound of claim 6 wherein both R₁ and R₃ are independently

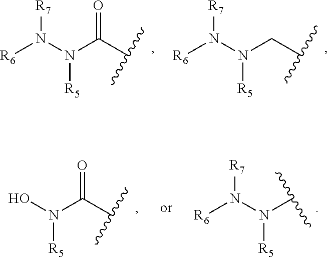

67. The compound of claim 13 wherein both R₁ and R₃ are independently

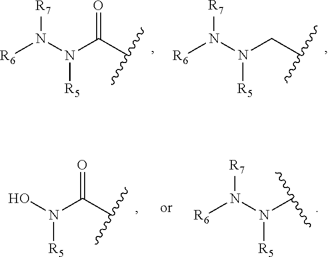

68. The compound of claim 1 wherein both R₁ and R₃ are independently

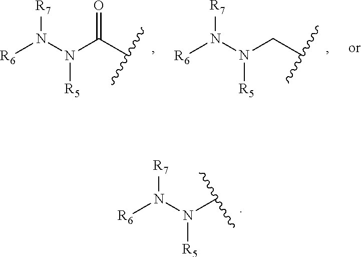

69. The compound of claim 6 wherein both R₁ and R₃ are independently

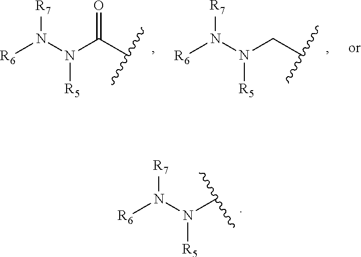

70. The compound of claim 13 wherein both R₁ and R₅ are independently

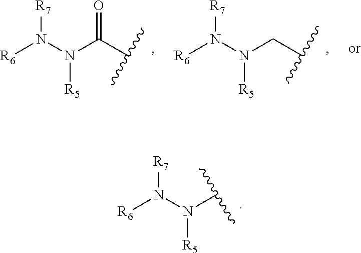

71. A method of treating a disease, disorder, or condition treatable by activation of STING, or mediated by STING protein activation comprising administering a therapeutically effective amount of one or more compounds of any one of claims 1 to 70.

72. The method of claim 71 wherein the disease, disorder, or condition is cancer.

73. The compound of claim 10 wherein G is N, X₁ is O, R₁ and R₃ are each 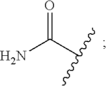 [SMILES: *C(N)=O]
R₂ is 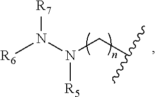
wherein n is 2, 3, or 4; R₅, R₆, and R₇ are each independently selected from H, C₁-C₆ alkyl, and 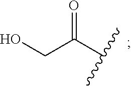 [SMILES: *C(=O)CO]
and Y₁ and Y₂ are each 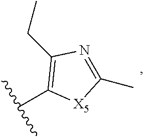
wherein X₅ is O.

74. The compound of claim 73 wherein the compound is 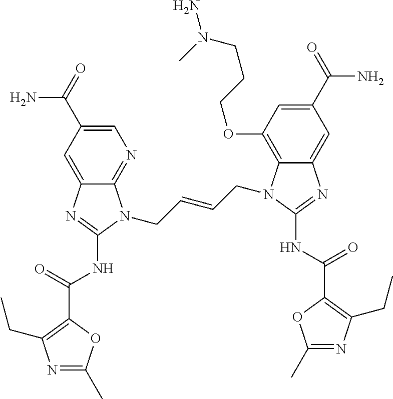 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(C)N)c21]
or a solvate, pharmaceutically acceptable salt, or tautomer thereof.

75. The compound of claim 73 selected from the group consisting of 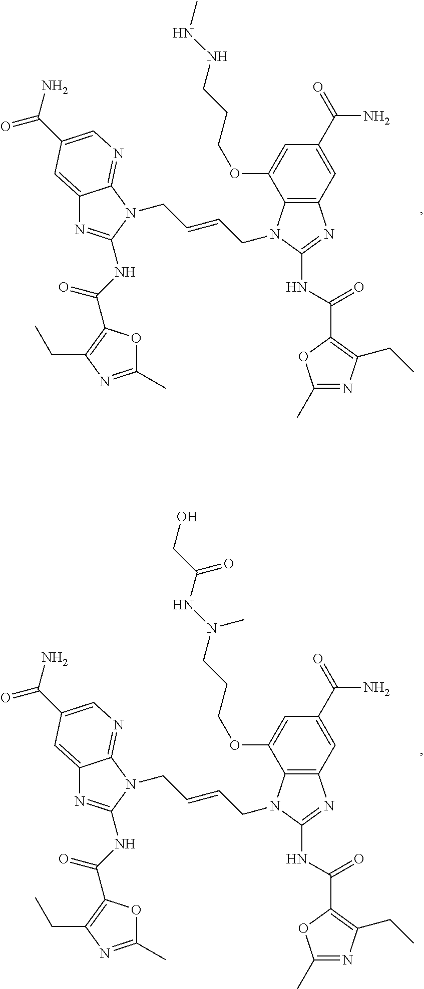 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCNNC)c21 ; CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(C)NC(=O)CO)c21] 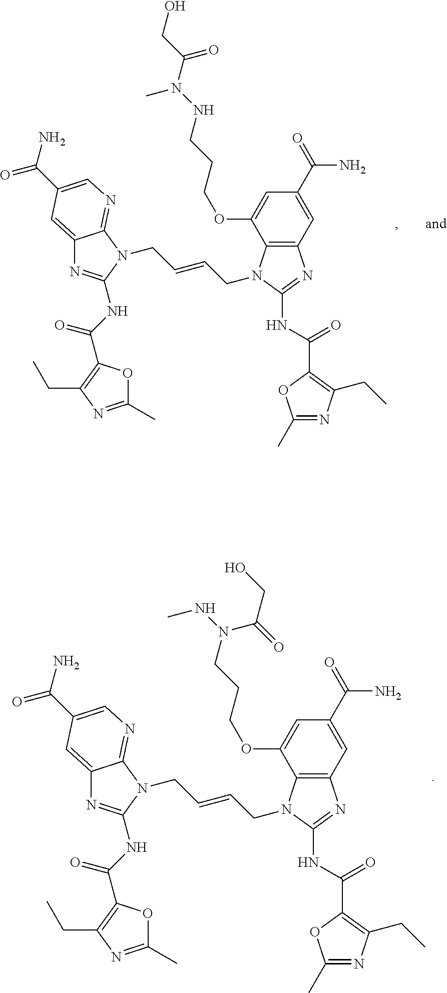 [SMILES: CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCNN(C)C(=O)CO)c21 ; CCc1nc(C)oc1C(=O)Nc1nc2cc(C(N)=O)cnc2n1C/C=C/Cn1c(NC(=O)c2oc(C)nc2CC)nc2cc(C(N)=O)cc(OCCCN(NC)C(=O)CO)c21]
